(19) United States
(12) Patent Application Publication
(10) Pub. No.: US 2016/0303110 A1
(43) Pub. Date: Oct. 20, 2016

(54) METHODS OF TREATING ABNORMAL MUSCULAR ACTIVITY

(71) Applicant: AUSPEX PHARMACEUTICALS, INC., La Jolla, CA (US)

(72) Inventor: Pratik SHAH, La Jolla, CA (US)

(73) Assignee: AUSPEX PHARMACEUTICALS, INC., La Jolla, CA (US)

(21) Appl. No.: 15/037,465

(22) PCT Filed: Nov. 21, 2014
(86) PCT No.: PCT/US14/66740

§ 371 Date: May 18, 2016

Related U.S. Application Data
(60) Provisional application No. 61/907,675, filed on Nov. 22, 2013.

(51) Int. Cl. A61K 31/4745; C07B 59/00; A61K 31/13; A61K 31/551; A61K 45/06; A61K 31/198

(52) U.S. Cl. CPC A61K 31/4745; A61K 45/06; A61K 31/198; A61K 31/13; A61K 31/551; C07B 59/002; C07B 2200/05

(57) ABSTRACT

Methods for treating abnormal muscular activity are disclosed. The methods may be performed remotely and permit monitoring of a subject outside a healthcare provider's office.

This application claims the benefit of priority of U.S. provisional application No. 61/907,675, filed Nov. 22, 2013, the disclosure of which is hereby incorporated by reference as if written herein in its entirety.

### BACKGROUND

The present disclosure relates to methods for treating abnormal muscular activity, more specifically to methods for treating abnormal muscular activity associated with at least one of bradykinesia, dyskinesia, and hyperkinesia.

Movement disorders can be classified into two basic categories: those characterized by disordered or excessive movement (referred to as “hyperkinesia” or “dyskinesia”), and those that are characterized by slowness, or a lack of movement (referred to as “hypokinesia,” “bradykinesia,” or “akinesia”). An example of a “hyperkinetic” movement disorder is a tremor or a tic while Parkinson's disease can be classified as “hypokinetic,” because it is often characterized by slow, deliberate movements, or even freezing in place.

Movement disorders include ataxia, corticobasal degeneration, dyskinesias (paroxysmal), dystonia (general, segmental, focal) including blepharospasm, spasmodic torticollis (cervical dystonia), writer's cramp (limb dystonia), laryngeal dystonia (spasmodic dysphonia), and oromandibular dystonia, essential tremor, hereditary spastic paraplegia, Huntington's Disease, multiple system atrophy (Shy Drager Syndrome), myoclonus, Parkinson's Disease, progressive supranuclear palsy, restless legs syndrome, Rett Syndrome, spasticity due to stroke, cerebral palsy, multiple sclerosis, spinal cord or brain injury, Sydenham's Chorea, tardive dyskinesia/dystonia, tics, Tourette's Syndrome, and Wilson's Disease.

Although medications and therapy for these disorders is available, doctors must observe a patient (for example by studying patient gait) to diagnose their problems and prescribe appropriate therapy. Treatment in any particular patient is an iterative process involving trial and error. A patient may require many doctor visits to assess the effectiveness of a medication, and optimize its dosage. These observations require considerable time, take place in an artificial environment, and are subject to the visual judgment of the physician.

Thus, there remains a need for improved methods for the diagnosis and treatment of abnormal muscular activity.

### SUMMARY

Accordingly, the inventors herein disclose new methods for assessing and treating abnormal muscular activity. The methods may be performed remotely and permit monitoring of a subject at home and in the community for un-biased, real-time analysis of a muscular activity disorder.

Provided is method of treating abnormal muscular activity in a subject in need thereof comprising the steps of:
a. measuring muscular activity data in the subject with at least one accelerometer;
b. processing the measured muscular activity data to distinguish between normal muscular activity and abnormal muscular activity in the subject;
c. transmitting the processed muscular activity data to a remote access unit;
d. retrieving the processed muscular activity data from the remote access unit;
e. determining a level of abnormal muscular activity in the subject; and
f. treating the subject based upon the level of the subject's abnormal muscular activity as determined in step e.

### DETAILED DESCRIPTION

### Abbreviations and Definitions

To facilitate understanding of the disclosure, a number of terms and abbreviations as used herein are defined below as follows:

The singular forms “a,” “an,” and “the” may refer to plural articles unless specifically stated otherwise.

When ranges of values are disclosed, and the notation “from n1 . . . to n2” or “n1-n2” is used, where n1 and n2 are the numbers, then unless otherwise specified, this notation is intended to include the numbers themselves and the range between them. This range may be integral or continuous between and including the end values.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

As used herein, the term “abnormal” refers to an activity or feature that differs from a normal activity or feature.

As used herein, the term “abnormal muscular activity” refers to muscular activity that differs from the muscular activity in a healthy subject. The abnormal activity may be decreased or increased in comparison to normal activity. An increase in muscular activity can result in excessive abnormal movements, excessive normal movements, or a combination of both.

The term “accelerometer” is defined to include any electronics components that measure the three dimensional movement, including gyros and related products.

The term “processing” refers to gathering, manipulating, storing, retrieving, and classifying the measured data. These steps may be performed by a microprocessor that includes one or more processing elements that are adapted to perform the recited operations. Thus, a processor may comprise all or part of one or more integrated circuits, firmware code, and/or software code that receive electrical signals from various sources and generate appropriate responses. In some embodiments, all processing elements that comprise the processor are located together. In other embodiments, the elements of a processor may spread across multiple devices in multiple locations.

The term “remote access unit” refers to a unit having a remote connection to the muscular activity measurement device. The unit may perform any of the steps of manipulating, storing, retrieving, and classifying the measured data. It may also communicate with the measurement device wirelessly. The remote access unit may feature a user interface to display raw or processed measured data. An advantage of the invention is that the muscular activity may be measured in a patient's home setting, and the data objectively evaluated by a physician in their office.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease”, “syndrome”, and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial

The term “VMAT2” refers to vesicular monoamine transporter 2, an integral membrane protein that acts to transport monoamines—particularly neurotransmitters such as dopamine, norepinephrine, serotonin, and histamine—from cellular cytosol into synaptic vesicles.

The term “VMAT2-mediated disorder,” refers to a disorder that is characterized by abnormal VMAT2 activity. A VMAT2-mediated disorder may be completely or partially mediated by modulating VMAT2. In particular, a VMAT2-mediated disorder is one in which inhibition of VMAT2 results in some effect on the underlying disorder e.g., administration of a VMAT2 inhibitor results in some improvement in at least some of the patients being treated.

The term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate

### Compositions

### Tetrabenazine and Metabolites

Tetrabenazine (Nitoman, Xenazine, Ro 1-9569), 1,3,4,6,7,11b-Hexahydro-9,10-dimethoxy-3-(2-methylpropyl)-2H-benzo[a]quinoline, is a vesicular monoamine transporter 2 (VMAT2) inhibitor. Tetrabenazine is commonly prescribed for the treatment of Huntington's disease (Savani et al., Neurology 2007, 68(10), 797; and Kenney et al., Expert Review of Neurotherapeutics 2006, 6(1), 7-17).

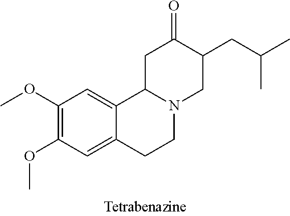 [SMILES: COc1cc2c(cc1OC)C1CC(=O)C(CC(C)C)CN1CC2]

In vivo, tetrabenazine is rapidly and extensively metabolized to its reduced form, dihydrotetrabenazine (CAS #3466-75-9), 1,3,4,6,7,11b-hexahydro-9,10-dimethoxy-3-(2-methylpropyl)-2H-benzo[a]quinolizin-2-ol. Dihydrotetrabenazine is a VMAT2 inhibitor and an active metabolite of tetrabenazine. Dihydrotetrabenazine is currently under investigation for the treatment of Huntington's disease, hemiballismus, senile chorea, tic disorders, tardive dyskinesia, dystonia, Tourette's syndrome, depression, cancer, rheumatoid arthritis, psychosis, multiple sclerosis, and asthma. WO 2005077946; WO 2007017643; WO 2007017654; WO 2009056885; WO 2010026434; and Zheng et al., The AAPS Journal, 2006, (8)4, E682-692.

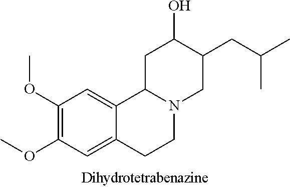 [SMILES: COc1cc2c(cc1OC)C1CC(O)C(CC(C)C)CN1CC2]

NBI-98854 (CAS #1025504-59-9), (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate, is a VMAT2 inhibitor. NBI-98854 is currently under investigation for the treatment of movement disorders including tardive dyskinesia. WO 2008058261; WO 2011153157; and U.S. Pat. No. 8,039,627. NBI-98854, a valine ester of (+)-α-dihydrotetrabenazine, in humans is slowly hydrolyzed to (+)-α-dihydrotetrabenazine which is an active metabolite of tetrabenazine.

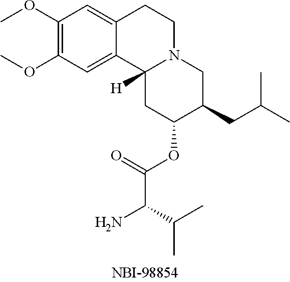 [SMILES: COc1cc2c(cc1OC)[C@H]1C[C@@H](OC(=O)[C@@H](N)C(C)C)[C@H](CC(C)C)CN1CC2]

A racemic mixture of [(3R,11bR)/(3S,11bS)]-3-(2-hydroxy-2-methyl-propyl)-9,10-di(methoxy-d₃)-1,3,4,6,7,11b-hexahydro-pyrido[2,1-a]isoquinolin-2-one (d₆-Tetrabenazine Metabolite M4-structures shown below)

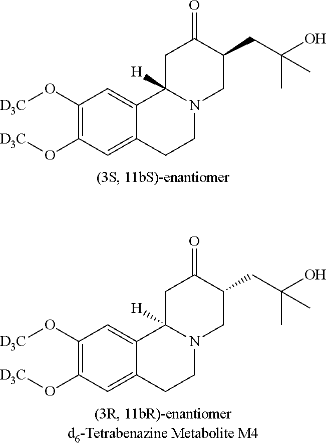 [SMILES: [2H]C([2H])([2H])Oc1cc2c(cc1OC([2H])([2H])[2H])[C@@H]1CC(=O)[C@@H](CC(C)(C)O)CN1CC2 ; [2H]C([2H])([2H])Oc1cc2c(cc1OC([2H])([2H])[2H])[C@H]1CC(=O)[C@H](CC(C)(C)O)CN1CC2]

and a diastereomeric mixture of 3-(2-Hydroxy-9,10-di(methoxy-d₃)-1,3,4,6,7,11b-hexahydro-2H-pyrido[2,1-a]isoquinolin-3-yl)-2-methyl-propionic acid (d₆-Tetrabenazine Metabolite M1-structures shown below)

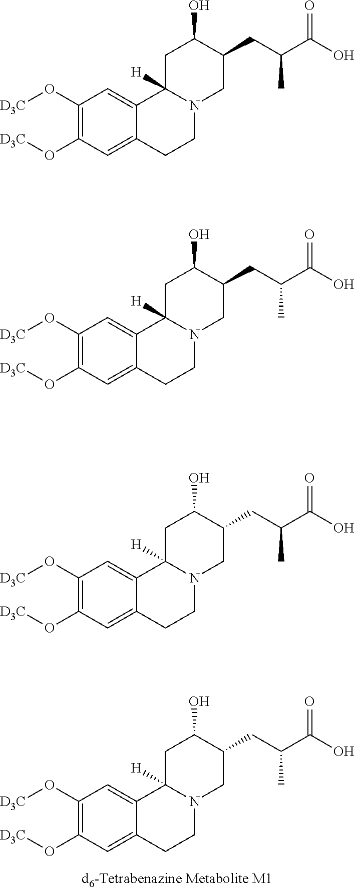 [SMILES: [2H]C([2H])([2H])Oc1cc2c(cc1OC([2H])([2H])[2H])[C@@H]1C[C@@H](O)[C@@H](C[C@H](C)C(=O)O)CN1CC2 ; [2H]C([2H])([2H])Oc1cc2c(cc1OC([2H])([2H])[2H])[C@@H]1C[C@@H](O)[C@@H](C[C@@H](C)C(=O)O)CN1CC2 ; [2H]C([2H])([2H])Oc1cc2c(cc1OC([2H])([2H])[2H])[C@H]1C[C@H](O)[C@H](C[C@H](C)C(=O)O)CN1CC2 ; [2H]C([2H])([2H])Oc1cc2c(cc1OC([2H])([2H])[2H])[C@H]1C[C@H](O)[C@H](C[C@@H](C)C(=O)O)CN1CC2]

are metabolites of d₆-tetrabenazine and/or d₆-dihydrotetrabenazine. d₆-Tetrabenazine and d₆-dihydrotetrabenazine, as well as the M1 and M4 metabolites, are VMAT2 inhibitors. d₆-Tetrabenazine and d₆-dihydrotetrabenazine are currently under investigation for the treatment of Huntington's disease and other VMAT2-mediated disorders. U.S. Pat. No. 8,524,733, US 20100130480, and US 20120003330.

Tetrabenazine, dihydrotetrabenazine, and NBI-98854 are subject to extensive oxidative metabolism, including 0-demethylation of the methoxy groups, as well as hydroxylation of the isobutyl group (Schwartz et al., Biochem. Pharmacol., 1966, 15, 645-655). Adverse effects associated with the administration of tetrabenazine, dihydrotetrabenazine, and/or NBI-98854 include neuroleptic malignant syndrome, drowsiness, fatigue, nervousness, anxiety, insomnia, agitation, confusion, orthostatic hypotension, nausea, dizziness, depression, and Parkinsonism.

Tetrabenazine, dyhydrotetrabenazine, and NBI-98854 are VMAT2 inhibitors. The carbon-hydrogen bonds of tetrabenazine, dyhydrotetrabenazine, and NBI-98854 contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of tetrabenazine, dyhydrotetrabenazine, and/or NBI-98854 in comparison with tetrabenazine, dyhydrotetrabenazine, and/or NBI-98854 having naturally occurring levels of deuterium.

Based on discoveries made in our laboratory, as well as considering the literature, tetrabenazine, dyhydrotetrabenazine, and/or NBI-98854 are metabolized in humans at the isobutyl and methoxy groups. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of tetrabenazine, dyhydrotetrabenazine, and/or NBI-98854 and attenuate interpatient variability.

### Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C)-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT} The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy Eact for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C-¹H bond. If a C-¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C-¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Tetrabenazine, dyhydrotetrabenazine, and NBI-98854 are VMAT2 inhibitors. The carbon-hydrogen bonds of tetrabenazine, dyhydrotetrabenazine, and NBI-98854 contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of tetrabenazine, dyhydrotetrabenazine, and/or NBI-98854 in comparison with tetrabenazine, dyhydrotetrabenazine, and/or NBI-98854 having naturally occurring levels of deuterium.

Based on discoveries made in our laboratory, as well as considering the literature, tetrabenazine, dyhydrotetrabenazine, and/or NBI-98854 are metabolized in humans at the isobutyl and methoxy groups. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of tetrabenazine, dyhydrotetrabenazine, and/or NBI-98854 and attenuate interpatient variability.

Novel compounds and pharmaceutical compositions, certain of which have been found to inhibit VMAT2 have been discovered, together with methods of synthesizing and using the compounds, including methods for the treatment of VMAT2-mediated disorders in a patient by administering the compounds as disclosed herein.

### Deuterium Enriched Tetrabenazine Analogues

In certain embodiments of the present invention, compounds have structural Formula I:

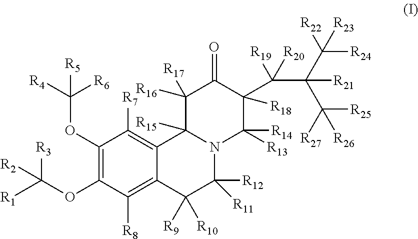

or a salt, solvate, or prodrug thereof, wherein:
R₁-R₂₇ are independently selected from the group consisting of hydrogen and deuterium; and
at least one of R₁-R₂₇ is deuterium.

In certain embodiments, Formula I can include a single enantiomer, a mixture of the (+)-enantiomer and the (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof.

In certain embodiments of the present invention, compounds have structural Formula II:

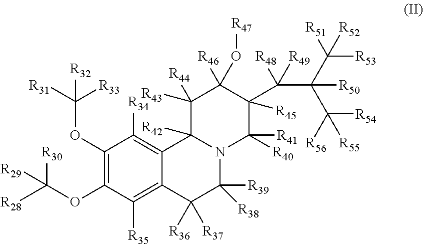

or a salt thereof, wherein:
R₂₈-R₄₆ and R₄₈-R₅₆ are independently selected from the group consisting of hydrogen and deuterium;
R₄₇ is selected from the group consisting of hydrogen, deuterium, —C(O)O-alkyl and —C(O)—C₁₋₆ alkyl, or a group cleavable under physiological conditions, wherein said alkyl or C₁₋₆ alkyl is optionally substituted with one or more substituents selected from the group consisting of —NH—C(NH)NH2, —CO₂H,—CO₂alkyl, —SH, —C(O)NH₂, —NH₂, phenyl, —OH, 4-hydroxyphenyl, imidazolyl, and indolyl, and any R46 substituent is further optionally substituted with deuterium; and
at least one of R₂₈-R₅₆ is deuterium or contains deuterium.

In certain embodiments, the compounds of Formula II have alpha stereochemistry.

In further embodiments, the compounds of Formula II have beta stereochemistry.

In yet further embodiments, the compounds of Formula II are a mixture of alpha and beta stereoisomers. In yet further embodiments, the ratio of alpha/beta stereoisomers is at least 100:1, at least 50:1, at least 20:1, at least 10:1, at least 5:1, at least 4:1, at least 3:1, or at least 2:1. In yet further embodiments, the ratio of beta/alpha stereoisomers is at least 100:1, at least 50:1, at least 20:1, at least 10:1, at least 5:1, at least 4:1, at least 3:1, or at least 2:1.

In certain embodiments, if R₅₀-R₅₆ are deuterium, at least one of R₁-R₄₉ is deuterium.

In certain embodiments of the present invention, compounds have structural Formula III:

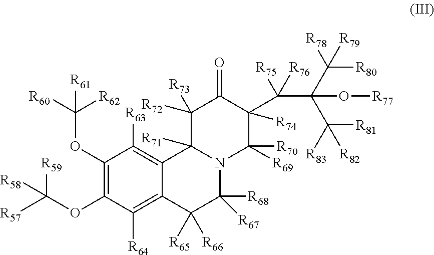

or a salt, stereoisomer, or racemic mixture thereof, wherein:
R₅₇-R₈₃ are independently selected from the group consisting of hydrogen and deuterium; and
at least one of R₅₇-R₈₃ is deuterium.

In certain embodiments of the present invention, compounds have structural Formula IV:

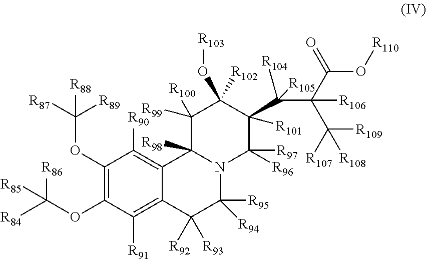

or a salt, diastereomer, or mixture of diastereomers thereof, wherein:
R₈₄-R₁₁₀ are independently selected from the group consisting of hydrogen and deuterium; and
at least one of R₈₄-R₁₁₀ is deuterium.

Certain compounds disclosed herein may possess useful VMAT2 inhibiting activity, and may be used in the treatment or prophylaxis of a disorder in which VMAT2 plays an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for inhibiting VMAT2. Other embodiments provide methods for treating a VMAT2-mediated disorder in a patient in need of such treatment, comprising administering to said patient a therapeutically effective amount of a compound or composition according to the present invention. Also provided is the use of certain compounds disclosed herein for use in the manufacture of a medicament for the prevention or treatment of a disorder ameliorated by the inhibition of VMAT2.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₁₁₀ or the symbol “D”, when used to represent a given position in a drawing of a molecular structure, means that the specified position is enriched with deuterium above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as D-isomers and L-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The terms “alpha-dihydrotetrabenazine”, “α-dihydrotetrabenazine”, or the terms “alpha” or “alpha stereoisomer” or the symbol “a” as applied to dihydrotetrabenazine refers to either of the dihydrotetrabenazine stereoisomers having the structural formulas shown below, or a mixture thereof:

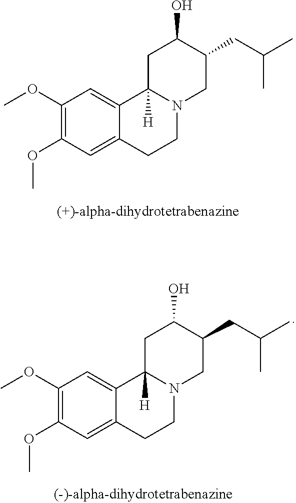

The terms “alpha” or “alpha stereoisomer” or the symbol “a” as applied to a compound of Formula II refers to either of the stereoisomers of compounds of Formula II shown below, or a mixture thereof:

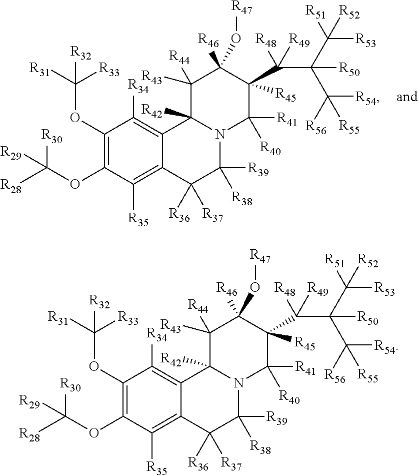

The terms “beta-dihydrotetrabenazine”, “β-dihydrotetrabenazine”, or the terms “beta” or “beta stereoisomer” or the symbol “β” as applied to dihydrotetrabenazine refers to either of the dihydrotetrabenazine stereoisomers having the structural formulas shown below, or a mixture thereof:

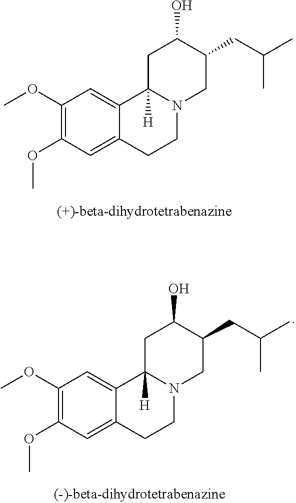

The terms “beta” or “beta stereoisomer” or the symbol “β” as applied to a compound of Formula II refers to either of the stereoisomers of compounds of Formula II shown below, or a mixture thereof:

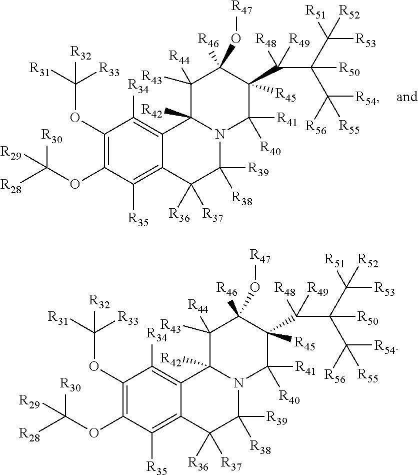

The terms “3S,11bS enantiomer” or the term “3R,11bR enantiomer” refers to either of the d₆-tetrabenazine M4 metabolite stereoisomers having the structural formulas shown below:

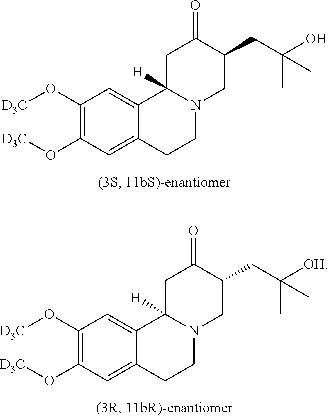 [SMILES: [2H]C([2H])([2H])Oc1cc2c(cc1OC([2H])([2H])[2H])[C@@H]1CC(=O)[C@@H](CC(C)(C)O)CN1CC2 ; [2H]C([2H])([2H])Oc1cc2c(cc1OC([2H])([2H])[2H])[C@H]1CC(=O)[C@H](CC(C)(C)O)CN1CC2]

In certain embodiments, a chemical structure may be drawn as either the 3S,11bS enantiomer or the 3R,11bR enantiomer, but the text of the specification may indicate that the 3S,11bS enantiomer, the 3R,11bR enantiomer, a racemic mixture thereof, or all of the foregoing may be intended to be described.

The terms “(3S,11bS)-enantiomer” or “(3R,11bR)-enantiomer” or the as applied to a compound of Formula I refers to either of the stereoisomers of compounds of Formula III shown below:

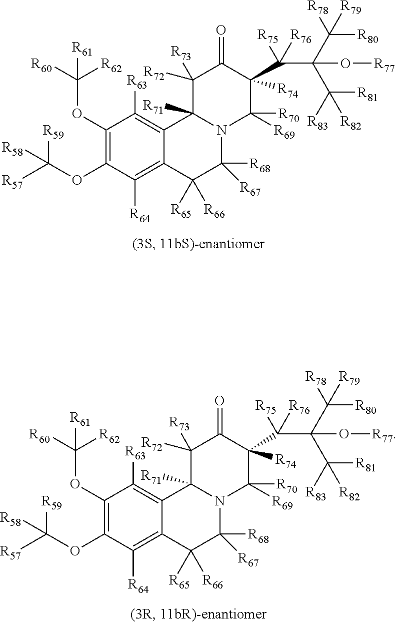

The term “mixture of diastereomers” refers to either of the d₆-tetrabenazine M1 metabolite stereoisomers having the structural formulas shown below:

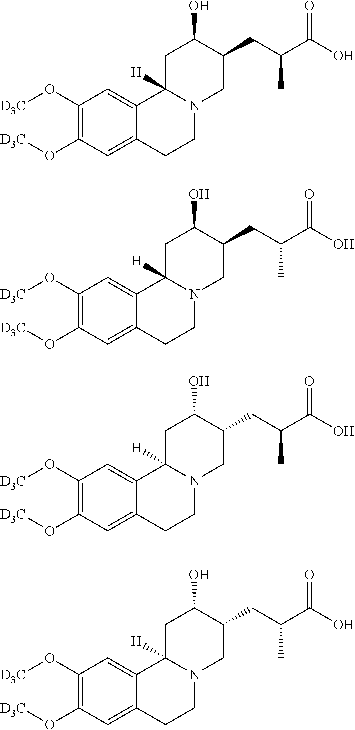 [SMILES: [2H]C([2H])([2H])Oc1cc2c(cc1OC([2H])([2H])[2H])[C@@H]1C[C@@H](O)[C@@H](C[C@H](C)C(=O)O)CN1CC2 ; [2H]C([2H])([2H])Oc1cc2c(cc1OC([2H])([2H])[2H])[C@@H]1C[C@@H](O)[C@@H](C[C@@H](C)C(=O)O)CN1CC2 ; [2H]C([2H])([2H])Oc1cc2c(cc1OC([2H])([2H])[2H])[C@H]1C[C@H](O)[C@H](C[C@H](C)C(=O)O)CN1CC2 ; [2H]C([2H])([2H])Oc1cc2c(cc1OC([2H])([2H])[2H])[C@H]1C[C@H](O)[C@H](C[C@@H](C)C(=O)O)CN1CC2]

In certain embodiments, a chemical structure may be drawn as one of the diastereomers shown above, but the text of the specification may indicate that each individual diastereomer or a mixture thereof, or all of the foregoing may be intended to be described.

The term “mixture of diastereomers” as applied to a compound of Formula IV refers to a mixture of the stereoisomers of compounds of Formula IV shown below:

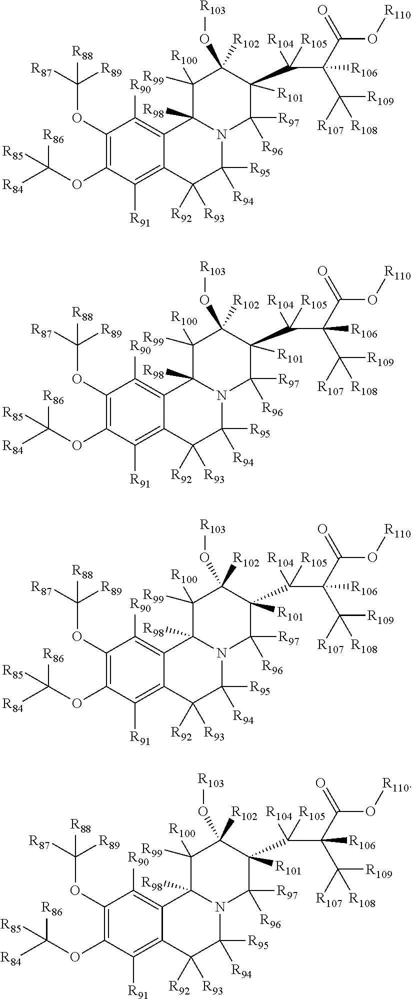

### Methods

Thus, in various embodiments, the present invention provides a method of treating abnormal muscular activity in a subject in need thereof comprising the steps of:
measuring muscular activity data in the subject with at least one accelerometer;
processing the measured muscular activity data to distinguish between normal muscular activity and abnormal muscular activity in the subject;
transmitting the processed muscular activity data to a remote access unit;
retrieving the processed muscular activity data from the remote access unit;
determining a level of abnormal muscular activity in the subject; and
treating the subject based upon the level of the subject's abnormal muscular activity as determined in above.

In some embodiments, at least one accelerometer is used to detect muscular activity. Accelerometers are well known in the art. An accelerometer may sense changes in velocity directly through interrogation or receipt of signal from an inertial transducer. An accelerometer may also calculate changes in velocity from data received from position sensing transducers. Accelerometers are often electromechanical devices and can measure the static gravitational force or dynamic forces caused by changes in speed and/or direction (changes in velocity). Accelerometers can utilize the piezoelectric effect and can detect acceleration in three orthogonal axis, as well as rotation about the axis. Accelerometers have been utilized in medical devices as well—see for example issued U.S. Pat. No. 5,233,984 and U.S. 5,593,431.

Multiple accelerometers may detect activity or motion at separate locations on a subject. For example, as a subject moves, an accelerometer located on the torso of a subject detects the motion of the torso, and an accelerometer located on the head detects the motion of the head of subject. In the case in which accelerometers comprise multi-axis accelerometers, the accelerometers detect the motion of the head and torso in terms of magnitude and direction. The accelerometers may generate signals as a function of the detected motion, and a processor may compare the motion of the head relative to the torso. The accelerometers may be located elsewhere on a subject, such as a limb.

As mentioned above, using relative motion provides a different frame of reference. More specifically, instead of the frame of reference being no motion (i.e., stillness), the frame of reference is another accelerometer. This new frame of reference from the perspective of another accelerometer allows the processor to ignore motions that are experienced by all portions of the subject, thus making it easier to detect, for example, motions that represent conditions of abnormal muscular activity. In other words, using the new frame of reference provided by analyzing the relative motion between two or more accelerometers, the processor may ignore motion that is experienced by both the accelerometers. For the example, if a subject experiences a bumpy plane ride, both of the activity sensors experience the motion due to the turbulence. When compared to one another (e.g., subtracted) these detected motions may be substantially eliminated, leaving only the motions of the accelerometers that are different, such as the motions caused by a tremor or a seizure. In this manner, the new frame of reference provided by analyzing relative motion allows for more accurate detection of movement disorders.

A processor may process the measured muscular activity data to distinguish between normal muscular activity and abnormal muscular activity in the subject. The processor may compare the magnitudes of the signals generated by the two or more accelerometers, the directionality of the signals generated by the two or more accelerometers, the frequency of signals generated by accelerometers or a combination thereof to calculate the relative motion. The processor may then analyze the relative motion to detect a condition of a movement disorder. For example, a processor may analyze a plurality of relative motion measurements computed over a window of time, e.g., over 15-20 relative motion measurements. The processor may detect abnormal muscular activity, such as a tremor or seizure, when the magnitude, frequency and/or the directionality of the relative motion measurements exceed a threshold for a consecutive number of measurements. For instance, the processor may detect a condition of the movement disorder when relative motion is detected between the two sensors for a threshold number of times over a period.

Alternatively, the processor may compare the relative motion over a window of time to one or more pre-defined patterns, and detect a condition of abnormal muscular activity when the relative motion measurements over the window of time substantially match one of the pre-defined patterns. The processor may determine the pre-defined patterns based on relative motion measurements computed during previous episodes, e.g., previous tremors or seizures, of a subject. Pre-defined patterns may also be determined based on sensor signals obtained from a population of subjects and/or clinical subjects during symptomatic movement episodes, e.g., tremors or seizures. In some embodiments, the processor may employ or include a neural network for identifying symptomatic movement. The neural network may be trained based on prior patient episodes and/or episodes gathered from other patients/subjects.

Alternatively, the processor may compute the pre-defined patterns based on a basic body model. The body model may, for example, represent information regarding a subject (e.g., height, weight and age), the position of accelerometers within the subject, or the like. The processor may, for example, receive the body model information from a physician or subject via one of programmers e.g., during initial configuration of the device. Alternatively, programmers may compute look-up tables based on the body model. The processor may use the body model information or other information generated from the body model to compute relative motions between two or more accelerometers or to analyze the relative motion to identify abnormal muscular activity. For example, the processor may use a variety of algorithms based on kinesiology and the biomechanics of the human body to predict relative motion measurements that are indicative of a condition of a muscular disorder for the particular subject. Such computations may account for other variables such as age, weight and height of patient.

As described above, a remote access device may receive the signal generated by accelerometers and compare the signals to compute the relative motion between the accelerometers. Additionally, the remote access device may analyze the relative motion using the techniques described above to determine whether the relative motion is indicative of a symptom of the abnormal muscular activity.

The magnitude, duration, and frequency of the abnormal muscular activity may be determined using the methods described above. The present method also anticipates methods that determine whether abnormal muscular activity occurs at all or occurs above a threshold (e.g., a control threshold). Thus, the method may provide a “yes or no” result without necessarily providing quantification of abnormal muscular activity is within the scope of the present disclosure. The method may involve quantitative or qualitative assessment of abnormal muscular activity.

If a subject is determined to have a high level of abnormal muscular activity, the subject may be treated to reduce the level of abnormal muscular activity. Treating the subject may include administering a therapeutically effective amount of a therapeutic agent to the subject. The dosage amount and frequency of the therapeutic agent may be adjusted based upon the magnitude, duration, and frequency of the determined level of abnormal muscular activity. The amounts and frequencies of dosage may be adjusted to minimize any side effects from the therapeutic agent. In certain aspects, this method may be used to monitor abnormal muscular activity associated with a therapy's unwanted side effect, and treatment adjusted based upon the level of the unwanted side effect.

In certain aspects, the abnormal muscular activity is associated with at least one of bradykinesia, dyskinesia, and hyperkinesia. In particular aspects, the abnormal muscular activity is associated with Huntington's disease.

In certain aspects, the abnormal muscular activity is associated with at least one of bradykinesia, dyskinesia, and hyperkinesia. In particular aspects, the abnormal muscular activity is associated with Huntington's disease.

In certain aspects, treating the subject may include administering a therapeutically effective amount of tetrabenazine or its metabolites. In particular aspects, treating the subject may include administering a therapeutically effective amount of a deuterium enriched tetrabenazine analogue as described herein.

### Formulation

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts. For a more complete discussion of the preparation and selection of salts, refer to “Handbook of Pharmaceutical Salts, Properties, and Use,” Stah and Wermuth, Ed., (Wiley-VCH and VHCA, Zurich, 2002) and Berge et al., J. Pharm. Sci. 1977, 66, 1-19.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject invention to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc., New York, N.Y., 2002; Vol. 126).

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject invention or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In certain embodiments, disclosed herein is an extended-release pharmaceutical formulation comprising, in a solid dosage form for oral delivery of between about 100 mg and about 1 g total weight:
between about 2 and about 18% of a compound as disclosed herein;
between about 70% and about 96% of one or more diluents;
between about 1% and about 10% of a water-soluble binder; and
between about 0.5 and about 2% of a surfactant.

In certain embodiments, the diluent or diluents are chosen from mannitol, lactose, and microcrystalline cellulose; the binder is a polyvinylpyrrolidone; and the surfactant is a polysorbate.

In certain embodiments, the extended-release pharmaceutical formulation comprises between about 2.5% and about 11% of a compound as disclosed herein.

In certain embodiments, the extended-release pharmaceutical formulation comprises: between about 60% and about 70% mannitol or lactose;
between about 15% and about 25% microcrystalline cellulose
about 5% of polyvinylpyrrolidone K29/32; and
between about 1 and about 2% of Tween 80.

In certain embodiments, the extended-release pharmaceutical formulation comprises:
between about 4% and about 9% of a compound as disclosed herein;
between about 60% and about 70% mannitol or lactose;
between about 20% and about 25% microcrystalline cellulose
about 5% of polyvinylpyrrolidone K29/32; and
about 1.4% of Tween 80.

In certain embodiments, disclosed herein is an extended-release pharmaceutical formulation comprising, in a solid dosage form for oral delivery of between about 100 mg and about 1 g total weight:
between about 70 and about 95% of a granulation of a compound as disclosed herein, wherein the active ingredient comprises between about 1 and about 15% of the granulation;
between about 5% and about 15% of one or more diluents;
between about 5% and about 20% of sustained-release polymer; and
between about 0.5 and about 2% of a lubricant.

In certain embodiments, the extended-release pharmaceutical formulation comprises:
between about 5% and about 15% of one or more spray-dried mannitol or spray-dried lactose;
between about 5% and about 20% of sustained-release polymer; and between about 0.5 and about 2% of a magnesium stearate.

In certain embodiments, the sustained-release polymer is chosen from a polyvinyl acetate-polyvinylpyrrolidone mixture and a poly(ethylene oxide) polymer.

In certain embodiments, the sustained-release polymer is chosen from Kollidon® SR, POLYOX® N60K, and Carbopol®.

In certain embodiments, the sustained-release polymer is Kollidon® SR.

In certain embodiments, the sustained-release polymer is POLYOX® N60K.

In certain embodiments, the sustained-release polymer is Carbopol®.

In certain embodiments, the extended-release pharmaceutical formulation comprises from about 5 mg to about 100 mg of a compound as disclosed herein.

In certain embodiments, the compounds disclosed herein can be formulated as extended-release pharmaceutical formulations as described in U.S. patent application Ser. No. 14/030,322, filed Sep. 18, 2013.

### Dosage

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period, including throughout the duration of the patient's life in order to ameliorate or otherwise control or limit the symptoms of the patient's disorder.

In the case wherein the patient's status does improve, upon the doctor's discretion the administration of the compounds may be given continuously or suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

### Administration

### Combination Therapy

The compounds disclosed herein may also be combined or used in combination with other agents useful in the treatment of VMAT2-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more dopamine precursors, DOPA decarboxylase inhibitors, catechol-O-methyl transferase (COMT) inhibitors, dopamine receptor agonists, neuroprotective agents, NMDA antagonists, and anti-psychotics.

In certain embodiments, the compounds disclosed herein can be combined with one or more dopamine precursors selected from the group consisting of levodopa and deuterated L-DOPA.

Deuterated L-DOPA derivatives are described in PCT Patent Application WO 2014122184, published on Aug. 14, 2014, which is hereby incorporated by reference as if written herein in its entirety.

In certain embodiments, said deuterated L-DOPA has the structural formula:

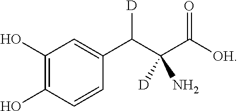 [SMILES: [2H]C(c1ccc(O)c(O)c1)[C@]([2H])(N)C(=O)O]

In certain embodiments, said deuterated L-DOPA has the structural formula:

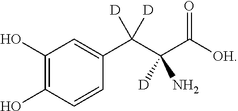 [SMILES: [2H]C([2H])(c1ccc(O)c(O)c1)[C@]([2H])(N)C(=O)O]

In certain embodiments, deuterated L-DOPA comprises a composition of compounds of structural formula V

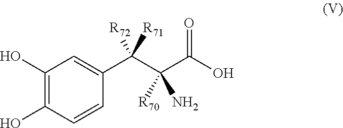

or a salt thereof, wherein:
in each compound of Formula V, R₇₀-R₇₂ are independently selected from the group consisting of hydrogen and deuterium;
the composition has deuterium enrichment of at least 10% at each of the positions R₇₀-R₇₂ in the compounds of Formula I;
the deuterium enrichment at the positions R₇₁ and R₇₂ is different from each other by at least 5%.

In certain embodiments, R₇₀ has deuterium enrichment of no less than 90%.

In certain embodiments, R₇₀ has deuterium enrichment of no less than 98%.

In certain embodiments, R₇₂ has deuterium enrichment of no less than 90%.

In certain embodiments, R₇₂ has deuterium enrichment of no less than 98%.

In certain embodiments, R₇₁ has deuterium enrichment of between about 78% and about 95%.

In certain embodiments, R₇₁ has deuterium enrichment of between about 78% and about 82%.

In certain embodiments, R₇₁ has deuterium enrichment of between about 88% and about 92%.

In certain embodiments, said DOPA decarboxylase inhibitor is carbidopa.

In certain embodiments, said catechol-O-methyl transferase (COMT) inhibitor is selected from the group consisting of entacapone and tolcapone.

In certain embodiments, said dopamine receptor agonist is selected from the group consisting of apomorphine, bromocriptine, ropinirole, and pramipexole.

In certain embodiments, said neuroprotective agent is selected from the group consisting of selegeline and riluzole.

In certain embodiments, said NMDA antagonist is amantidine.

In certain embodiments, said anti-psychotic is clozapine.

In certain embodiments, the compounds disclosed herein can be combined with one or more anti-psychotics, including, but not limited to, chlorpromazine, levomepromazine, promazine, acepromazine, triflupromazine, cyamemazine, chlorproethazine, dixyrazine, fluphenazine, perphenazine, prochlorperazine, thiopropazate, trifluoperazine, acetophenazine, thioproperazine, butaperazine, perazine, periciazine, thioridazine, mesoridazine, pipotiazine, haloperidol, trifluperidol, melperone, moperone, pipamperone, bromperidol, benperidol, droperidol, fluanisone, oxypertine, molindone, sertindole, ziprasidone, flupentixol, clopenthixol, chlorprothixene, thiothixene, zuclopenthixol, fluspirilene, pimozide, penfluridol, loxapine, clozapine, olanzapine, quetiapine, tetrabenazine, sulpiride, sultopride, tiapride, remoxipride, amisulpride, veralipride, levosulpiride, lithium, prothipendyl, risperidone, clotiapine, mosapramine, zotepine, pripiprazole, and paliperidone.

In certain embodiments, the compounds disclosed herein can be combined with one or more benzodiazepines (“minor tranquilizers”), including, but not limited to alprazolam, adinazolam, bromazepam, camazepam, clobazam, clonazepam, clotiazepam, cloxazolam, diazepam, ethyl loflazepate, estizolam, fludiazepam, flunitrazepam, halazepam, ketazolam, lorazepam, medazepam, dazolam, nitrazepam, nordazepam, oxazepam, potassium clorazepate, pinazepam, prazepam, tofisopam, triazolam, temazepam, and chlordiazepoxide.

In certain embodiments, the compounds disclosed herein can be combined with olanzapine or pimozide.

The compounds disclosed herein can also be administered in combination with other classes of compounds, including, but not limited to, anti-retroviral agents; CYP3A inhibitors; CYP3A inducers; protease inhibitors; adrenergic agonists; anti-cholinergics; mast cell stabilizers; xanthines; leukotriene antagonists; glucocorticoids treatments; local or general anesthetics; non-steroidal anti-inflammatory agents (NSAIDs), such as naproxen; antibacterial agents, such as amoxicillin; cholesteryl ester transfer protein (CETP) inhibitors, such as anacetrapib; anti-fungal agents, such as isoconazole; sepsis treatments, such as drotrecogin-steroidals, such as hydrocortisone; local or general anesthetics, such as ketamine; norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stablizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating VMAT2-mediated disorders in a subject in need of such treatment comprising administering to said subject an amount of a compound disclosed herein effective to reduce or prevent said disorder in the subject, in combination with at least one additional agent for the treatment of said disorder. In a related aspect, certain embodiments provide therapeutic compositions comprising at least one compound disclosed herein in combination with one or more additional agents for the treatment of VMAT2-mediated disorders.

In order that the disclosure described herein may be more fully understood, the following examples are set forth. It should be understood that these examples are for illustrative purposes only and are not to be construed as limiting this disclosure in any manner.

### EXAMPLES

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in WO 2005077946; WO 2008/058261; EP 1716145; Lee et al., J. Med. Chem., 1996, (39), 191-196; Kilbourn et al., Chirality, 1997, (9), 59-62; Boldt et al., Synth. Commun., 2009, (39), 3574-3585; Rishel et al., J. Org. Chem., 2009, (74), 4001-4004; DaSilva et al., Appl. Radiat. Isot., 1993, 44(4), 673-676; Popp et al., J. Pharm. Sci., 1978, 67(6), 871-873; Ivanov et al., Heterocycles 2001, 55(8), 1569-1572; U.S. Pat. No. 2,830,993; U.S. Pat. No. 3,045,021; WO 2007130365; WO 2008058261, which are hereby incorporated in their entirety, and references cited therein and routine modifications thereof. Compounds as disclosed herein can also be prepared as shown in any of the following schemes and routine modifications thereof.

The following schemes can be used to practice the present invention. Any position shown as hydrogen may optionally be replaced with deuterium.

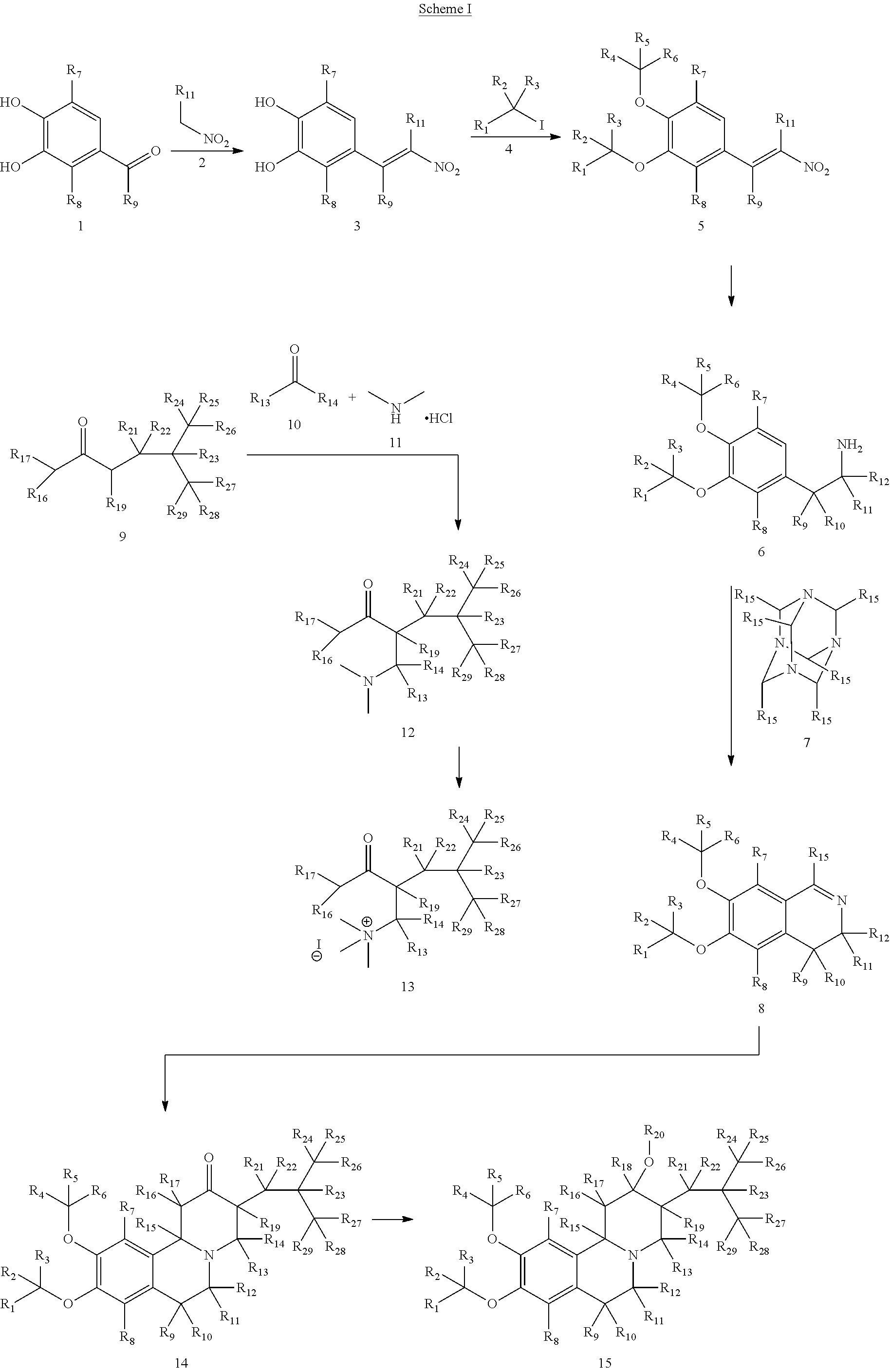

Compound 1 is reacted with compound 2 in an appropriate solvent, such as nitromethane, in the presence of an appropriate acid, such as ammonium acetate, at an elevated temperature to give compound 3. Compound 3 is reacted with compound 4 in the presence of an appropriate base, such as potassium carbonate, in an appropriate solvent, such as N,N-dimethylformamide, at an elevated temperature to afford compound 5. Compound 5 is reacted with an appropriate reducing reagent, such as lithium aluminum hydride, in an appropriate solvent, such as tetrahyrdofuran, at an elevated temperature to give compound 6. Compound 6 is reacted with compound 7 in the presence of an appropriate acid, such as trifluoroacetic acid, in an appropriate solvent, such as acetic acid, at an elevated temperature to give compound 8. Compound 9 is reacted with compound 10 and compound 11, in an appropriate solvent, such as methanol, at an elevated temperature to afford compound 12. Compound 12 is reacted with an appropriate methylating agent, such as methyl iodide, in an appropriate solvent, such as ethyl acetate, to give compound 13. Compound 8 is reacted with compound 13 in an appropriate solvent, such as ethanol, at an elevated temperature to give compound 14. Compound 14 is reacted with an appropriate reducing agent, such as sodium borohydride, in an appropriate solvent, such as methanol, to give compound 15 of Formula I.

Deuterium can be incorporated to different positions synthetically, according to the synthetic procedures as shown in Scheme I, by using appropriate deuterated intermediates. For example, to introduce deuterium at one or more positions of R₁-R₆, compound 4 with the corresponding deuterium substitutions can be used. To introduce deuterium at one or more positions of R₇-R₉, compound 1 with the corresponding deuterium substitutions can be used. To introduce deuterium at one or more positions of R₁₀ and R₁₂, lithium aluminum deuteride can be used. To introduce deuterium at R₁₁, compound 2 with the corresponding deuterium substitution can be used. To introduce deuterium at one or more positions of R₁₃-R₁₄, compound 10 with the corresponding deuterium substitutions can be used. To introduce deuterium at R₁₅, compound 7 with the corresponding deuterium substitution can be used. To introduce deuterium at one or more positions of R₁₆-R₁₇, R₁₉, and R₂₁-R₂₉, compound 9 with the corresponding deuterium substitutions can be used. To introduce deuterium at R₁₈, sodium borodeuteride can be used.

Deuterium can be incorporated to various positions having an exchangeable proton, such as the hydroxyl O—H, via proton-deuterium equilibrium exchange. For example, to introduce deuterium at R₂₀, this proton may be replaced with deuterium selectively or non-selectively through a proton-deuterium exchange method known in the art.

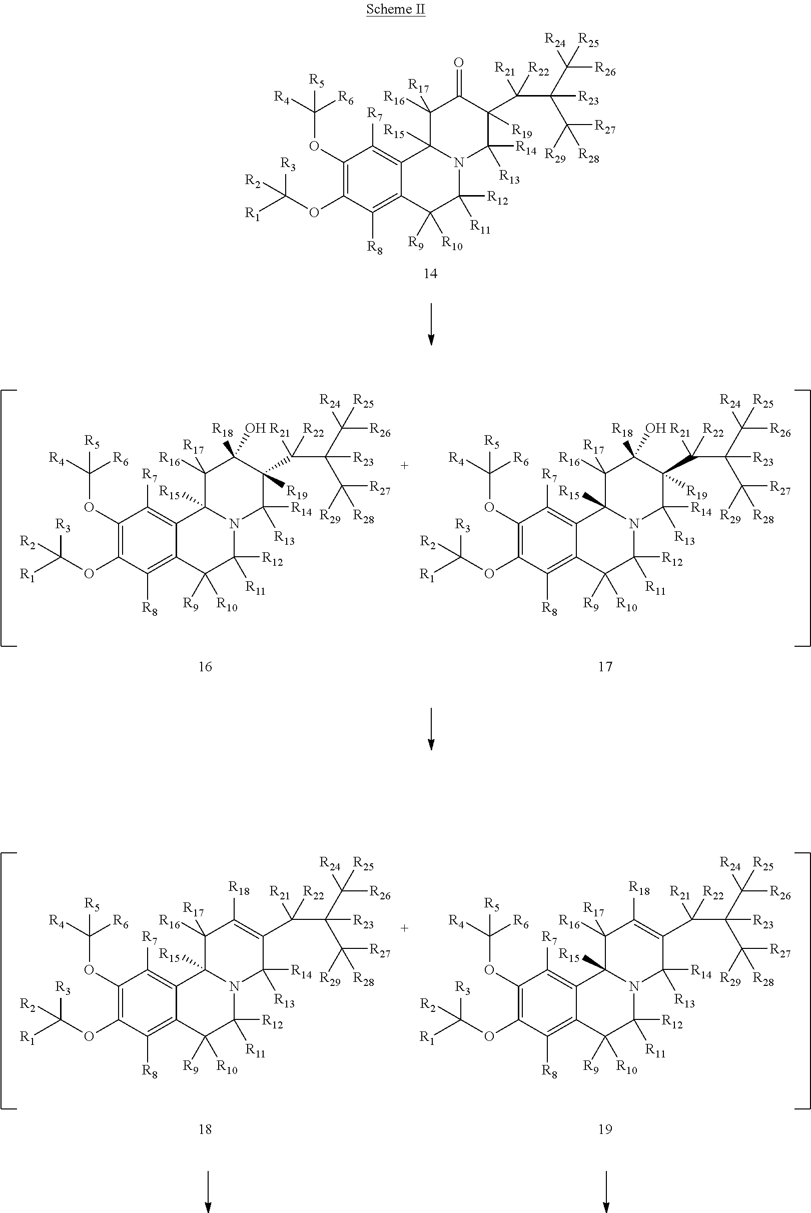

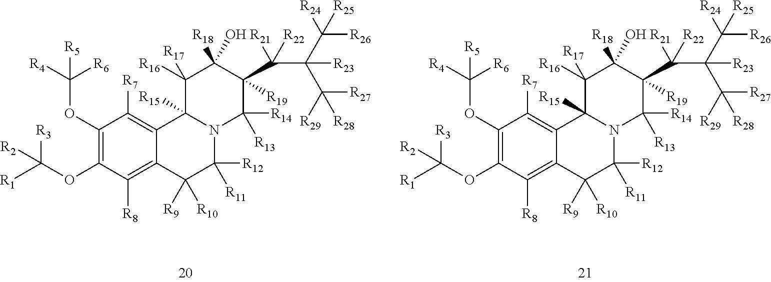

Compound 14 is reacted with an appropriate reducing agent, such as lithium tri-sec-butyl borohydride, in an appropriate solvent, such as ethanol, to give a mixture of compounds 16 and 17 of Formula II. Compounds 16 and 17 are reacted with an appropriate dehydrating reagent, such as phosphorous pentachloride, in an appropriate solvent, such as dichloromethane to afford a mixture of compounds 18 and 19. Compounds 18 and 19 are reacted with an appropriate hydroborating reagent, such as borane-tetrahydrofuran complex, in an appropriate solvent, such as tetrahyrdofuran, then oxidized with a mixture of sodium hydroxide and hydrogen peroxide, to give compounds 20 and 21 of Formula II. Mixtures of compounds 16 and 17 or 20 and 21 can be separated by chiral preparative chromatography of through the preparation of Mosher's esters (wherein the mixture is treated with R-(+)-3,3,3-trifluoro-2-methoxy-2-phenylpropanoic acid, an appropriate chlorinating agent, such as oxalyl chloride, and an appropriate base, such as 4-dimethylaminopyridine, in an appropriate solvent, such as dichloromethane, to give an epimeric mixture of R-(+)-3,3,3-trifluoro-2-methoxy-2-phenylpropanoate esters), which can be isolated via chromatography and then converted to the desired alcohol via hydrolysis (the Mosher's esters are treated with an appropriate base, such as sodium hydroxide, in an appropriate solvent, such as methanol, to give the desired compounds of Formula II).

Deuterium can be incorporated to different positions synthetically, according to the synthetic procedures as shown in Scheme II, by using appropriate deuterated intermediates. For example, to introduce deuterium at one or more positions of R₁-R₁₇ and R₂₁-R₂₉, compound 14 with the corresponding deuterium substitutions can be used. To introduce deuterium at Rig, lithium tri-sec-butyl borodeuteride can be used. To introduce deuterium at R₁₉, trideuteroborane can be used.

Deuterium can be incorporated to various positions having an exchangeable proton, such as the hydroxyl O—H, via proton-deuterium equilibrium exchange. For example, to introduce deuterium at R₂₀, this proton may be replaced with deuterium selectively or non-selectively through a proton-deuterium exchange method known in the art.

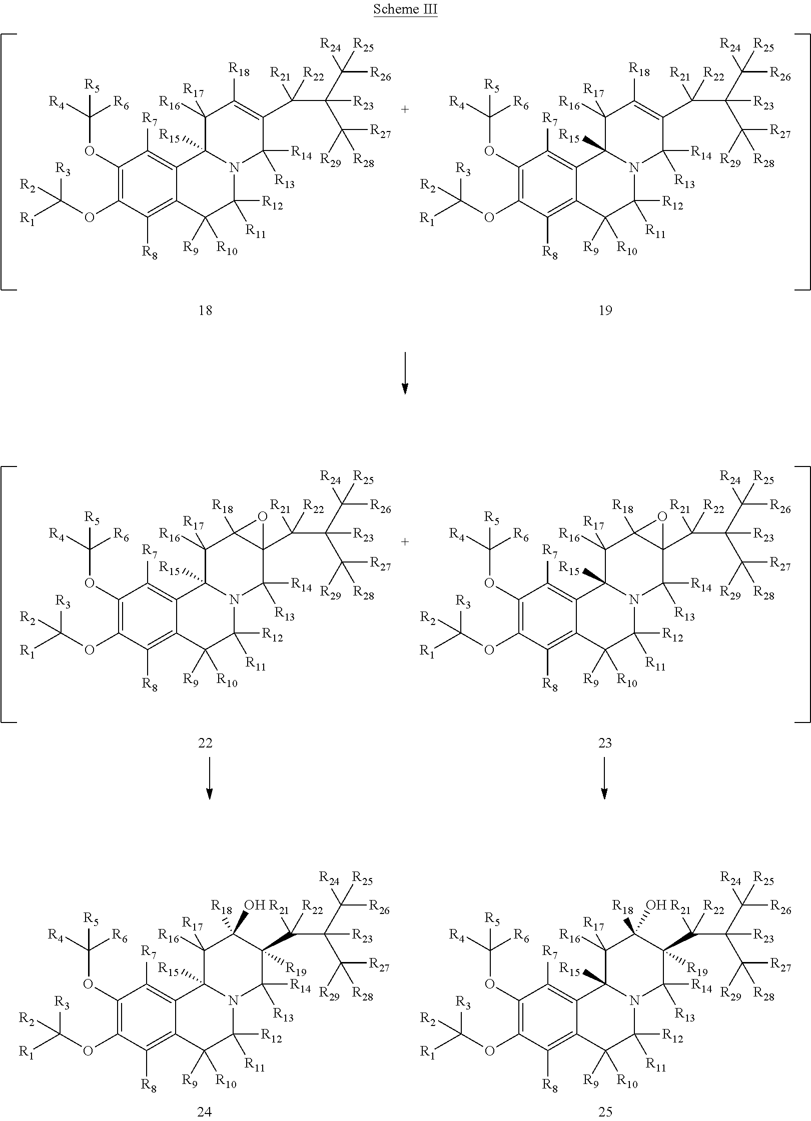

Compounds 18 and 19 (prepared as shown in Scheme II) are reacted with an appropriate peroxidizing agent, such as m-chloroperbenzoic acid, in the presence of an appropriate acid, such as perchloric acid, in an appropriate solvent, such as methanol, to give compounds 22 and 23. Compounds 22 and 23 are reacted with an appropriate reducing agent, such as borane-tetrahydrofuran complex, in an appropriate solvent, such as tetrahyrdofuran, then hydrolyzed with a mixture of sodium hydroxide and hydrogen peroxide, to give compounds 24 and 25 of Formula II. Mixtures of compounds 24 and 25 can be separated by chiral preparative chromatography of through the preparation of Mosher's esters (wherein the mixture is treated with R-(+)-3,3,3-trifluoro-2-methoxy-2-phenylpropanoic acid, an appropriate chlorinating agent, such as oxalyl chloride, and an appropriate base, such as 4-dimethylaminopyridine, in an appropriate solvent, such as dichloromethane, to give an epimeric mixture of R-(+)-3,3,3-trifluoro-2-methoxy-2-phenylpropanoate esters), which can be isolated via chromatography and then converted to the desired alcohol via hydrolysis (the Mosher's esters are treated with an appropriate base, such as sodium hydroxide, in an appropriate solvent, such as methanol, to give the desired compounds of Formula II).

Deuterium can be incorporated to different positions synthetically, according to the synthetic procedures as shown in Scheme III, by using appropriate deuterated intermediates. For example, to introduce deuterium at one or more positions of R₁-R₁₈ and R₂₁-R₂₉, compounds 18 and 19 with the corresponding deuterium substitutions can be used. To introduce deuterium at Rig, trideuteroborane can be used.

Deuterium can be incorporated to various positions having an exchangeable proton, such as the hydroxyl O—H, via proton-deuterium equilibrium exchange. For example, to introduce deuterium at R₂₀, this proton may be replaced with deuterium selectively or non-selectively through a proton-deuterium exchange method known in the art.

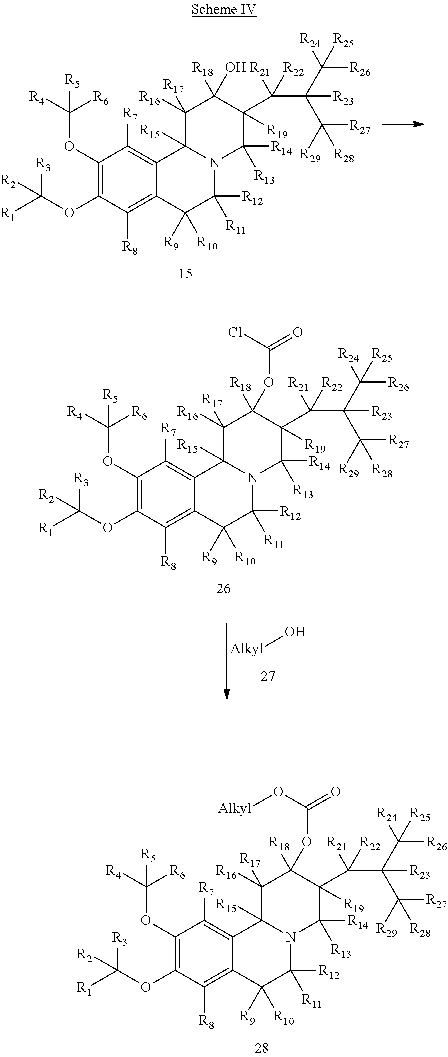

Compound 15 is reacted with an appropriate phosgene equivalent, such as triphosgene, in an appropriate solvent, such as dichloromethane, to give compound 26. Compound 26 is reacted with an appropriate alcohol, such as compound 27, in the presence of an appropriate base, such as 4-dimethylaminopyridine, to give compound 28 of Formula II (where R₂₂ is —C(O))-alkyl).

Deuterium can be incorporated to different positions synthetically, according to the synthetic procedures as shown in Scheme IV, by using appropriate deuterated intermediates. For example, to introduce deuterium at one or more positions of R₁-R₁₉ and R₂₁-R₂₉, compound 16 with the corresponding deuterium substitutions can be used. To introduce deuterium at R₂₀, compound 27 with the corresponding deuterium substitutions can be used.

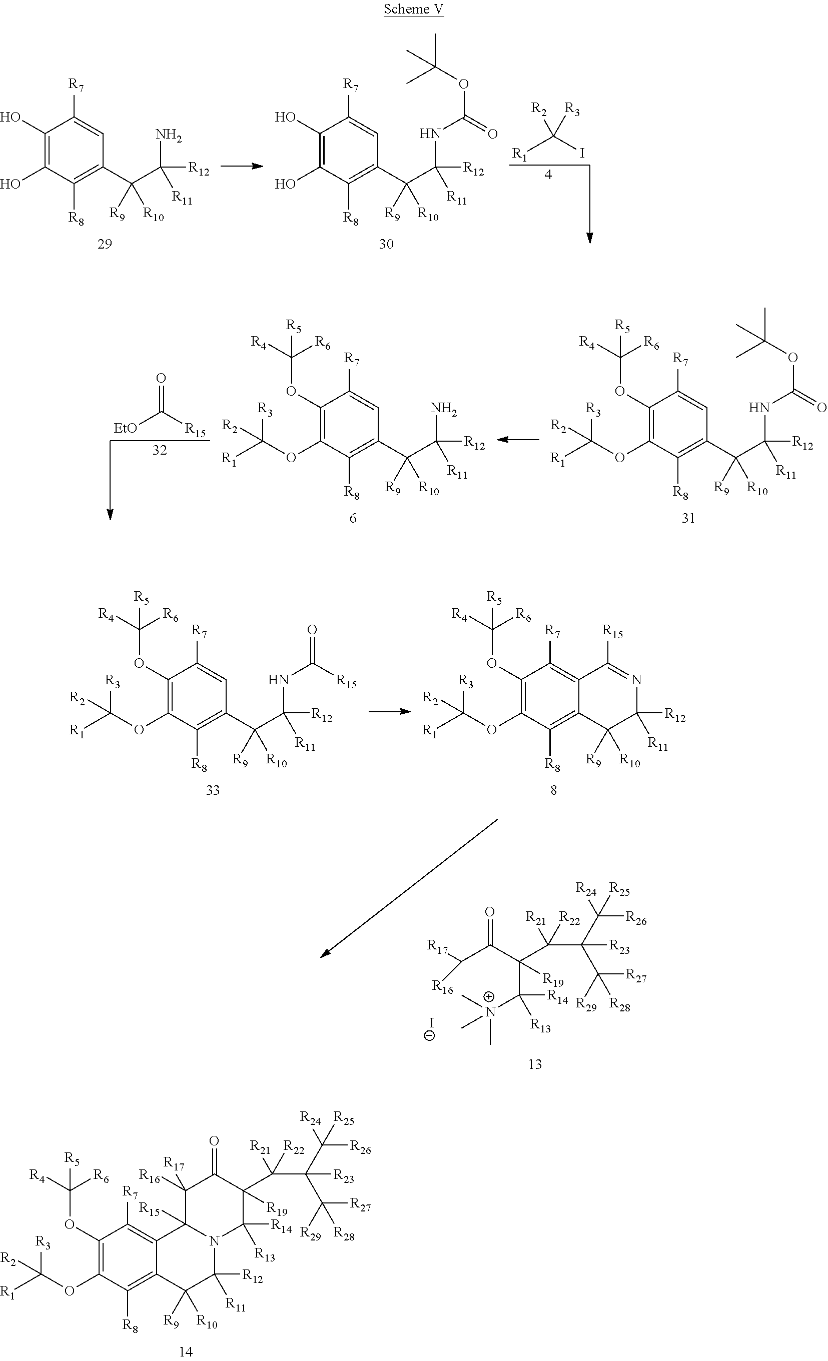

Compound 29 is reacted with an appropriate protecting agent, such as di-tert-butyl dicarbonate, in an appropriate solvent, such as a mixture of tetrahydrofuran and water, in the presence of an appropriate base, such as sodium carbonate, to give compound 30. Compound 30 is reacted with compound 4 in the presence of an appropriate base, such as potassium carbonate, in the presence of an appropriate catalyst, such as 18-crown-6, in an appropriate solvent, such as acetone, to afford compound 31. Compound 31 is reacted with an appropriate deprotecting agent, such as hydrogen chloride, in an appropriate solvent, such as ethyl acetate, to give compound 6. Compound 6 is reacted with compound 32 at an elevated temperature to give compound 33. Compound 33 is reacted with an appropriate dehydrating agent, such as phosphorous oxychloride, at an elevated temperature to afford compound 8. Compound 8 is reacted with compound 13 in an appropriate solvent, such as methanol, at an elevated temperature to give compound 14.

Deuterium can be incorporated to different positions synthetically, according to the synthetic procedures as shown in Scheme V, by using appropriate deuterated intermediates. For example, to introduce deuterium at one or more positions of R₁-R₆, compound 4 with the corresponding deuterium substitutions can be used. To introduce deuterium at one or more positions of R₇-R₁₂, compound 29 with the corresponding deuterium substitutions can be used. To introduce deuterium at R₁₅, compound 32 with the corresponding deuterium substitution can be used. To introduce deuterium at one or more positions of R₁₃-R₁₄, R₁₆-R₁₇, R₁₉, and R₂₁-R₂₉, compound 13 with the corresponding deuterium substitutions can be used.

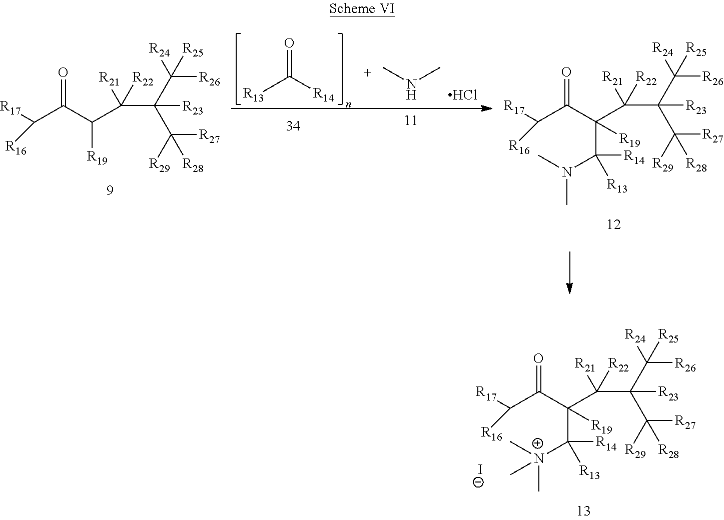

Compound 9 is reacted with compound 11 and compound 34 (paraformaldehyde and/or formaldehyde) in an appropriate solvent, such as ethanol, in the presence of an appropriate acid, such as hydrochloric acid, at an elevated temperature to give compound 12. Compound 12 is reacted with an appropriate methylating agent, such as methyl iodide, in an appropriate solvent, such as ethyl acetate, to give compound 13. Compound 8 is reacted with compound 13 in an appropriate solvent, such as dichloromethane, to give compound 13.

Deuterium can be incorporated to different positions synthetically, according to the synthetic procedures as shown in Scheme VI, by using appropriate deuterated intermediates. For example, to introduce deuterium at one or more positions of R₁₃-R₁₄, compound 10 with the corresponding deuterium substitutions can be used. To introduce deuterium at one or more positions of R₁₆-R₁₇, R₁₉, and R₂₁-R₂₉, compound 9 with the corresponding deuterium substitutions can be used.

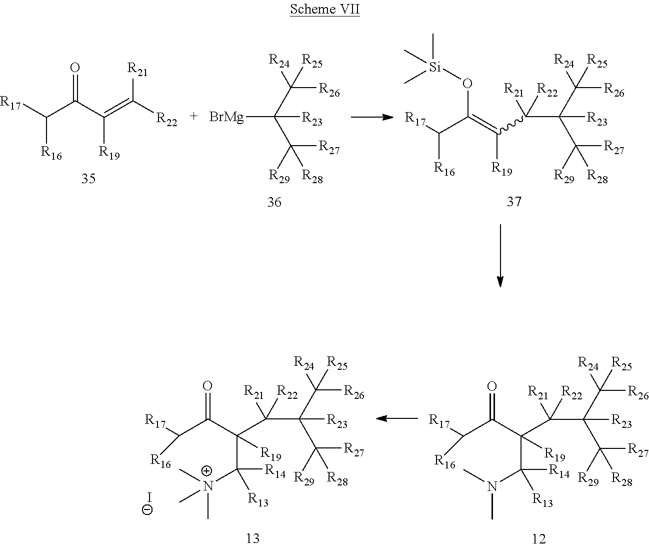

Compound 35 is reacted with compound 36 in an appropriate solvent, such as tetrahydrofuran, in the presence of an appropriate catalyst, such as cuprous iodide, and an appropriate co-solvent, such as hexamethylphosphorous triamide, then reacted with an appropriate protecting agent, such as trimethylsilyl chloride, and an appropriate base, such as triethylamine, to give compound 37. Compound 37 is reacted with an appropriate mannich base, such as N-methyl-N-methylenemethanaminium iodide, in an appropriate solvent, such as acetonitrile, to afford compound 12. Compound 12 is reacted with an appropriate methylating agent, such as methyl iodide, in an appropriate solvent, such as diethyl ether, to give compound 13.

Deuterium can be incorporated to different positions synthetically, according to the synthetic procedures as shown in Scheme VII, by using appropriate deuterated intermediates. For example, to introduce deuterium at one or more positions of R₁₆-R₁₇, Rig, and R₂₁-R₂₂, compound 35 with the corresponding deuterium substitutions can be used. To introduce deuterium at one or more positions of R₂₃-R₂₉, compound 36 with the corresponding deuterium substitutions can be used.

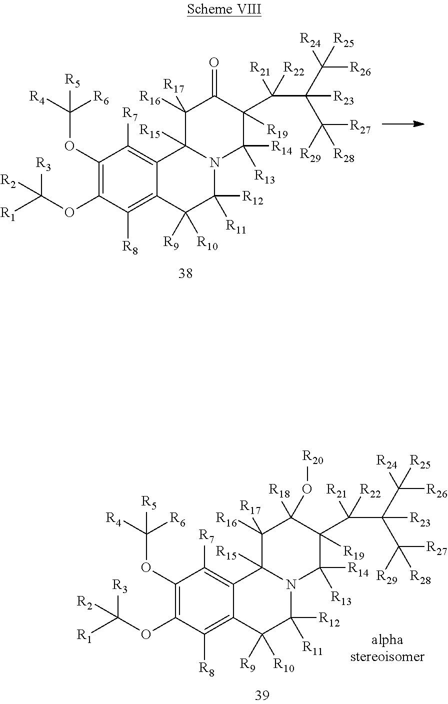

Compound 38 is reacted with an appropriate reducing agent, such as sodium borohydride, in an appropriate solvent, such as ethanol, to give compound 39 of Formula II having predominantly (˜4:1) alpha stereochemistry. The alpha stereoisomer can be further enriched by recrystalization from an appropriate solvent, such as ethanol.

Deuterium can be incorporated to different positions synthetically, according to the synthetic procedures as shown in Scheme I, by using appropriate deuterated intermediates. For example, to introduce deuterium at one or more positions of R₁-R₁₇, R₉₉, and R₂₁-R₂₉, compound 38 with the corresponding deuterium substitutions can be used. To introduce deuterium at Rig, sodium borodeuteride can be used.

Deuterium can be incorporated to various positions having an exchangeable proton, such as the hydroxyl O—H, via proton-deuterium equilibrium exchange. For example, to introduce deuterium at R₂₀, this proton may be replaced with deuterium selectively or non-selectively through a proton-deuterium exchange method known in the art.

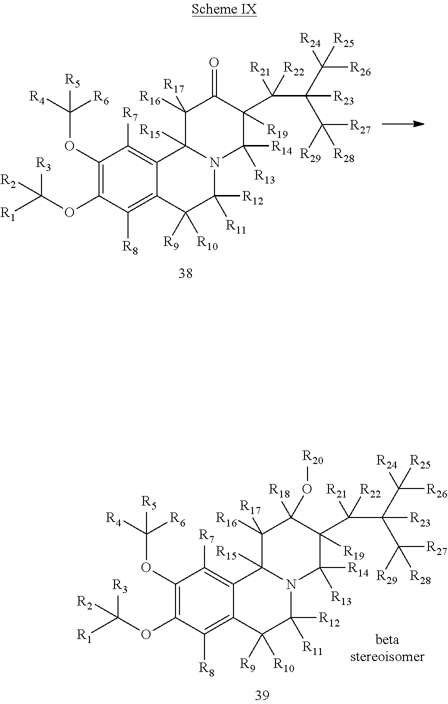

Compound 38 is reacted with an appropriate reducing agent, such as potassium tri-sec-butyl borohydride (K-selectride), in an appropriate solvent, such as tetrahydrofuran, to give compound 40 of Formula I having beta stereochemistry.

Deuterium can be incorporated to different positions synthetically, according to the synthetic procedures as shown in Scheme I, by using appropriate deuterated intermediates. For example, to introduce deuterium at one or more positions of R₁-R₁₇, R₉₉, and R₂₁-R₂₉, compound 38 with the corresponding deuterium substitutions can be used. To introduce deuterium at R₁₈, potassium tri-sec-butyl borodeuteride can be used.

Deuterium can be incorporated to various positions having an exchangeable proton, such as the hydroxyl O—H, via proton-deuterium equilibrium exchange. For example, to introduce deuterium at R₂₀, this proton may be replaced with deuterium selectively or non-selectively through a proton-deuterium exchange method known in the art.

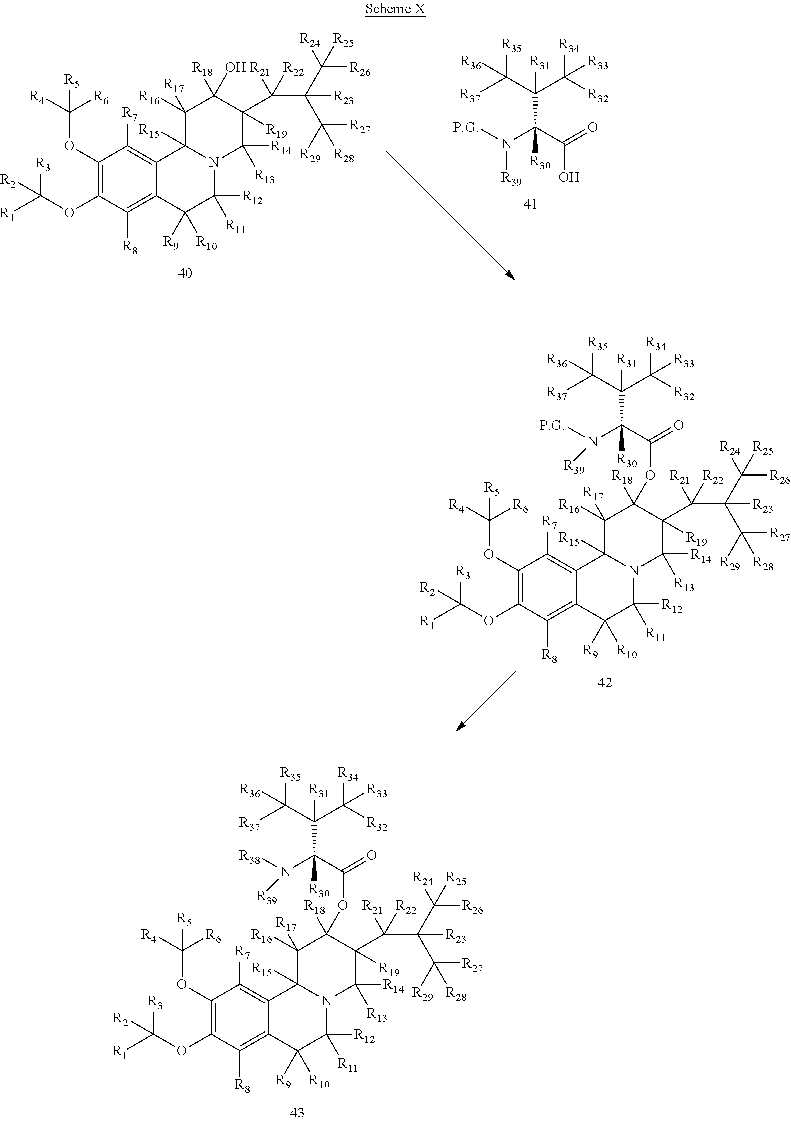

Compound 40 is reacted with compound 41 (wherein P.G. is an appropriate protecting group, such as carboxybenzoyl) in the presence of an appropriate coupling agent, such as dicyclohexylcarbodiimide (DCC), an appropriate catalyst, such as 4-dimethylaminopyridine (DMAP), in an appropriate solvent, such as dichloromethane, to give compound 42. Compound 42 is reacted with an appropriate deprotecting agent, such as a combination of hydrogen and an appropriate catalyst, such as palladium on carbon, in an appropriate solvent, such as methanol, to give compound 43 of Formula II.

Deuterium can be incorporated to different positions synthetically, according to the synthetic procedures as shown in Scheme I, by using appropriate deuterated intermediates. For example, to introduce deuterium at one or more positions of R₁-R₁₉ and R₂₁-R₂₉, compound 40 with the corresponding deuterium substitutions can be used. To introduce deuterium at one or more positions of R₃₀-R₃₇, compound 41 with the corresponding deuterium substitutions can be used.

Deuterium can be incorporated to various positions having an exchangeable proton, such as the hydroxyl O—H or amine N—Hs, via proton-deuterium equilibrium exchange. For example, to introduce deuterium at R₂₀ and R₃₈-R₃₉, these protons may be replaced with deuterium selectively or non-selectively through a proton-deuterium exchange method known in the art.

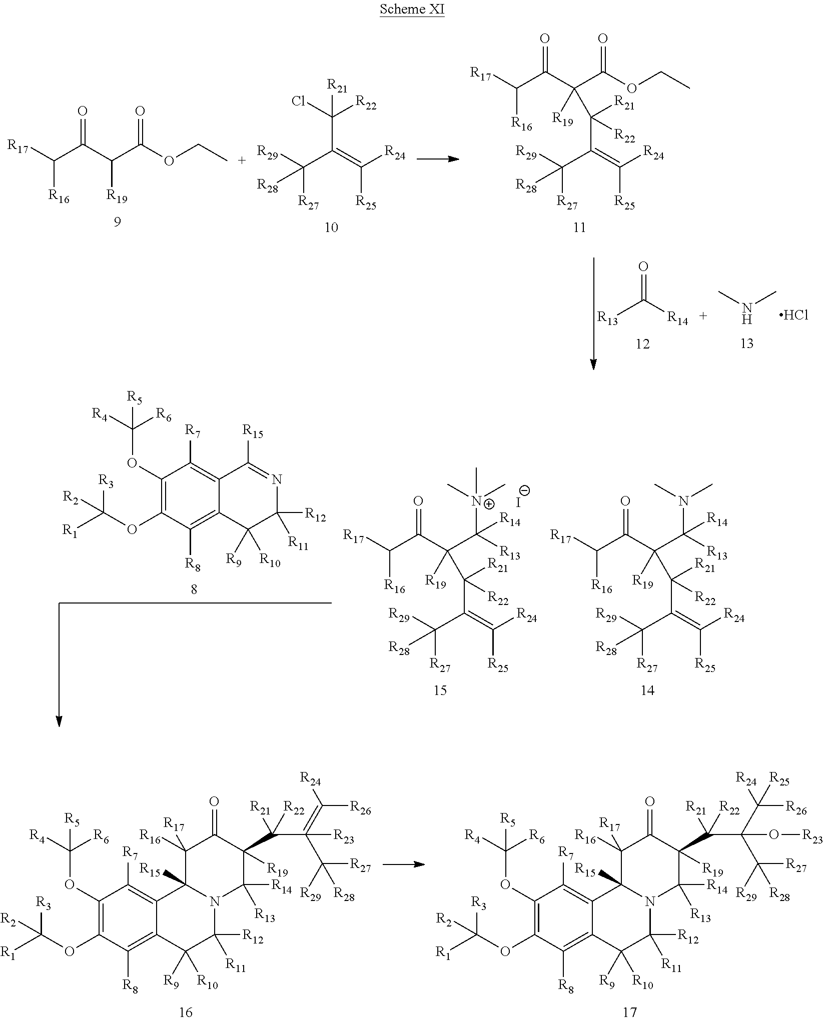

Compound 44 is reacted with compound 45 in the presence of an appropriate base, such as potassium carbonate, in the presence of an appropriate phase transfer catalyst, such as a combination of potassium iodide and tetrabutylammonium bromide, in an appropriate solvent, such as N,N-dimethylformamide, at an elevated temperature to afford compound 46. Compound 46 is reacted with an appropriate base, such as potassium hydroxide, then reacted with compound 47 and compound 48 in the presence of an appropriate acid, such as hydrochloric acid, and an appropriate phase transfer catalyst, such as tetrabutylammonium bromide, in an appropriate solvent, such as water, to afford compound 49. Compound 49 is reacted with an appropriate methylating agent, such as methyl iodide, in an appropriate solvent, such as methyl tert-butyl ether, to give compound 50. Compound 8 is reacted with compound 50 in an appropriate solvent, such as a mixture of methanol and water, at an elevated temperature to give compound 51. Compound 51 is reacted with an appropriate acid, such as sulfuric acid, in an appropriate solvent, such as water, to give compound 52 of Formula III.

Deuterium can be incorporated to different positions synthetically, according to the synthetic procedures as shown in Scheme I, by using appropriate deuterated intermediates. For example, to introduce deuterium at one or more positions of R₁-R₁₂ and R₁₅, compound 8 with the corresponding deuterium substitutions can be used. To introduce deuterium at one or more positions of R₁₃-R₁₄, compound 47 with the corresponding deuterium substitutions can be used. To introduce deuterium at one or more positions of R₁₆-R₁₇ and R₁₉, compound 44 with the corresponding deuterium substitutions can be used. To introduce deuterium at one or more positions of R₂₁-R₂₂, R₂₄-R₂₅, and R₂₇-R₂₉, compound 45 with the corresponding deuterium substitutions can be used. To introduce deuterium at one or more positions of R₂₃ and R₂₆, D₂SO₄ and/or D₂O can be used.

Deuterium can be incorporated to various positions having an exchangeable proton, such as the hydroxyl O—H, via proton-deuterium equilibrium exchange. For example, to introduce deuterium at R₂₃, this proton may be replaced with deuterium selectively or non-selectively through a proton-deuterium exchange method known in the art.

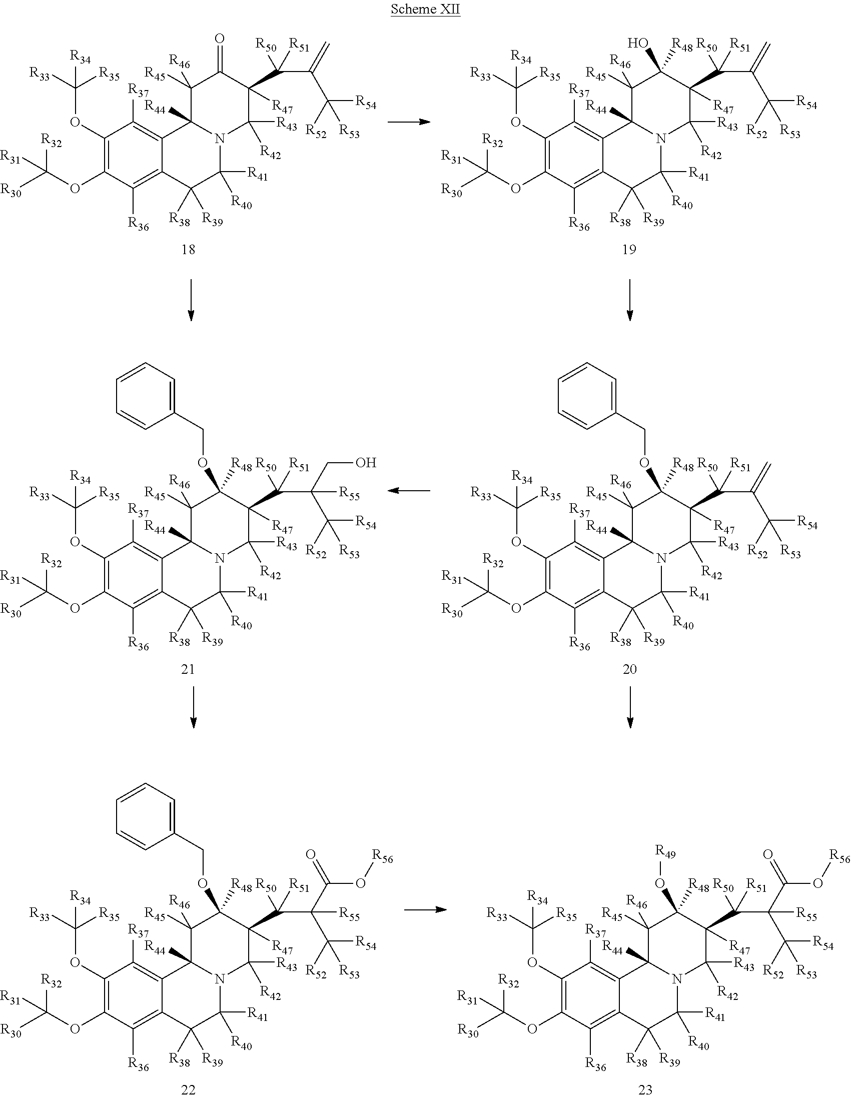

Compound 53 is reacted with an appropriate reducing agent, such as lithium tri-sec-butyl borohydride, in an appropriate solvent, such as tetrahydrofuran, to give compound 54. Compound 54 is reacted with an appropriate protecting agent, such as benzyl bromide, in the presence of an appropriate base, such as sodium hydride, in an appropriate solvent, such as tetrahydrofuran to give compound 55. Compound 55 is reacted with an appropriate hydroborating reagent, such as borane-dimethylsulfide complex, in an appropriate solvent, such as tetrahyrdofuran, then reacted with an appropriate base, such as aqueous sodium hydroxide, to give compound 56. Compound 56 is reacted with an appropriate oxidizing agent, such as Jones reagent (an aqueous solution of chromium trioxide and sulfuric acid), in an appropriate solvent, such as acetone, to give compound 57. Compound 57 is reacted with an appropriate deprotecting agent, such as a mixture of palladium on carbon and hydrogen gas, in an appropriate solvent, such as methanol, to give compound 58 of Formula IV.

Deuterium can be incorporated to different positions synthetically, according to the synthetic procedures as shown in Scheme II, by using appropriate deuterated intermediates. For example, to introduce deuterium at one or more positions of R₃₀-R₄₇ and R₅₀-R₅₄, compound 53 with the corresponding deuterium substitutions can be used. To introduce deuterium at R₄₈, lithium tri-sec-butyl borodeuteride can be used. To introduce deuterium at R₅₅, trideuteroborane can be used.

Deuterium can be incorporated to various positions having an exchangeable proton, such as the hydroxyl O—H or carboxyl O—H, via proton-deuterium equilibrium exchange. For example, to introduce deuterium at R₄₉ and/or R₅₆, these protons may be replaced with deuterium selectively or non-selectively through a proton-deuterium exchange method known in the art.

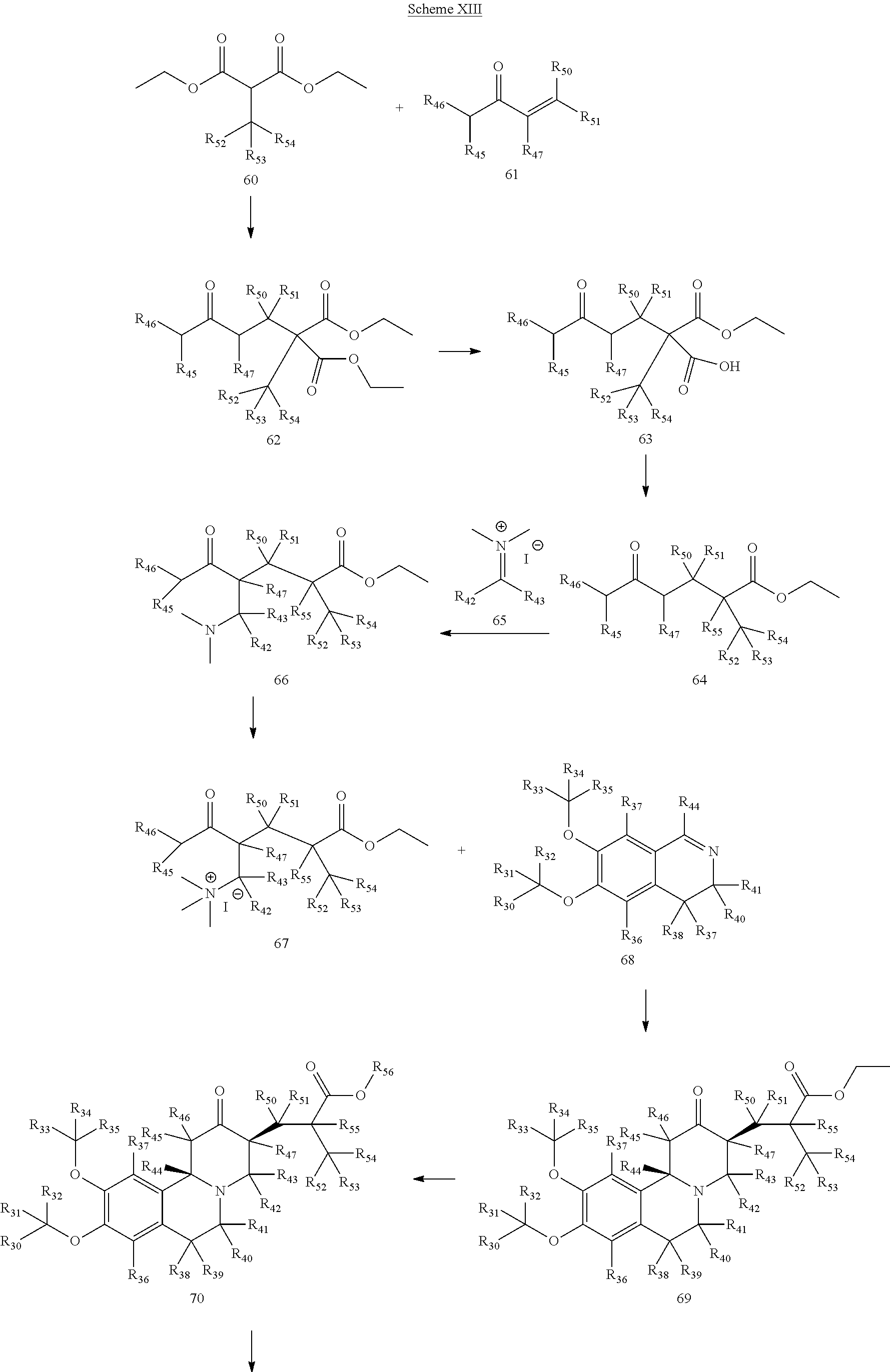

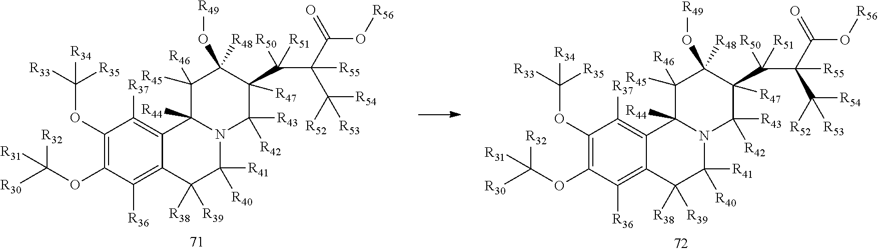

Compound 60 is reacted with compound 61 in the presence of an appropriate base, such as potassium carbonate, in an appropriate solvent, such as dichloromethane, at an elevated temperature to afford compound 62. Compound 62 is reacted with an appropriate base, such as sodium hydroxide, in an appropriate solvent, such as a mixture of ethanol and water, to afford compound 63. Compound 63 is heated to an elevated temperature in an appropriate solvent, such as a mixture of dimethylsulfoxide and water, to give compound 64. Compound 64 is reacted with an appropriate silating agent, such as trimethylsilyl iodide, in the presence of an appropriate base, such as hexamethyldisilazide, to give an intermediate silyl enol ether which is reacted with compound 65 in an appropriate solvent, such as acetonitrile, to afford compound 66. Compound 66 is reacted with an appropriate methylating agent, such as methyl iodide, to give compound 67. Compound 67 is reacted with compound 68 in an appropriate solvent, such as ethanol, at an elevated temperature to give compound 69. Compound 69 is reacted with an appropriate base, such as lithium hydroxide, in an appropriate solvent, such as a mixture of tetrahydrofuran and water, to afford compound 70. Compound 70 is reacted with an appropriate reducing agent, such as potassium tri-sec-butyl borohydride (K-selectride), in an appropriate solvent, such as tetrahydrofuran, to give compound 71 as a mixture of diastereomers. Compound 71 is recrystallized from water, to give compound 72 of Formula III.

Deuterium can be incorporated to different positions synthetically, according to the synthetic procedures as shown in Scheme I, by using appropriate deuterated intermediates. For example, to introduce deuterium at one or more positions of R₅₂-R₅₄, compound 60 with the corresponding deuterium substitutions can be used. To introduce deuterium at one or more positions of R₄₅-R₄₇ and R₅₀-R₅₁, compound 61 with the corresponding deuterium substitutions can be used. To introduce deuterium at R₅₅, D₂O can be used. To introduce deuterium at one or more positions of R₄₂-R₄₃, compound 65 with the corresponding deuterium substitutions can be used. To introduce deuterium at one or more positions of R₃₀-R₄₁ and R₄₄, compound 68 with the corresponding deuterium substitutions can be used. introduce deuterium at R₅₅, potassium tri-sec-butyl borodeuteride can be used.

Deuterium can be incorporated to various positions having an exchangeable proton, such as the hydroxyl O—H or carboxyl O—H, via proton-deuterium equilibrium exchange. For example, to introduce deuterium at R₄₉ and/or R₅₆, these protons may be replaced with deuterium selectively or non-selectively through a proton-deuterium exchange method known in the art.

The invention is further illustrated by the following examples. All IUPAC names were generated using CambridgeSoft's ChemDraw 10.0.

### Example 1

### D₆-(±)-3-Isobutyl-9,10-dimethoxy-3,4,6,7-tetrahydro-1H-pyrido[2,1-a]isoquinolin-2(11bH)-one((±)-Tetrabenazine-d₆)

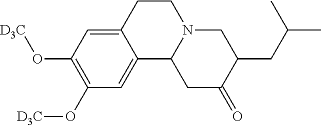 [SMILES: [2H]C([2H])([2H])Oc1cc2c(cc1OC([2H])([2H])[2H])C1CC(=O)C(CC(C)C)CN1CC2]

### Step 1

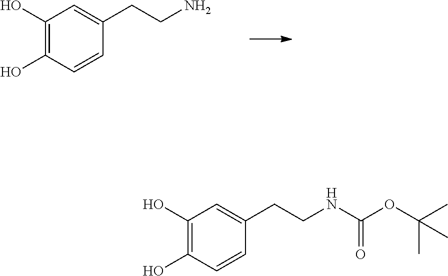 [SMILES: NCCc1ccc(O)c(O)c1 ; CC(C)(C)OC(=O)NCCc1ccc(O)c(O)c1]

### Tert-butyl 3,4-dihydroxyphenethylcarbamate

A solution of dopamine hydrochloride (209 g, 1.11 mol, 1.00 equiv), sodium carbonate (231 g, 2.75 mol, 2.50 equiv) and di-tert-butyl dicarbonate (263 g, 1.21 mol, 1.10) in 2.4 L tetrahydrofuran/water (5:1) was stirred at 20° C. for 2.5 h. After the starting material was consumed completedly, the reaction was diluted with ethyl acetate (2 L) and washed with water (2×600 mL). The organic phase was dried over sodium sulfate, filtered and concentrated under reduced pressure until two volumes of solvent was left. The precipitated solid was isolated by filtration and dried under vacuum to give 254 g (91%) of tert-butyl 3,4-dihydroxyphenethylcarbamate as white solid. ¹H-NMR (300 MHz, CDCl₃) δ 8.72 (s, 1H), 8.62 (s, 1H), 6.79 (m, 1H), 6.62 (m, 1H), 6.51 (m, 1H), 6.40 (m, 1H), 3.03 (m, 2H), 2.50 (m, 2H), 1.37 (s, 1H). LC-MS: m/z=254 (MH)⁺.

### Step 2

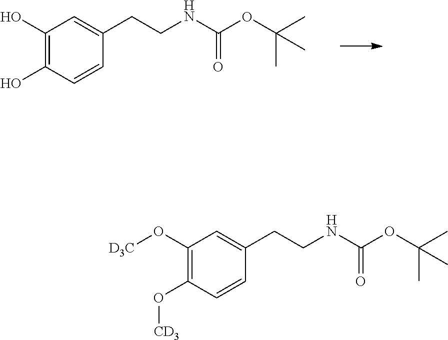 [SMILES: CC(C)(C)OC(=O)NCCc1ccc(O)c(O)c1 ; [2H]C([2H])([2H])Oc1ccc(CCNC(=O)OC(C)(C)C)cc1OC([2H])([2H])[2H]]

### D₆-tert-butyl 3,4-dimethoxyphenethylcarbamate

A solution of tert-butyl 3,4-dihydroxyphenethylcarbamate (127 g, 397 mmol, 1.00 equiv), potassium carbonate (359.3 g, 2.604 mmol, 3.00 equiv) and 18-crown-6 (1,4,7,10,13,16-hexaoxacyclooctadecane) (68.64 g, 0.26 mmol, 0.03 equiv) in acetone (800 mL) was stirred at 38° C. After 30 min., CD₃I (362 g, 2.604 mmol, 3.00 equiv) was added to the reaction, and the mixture was stirred at 38° C. for 12 h. Then an additional CD₃I (120 g, 0.868 mmol, 1.00 equiv) was added to the solution and the solution was stirred for 5 h. Then the mixture was cooled to room temperature and the solid was filtered. The filtrate was concentrated under vacuum. The resultant solid was dissolved in H₂O (300 mL) and extracted with EA (3×300 mL), the organic layers was combined and concentrated under vacuum to give 114 g (79%) of d₆-tert-butyl 3,4-dimethoxyphenethylcarbamate as white solid. ¹H-NMR (300 MHz, CDCl₃) δ 7.39 (m, 5H), 6.82 (m, 1H), 6.73 (m, 2H), 5.12 (s, 1H), 3.45 (m, 2H), 2.77 (m, 2H). LC-MS: m/z=288 (MH)⁺.

### Step 3

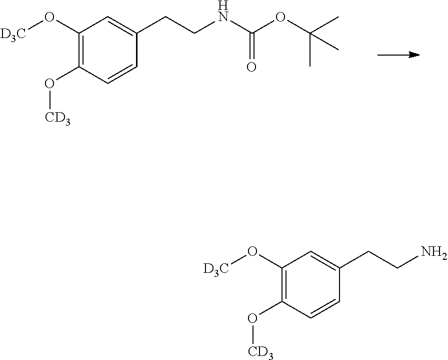 [SMILES: [2H]C([2H])([2H])Oc1ccc(CCNC(=O)OC(C)(C)C)cc1OC([2H])([2H])[2H] ; [2H]C([2H])([2H])Oc1ccc(CCN)cc1OC([2H])([2H])[2H]]

### D₆-2-(3,4-dimethoxyphenyl)ethanamine

A solution of d₆-tert-butyl 3,4-dimethoxyphenethylcarbamate (128 g, 455.26 mmol, 1.00 equiv) in ethyl acetate (1.5 L) was stirred at room temperature. Then HCl gas was introduced into the reaction mixture for 2 h. The precipitated solid was isolated by filtration. The solid was dissolved in 300 mL of water. The pH value of the solution was adjusted to 12 with sodium hydroxide (solid). The resulting solution was stirred for 1 h at 5-10° C. The resulting solution was extracted with 6×800 mL of ethyl acetate and the organic layers combined, dried over sodium sulfate, and concentrated under vacuum to give 64 g (78%) of d₆-2-(3,4-dimethoxyphenyl)ethanamine as yellow oil. ¹H-NMR (300 MHz, CDCl₃) δ 6.77 (m, 3H), 3.89 (s, 3H), 3.87 (s, 3H), 2.96 (m, 2H), 2.71 (m, 2H), 1.29 (s, 2H). LC-MS: m/z=182 (MH)⁺.

### Step 4

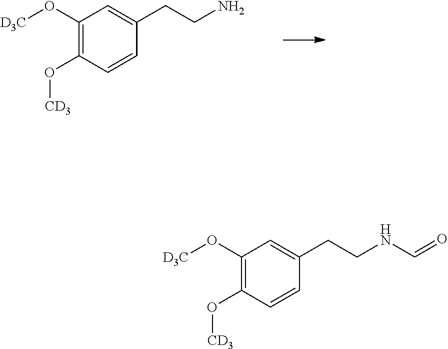 [SMILES: [2H]C([2H])([2H])Oc1ccc(CCN)cc1OC([2H])([2H])[2H] ; [2H]C([2H])([2H])Oc1ccc(CCNC=O)cc1OC([2H])([2H])[2H]]

### D₆-N-[2-(3,4-dimethoxy-phenyl)ethyl]formamide

A solution of d₆-2-(3,4-dimethoxyphenyl)ethanamine (69 g, 368 mmol, 1.00 equiv) in ethyl formate (250 mL) was heated under reflux overnight. The solution was concentrated under vacuum to give 71 g (91%) of d₆-N-[2-(3,4-dimethoxy-phenyl)ethyl]formamide as yellow solid. The crude solid was used in next step without purification. ¹H-NMR (300 MHz, CDCl₃) δ 8.17 (s, 1H), 6.81 (m, 3H), 5.53 (br, 1H). 3.59 (m, 2H), 2.81 (t, 2H, J=6.9 Hz). LC-MS: m/z=216 (MH)⁺.

### Step 5

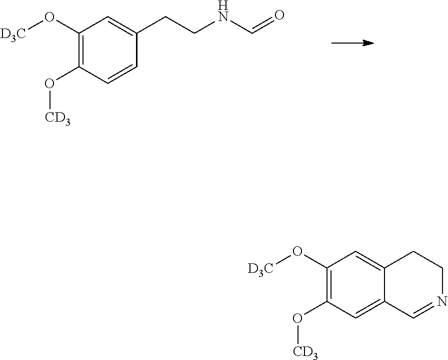 [SMILES: [2H]C([2H])([2H])Oc1ccc(CCNC=O)cc1OC([2H])([2H])[2H] ; [2H]C([2H])([2H])Oc1cc2c(cc1OC([2H])([2H])[2H])CCN=C2]

### D₆-6,7-dimethoxy-3,4-dihydroisoquinoline

A solution of d₆-N-[2-(3,4-dimethoxy-phenyl)ethyl]formamide (71 g, 329 mmol, 1.00 equiv) in phosphorus oxychloride (100 mL) was stirred at 105° C. for 1 h. Then the solution was concentrated under vacuum to remove phosphorus oxychloride. The residual oil was dissolved in ice/water. The solution was made basic with potassium carbonate with cooling. The basic aqueous solution was extracted with dichloromethane. The collected organic phase was dried using sodium sulfate and then filtered. The dichloromethane was removed by concentration under vacuum to give an orange oil. Purification by silica gel (ethyl acetate:petroleum ether=1:1˜ethyl acetate) to give 43 g (66%) of d₆-6,7-dimethoxy-3,4-dihydroisoquinoline as orange solid (yield 66%). ¹H-NMR (300 MHz, CDCl₃) δ 8.24 (s, 1H), 6.82 (s, 1H), 6.68 (s, 1H), 3.74 (m, 2H), 2.69 (t, 2H, J=7.2 Hz). LC-MS: m/z=198 (MH)⁺.

### Step 6

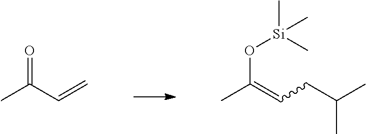

### Trimethyl(5-methylhex-2-en-2-yloxy)silane

To a cold (−78° C.), stirred solution of i-PrMgBr (500 mL of 2 M solution in tetrahydrofuran, 1 mol, 1.00 equiv) in anhydrous tetrahydrofuran (1 L) was added CuI (19.02 g, 0.1 mol, 0.10 equiv) and the resultant mixture was stirred for 15 min at −78° C. Anhydrous hexamethylphosphorous triamide (358.4 g, 2 mmol, 2 equiv) was added and after 20 min, a solution of methyl vinyl ketone (70 g, 0.1 mol, 1.00 equiv), trimethylsilyl chloride (217 g, 0.2 mol, 2.00 equiv), in tetrahydrofuran (200 mL) was added dropwise over 30 min. After the reaction mixture was stirred at −78° C. for 1 h, triethylamine (20.2 g, 200 mmol, 2.00 equiv) was added and the resulting mixture stirred for 10 min at 0° C. To this was added tert-butyl methyl ether (2 L), and the solution was washed with 5% ammonia solution (6×300 mL). Then the organic phase was dried over sodium sulfate and concentrated under vacuum at 25° C. to give 155 g crude product as yellow liquid. The liquid was purified by distilling (64-68° C./40 mmHg) to provide 118 g (63.3%) of trimethyl(5-methylhex-2-en-2-yloxy)silane (E:Z=56:44) as a colorless oil. ¹H-NMR (300 MHz, d₆-DMSO) δ 4.58 (m, 0.56H), 4.43 (m, 0.44H), 1.73 (s, 1.69H), 1.66 (s, 1.32H), 1.53 (m, 1H), 0.84 (m, 6H), 0.15 (m, 9H).

### Step 7

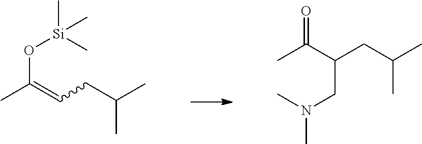

### 3-[(Dimethylamino)methyl]-5-methylhexan-2-one

To a stirred solution of trimethyl(5-methylhex-2-en-2-yloxy)silane (118 g, 633 mmol, 1.00 equiv) in anhydrous acetonitrile (800 mL) was added N-methyl-N-methylenemethanaminium iodide (128.8 g, 696.3 mmol, 1.10 equiv) in several batches and the resultant mixture was stirred at 20° C. overnight. Then the solution was concentrated under vacuum to remove the solvent. The residue was dissolved in 400 mL 1 N HCl (aq.) and extracted with tert-butyl methyl ether. Then the water phase was basiced with 2 N aq. NaOH and extracted with tert-butyl methyl ether. The organic phase was dried and concentrated under vacuum. The liquid was purified by distilling (80° C./0.5 mmHg) to provide 50 g (46%) of 3-[(dimethylamino)methyl]-5-methylhexan-2-one as a colorless oil. ¹H-NMR (300 MHz, d₆-DMSO) δ 0.92 (d, 3H), 0.98 (d, 3H), 1.11-1.23 (m, 1H), 1.23-1.38 (m, 1H), 1.54-1.70 (m, 1H), 2.30 (s, 3H), 3.01 (s, 9H), 3.10-3.32 (m, 2H), 3.81-3.88 (m, 1H).

### Step 8

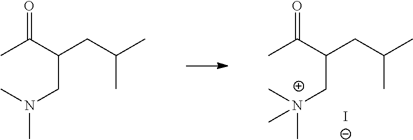

### 2-Acetyl-N,N,N,4-tetramethylpentan-1-aminium iodide

A solution of 3-[(dimethylamino)methyl]-5-methylhexan-2-one (50 g, 15.00 mmol, 1.00 equiv) and methyl iodide (4.26 g, 30.00 mmol, 2.00 equiv) in 50 mL diethyl ether was stirred overnight at room temperature. The precipitated solid was isolated by filtration and dried under vacuum to give 79 g (86%) of 2-acetyl-N,N,N,4-tetramethylpentan-1-aminium iodide as white solid. ¹H-NMR (300 MHz, d₆-DMSO) δ 0.89-0.98 (m, 6H), 1.11-1.20 (m, 1H), 1.40 (m, 1H), 1.66 (m, 1H), 2.30 (s, 3H), 3.01 (s, 9H), 3.21 (m, 2H), 3.85 (m, 1H).

### Step 9

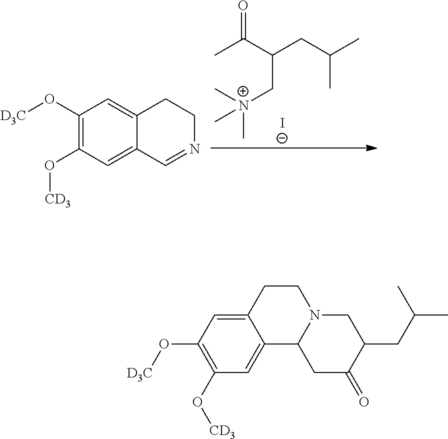 [SMILES: [2H]C([2H])([2H])Oc1cc2c(cc1OC([2H])([2H])[2H])CCN=C2 ; CC(=O)C(CC(C)C)C[N+](C)(C)C ; [I-] ; [2H]C([2H])([2H])Oc1cc2c(cc1OC([2H])([2H])[2H])C1CC(=O)C(CC(C)C)CN1CC2]

D₆-(±)-tetrabenazine:

A solution of d₆-6,7-dimethoxy-3,4-dihydroisoquinoline (33.4 g, 169 mmol, 1.10 equiv) and 2-acetyl-N,N,N,4-tetramethylpentan-1-aminium iodide (48 g, 153 mmol, 1.00 equiv) in 300 ml of methanol was heated under reflux for 48 h. Then 150 mL water was added. The solution was cooled to room temperature. The precipitated solid was isolated by filtration and dried under vacuum to give 38 g of crude d₆-tetrabenazine as yellow solid. The crude tetrabenazine was dissolved in tert-butyl methyl ether (15 volumes), the mixture was heated until the solid was almost dissolved. The yellow solid which was unsolvable was filtered. The filtrate was concentrated under vacuum until 2 volumes tert-butyl methyl ether was left. The solid was filtered and collected. The above solid was dissolved in ethanol (4 volumes), then the mixture was heated until the solid was dissolved. The solution was stirred and cooled to room temperature at the rate of 20° C./h. Then the mixture was stirred at 0° C. for 1 h. The precipitated solid was isolated by filtration and dried under vacuum to give 25 g (50.4%) of tetrabenazine-d₆ as white solid. ¹H-NMR (300 MHz, CD₂Cl₂) δ 6.61 (s, 1H), 6.55 (s, 1H), 3.84 (s, 3H), 3.82 (s, 3H), 3.50 (d, 1H, J=12 Hz), 3.27 (dd, 1H, J=11.4 Hz, J=6.3 Hz), 3.11 (m, 2H), 2.84 (dd, 1H, J=10.5 Hz, J=3 Hz), 2.74 (m, 2H), 2.56 (m, 2H), 2.31 (t, 1H, J=12 Hz), 1.76 (m, 1H), 1.63 (m, 1H), 0.98 (m, 1H), 0.89 (m, 6H). LC-MS: m/z=324 (MH)⁺.

### Example 2

### D₆-(±)-alpha-3-Isobutyl-9,10-dimethoxy-2,3,4,6,7,11b-hexahydro-1H-pyrido[2,1-a]isoquinolin-2-ol ((±)-alpha-dihydrotetrabenazine-d₆)

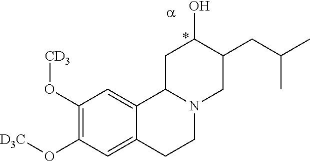 [SMILES: [2H]C([2H])([2H])Oc1cc2c(cc1OC([2H])([2H])[2H])C1CC(O)C(CC(C)C)CN1CC2]

### Step 1

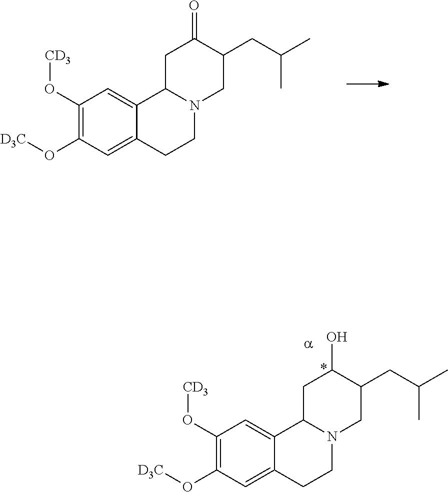 [SMILES: [2H]C([2H])([2H])Oc1cc2c(cc1OC([2H])([2H])[2H])C1CC(=O)C(CC(C)C)CN1CC2 ; [2H]C([2H])([2H])Oc1cc2c(cc1OC([2H])([2H])[2H])C1CC(O)C(CC(C)C)CN1CC2]

D₆-(±)-alpha-dihydrotetrabenazine:

To d₆-(±)-tetrabenazine (2 g, 6.18 mmol, 1.00 equiv) in 20 mL of ethanol at 0° C., was added NaBH₄ (470 mg, 12.36 mmol, 2.00 equiv) in several batches at 0° C. The reaction mixture was allowed to stir for 60 min at room temperature. The excess solvent was carefully removed under vacuum, and the residue was dissolved in 50 mL dichloromethane and washed with three portions of saturated aqueous brine. The combined organic extracts were dried over sodium sulfate, filtered, and concentrated under reduced pressure to provide a white solid. The solid was further purified by recrystallization from ethanol to afford 610 mg of d₆-(±)-alpha-dihydrotetrabenazine (30%) as a white solid. ¹H-NMR (300 MHz, CDCl₃) δ 6.68 (s, 1H), 6.59 (s, 1H), 3.42 (m, 1H), 3.42 (m, 4H), 2.63 (m, 2H), 2.49 (m, 1H), 2.01 (t, 1H, J=11.4 Hz), 1.75 (m, 2H), 1.56 (m, 3H), 1.05 (dd, 1H, J=9.9 Hz, J=13.8 Hz), 0.95 (m, 6H). MS: m/z=326 [M+H]⁺.

### Example 3

### D₆-(±)-beta-3-Isobutyl-9,10-dimethoxy-2,3,4,6,7,11b-hexahydro-1H-pyrido[2,1-a]isoquinolin-2-ol ((±)-beta-dihydrotetrabenazine-d₆)

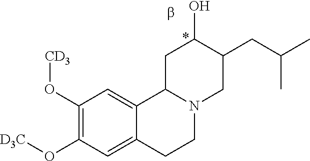 [SMILES: [2H]C([2H])([2H])Oc1cc2c(cc1OC([2H])([2H])[2H])C1CC(O)C(CC(C)C)CN1CC2]

### Step 1

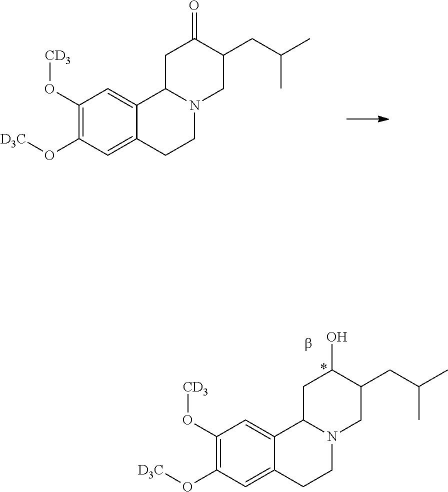 [SMILES: [2H]C([2H])([2H])Oc1cc2c(cc1OC([2H])([2H])[2H])C1CC(=O)C(CC(C)C)CN1CC2 ; [2H]C([2H])([2H])Oc1cc2c(cc1OC([2H])([2H])[2H])C1CC(O)C(CC(C)C)CN1CC2]

### D₆-(±)-beta-dihydrotetrabenazine

To d₆-(±)-tetrabenazine (1 g, 3.1 mmol, 1.00 equiv) in 20 mL of tetrahydrofuran at 0° C., was added dropwise potassium tri-sec-butyl borohydride (K-selectride) (1 M in tetrahydrofuran) (6.2 mL, 1.00 equiv) at 0° C. The reaction mixture was allowed to stir for 60 min at 0° C. HPLC showed that the reaction was completed. Then the mixture was poured into ice/water (30 mL). The solution was concentrated under vacuum to remove tetrahydrofuran and then extracted with dichloromethane. The combined organic extracts were dried over sodium sulfate, filtered, and concentrated under reduced pressure to provide white solid. The solid was purified by Prep-HPLC to afford 640 mg d₆-(±)-beta-dihydrotetrabenazine (63%) as white solid. ¹H-NMR (300 MHz, CDCl₃) δ 6.69 (s, 1H), 6.60 (s, 1H), 4.10 (s, 1H), 3.54 (m, 1H), 3.21 (m, 1H), 2.99 (m, 1H), 2.65 (m, 3H), 2.51 (m, 2H), 2.02 (m, 1H), 1.73 (m, 2H), 1.52 (m, 1H), 1.23 (m, 2H). MS: m/z=326 [M+H]⁺.

### Example 4

### 2-Acetyl-N,N,N,4-tetramethylpentan-1-aminium iodide

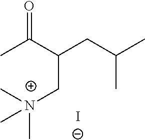

### Step 1

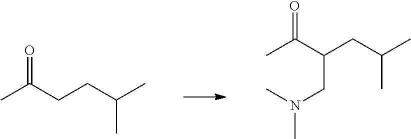

### 3-[(Dimethylamino)methyl]-5-methylhexan-2-one

A mixture of dimethylamine hydrochloride (3.78 kg, 46.22 mol, 1.30 equiv), paraformaldehyde (1.45 kg, 48.35 mol, 1.36 equiv), 5-methyl-2-hexanone (4.06 kg, 35.55 mol, 1.00 equiv) and conc. HCl (284 mL) in 95% ethanol (14.6 L) was refluxed for 24 hours under N2. Then ethanol was removed under reduced pressure. The orange-yellow residue was diluted with 5 L water and extracted with tert-butyl methyl ether (2×5.2 L). The pH value of aqueous layers was adjusted to 9 with 20% NaOH. The resulting solution was extracted with ethyl acetate (2×4 L). The organic layers was combined and concentrated under vacuum to give 1150 g of crude product as a yellow liquid (GC showed that 7% of the undesired isomer was contained). This was marked as product A. The pH value of above aqueous layers was adjusted to 9 with 20% NaOH again. The resulting solution was extracted with ethyl acetate (2×4 L). The organic layers was combined and concentrated under vacuum to give 1350 g of crude product as a yellow liquid (GC showed that 15% of the undesired isomer was contained). This was marked as product B. The product A was diluted with 3 L ethyl acetate, and 50 g toluenesulfonic acid was added, then the solution was stirred overnight at rt. The precipitated solid was removed. The filtrate was washed with water (2×400 mL) and 5% aqueous NaOH (200 mL). The product B was diluted with 3.5 L ethyl acetate, and 200 g toluenesulfonic acid was added, then the solution was stirred overnight at rt. The precipitated solid was removed and the filtrate was washed with water (2×400 mL) and 5% aqueous NaOH (200 mL). The two parts of above organic phase was dried over sodium sulfate and concentrated under vacuum to give 2.2 kg of 3-[(dimethylamino)methyl]-5-methylhexan-2-one (36%) as yellow liquid. (2% of the undesired isomer was found by GC). ¹H-NMR (300 MHz, d₆-DMSO) δ 0.92 (d, 3H), 0.98 (d, 3H), 1.11-1.23 (m, 1H), 1.23-1.38 (m, 1H), 1.54-1.70 (m, 1H), 2.30 (s, 3H), 3.01 (s, 9H), 3.10-3.32 (m, 2H), 3.81-3.88 (m, 1H). MS: m/z=172 [M+H]⁺.

### Step 2

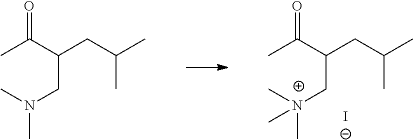

### 2-Acetyl-N,N,N,4-tetramethylpentan-1-aminium iodide

A solution of 3-[(dimethylamino)methyl]-5-methylhexan-2-one (2.2 kg, 12.84 mol, 1.00 equiv) in dichloromethane (10 L) was dropwised a solution of methyl iodide (2 kg, 14.12 mol, 1.1 equiv) in dichloromethane (2 L) at 5˜10° C. Then the solution was stirred overnight at rt. The reaction was monitored by LCMS until completion of reaction (3-[(dimethylamino)methyl]-5-methylhexan-2-one<5.0%). The precipitated solid was isolated by filtration and dried under vacuum to give 3.5 kg (87%) of 2-Acetyl-N,N,N,4-tetramethylpentan-1-aminium iodide as white solid. ¹H-NMR (300 MHz, d₆-DMSO) δ 0.89-0.98 (m, 6H), 1.11-1.20 (m, 1H), 1.40 (m, 1H), 1.66 (m, 1H), 2.30 (s, 3H), 3.01 (s, 9H), 3.21 (m, 2H), 3.85 (m, 1H). MS: m/z=186 [M+H]⁺.

### Example 5

### d₆-3-(2-hydroxy-2-methylpropyl)-9,10-dimethoxy-3,4,6,7-tetrahydro-1H-pyrido[2,1-a]isoquinolin-2(11bH)-one (racemic mixture of -(3S,11bS) and -(3R,11bR) enantiomers)

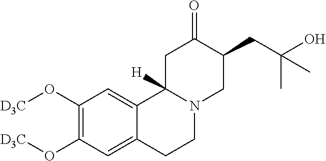 [SMILES: [2H]C([2H])([2H])Oc1cc2c(cc1OC([2H])([2H])[2H])[C@@H]1CC(=O)[C@@H](CC(C)(C)O)CN1CC2]

### Step 1

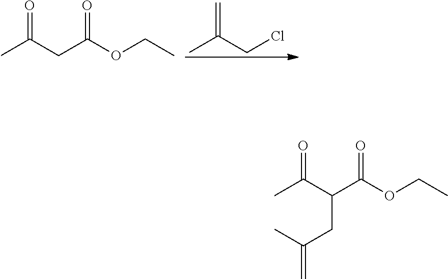

### Ethyl 2-acetyl-4-methylpent-4-enoate

To a solution of ethyl acetoacetate (500 g, 3.84 mol, 1.00 eq), potassium iodide (63.8 g, 0.384 mol, 0.10 eq), tetrabutylammonium bromide (136.2 g, 0.422 mol, 0.11 eq), and K2CO₃ (631.9 g, 4.57 mol, 1.19 eq) in dimethylformamide (1.5 L) was heated to 40-50° C. At this temperature, 3-chloro-2-methyl-1-propene (382.6 g, 4.22 mol, 1.10 eq) was added. The reaction mixture was heated to 65-75° C. and stirred for 6 hrs. Then the reaction mixture was cool to 25-35° C. and quenched with water (5.00 L). The product was extracted with toluene (2×2.00 L), and the combined toluene layers were washed with water (2×1.5 L) and concentrated under vacuum at 50-55° C. to give 707 g of ethyl 2-acetyl-4-methylpent-4-enoate (quantitative yield) as a brown liquid.

### Step 2

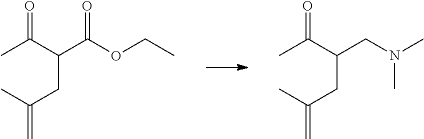

### 3-((Dimethylamino)methyl)-5-methylhex-5-en-2-one

To a solution of potassium hydroxide (234.5 g, 4.18 mol, 1.10 eq) in water (4.2 L) was added ethyl 2-acetyl-4-methylpent-4-enoate (700 g, 3.80 mol, 1.0 eq) and stirred at 25-35° C. for 4 hrs. The reaction mixture was washed with methyl tert-butyl ether (2×2.80 L). The pH of the aqueous layer was adjusted to 6.8-7.2 using concentrated hydrochloric acid. Then dimethylamine hydrochloride (464.8 g, 5.70 mol, 1.5 eq), 37% formaldehyde solution (474 mL, 6.36 mol, 1.675 eq) and tetrabutylammonium bromide (122.5 g, 0.38 mol, 0.10 eq) were added. Concentrated hydrochloric acid was added to the reaction mixture at 25-35° C. for 60-90 minutes until the pH of the reaction mixture was <1. Then the reaction mixture was stirred at 25-35° C. for 15 hrs. The reaction mixture was washed with methyl tert-butyl ether (2×2.8 L). The pH of the aqueous layer was adjusted to 9-10 by using 20% potassium hydroxide solution. Then the product was extracted with ethyl acetate (3×2.8 L). The ethyl acetate layer was washed with water (2×2.1 L), followed by 10% ammonium chloride solution (2×3.5 L). Then the ethyl acetate layer was treated with activated carbon (5% w/w), filtered through a bed of celite which was washed with ethyl acetate (350 mL). The filtrate was dried over sodium sulfate and distilled under vacuum at 40-45° C. to give 122 g of 3-((dimethylamino)methyl)-5-methylhex-5-en-2-one as a brown liquid (19% yield).

### Step 3

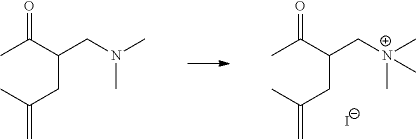

### 2-Acetyl-N,N,N,4-tetramethylpent-4-en-1-aminium iodide

To a solution of 3-((dimethylamino)methyl)-5-methylhex-5-en-2-one (40 g, 0.236 mol, 1.00 eq) in methyl tert-butyl ether (600 L) was added methyl iodide (77.25 g, 0.544 mol, 2.30 eq) at 0-10° C. for 1-2 hrs. Then the reaction mixture was stirred at 25-35° C. for 15 hrs and at 40-42° C. for 6 hrs. The reaction mixture was cooled to 25-35° C., filtered, and washed with methyl tert-butyl ether (400 L) to give 54 g of 2-acetyl-N,N,N,4-tetramethylpent-4-en-1-aminium iodide as off white solid (73.3% yield).

### Step 4

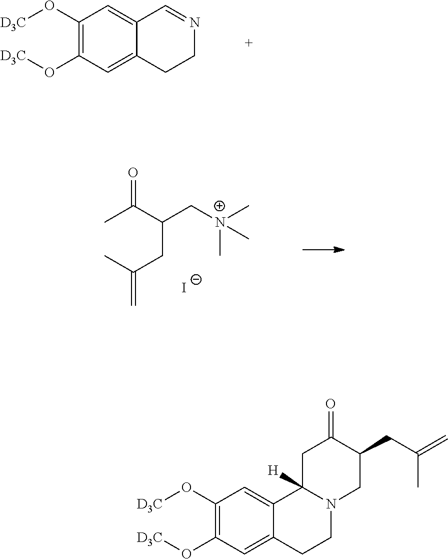 [SMILES: [2H]C([2H])([2H])Oc1cc2c(cc1OC([2H])([2H])[2H])CCN=C2 ; C=C(C)CC(C[N+](C)(C)C)C(C)=O ; [I-] ; [2H]C([2H])([2H])Oc1cc2c(cc1OC([2H])([2H])[2H])[C@@H]1CC(=O)[C@@H](CC(=C)C)CN1CC2]

### D₆-9,10-dimethoxy-3-(2-methylallyl)-3,4,6,7-tetrahydro-1H-pyrido[2,1-a]isoquinolin-2(11bH)-one (racemic mixture of -(3S,11bS) and -(3R,11bR) enantiomers)

To a solution of d₆-6,7-dimethoxy-3,4-dihydroisoquinoline (35 g, 0.149 mol, 1.00 eq) and 2-acetyl-N,N,N,4-tetramethylpent-4-en-1-aminium iodide (50.34 g, 0.161 mol, 1.08 eq) in 3:1 methanol water (210 mL) was added K₂CO₃ (20.71 g, 0.149 mol, 1.00 eq). The reaction mixture was heated to 40-45° C. for 30 hrs. Then the reaction mixture was cooled to room temperature (25-35° C.) and water was added (105 mL). The reaction mixture was stirred for 30 minutes. The precipitated solid was filtered, washed with water (105 mL), and dried to give 42 g of crude d₆-(3S,11bS)-9,10-dimethoxy-3-(2-methylallyl)-3,4,6,7-tetrahydro-1H-pyrido[2,1-a]isoquinolin-2(11bH)-one as a yellow solid. The crude product upon recrystallization using ethanol (3 volumes) gave 38 g d₆-(3S,11bS)-9,10-dimethoxy-3-(2-methylallyl)-3,4,6,7-tetrahydro-1H-pyrido[2,1-a]isoquinolin-2(11bH)-one (36% yield) as an off-white solid.

### Step 5

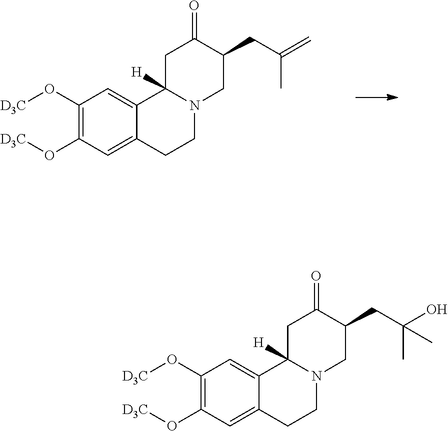 [SMILES: [2H]C([2H])([2H])Oc1cc2c(cc1OC([2H])([2H])[2H])[C@@H]1CC(=O)[C@@H](CC(=C)C)CN1CC2 ; [2H]C([2H])([2H])Oc1cc2c(cc1OC([2H])([2H])[2H])[C@@H]1CC(=O)[C@@H](CC(C)(C)O)CN1CC2]

### D₆-3-(2-hydroxy-2-methylpropyl)-9,10-dimethoxy-3,4,6,7-tetrahydro-1H-pyrido[2,1-a]isoquinolin-2(11bH)-one (racemic mixture of -(3S,11bS) and -(3R,11bR) enantiomers)

d₆-(3S,11bS)-9,10-dimethoxy-3-(2-methylallyl)-3,4,6,7-tetrahydro-1H-pyrido[2,1-a]isoquinolin-2(11bH)-one (2 g, 0.0062 mol, 1.00 eq) was taken up in aqueous sulfuric acid (3.6 M, 40 mL) and stirred for 18 hrs at 25-35° C. The reaction mixture was cooled to 0-5° C. and adjusted to pH to 9-10 by using 5% NaOH solution. The product was extracted with ethyl acetate (2×75 mL). The ethyl acetate layer was washed with water (2×25 mL). The ethyl acetate layer was dried with sodium sulfate and distilled under vacuum at 40-45° C. to give 2 g of crude d₆-(3S,11bS)-3-(2-hydroxy-2-methylpropyl)-9,10-dimethoxy-3,4,6,7-tetrahydro-1H-pyrido[2,1-a]isoquinolin-2(11bH)-one (94.7%) as an off white solid. This crude compound was purified by recrystallization from ethanol (12 mL) to give 0.78 g of pure d₆-(3S,11bS)-3-(2-hydroxy-2-methylpropyl)-9,10-dimethoxy-3,4,6,7-tetrahydro-1H-pyrido[2,1-a]isoquinolin-2(11bH)-one as an white solid (36.9% yield).

### Example 6

### d₆-3-(2-hydroxy-2-methylpropyl)-9,10-dimethoxy-3,4,6,7-tetrahydro-1H-pyrido[2,1-a]isoquinolin-2(11bH)-one (mixture of diastereomers)

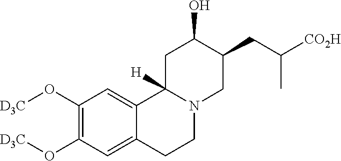 [SMILES: [2H]C([2H])([2H])Oc1cc2c(cc1OC([2H])([2H])[2H])[C@@H]1C[C@@H](O)[C@@H](CC(C)C(=O)O)CN1CC2]

### Step 1

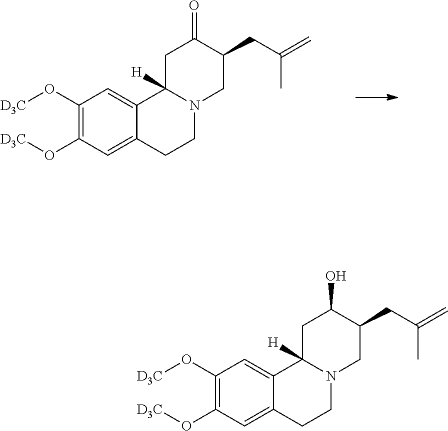 [SMILES: [2H]C([2H])([2H])Oc1cc2c(cc1OC([2H])([2H])[2H])[C@@H]1CC(=O)[C@@H](CC(=C)C)CN1CC2 ; [2H]C([2H])([2H])Oc1cc2c(cc1OC([2H])([2H])[2H])[C@@H]1C[C@@H](O)[C@@H](CC(=C)C)CN1CC2]

### D₆-9,10-dimethoxy-3-(2-methylallyl)-2,3,4,6,7,11b-hexahydro-1H-pyrido[2,1-a]isoquinolin-2-ol (mixture of diastereomers)

To a solution of (3S,11bS)-9,10-dimethoxy-3-(2-methylallyl)-3,4,6,7-tetrahydro-1H-pyrido[2,1-a]isoquinolin-2(11bH)-one (20 g, 0.0623 mol, 1.00 eq) in tetrahydrofuran (300 mL) was added potassium sec-butylborohydride (1M) (74.76 mL, 0.0747 mol, 1.2 eq) at 0-5° C. for 30 minutes and the reaction mixture was stirred for 30 minutes. Water (200 mL) was added to the reaction mixture and stirred for 15 minutes. The reaction mixture was concentrated under vacuum at 40° C. until complete removal of tetrahydrofuran. The precipitated solid was filtered and washed with water (400 mL) to give 19.6 g [00116] d₆-(2R,3S,11bS)-9,10-dimethoxy-3-(2-methylallyl)-2,3,4,6,7,11b-hexahydro-1H-pyrido[2,1-a]isoquinolin-2-ol as an orange solid (97.4% yield).

### Step 2

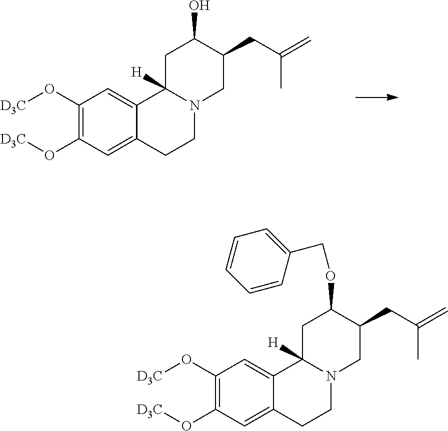 [SMILES: [2H]C([2H])([2H])Oc1cc2c(cc1OC([2H])([2H])[2H])[C@@H]1C[C@@H](O)[C@@H](CC(=C)C)CN1CC2 ; [2H]C([2H])([2H])Oc1cc2c(cc1OC([2H])([2H])[2H])[C@@H]1C[C@@H](OCc3ccccc3)[C@@H](CC(=C)C)CN1CC2]

### D₆-2-(benzyloxy)-9,10-dimethoxy-3-(2-methylallyl)-2,3,4,6,7,11b-hexahydro-1H-pyrido[2,1-a]isoquinoline (mixture of diastereomers)

To a solution of d₆-(2R,3S,11bS)-9,10-dimethoxy-3-(2-methylallyl)-2,3,4,6,7,11b-hexahydro-1H-pyrido[2,1-a]isoquinolin-2-ol (22 g, 0.0681 mol, 1.00 eq) in dimethylforamide (220 mL) was added sodium hydride portion wise at 0-5° C. under a nitrogen atmosphere. The reaction mixture was slowly heated to 25-35° C. and stirred for 1 hr. Benzyl bromide (8.14 mL, 0.06811, 1.00 eq) was added to the reaction mass at 0-5° C. over 20 minutes and stirred for 30 minutes. The reaction mixture was quenched with cold water (440 mL) at 0-5° C. and the compound was extracted with ethyl acetate (2×220 mL and 1×110 mL). The combined organic layers were washed with water (3×110 mL), dried over sodium sulfate, and distilled under vacuum at 40-45° C. to give crude d₆-(2R,3S,11bS)-2-(benzyloxy)-9,10-dimethoxy-3-(2-methylallyl)-2,3,4,6,7,11b-hexahydro-1H-pyrido[2,1-a]isoquinoline in quantitative yield as a dark brown thick liquid. Purification by chromatography (25% ethyl acetate in hexane) gave 8.62 g of d₆-(2R,3S,11bS)-2-(benzyloxy)-9,10-dimethoxy-3-(2-methylallyl)-2,3,4,6,7,11b-hexahydro-1H-pyrido[2,1-a]isoquinoline as a pale yellow solid (30.6% yield).

### Step 3

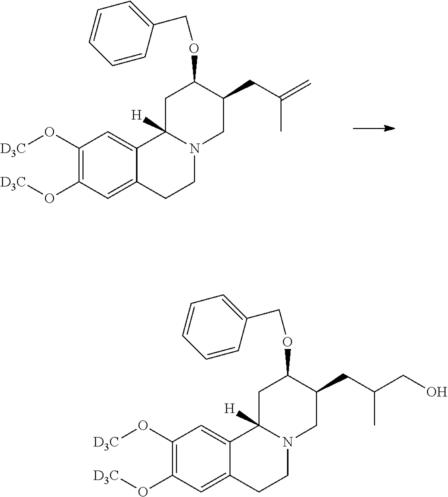 [SMILES: [2H]C([2H])([2H])Oc1cc2c(cc1OC([2H])([2H])[2H])[C@@H]1C[C@@H](OCc3ccccc3)[C@@H](CC(=C)C)CN1CC2 ; [2H]C([2H])([2H])Oc1cc2c(cc1OC([2H])([2H])[2H])[C@@H]1C[C@@H](OCc3ccccc3)[C@@H](CC(C)CO)CN1CC2]

### D₆-2-(benzyloxy)-9,10-dimethoxy-2,3,4,6,7,11b-hexahydro-1H-pyrido[2,1-a]isoquinolin-3-yl)-2-methylpropan-1-ol (mixture of diastereomers)

To a solution of d₆-(2R,3S,11bS)-2-(benzyloxy)-9,10-dimethoxy-3-(2-methylallyl)-2,3,4,6,7,11b-hexahydro-1H-pyrido[2,1-a]isoquinoline (11 g, 0.0266 mol, 1.00 eq) in tetrahydrofuran (110 mL) was added borane-dimethylsulfide (4.79 mL, 0.0479 mol, 1.8 eq, 10 M solution) over 30 minutes at 0-5° C. under nitrogen atmosphere. The reaction mixture was stirred overnight at 25-30° C. The reaction mixture was quenched with 3M NaOH solution (22 mL) at 0-5° C. The reaction mixture was concentrated under vacuum at 40° C. until complete removal of tetrahydrofuran and co-distilled twice with diethyl ether (2×110 mL). 3 M aqueous NaOH solution (55 mL) was added to the remaining residue and heated to 80-90° C. for 2 hrs. The reaction mixture was cooled to 25-30° C. and the product was extracted with ethyl acetate (3×110 mL). The combined organic layers were washed with water (3×110 mL), dried over sodium sulfate, and distilled under vacuum at 40-45° C. to give 11.74 g of crude d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropan-1-ol as a dark brown viscous liquid (quantitative yield). Purification of the crude product by chromatography (1% methanol in ethyl acetate) gave 3.26 g of d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropan-1-ol as a brown viscous liquid which solidified upon standing overnight (28.4% yield).

### Step 4

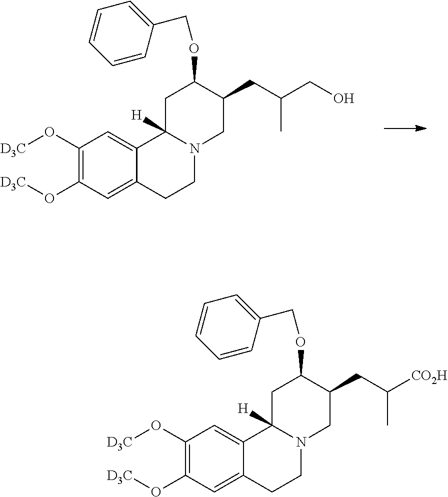 [SMILES: [2H]C([2H])([2H])Oc1cc2c(cc1OC([2H])([2H])[2H])[C@@H]1C[C@@H](OCc3ccccc3)[C@@H](CC(C)CO)CN1CC2 ; [2H]C([2H])([2H])Oc1cc2c(cc1OC([2H])([2H])[2H])[C@@H]1C[C@@H](OCc3ccccc3)[C@@H](CC(C)C(=O)O)CN1CC2]

### D₆-2-(benzyloxy)-9,10-dimethoxy-2,3,4,6,7,11b-hexahydro-1H-pyrido[2,1-a]isoquinolin-3-yl)-2-methylpropanoic acid (mixture of diastereomers)

To a solution of d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropan-1-ol (3.2 g, 0.00742 mol, 1.00 eq) in acetone (64 mL) was added freshly prepared Jones reagent at 20° C. in 30 minutes. The reaction mixture was stirred at 20° C. for 30 minutes. The liquid layer was decanted and to the remaining green color gummy mass, acetone (64 mL) was added, stirred for 30 minutes, and decanted. The pH of the combined acetone layers were adjusted to 7 using saturated sodium bicarbonate solution (20 mL). The solids were filtered and washed with acetone (60 mL). The filtrate was distilled under vacuum at 35° C. until complete removal of acetone. The remaining aqueous layer was saturated with sodium chloride and extracted with ethyl acetate (5×60 mL). The combined organic layers were dried over sodium sulfate and concentrated under vacuum at 40-45° C. to give 1.5 g of crude d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropanoic acid (45.4% yield). Purification of the crude product by recrystallization from ethyl acetate (1 volume) gave 0.43 g d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropanoic acid as a pale green solid (13% yield).

Preparation of Jones Reagent:

To a solution of CrO₃ (1.11 g, 0.0111 mol, 1.5 eq) in water (2.04 mL) was added concentrated sulfuric acid (0.928 mL) at 25-30° C. To the reaction mixture, water (1 mL) was added to dissolve the remaining salts. This reagent (orange color clear liquid) was prepared afresh and used for the oxidation reaction.

### Step 5

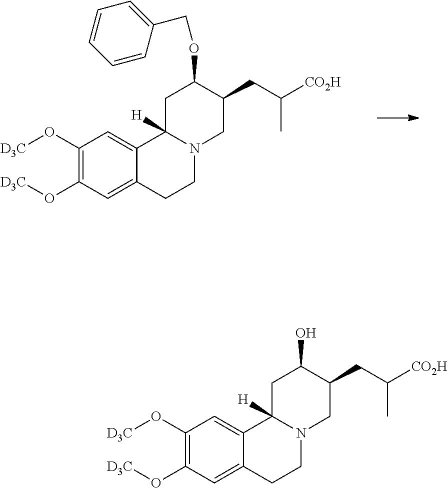 [SMILES: [2H]C([2H])([2H])Oc1cc2c(cc1OC([2H])([2H])[2H])[C@@H]1C[C@@H](OCc3ccccc3)[C@@H](CC(C)C(=O)O)CN1CC2 ; [2H]C([2H])([2H])Oc1cc2c(cc1OC([2H])([2H])[2H])[C@@H]1C[C@@H](O)[C@@H](CC(C)C(=O)O)CN1CC2]

### D₆-3-(2-hydroxy-2-methylpropyl)-9,10-dimethoxy-3,4,6,7-tetrahydro-1H-pyrido[2,1-a]isoquinolin-2(11bH)-one (mixture of diastereomers)

To a solution of d₆-3-((2R,3S,11bS)-2-(benzyloxy)-9,10-dimethoxy-2,3,4,6,7,11b-hexahydro-1H-pyrido[2,1-a]isoquinolin-3-yl)-2-methylpropanoic acid (0.5 g, 0.0011 mol, 1.00 eq) in methanol (150 mL) was added 20% Pd/C (0.25 g, 50% w/w). The reaction mixture was heated to 50-55° C. for 16 hrs. The reaction mixture was cooled to room temperature (25-35° C.), filtered through a celite bed which was washed with methanol (150 mL). The filtrate was distilled under vacuum at 40-45° C. to give 0.39 g of crude d₆-3-((2R,3S,11bS)-2-hydroxy-9,10-dimethoxy-2,3,4,6,7,11b-hexahydro-1H-pyrido[2,1-a]isoquinolin-3-yl)-2-methylpropanoic acid as off-white solid (quantitative yield). This crude compound was purified by preparative HPLC to obtain 70 mg of d₆-3-((2R,3S,11bS)-2-hydroxy-9,10-dimethoxy-2,3,4,6,7,11b-hexahydro-1H-pyrido[2,1-a]isoquinolin-3-yl)-2-methylpropanoic acid as a white solid (17.5% yield).

### Example 7

### d₆-3-(2-hydroxy-2-methylpropyl)-9,10-dimethoxy-3,4,6,7-tetrahydro-1H-pyrido[2,1-a]isoquinolin-2(11bH)-one (racemic mixture)

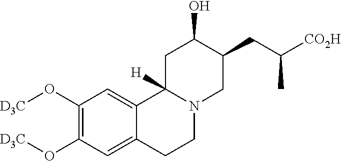 [SMILES: [2H]C([2H])([2H])Oc1cc2c(cc1OC([2H])([2H])[2H])[C@@H]1C[C@@H](O)[C@@H](C[C@H](C)C(=O)O)CN1CC2]

### Step 1

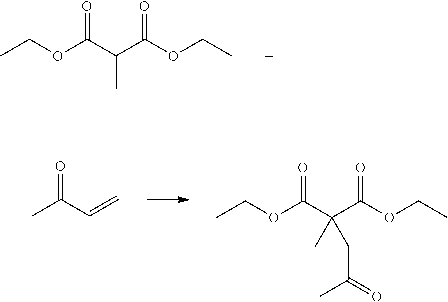

### 1,3-Diethyl 2-methyl-2-(3-oxobutyl)propanedioate

To a solution of 1,3-diethyl 2-methylpropanedioate (500 g, 2.87 mol, 1.00 equiv) in dichloromethane (5000 mL) were added but-3-en-2-one (302 g, 4.31 mol, 1.50 equiv) and potassium carbonate (793 g, 5.74 mol, 2.00 equiv). The resulting solution was stirred for 48 h at 25° C. The reaction mixture was then quenched by the addition of water (5 L). The dichloromethane layer was separated. The resulting aqueous solution was extracted with dichloromethane (2×1000 mL). The organic layers were combined, washed with hydrochloric acid (1M, 2×2000 mL), water (1×2000 mL), brine (1×2000 mL), dried over anhydrous sodium sulfate, filtered and concentrated under vacuum to afford 590 g (crude, 91% yield) of 1,3-diethyl 2-methyl-2-(3-oxobutyl)propanedioate as a light yellow oil.

¹H NMR (400 MHz, CDCl₃) δ: 4.22-4.14 (m, 4H), 2.52-2.48 (m, 2H), 2.15 (s, 3H), 2.14-2.11 (m, 2H), 1.40 (s, 3H), 1.27-1.24 (m, 6H).

### Step 2

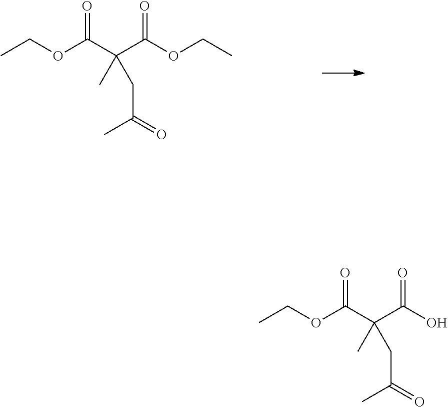 [SMILES: CCOC(=O)C(C)(CC(C)=O)C(=O)OCC ; CCOC(=O)C(C)(CC(C)=O)C(=O)O]

### 2-(Ethoxycarbonyl)-2-methyl-5-oxohexanoic acid

To a solution of 1,3-diethyl 2-methyl-2-(3-oxobutyl)propanedioate (415 g, 1.70 mol, 1.00 equiv) in ethanol (2500 mL) was added aqueous sodium hydroxide solution (10%, 71 g, 1.05 equiv) dropwise with stifling at 0° C. in 15 min. The resulting solution was stirred for 3 h at 25° C. Ethanol was removed under vacuum. The aqueous solution was diluted with water (1000 mL) and washed with ethyl acetate (3×500 mL). The pH of the solution was adjusted to 2 with hydrochloric acid solution (10%). The resulting solution was extracted with ethyl acetate (4×600 mL). The organic layers were combined, washed with water (1×1000 mL), brine (2×1000 mL), dried over anhydrous sodium sulfate and concentrated under vacuum to afford 350 g (95% yield) of 2-(ethoxycarbonyl)-2-methyl-5-oxohexanoic acid as a light yellow oil.

¹H NMR (400 MHz, CDCl₃) δ: 4.16-4.10 (m, 2H), 2.52-2.42 (m, 2H), 2.25 (s, 3H), 1.92-1.83 (m, 1H), 1.79-1.70 (m, 1H), 1.28-1.23 (m, 3H), 1.18-1.15 (m, 3H).

### Step 3

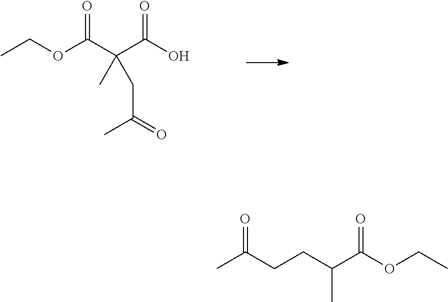

### Ethyl 2-methyl-5-oxohexanoate

2-(ethoxycarbonyl)-2-methyl-5-oxohexanoic acid (350 g, 1.62 mol, 1.00 equiv) was dissolved in dimethylsulfoxide (2000 mL) and water (20 mL). The resulting solution was stirred for 2 h at 160° C. The reaction mixture was then quenched by the addition of water/ice (3000 mL). The resulting solution was extracted with ethyl acetate (4×600 mL) and the organic layers were combined, washed with water (2×1000 mL), brine (2×1000 mL), dried over anhydrous sodium sulfate and concentrated under vacuum to afford 185 g (66% yield) of ethyl 2-methyl-5-oxohexanoate as a light yellow oil.

¹H NMR (400 MHz, CDCl₃) δ: 4.19-4.10 (m, 2H), 2.52-2.42 (m, 2H), 2.13 (s, 3H), 1.92-1.83 (m, 1H), 1.79-1.77 (m, 1H), 1.29-1.21 (m, 3H), 1.18-1.16 (m, 3H).

### Step 4

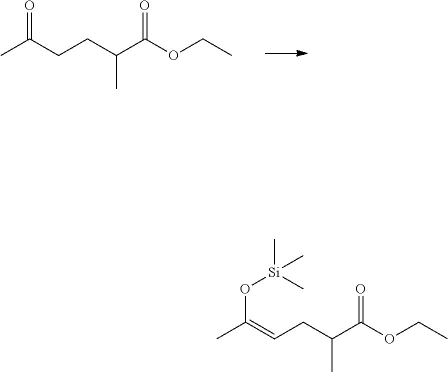 [SMILES: CCOC(=O)C(C)CCC(C)=O ; CCOC(=O)C(C)C/C=C(/C)O[Si](C)(C)C]

### Ethyl (4Z)-2-methyl-5-[(trimethylsilyl)oxy]hex-4-enoate

To a solution of ethyl 2-methyl-5-oxohexanoate (180 g, 1.05 mol, 1.00 equiv), in dichloromethane (2000 mL) was added hexamethyldisilazide (505 g, 3.13 mol, 3.00 equiv) under an atmosphere of nitrogen followed by the addition of trimethylsilyl iodide (209 g, 1.04 mol, 1.00 equiv) dropwise with stirring at −30 to −20° C. in 30 min. The reaction temperature was allowed to rise to 25° C. and stirred for 5 h at 25° C. The reaction mixture was then quenched by the addition of cooled sat. NaHCO₃ (2 L). Dichloromethane layer was separated and the resulting aqueous solution was extracted with dichloromethane (2×500 mL). The organic layers were combined, washed with water (6×1000 mL), brine (1×1000 mL), dried over anhydrous sodium sulfate and concentrated under vacuum to afford 230 g (crude, 90% yield) of ethyl (4Z)-2-methyl-5-[(trimethylsilyl)oxy]hex-4-enoate as a yellow oil.

¹H NMR (300 MHz, CDCl₃) δ: 4.59-4.36 (m, 1H), 4.14-4.03 (m, 2H), 2.42-2.13 (m, 3H), 1.75 (s, 3H), 1.26-1.21 (m, 3H), 1.16-1.10 (m, 3H), 0.20-0.11 (m, 9H).

### Step 5

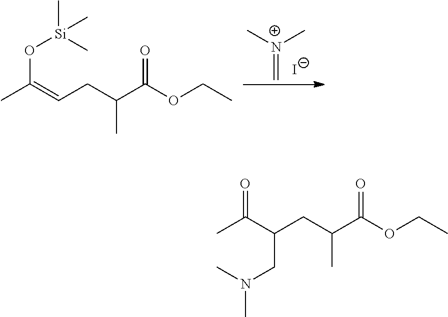

### Ethyl 4-[(dimethylamino)methyl]-2-methyl-5-oxohexanoate

To a solution of (4Z)-2-methyl-5-[(trimethylsilyl)oxy]hex-4-enoate (230 g, 941.07 mmol, 1.00 equiv) in acetonitrile (1500 mL) was added dimethyl(methylidene)azanium iodide (174.4 g, 942.67 mmol, 1.00 equiv) in several batches at 0° C. in 20 min. The resulting solution was stirred for 20 h at 25° C. under an atmosphere of nitrogen. The resulting mixture was concentrated under vacuum. The residue was purified by SiO₂ chromatography eluted with ethyl acetate/petroleum ether (1:1) to afford 160 g (74% yield) of ethyl 4-[(dimethylamino)methyl]-2-methyl-5-oxohexanoate as a dark red oil.

¹H NMR (300 MHz, CDCl₃) δ: 4.18-4.06 (m, 2H), 2.78-2.34 (m, 4H), 2.23-2.20 (m, 6H), 2.18-2.15 (m, 2H), 1.98-1.94 (m, 1H), 1.75-1.65 (m, 1H), 1.30-1.21 (m, 4H), 1.17-1.13 (m, 3H). LC-MS: m/z=230 [M+H]⁺.

### Step 6

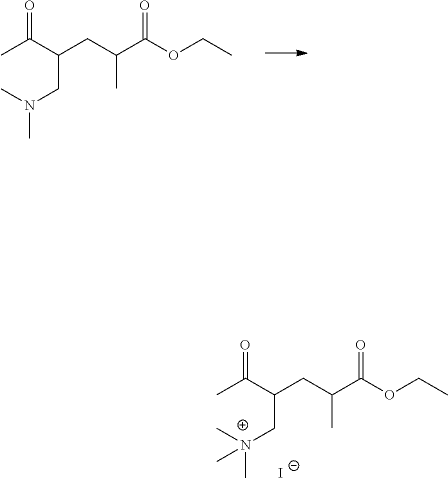 [SMILES: CCOC(=O)C(C)CC(CN(C)C)C(C)=O ; CCOC(=O)C(C)CC(C[N+](C)(C)C)C(C)=O ; [I-]]

### (2-Acetyl-5-ethoxy-4-methyl-5-oxopentyl)trimethylazanium iodide

Ethyl 4-[(dimethylamino)methyl]-2-methyl-5-oxohexanoate (160 g, 697.73 mmol, 1.00 equiv) was dissolved in iodomethane (992 g, 6.99 mol, 10.00 equiv) and the resulting solution was stirred for 15 h at 25° C. under an atmosphere of nitrogen. The resulting mixture was concentrated under vacuum to give 180 g (crude, 69% yield) of (2-acetyl-5-ethoxy-4-methyl-5-oxopentyl)trimethylazanium iodide as a dark red oil.

### Step 7

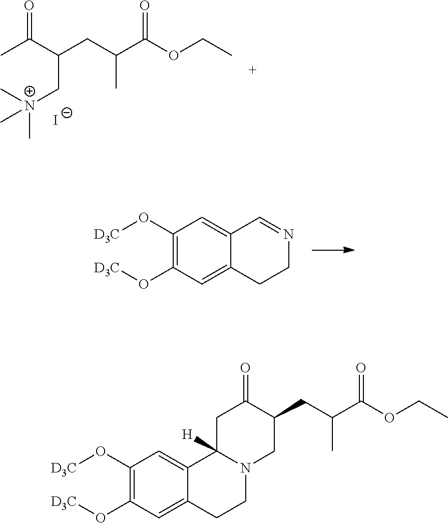 [SMILES: CCOC(=O)C(C)CC(C[N+](C)(C)C)C(C)=O ; [I-] ; [2H]C([2H])([2H])Oc1cc2c(cc1OC([2H])([2H])[2H])CCN=C2 ; [2H]C([2H])([2H])Oc1cc2c(cc1OC([2H])([2H])[2H])[C@@H]1CC(=O)[C@@H](CC(C)C(=O)OCC)CN1CC2]

### Ethyl 3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoate

To a solution of 6,7-bis(d₃)methoxy-3,4-dihydroisoquinoline (40 g, 202.77 mmol, 1.00 equiv) in ethanol (400 mL) was added (2-acetyl-5-ethoxy-4-methyl-5-oxopentyl)trimethylazanium iodide (113 g, 304.37 mmol, 1.50 equiv). The resulting solution was stirred for 30 h at 90° C. under an atmosphere of nitrogen. The reaction progress was monitored by LCMS. The resulting mixture was concentrated under vacuum. The residue was dissolved in ethyl acetate (1000 mL), washed with brine (2×500 mL), dried over anhydrous sodium sulfate and concentrated under vacuum. The residue was purified by SiO₂ chromatography eluted with ethyl acetate/petroleum ether (1:1) to afford 40 g (52% yield) of ethyl 3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoate as a light yellow solid.

¹H NMR (300 MHz, DMSO-d₆) δ: 6.67 (s, 2H), 4.09-4.01 (m, 2H), 3.49-3.45 (m, 1H), 3.32-3.24 (m, 1H), 3.12-3.08 (m, 1H), 2.92-2.81 (m, 2H), 2.69-2.61 (m, 2H), 2.49-2.31 (m, 5H), 1.20-1.10 (m, 4H), 1.06-1.14 (m, 3H). LC-MS: m/z=382 [M+H]⁺.

### Step 8

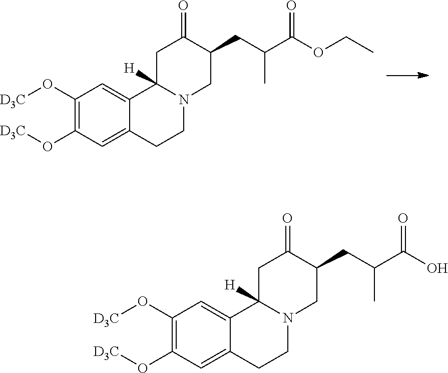 [SMILES: [2H]C([2H])([2H])Oc1cc2c(cc1OC([2H])([2H])[2H])[C@@H]1CC(=O)[C@@H](CC(C)C(=O)OCC)CN1CC2 ; [2H]C([2H])([2H])Oc1cc2c(cc1OC([2H])([2H])[2H])[C@@H]1CC(=O)[C@@H](CC(C)C(=O)O)CN1CC2]

### 3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoic acid

To a solution of ethyl-3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoate (42 g, 110.09 mmol, 1.00 equiv) in tetrahydrofuran (400 mL) and water (200 mL) was added LiOH (6.6 g, 275.57 mmol, 2.50 equiv). The resulting solution was stirred for 3 h at 25° C. Tetrahydrofuran was removed under vacuum. The resulting aqueous solution was washed with ethyl acetate (3×200 mL). The pH of the aqueous solution was adjusted to 5-6 with hydrogen chloride (2 mol/L). The solid was collected by filtration, dried in an oven to afford 32 g (82% yield) of 3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoic acid as an off-white solid.

¹H NMR (300 MHz, DMSO-d₆) δ: 12.12 (brs, 1H), 6.68 (s, 2H), 3.46 (m, 1H), 3.25-3.21 (m, 1H), 3.19-3.06 (m, 1H), 3.00-2.83 (m, 2H), 2.69-2.60 (m, 2H), 2.50-2.30 (m, 3H), 1.81-1.71 (m, 1H), 1.37-1.28 (m, 1H), 1.07 (m, 3H). LC-MS: m/z=354 [M+H]⁺.

### Step 9

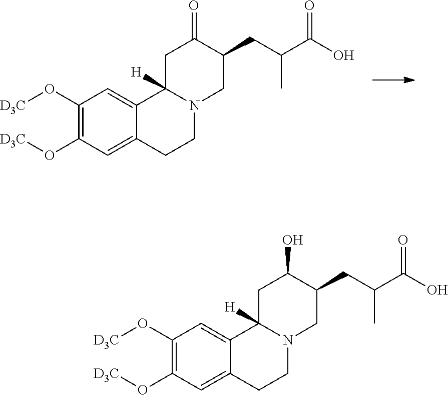 [SMILES: [2H]C([2H])([2H])Oc1cc2c(cc1OC([2H])([2H])[2H])[C@@H]1CC(=O)[C@@H](CC(C)C(=O)O)CN1CC2 ; [2H]C([2H])([2H])Oc1cc2c(cc1OC([2H])([2H])[2H])[C@@H]1C[C@@H](O)[C@@H](CC(C)C(=O)O)CN1CC2]

### 3-[((2S,3R,11bR)/(2R,3S,11bS)-2-hydroxy-9,10-bis(d₃)methoxy-1H,2H,3H,4H,6H,7H,11bH-pyrido[2,1-a]isoquinolin-3-yl]-2-methylpropanoic acid

To a suspension of 3-[(3R,11bR)/(3S,11bS)-9,10-bis(d₃)methoxy-2-oxo-1H,2H,3H,4H,6H,7H,11bH-pyrido[2,1-a]isoquinolin-3-yl]-2-methylpropanoic acid (36.8 g, 104.12 mmol, 1.00 equiv) in tetrahydrofuran (400 mL) under an atmosphere of nitrogen was added K-selectride (1 M in THF, 208 mL, 2.00 equiv) dropwise with stirring at −30-20° C. in 30 min. The resulting suspension was stirred for 2 h at −10-0° C. and turned into a solution. The reaction progress was monitored by LCMS. The reaction mixture was then quenched by the addition of water/ice (300 mL). The reaction mixture was concentrated under vacuum to remove THF. The resulting aqueous solution was extracted with dichloromethane (3×100 mL) and the pH of the aqueous layers were adjusted to 6 with hydrogen chloride (2N). The solid was collected by filtration, dried in an oven under reduced pressure to afford 20 g (54% yield, 63% purity) of 3-[(2S,3R,11bR)/(2R,3S,11bS)-2-hydroxy-9,10-bis(d₃)methoxy-1H,2H,3H,4H,6H,7H, 11bH-pyrido[2,1-a]isoquinolin-3-yl]-2-methylpropanoic acid as a colorless solid.

LC-MS: m/z=356 [M+H]⁺.

### Step 10

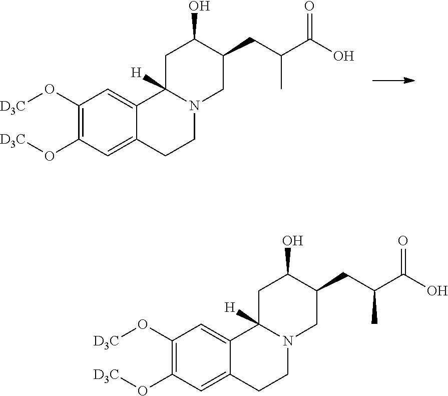 [SMILES: [2H]C([2H])([2H])Oc1cc2c(cc1OC([2H])([2H])[2H])[C@@H]1C[C@@H](O)[C@@H](CC(C)C(=O)O)CN1CC2 ; [2H]C([2H])([2H])Oc1cc2c(cc1OC([2H])([2H])[2H])[C@@H]1C[C@@H](O)[C@@H](C[C@H](C)C(=O)O)CN1CC2]

### (2R)-3-[(2S,3R,11bR)/(2R,3S,11bS)-2-hydroxy-9,10-bis(d₃)methoxy-1H,2H,3H,4H,6H,7H,11bH-pyrido[2,1-a]isoquinolin-3-yl]-2-methylpropanoic acid

The solid 3-[(2S,3R,11bR)/(2R,3S,11bS)-2-hydroxy-9,10-bis(d₃)methoxy-1H,2H,3H,4H,6H,7H,11bH-pyrido[2,1-a]isoquinolin-3-yl]-2-methylpropanoic acid (20 g, 56.27 mmol, 1.00 equiv) was dissolved in 1000 mL of aqueous sodium hydroxide solution (0.5 M) and the pH of the solution was adjusted to 8 with hydrochloric acid (2N). The solid was precipitated from water, then the pH value of the suspension was adjusted to 4 with hydrochloric acid (0.5 N). The solid was dissolved. A sodium hydroxide solution (0.5 N) was used to adjust the pH od the solution to 7 immediately. The solid precipitated and were collected by filtration. LCMS showed the purity of the product was 87%. This process was repeated two times and the purity of the product was 96% in LCMS. Then the product was suspended in ethanol (200 mL) and stirred for 20 min at 70° C. The solid was collected by filtration to afford 8 g (40% yield, 98% purity) of (2R)-3-[(2S,3R,11bR)/(2R,3S,11bS)-2-hydroxy-9,10-bis(d₃)methoxy-1H,2H,3H,4H,6H,7H,11bH-pyrido[2,1-a]isoquinolin-3-yl]-2-methylpropanoic acid as a colorless solid.

¹H NMR (300 MHz, DMSO-d₆) δ: 6.62 (s, 1H), 6.61 (s, 1H), 4.54 (brs, 1H), 3.90 (s, 1H), 3.49-3.46 (m, 1H), 2.91-2.83 (m, 2H), 2.55-2.54 (m, 1H), 2.45-2.27 (m, 5H), 1.61-1.60 (m, 1H), 1.39-1.32 (m, 3H), 1.07-1.05 (m, 3H). LC-MS: m/z=356 [M+H]⁺.

The following compounds can generally be made using the methods described above. It is expected that these compounds when made will have activity similar to those described in the examples above.

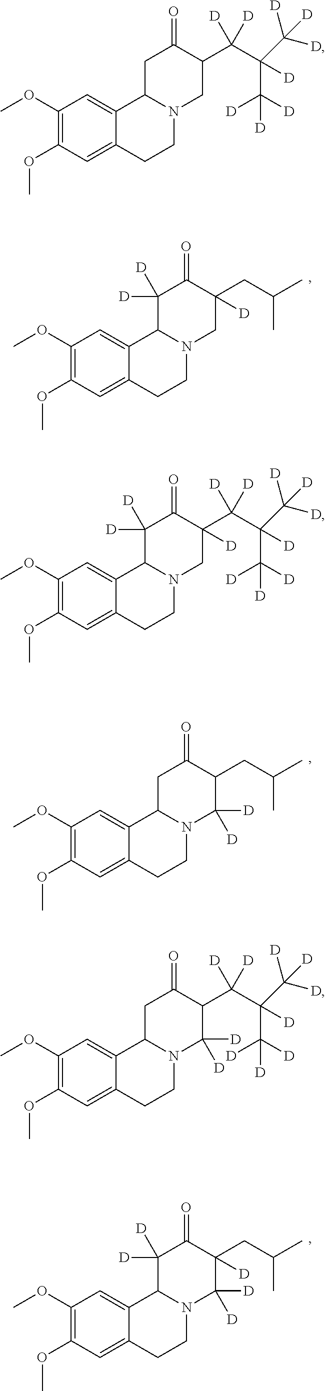

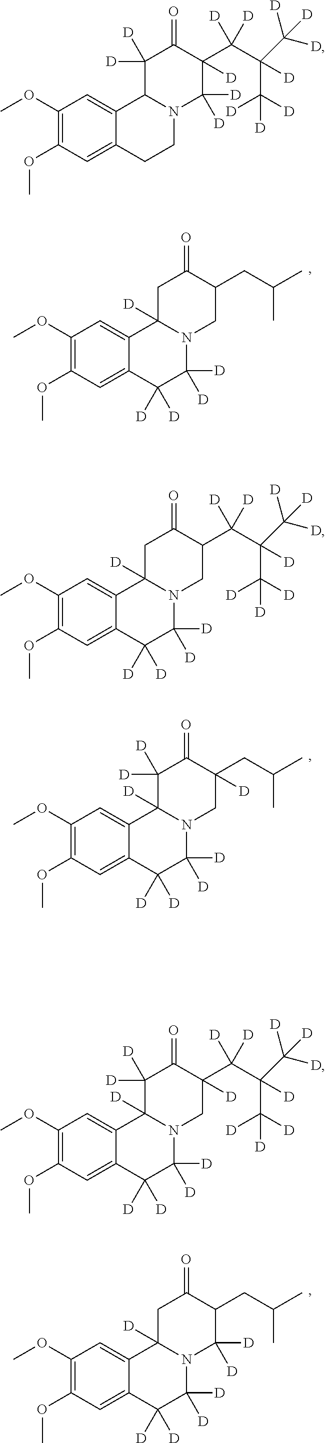

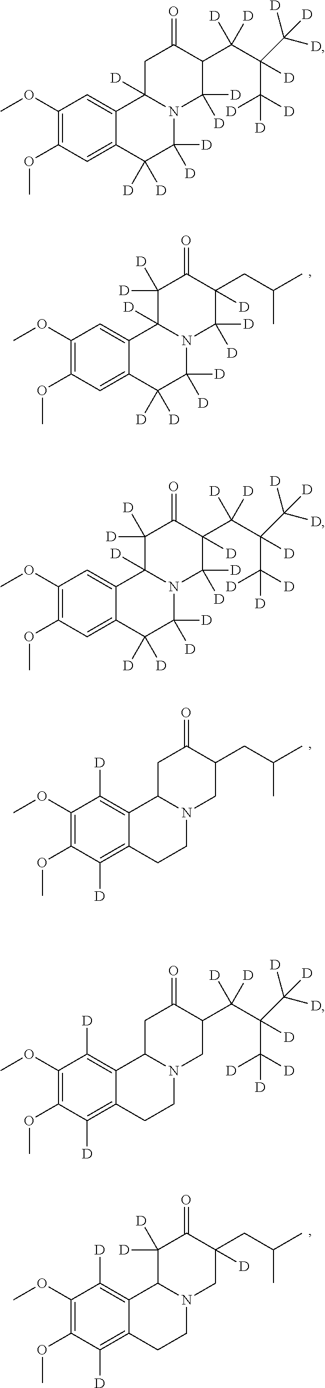

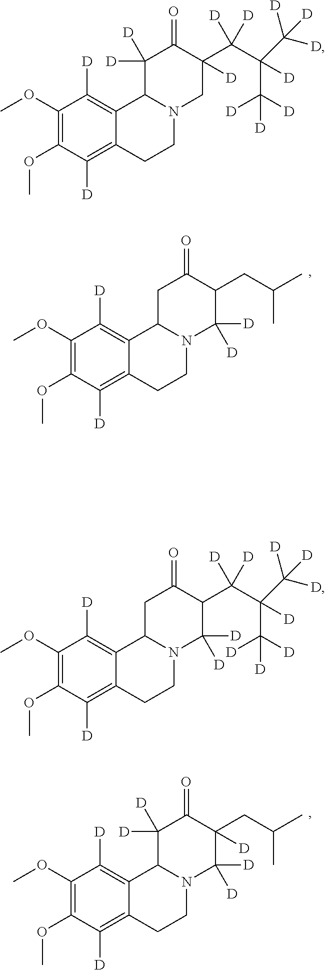

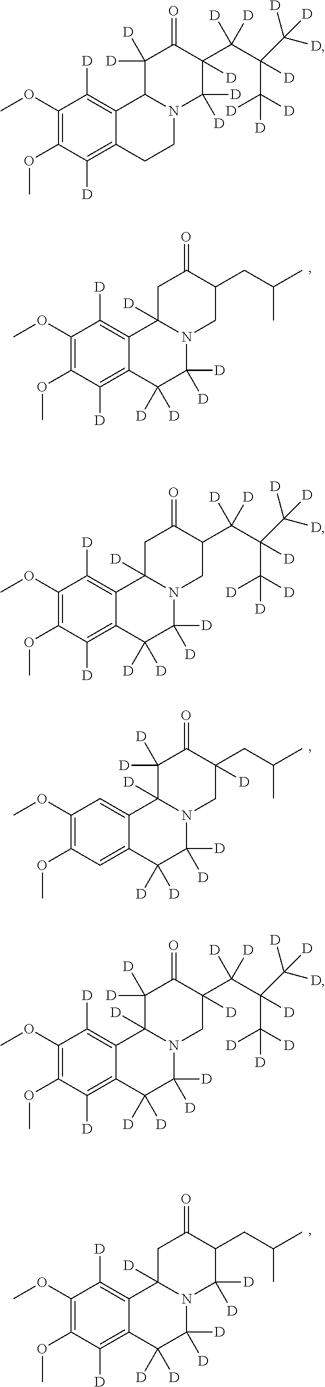

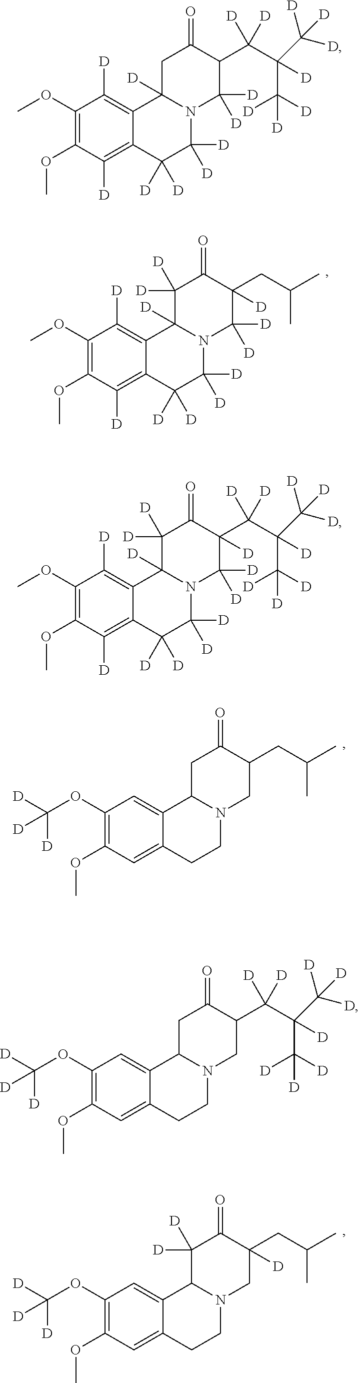

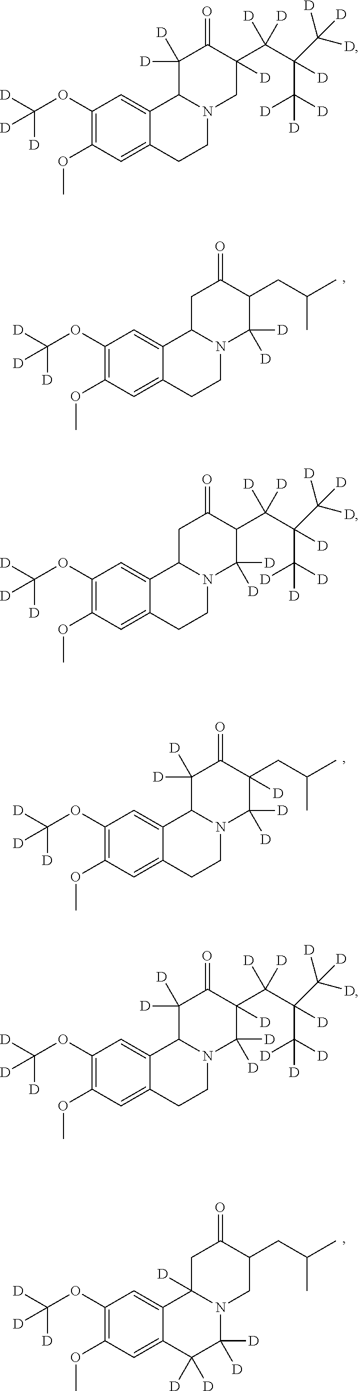

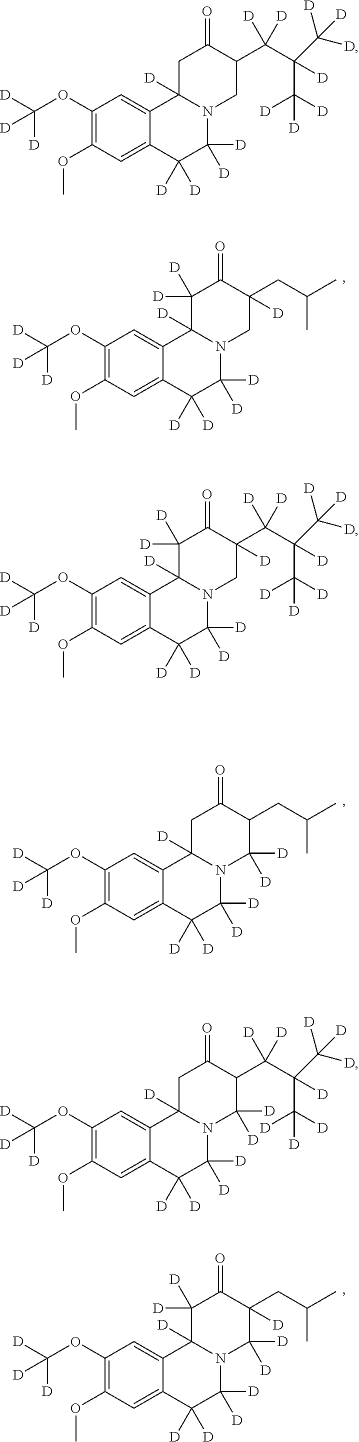

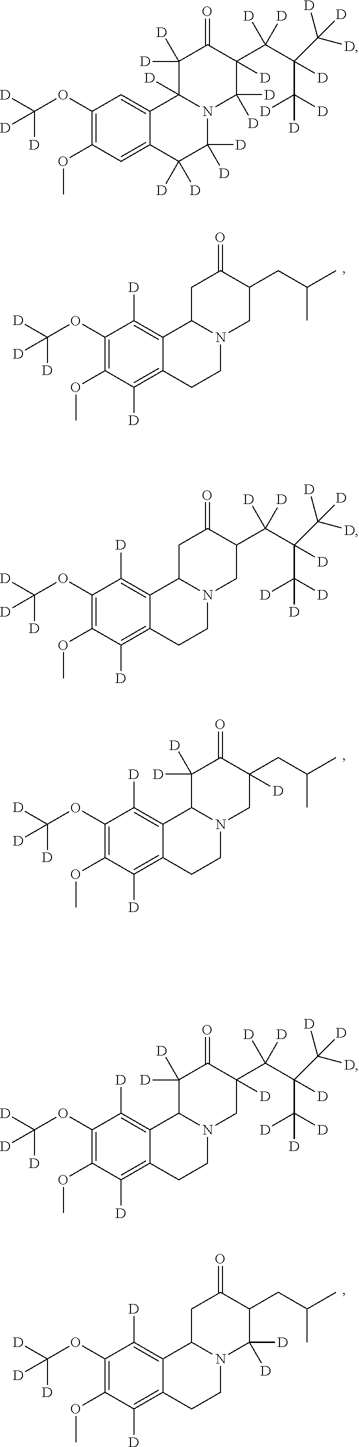

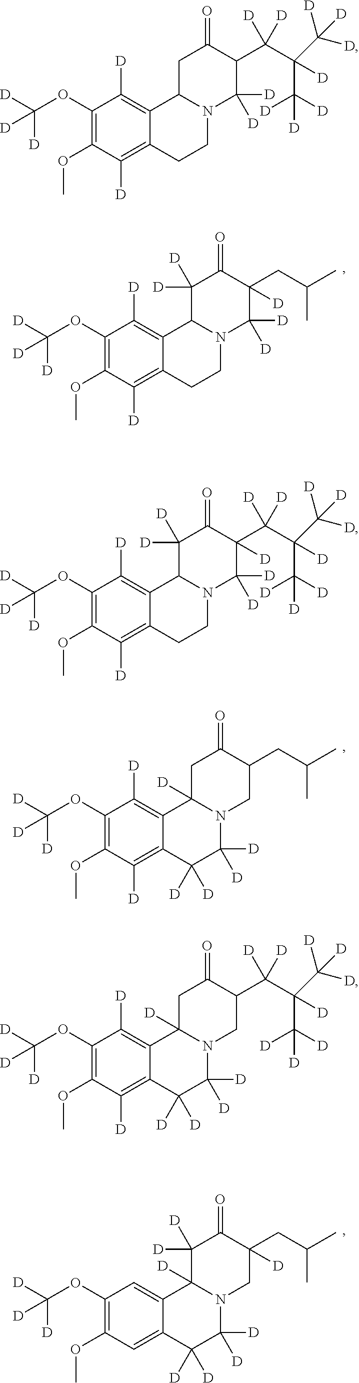

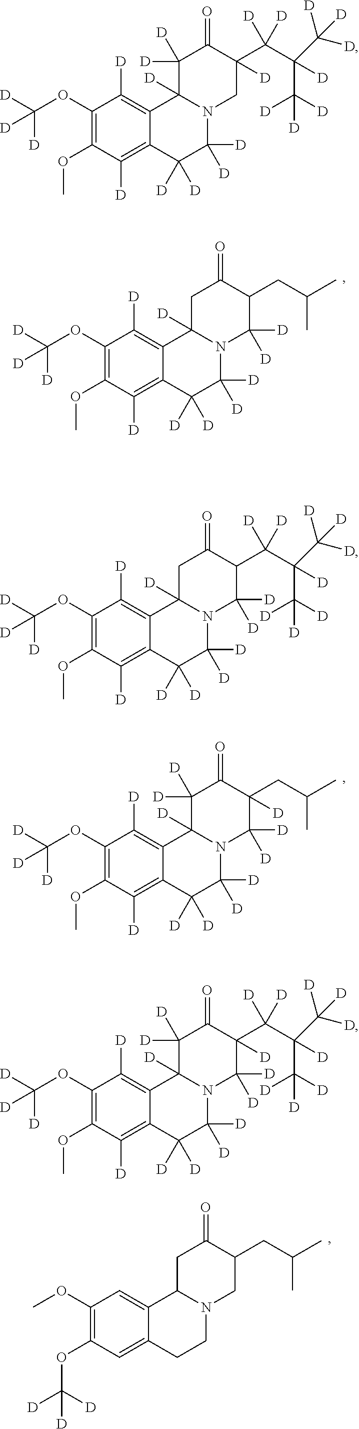

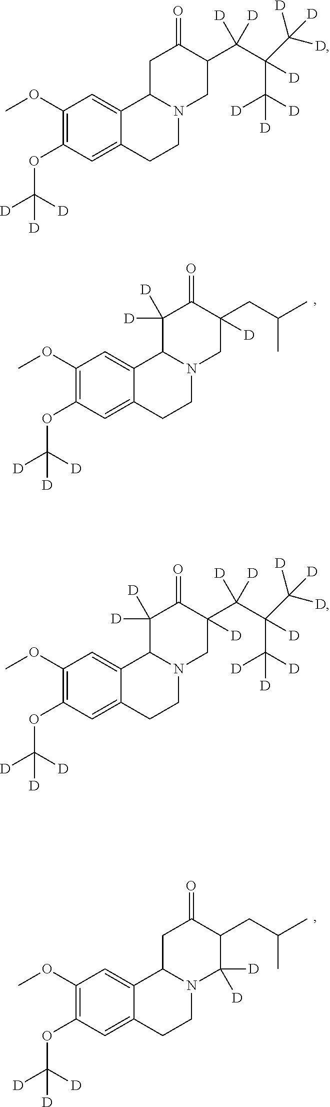

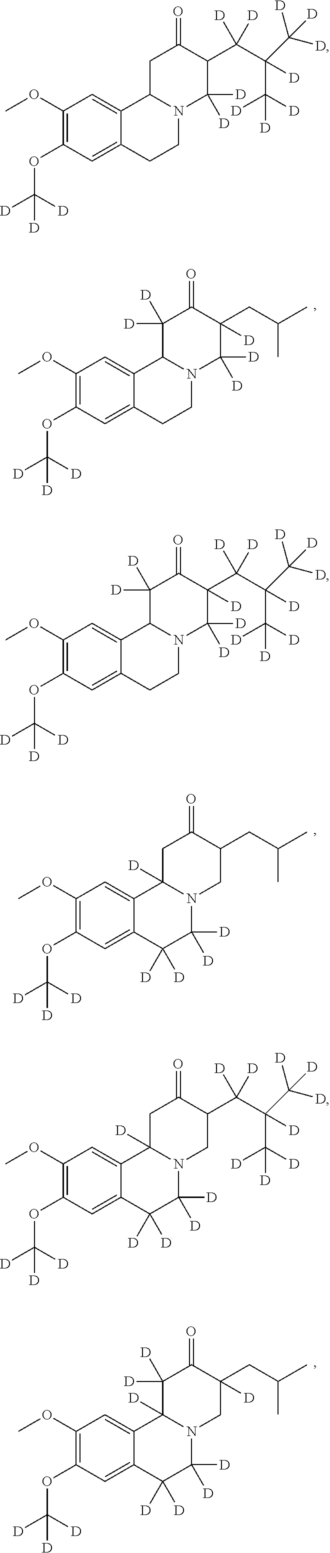

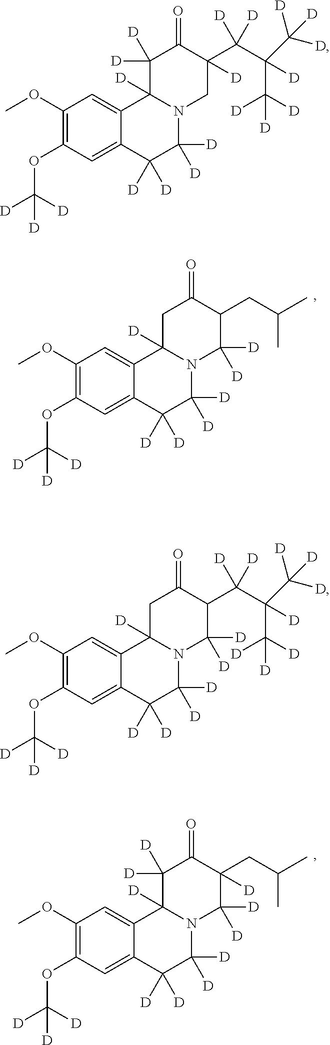

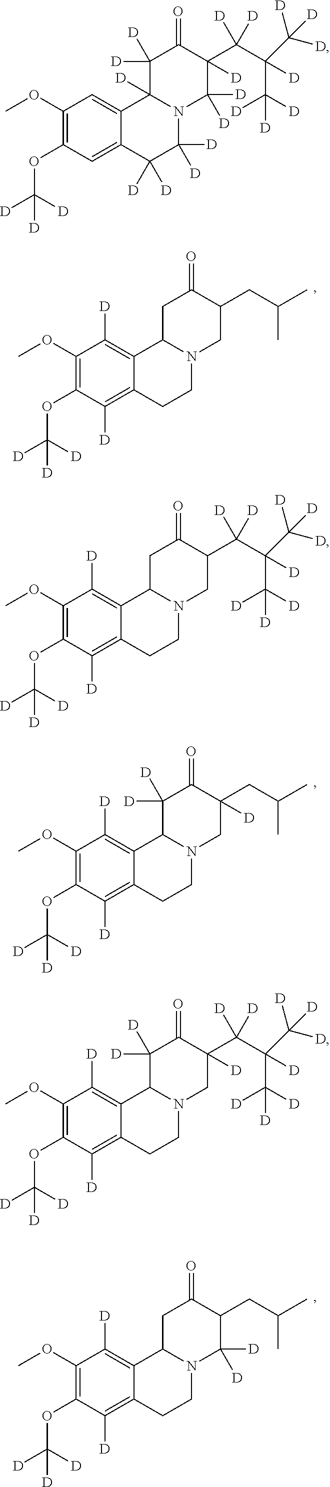

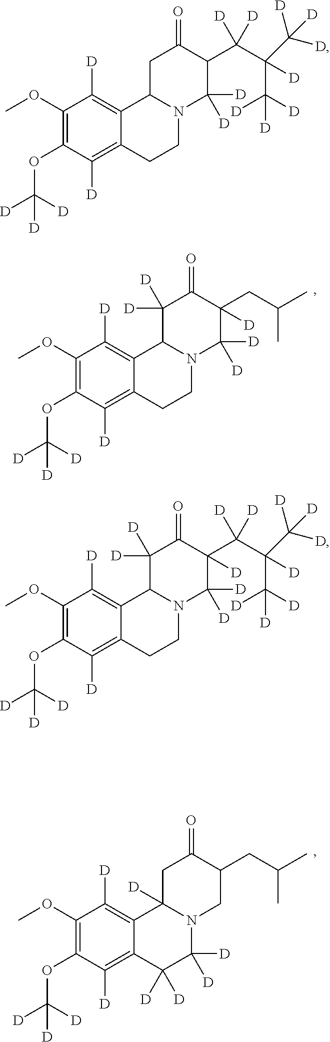

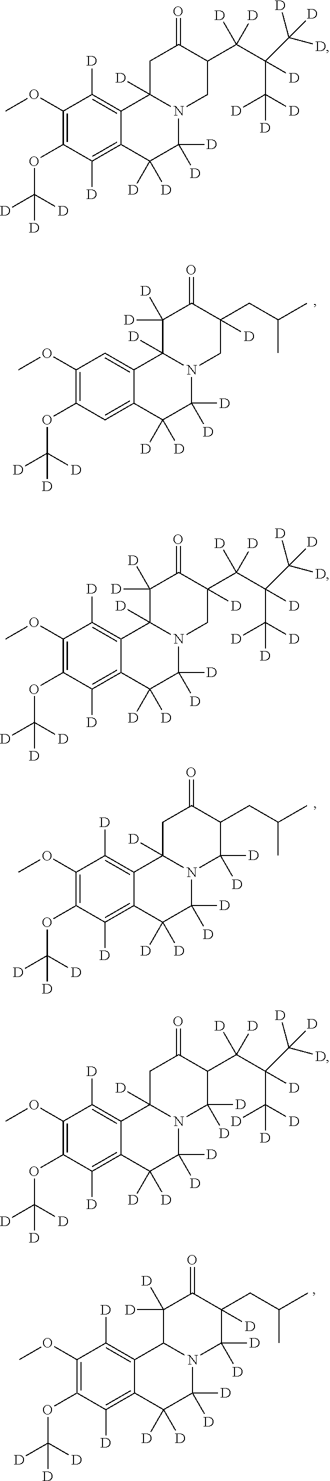

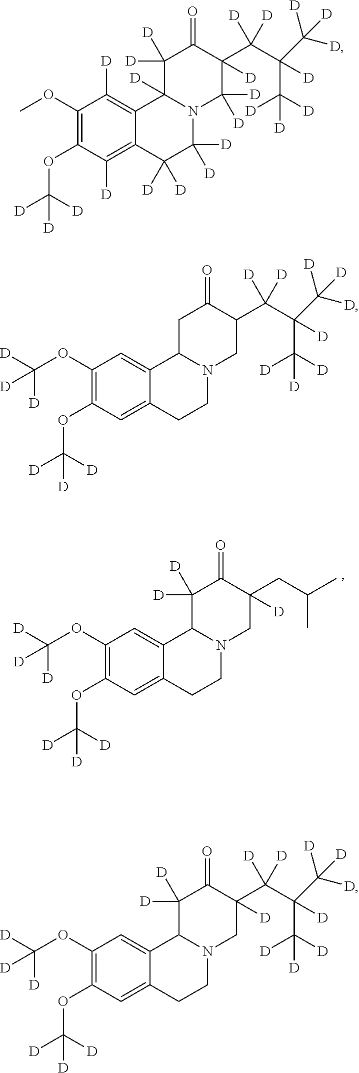

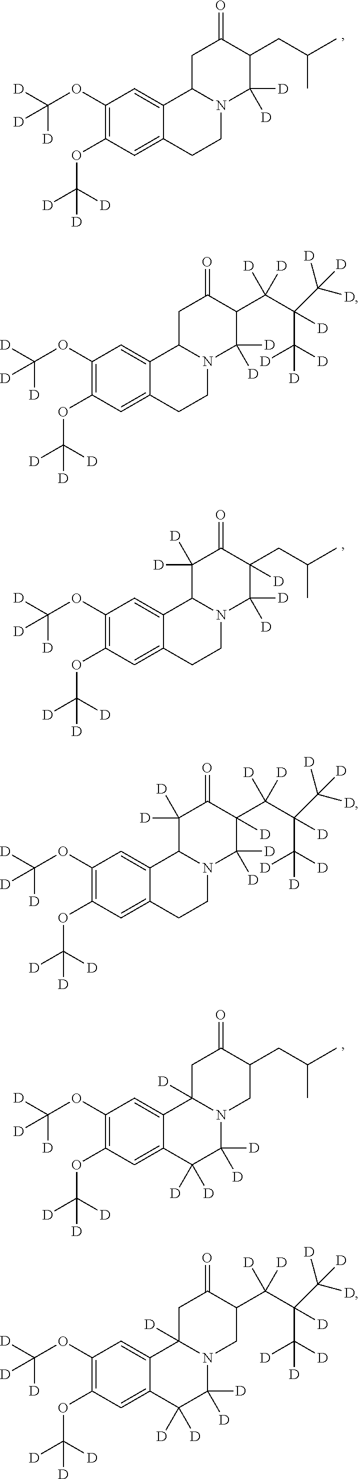

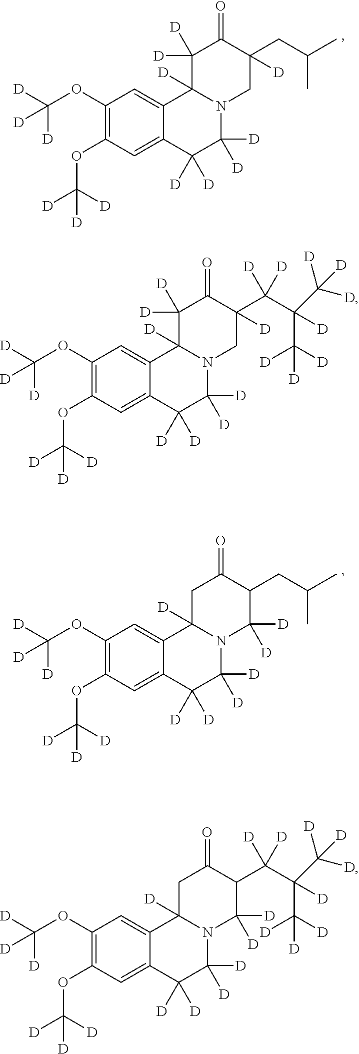

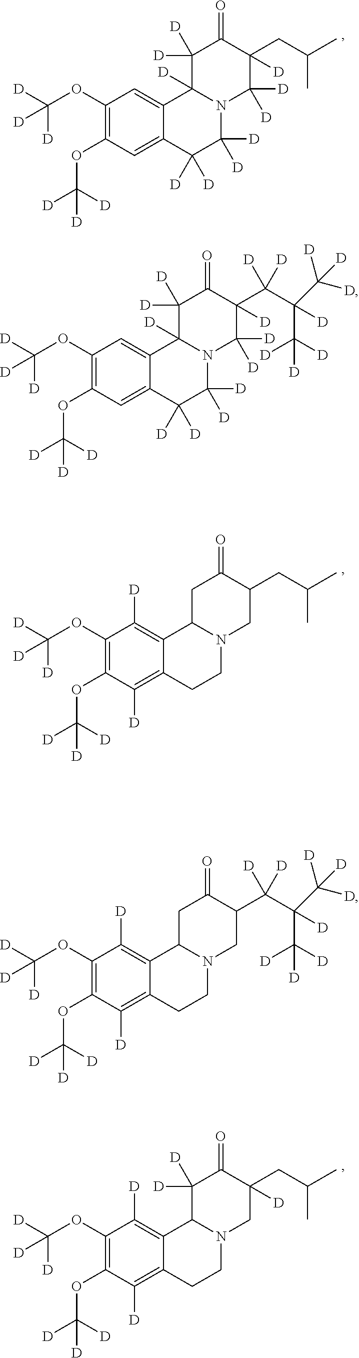

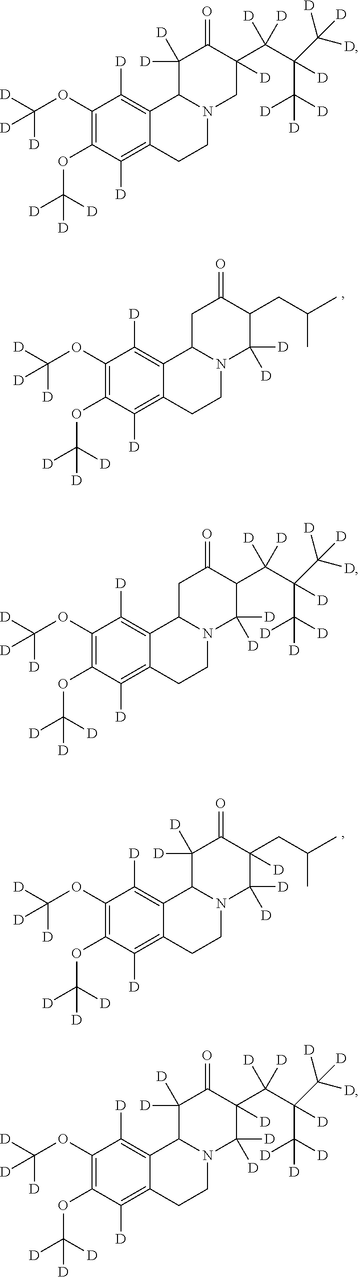

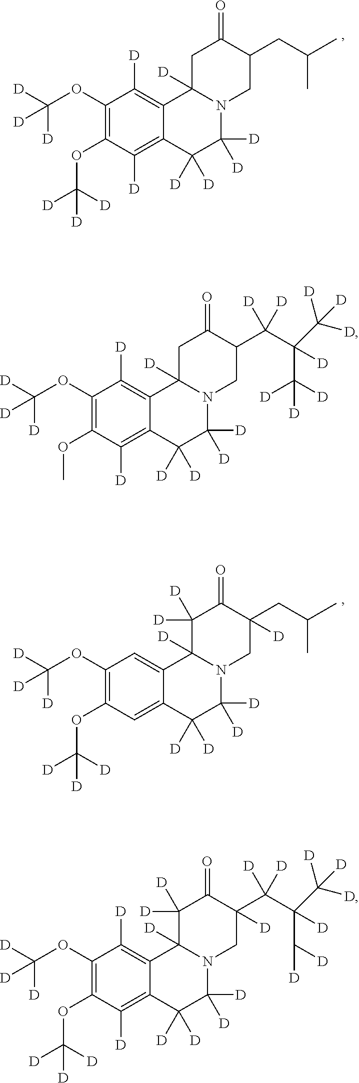

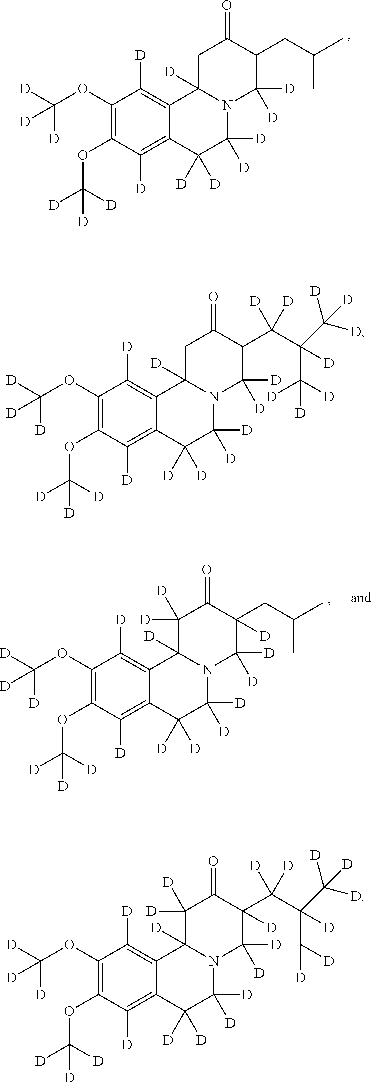

The following compounds can generally be made using the methods described above. It is expected that these compounds when made will have activity similar to those described in the examples above.

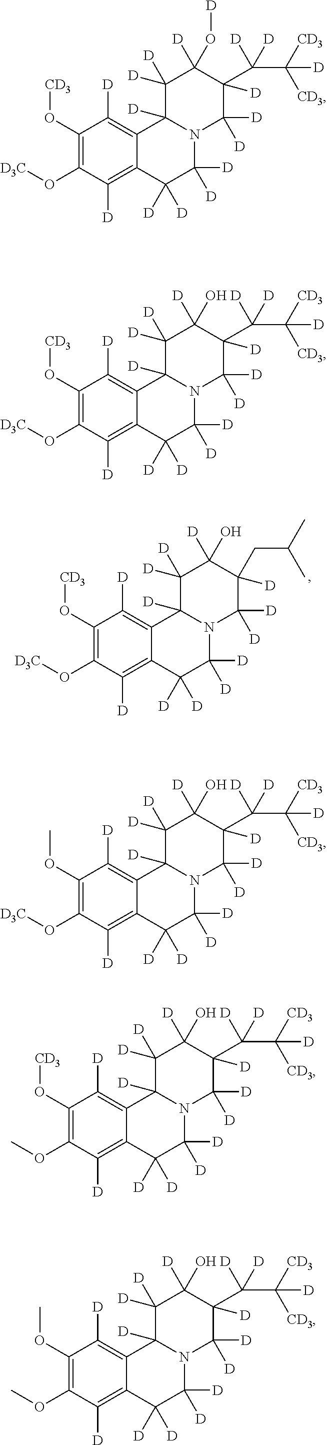

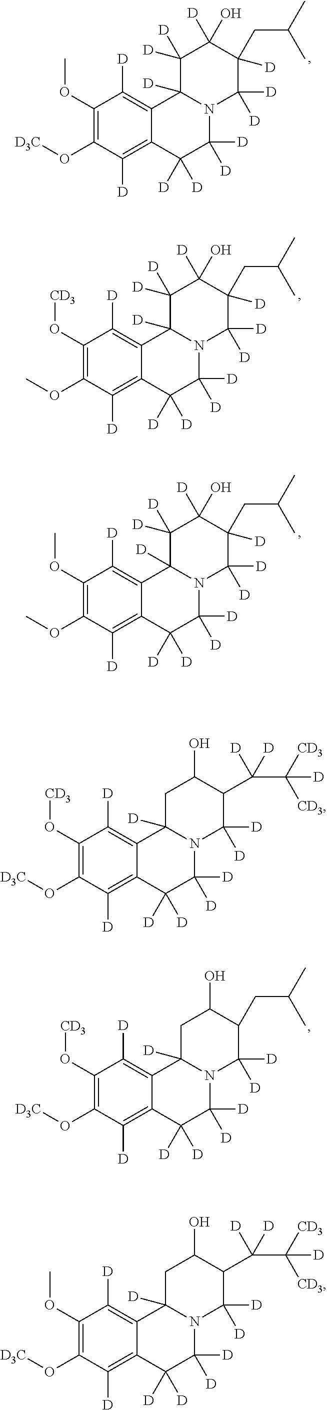

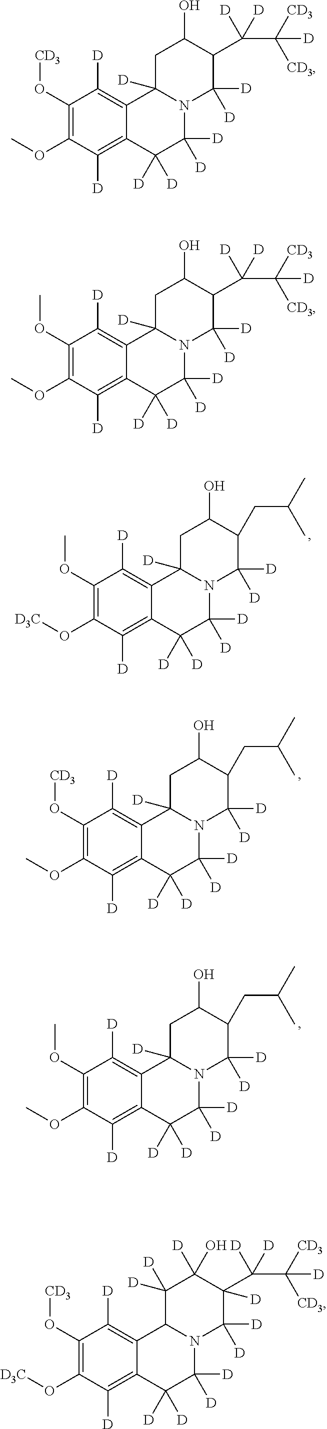

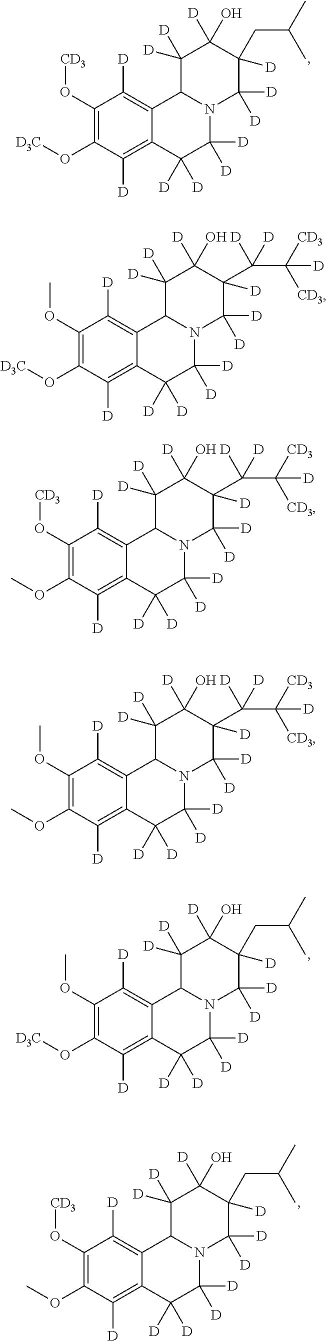

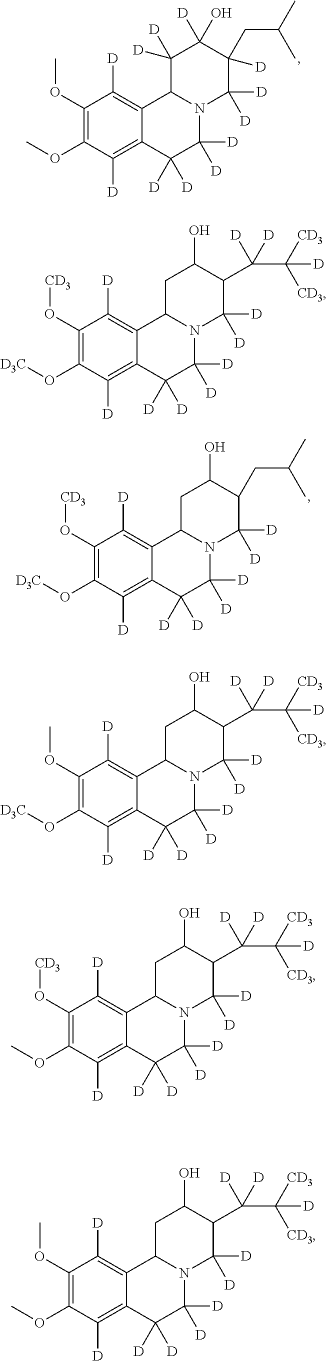

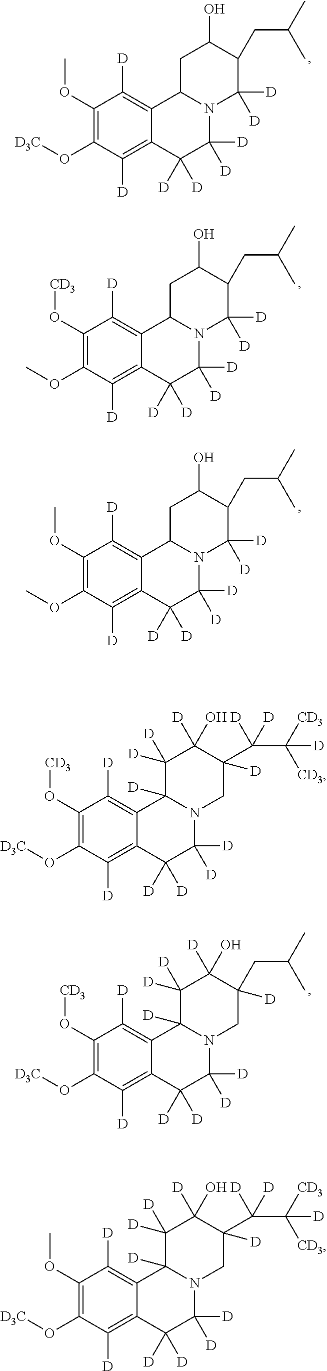

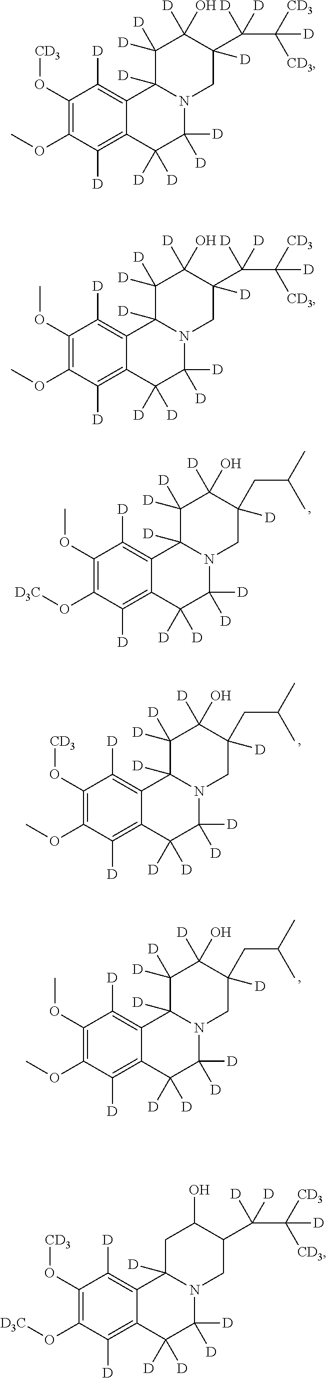

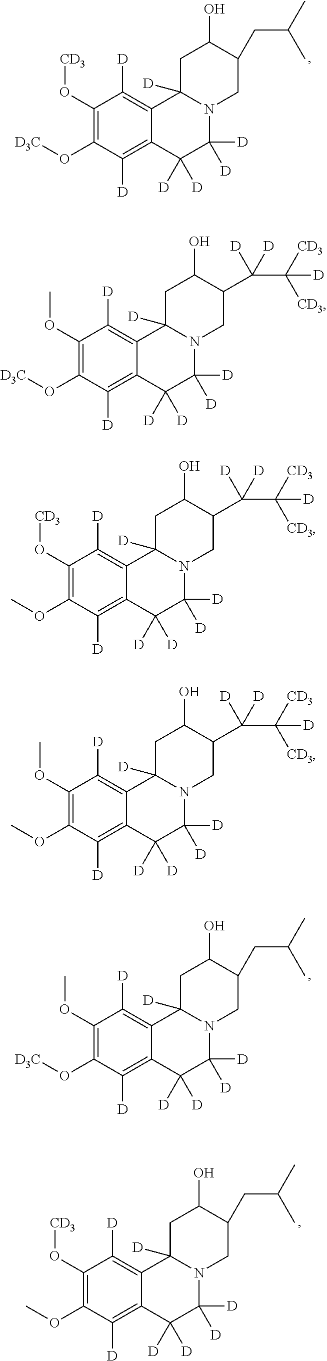

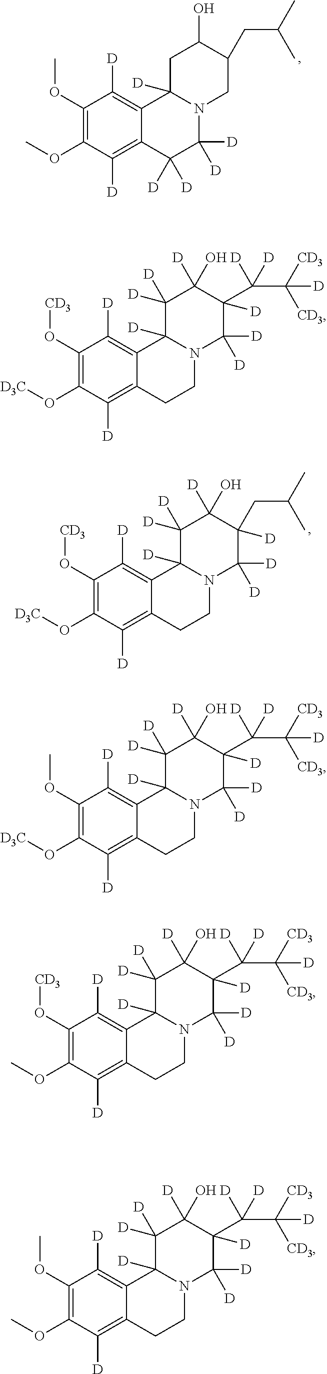

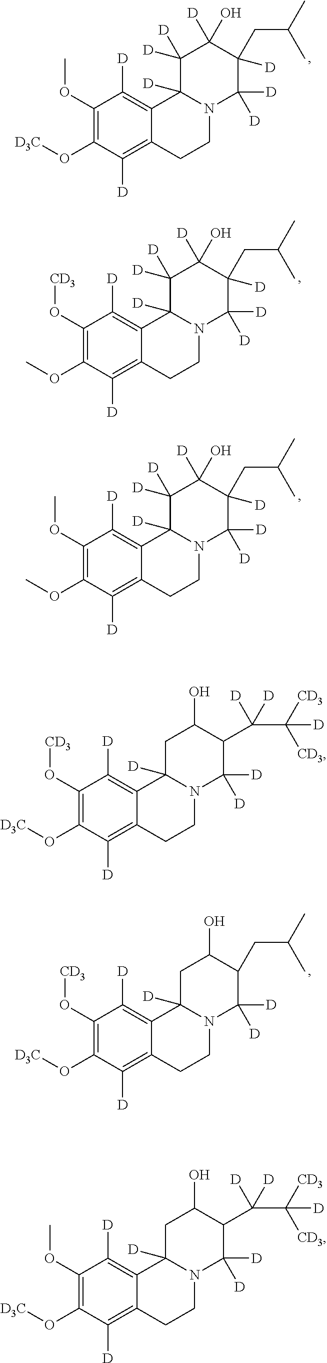

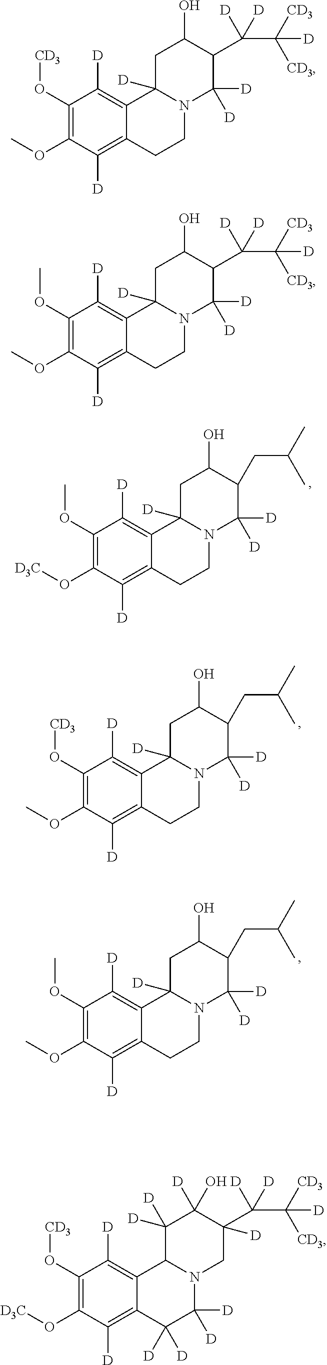

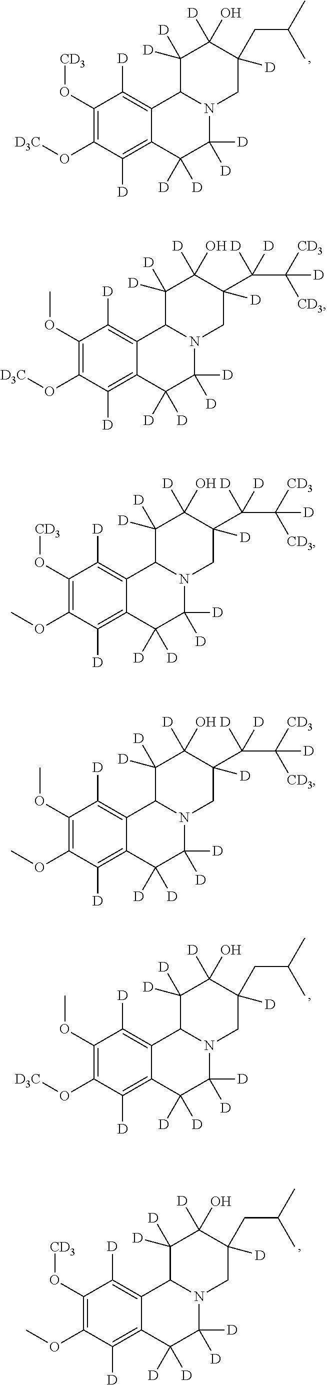

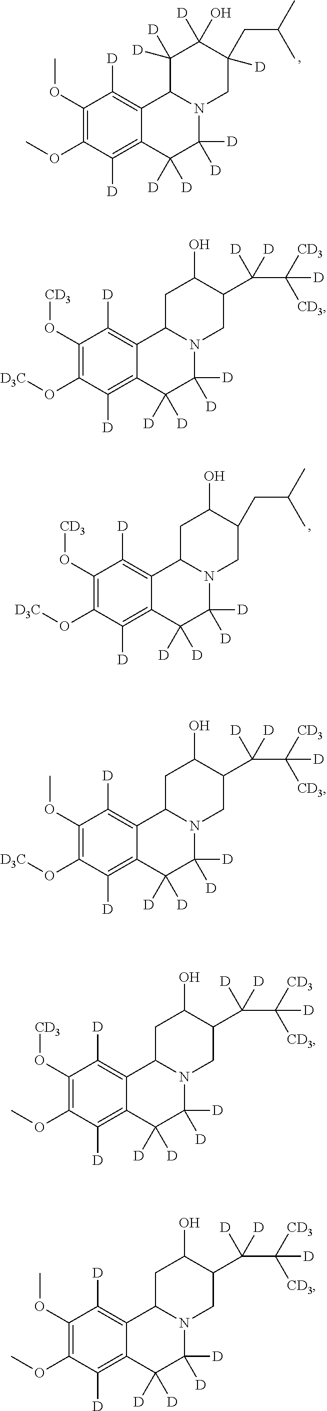

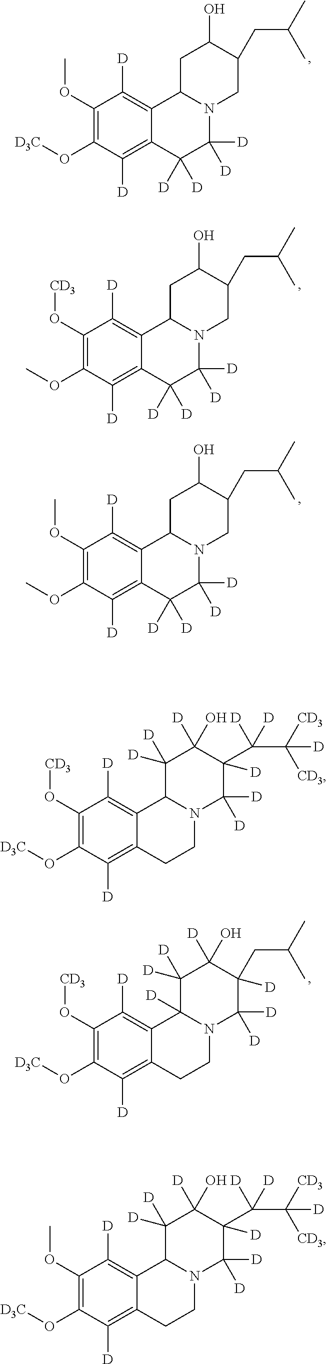

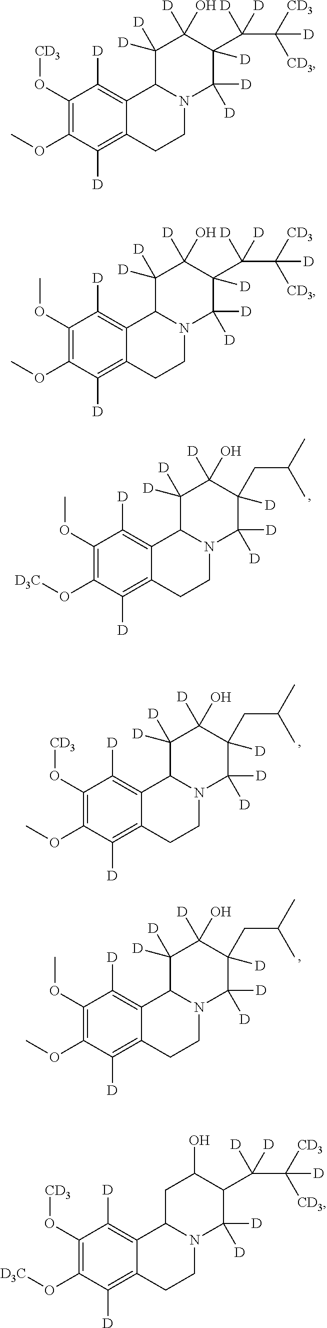

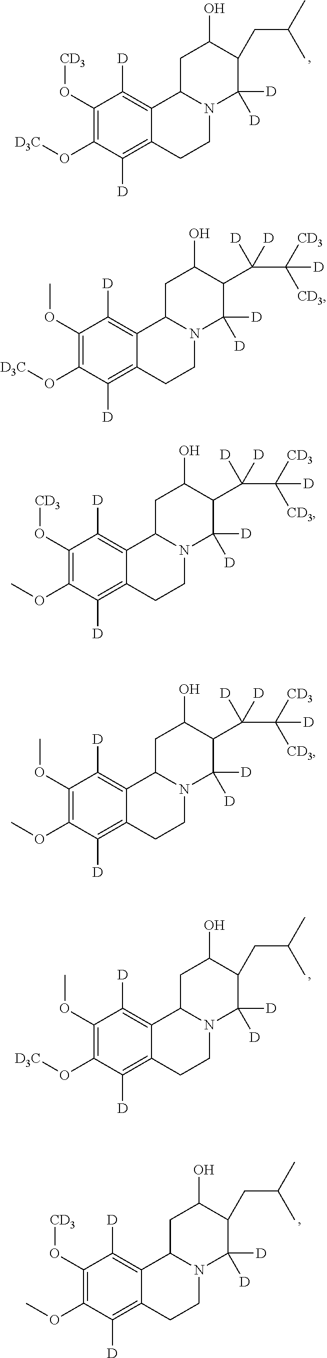

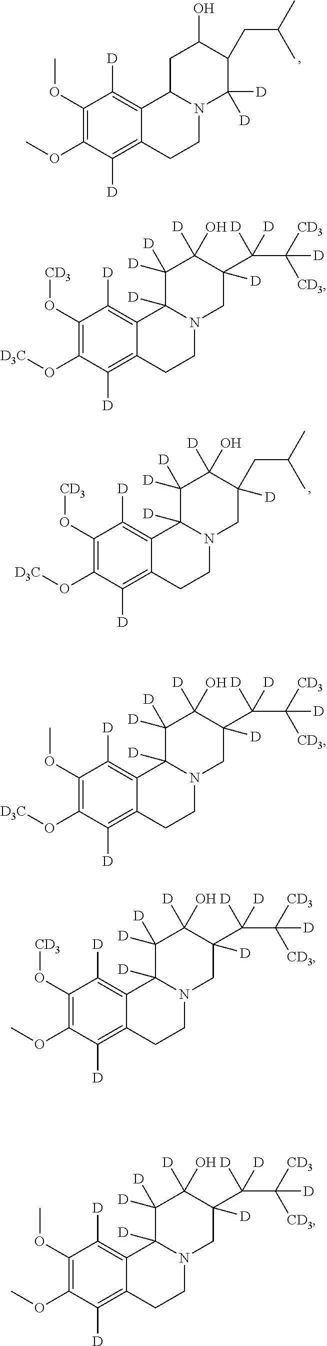

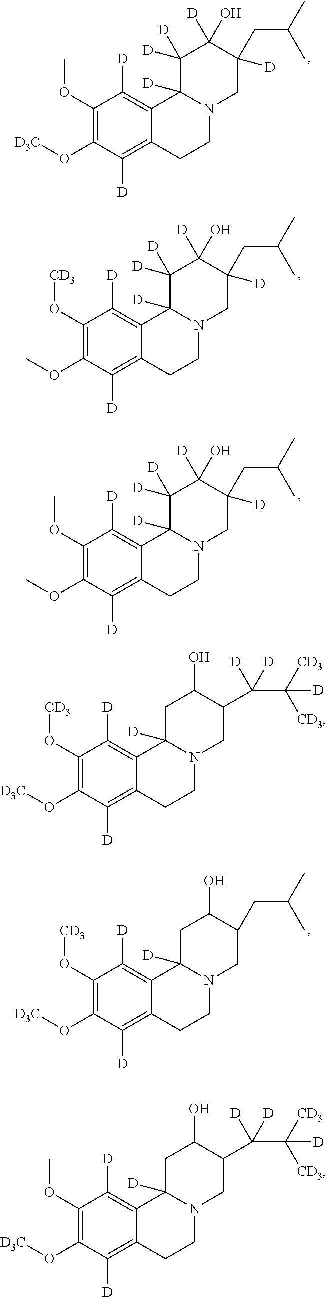

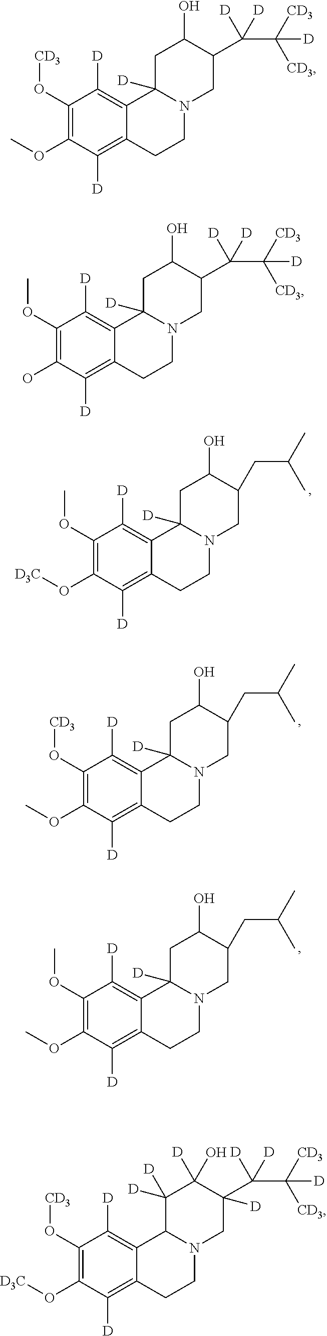

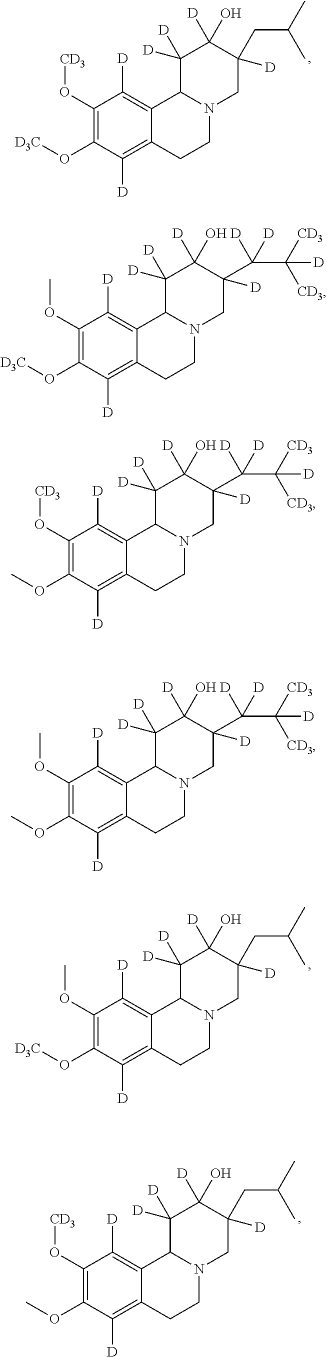

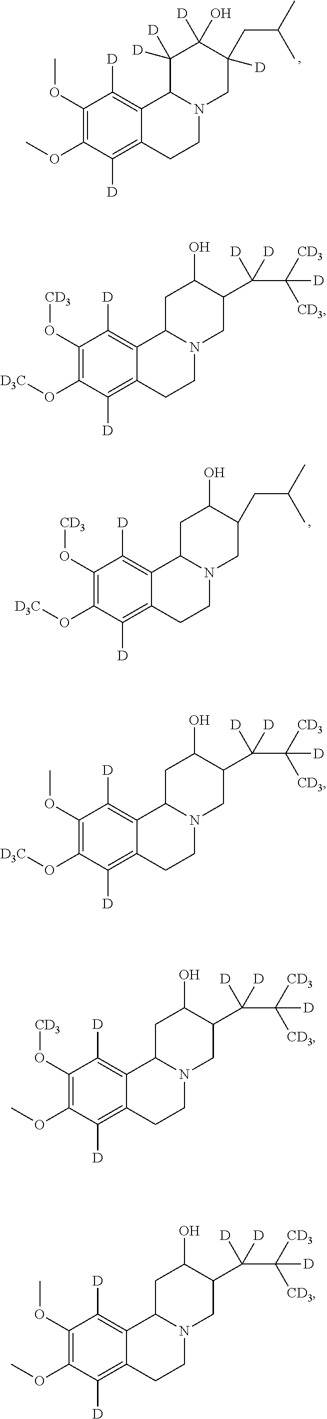

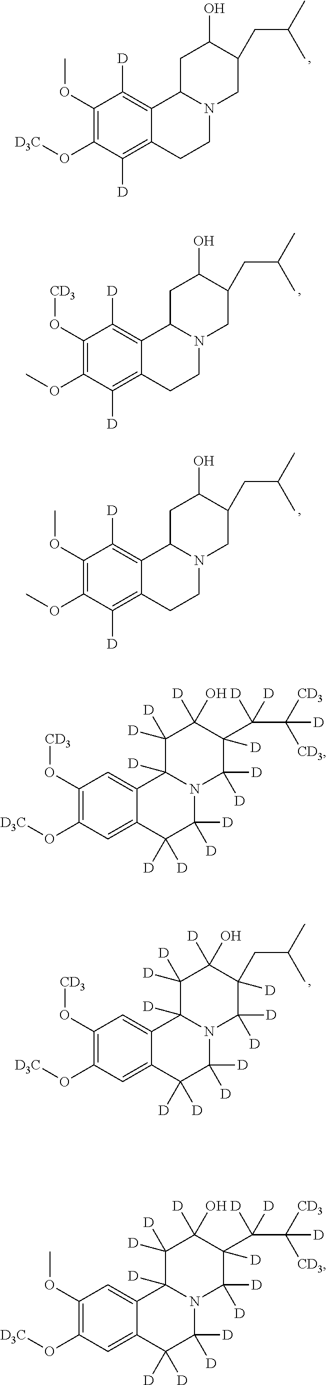

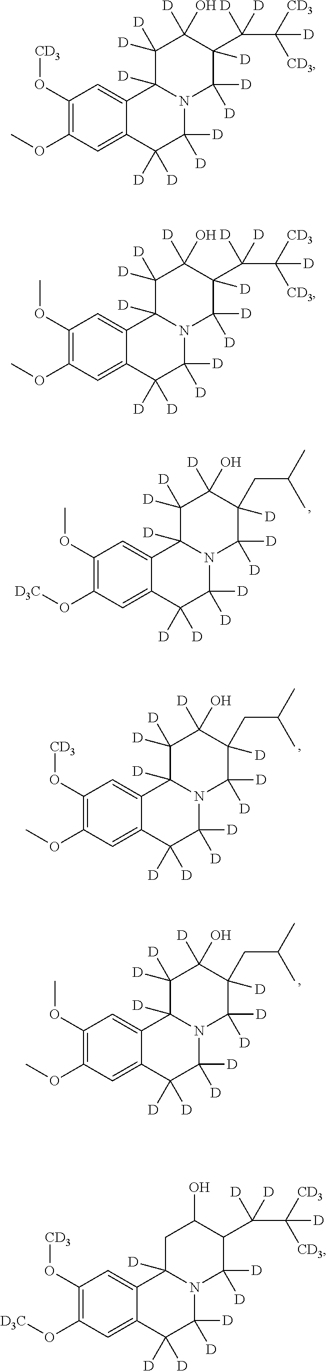

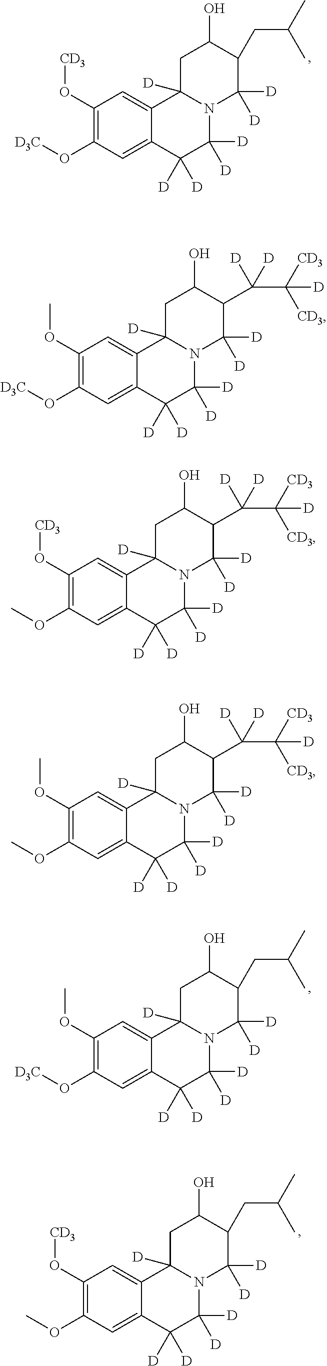

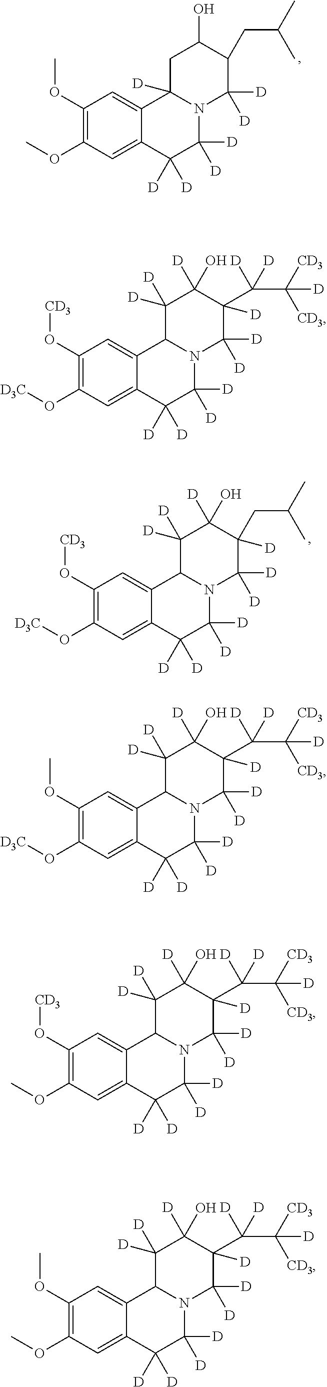

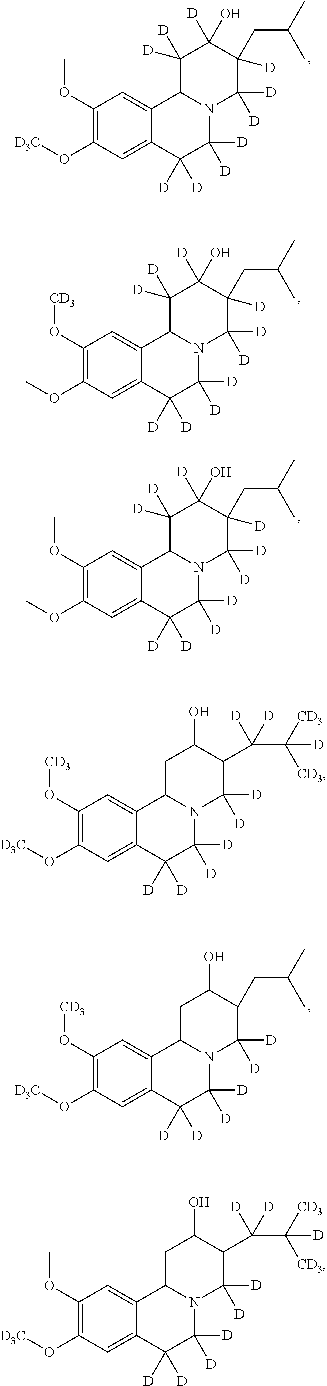

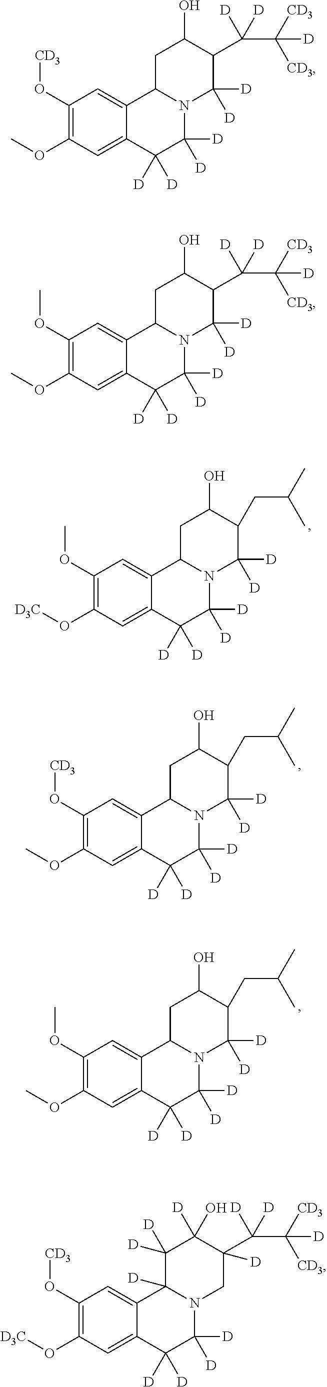

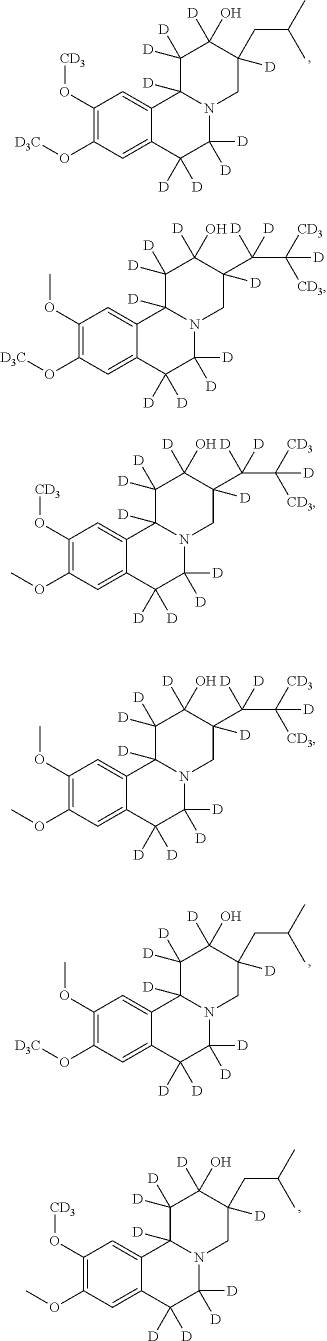

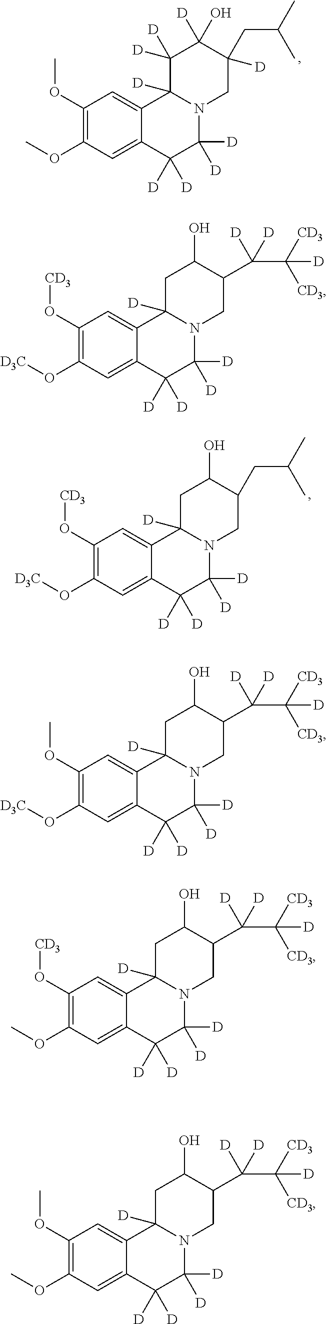

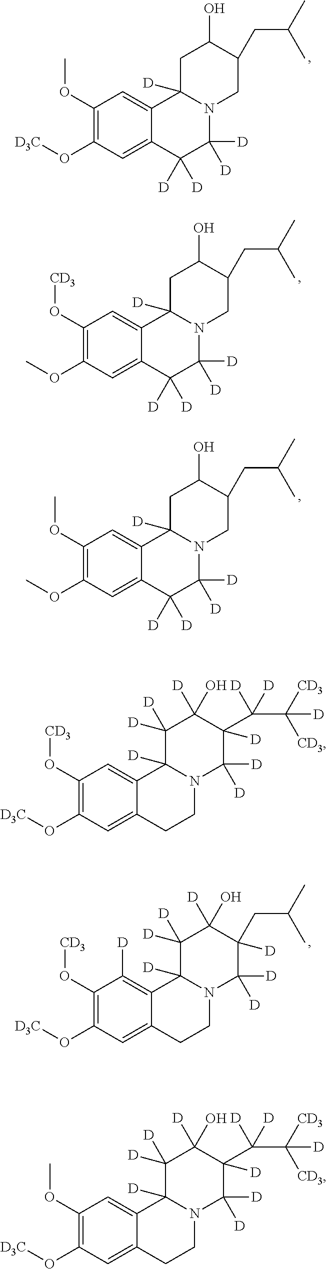

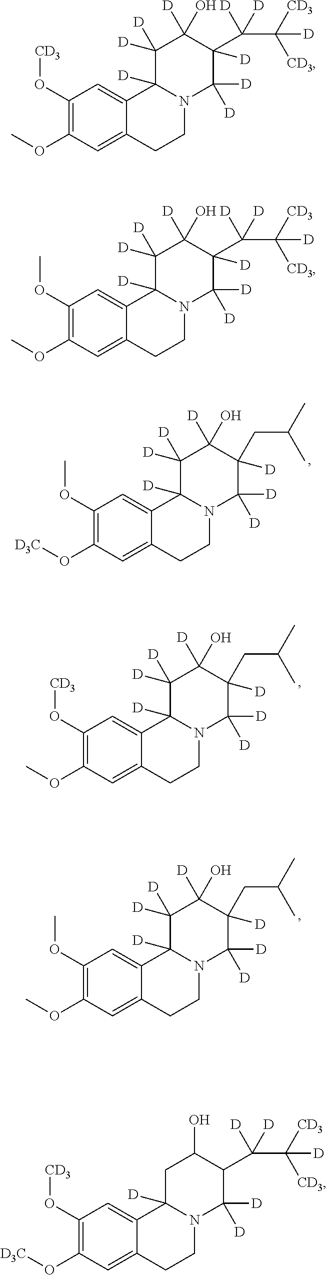

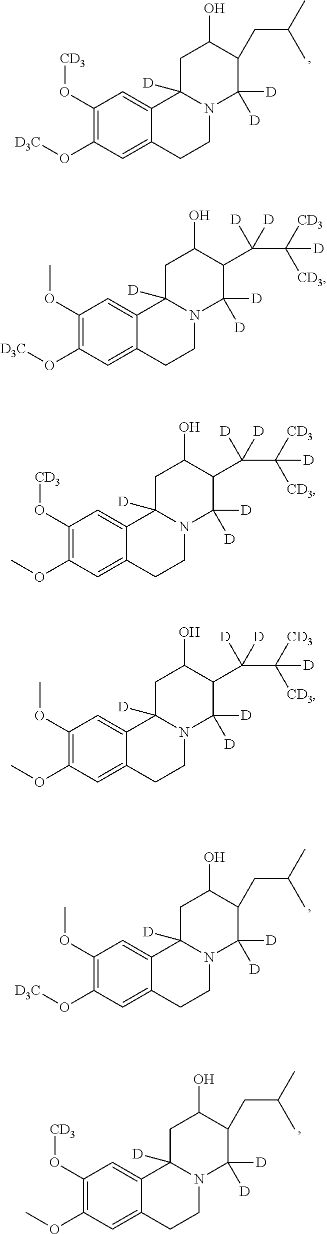

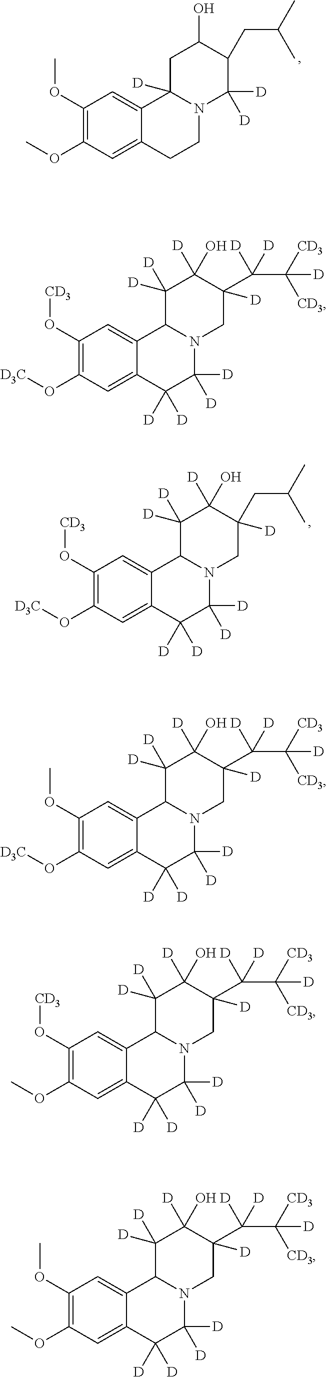

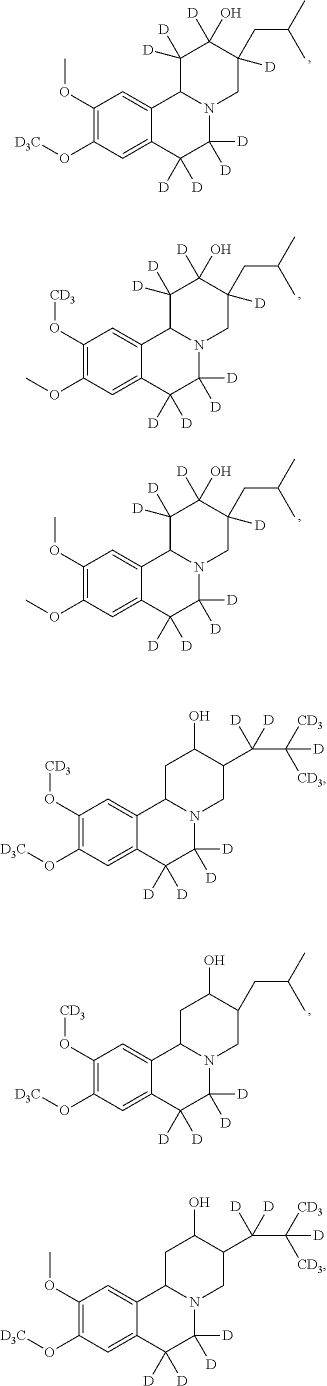

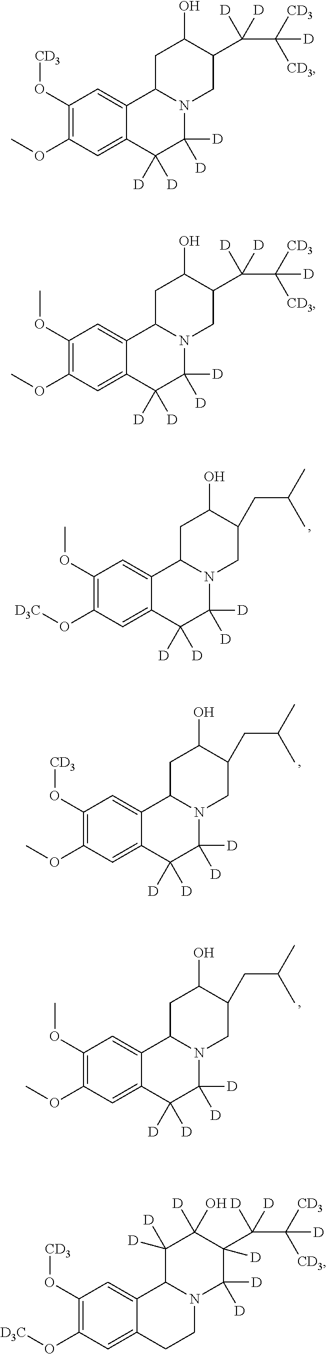

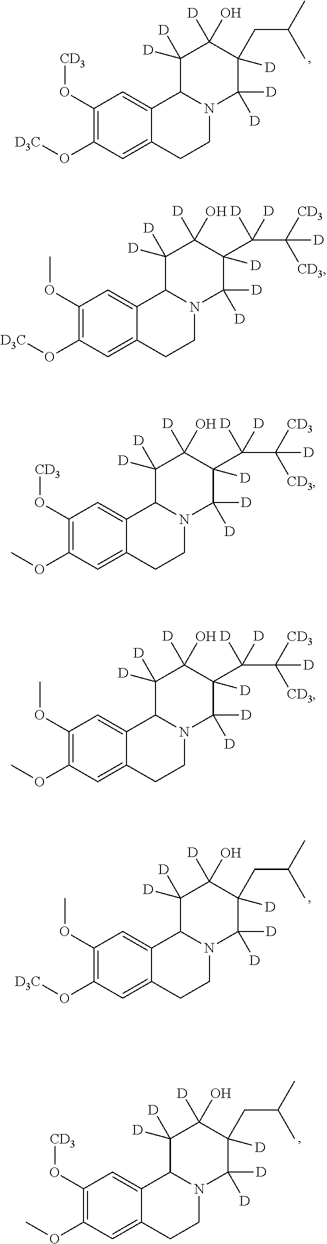

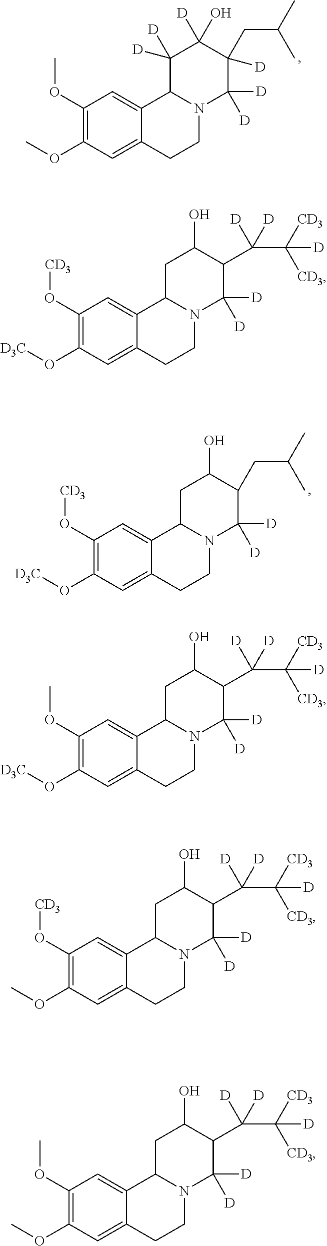

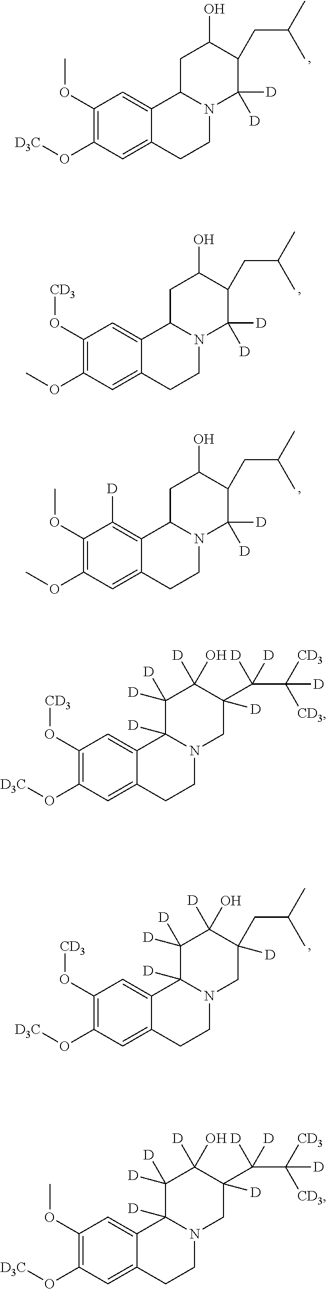

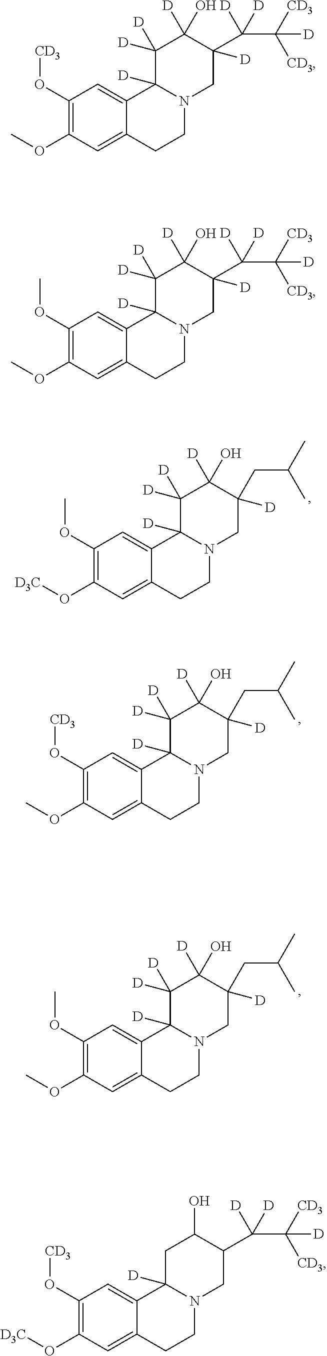

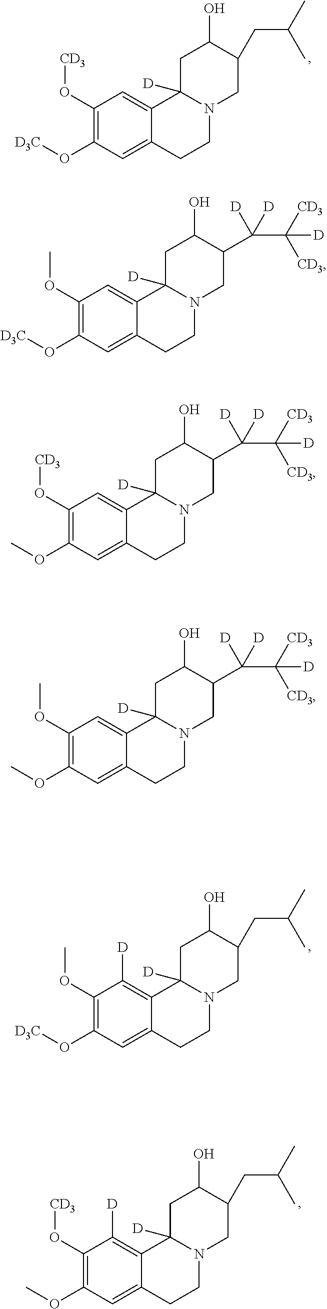

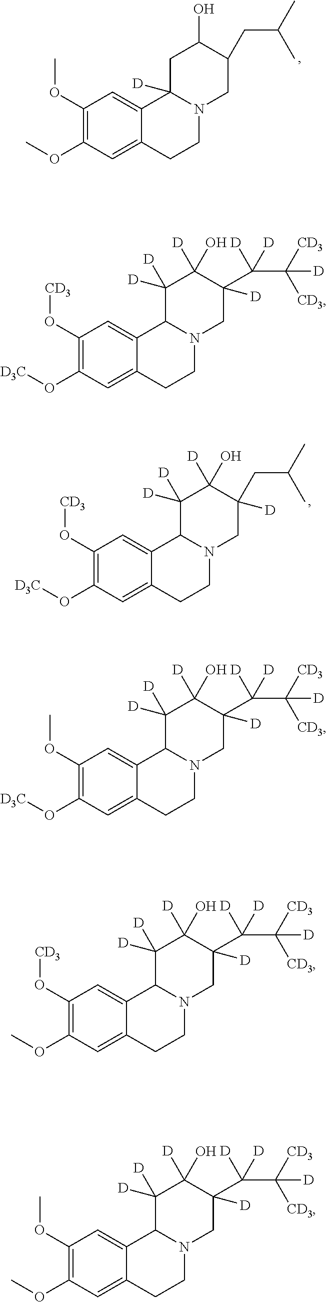

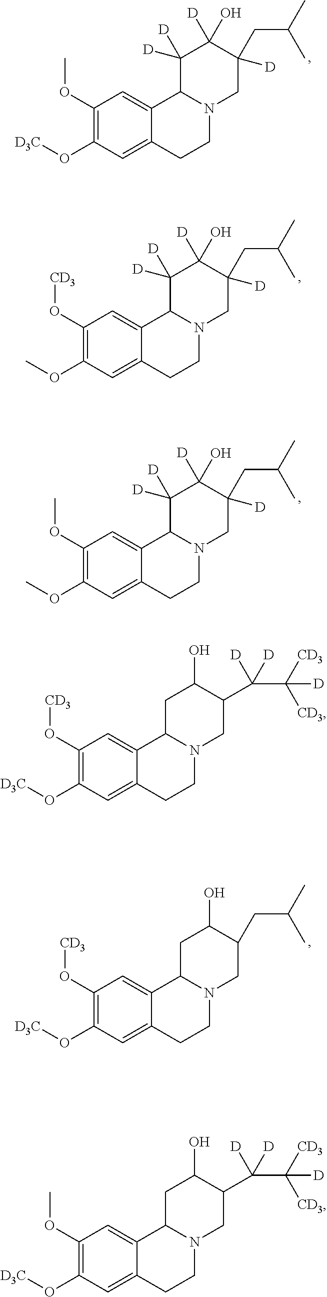

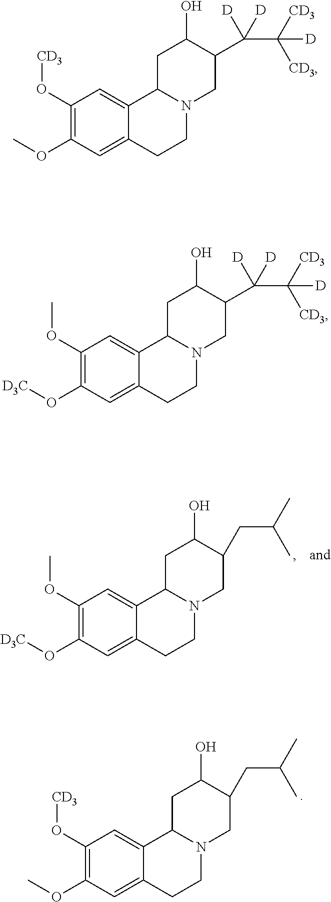

Non-limiting examples include the following compounds and pharmaceutically acceptable salts thereof:

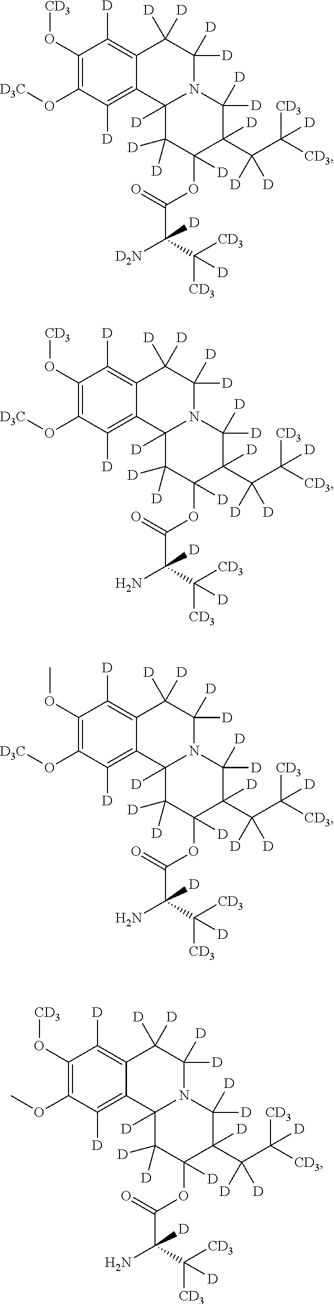

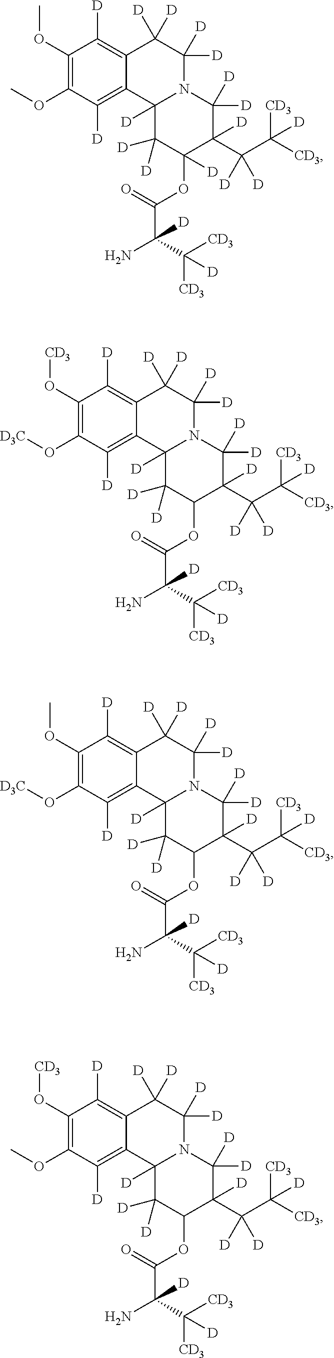

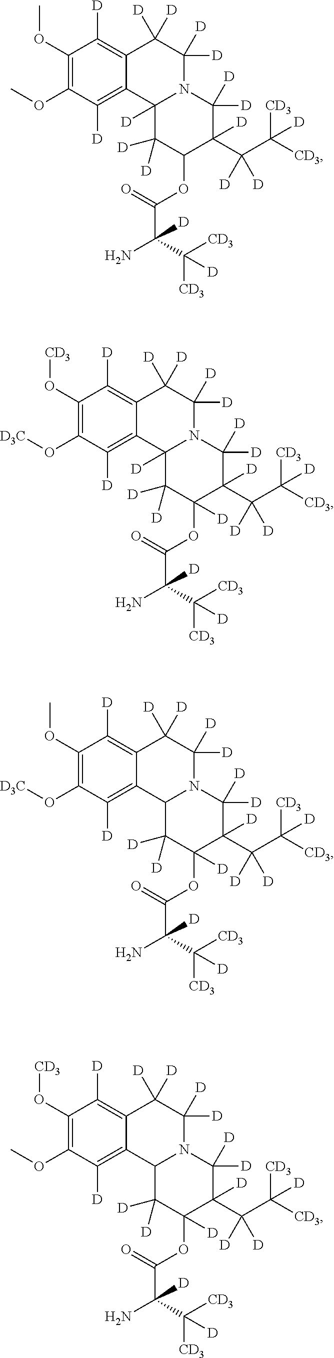

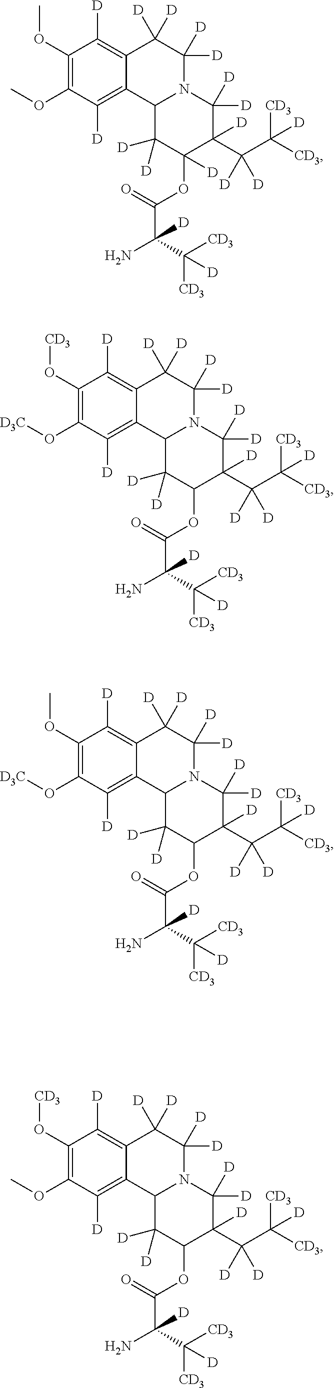

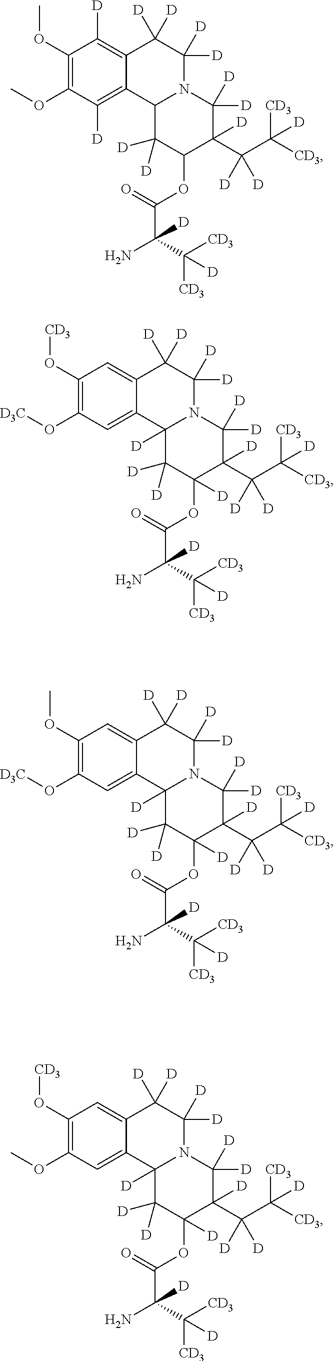

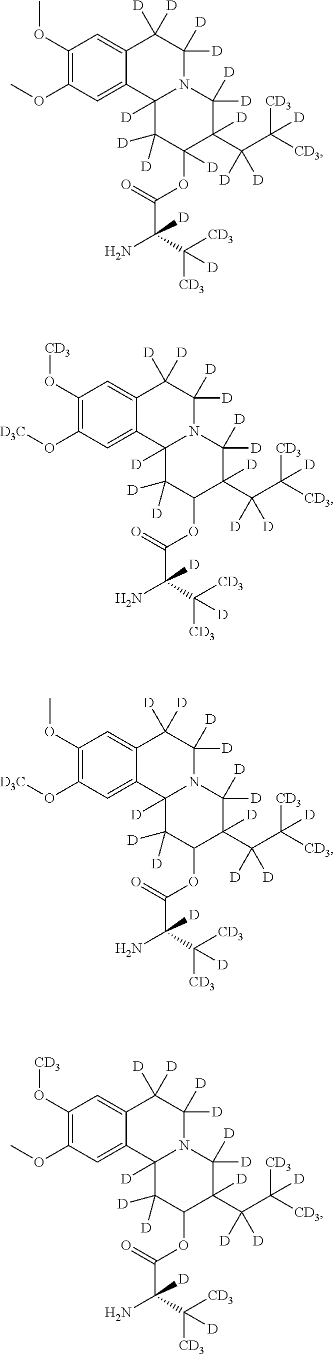

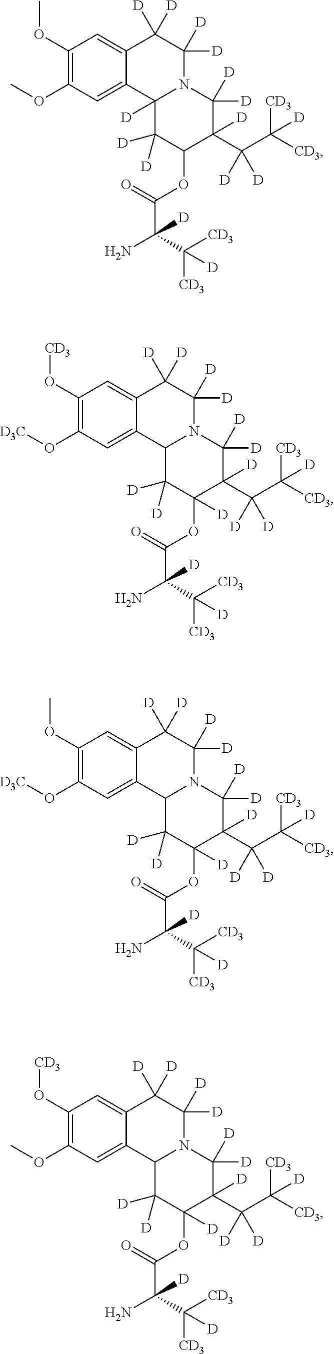

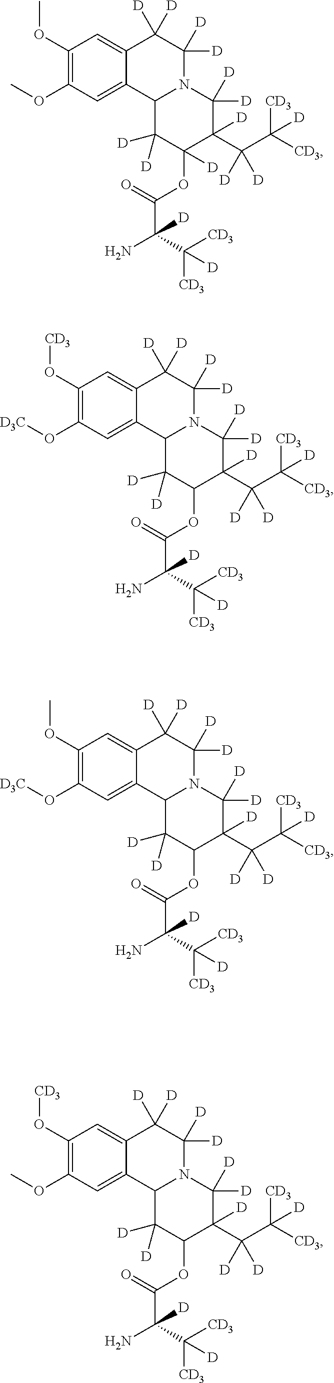

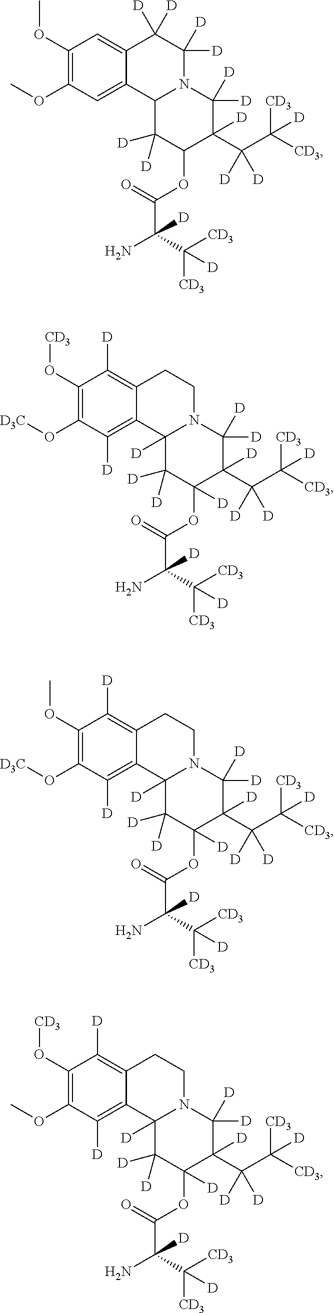

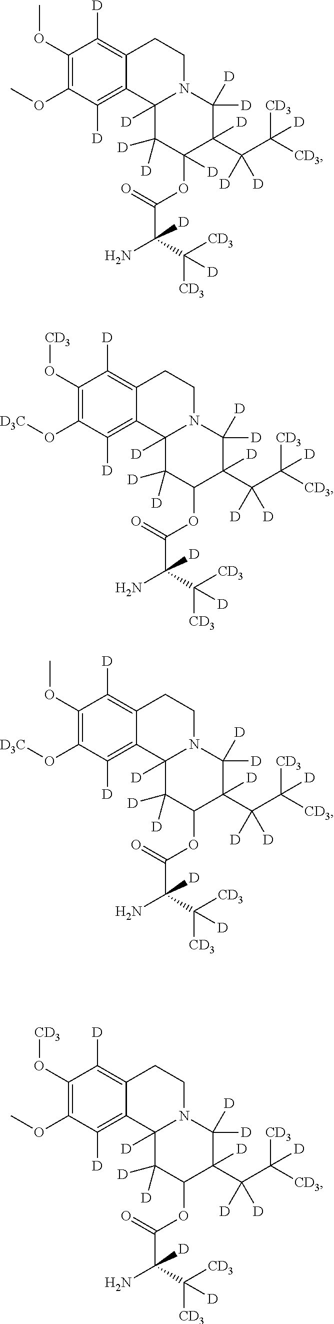

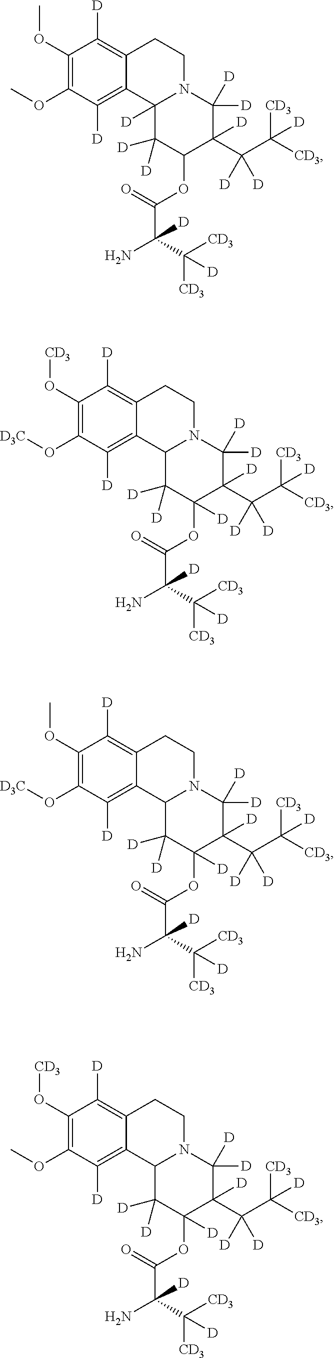

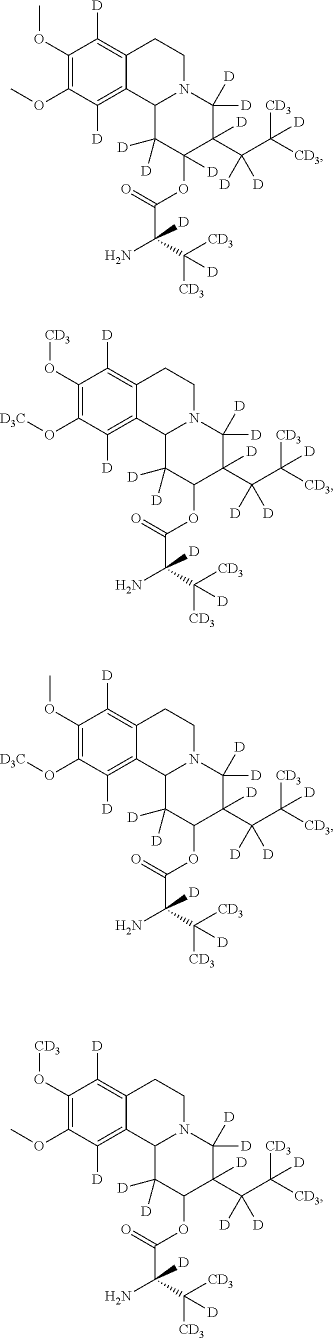

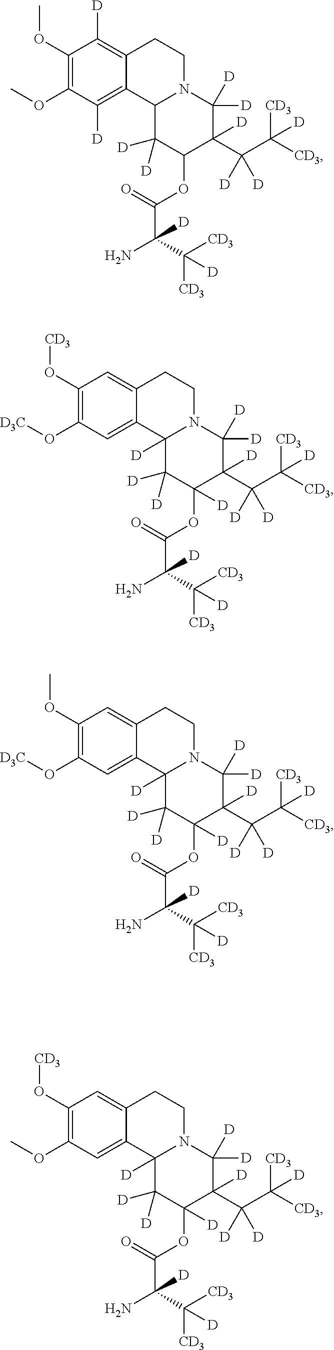

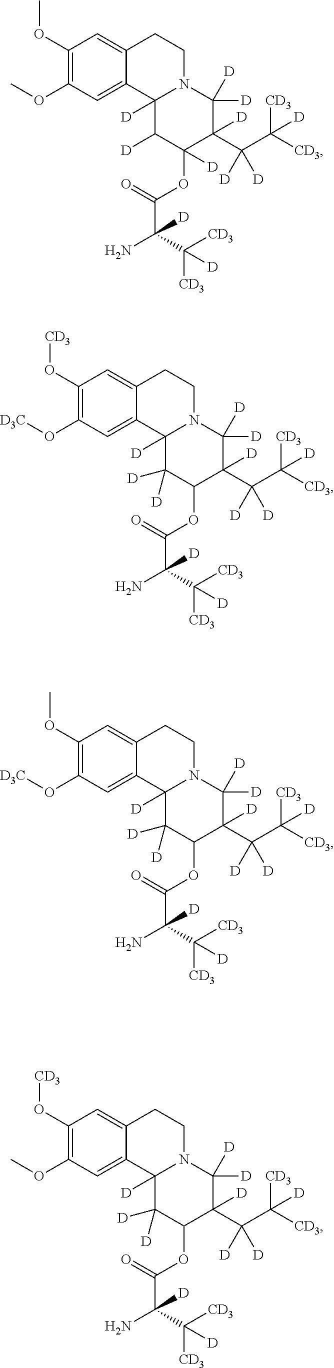

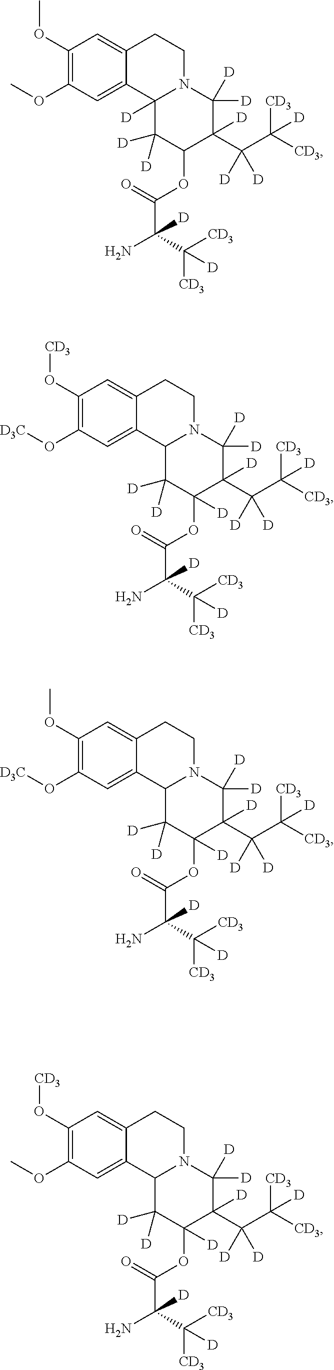

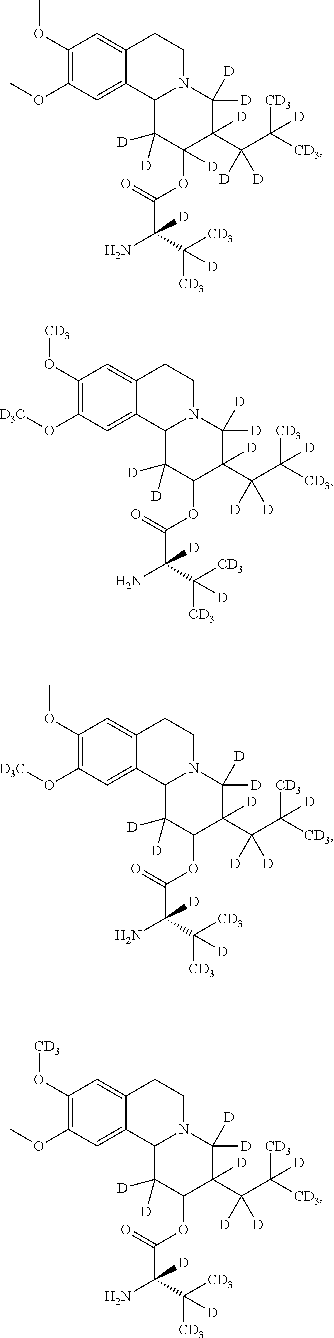

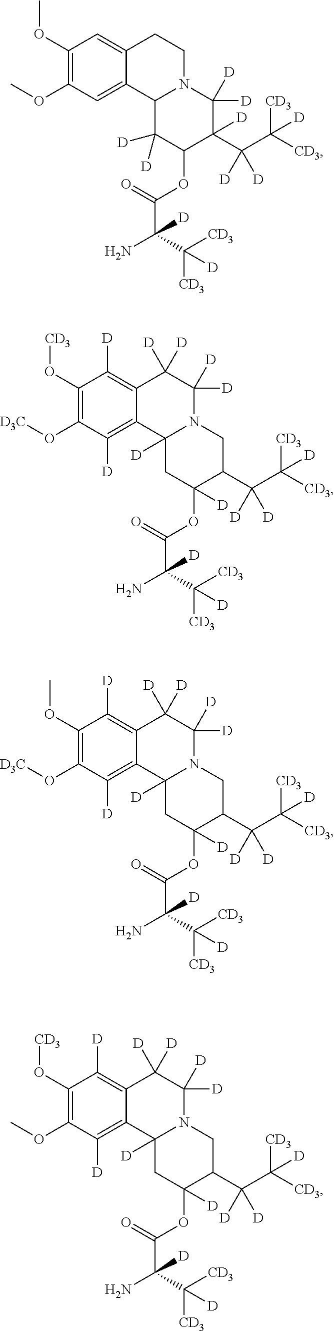

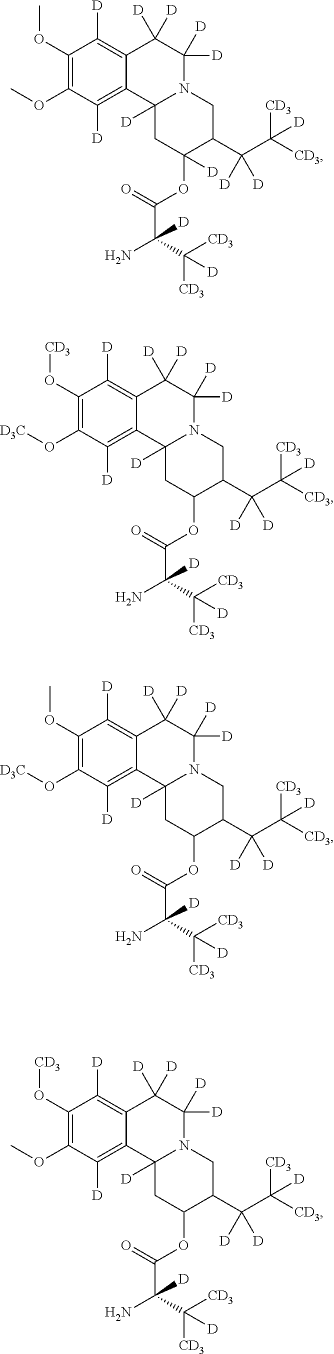

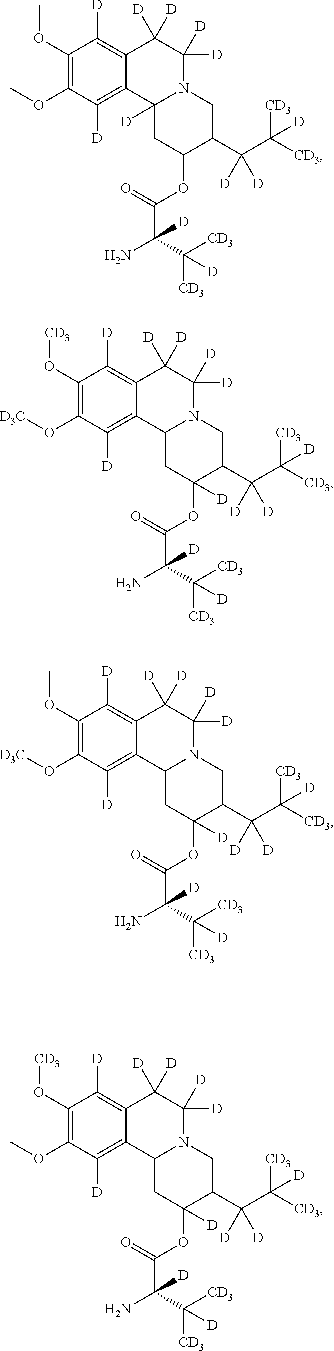

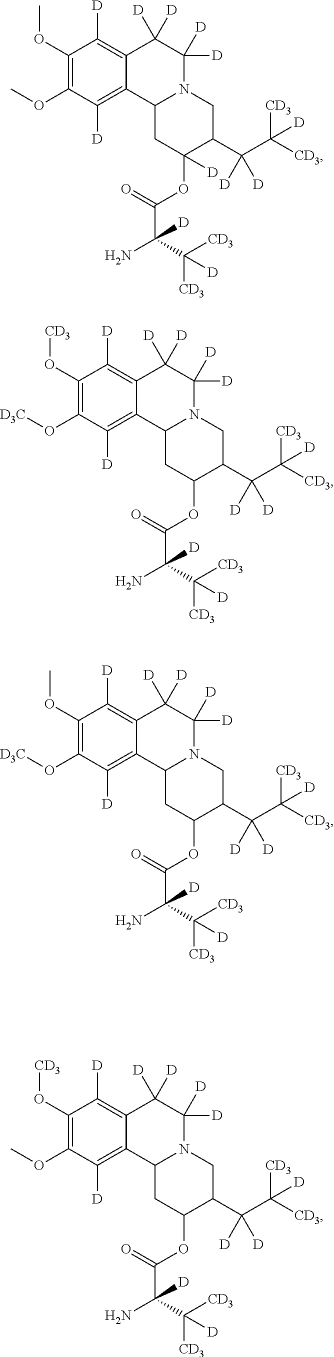

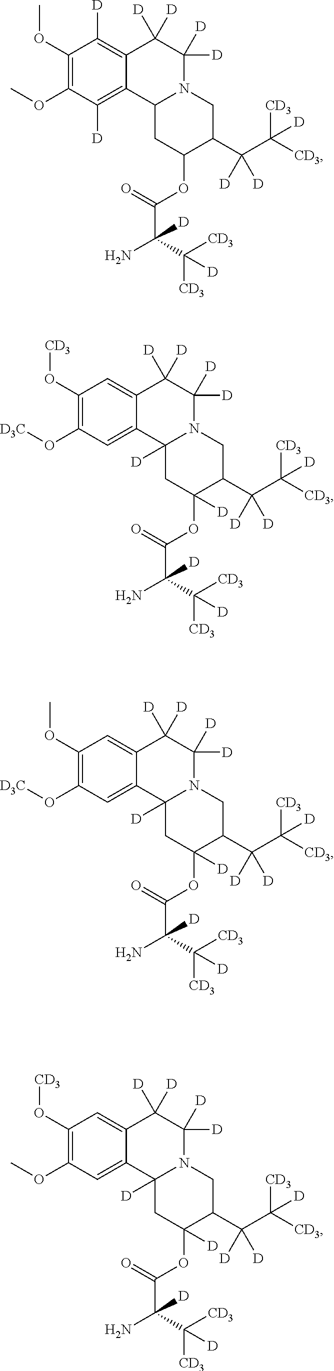

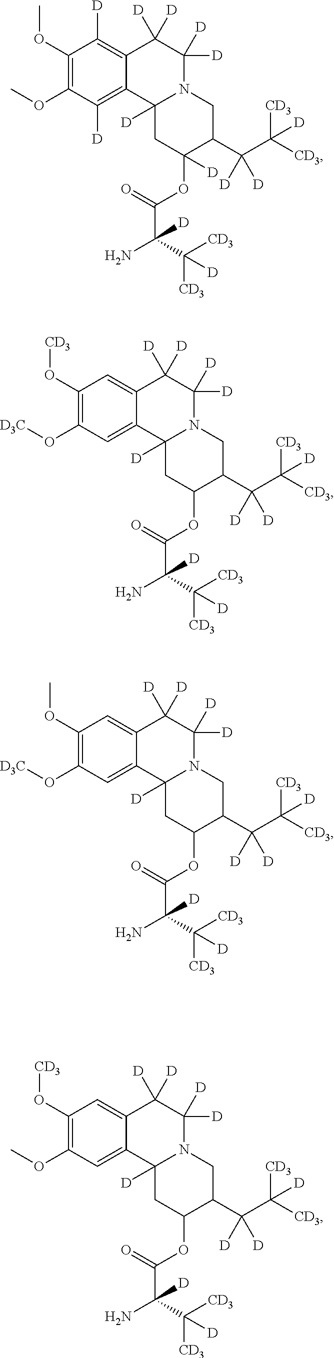

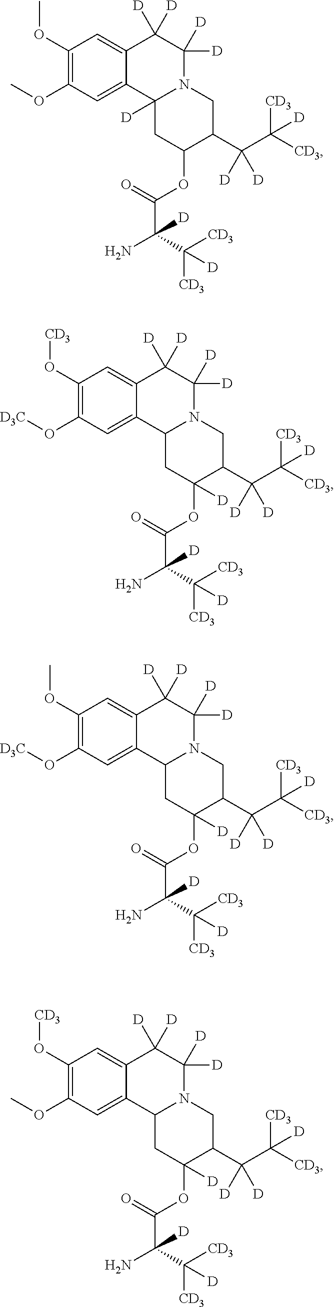

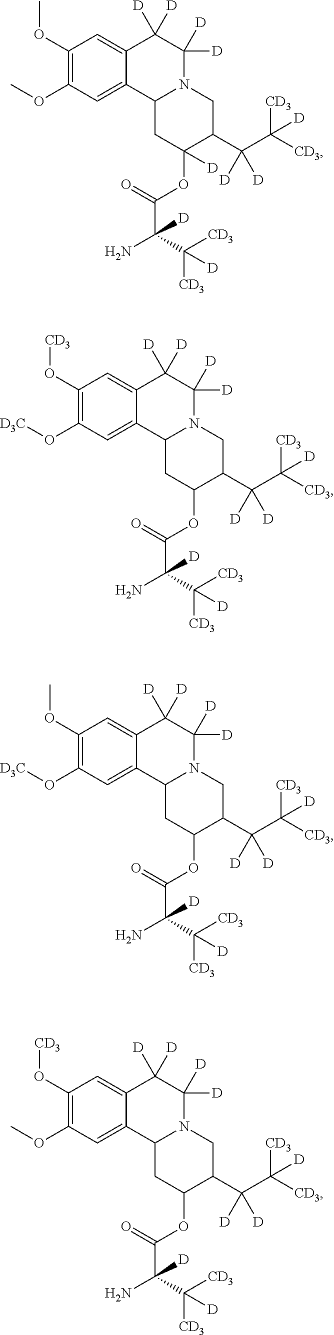

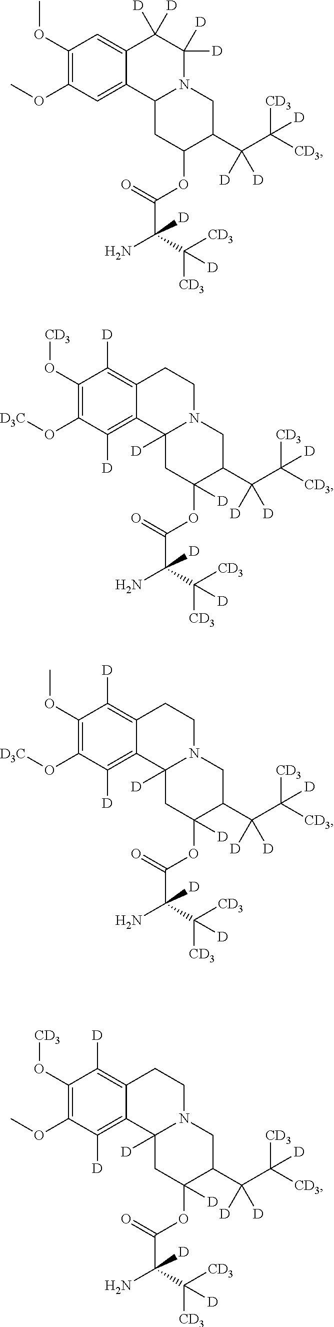

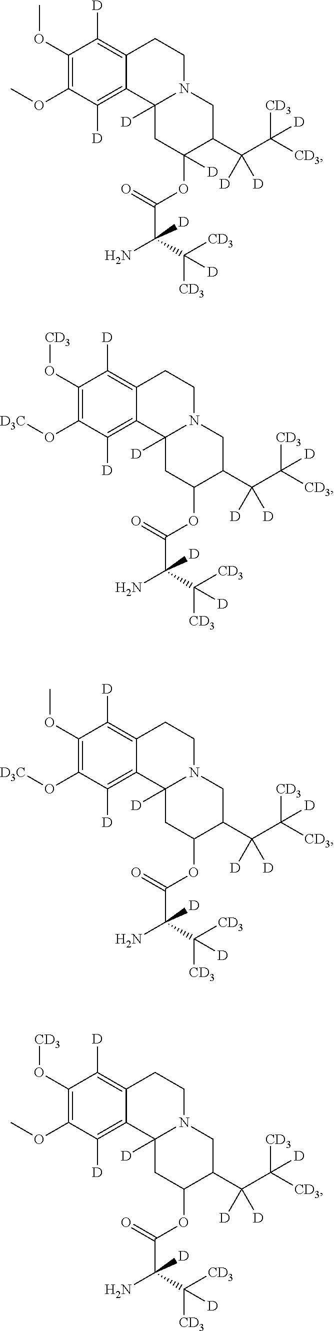

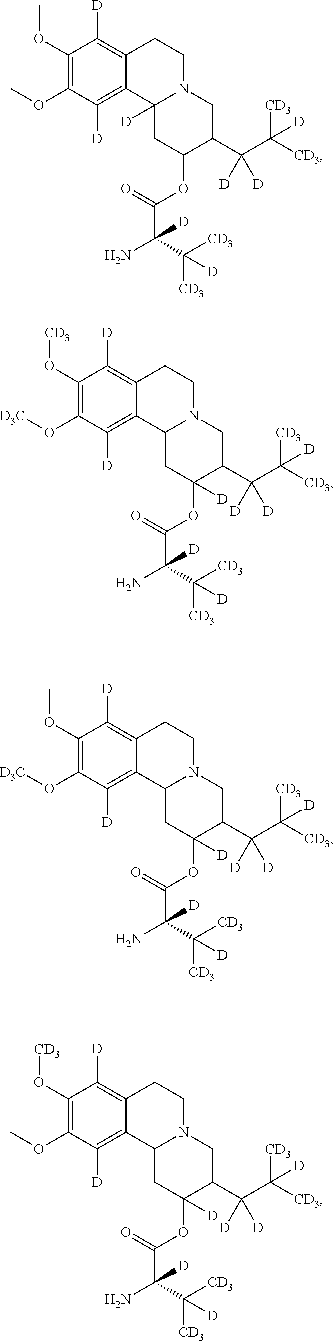

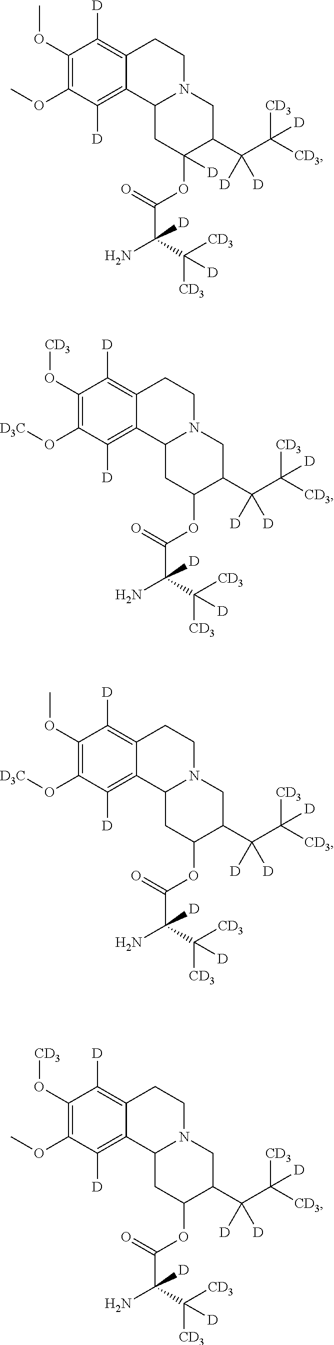

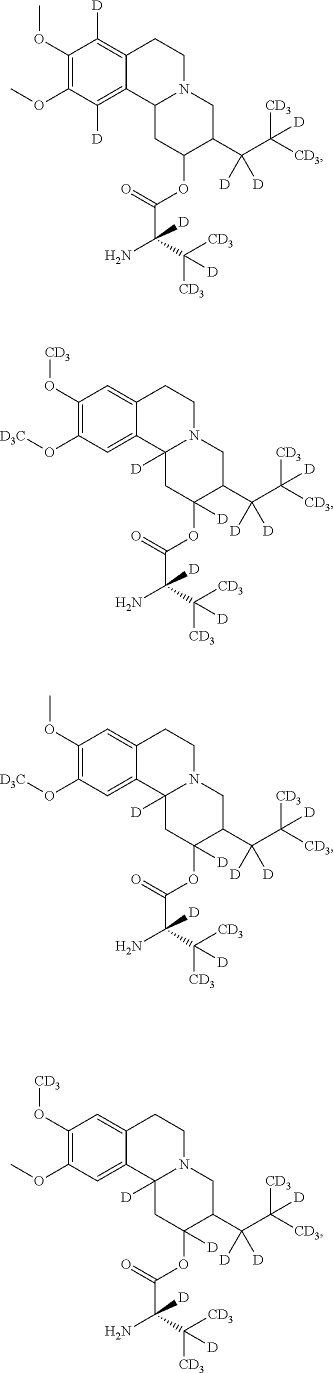

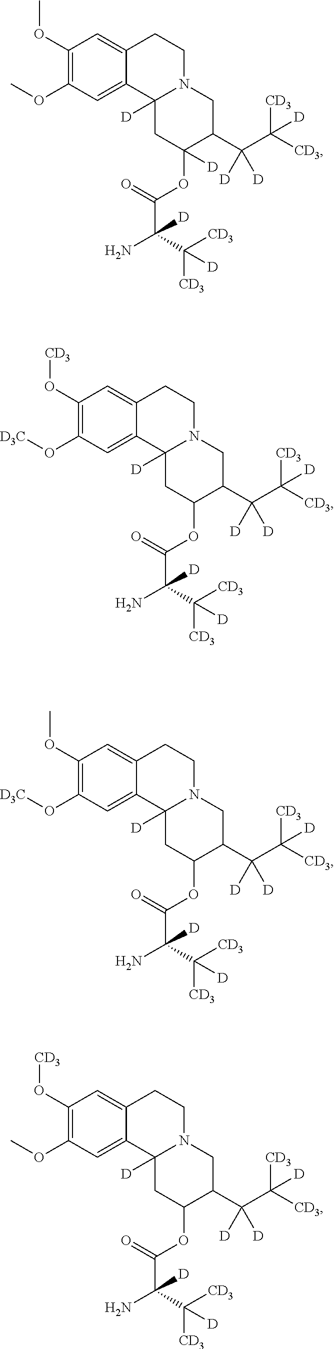

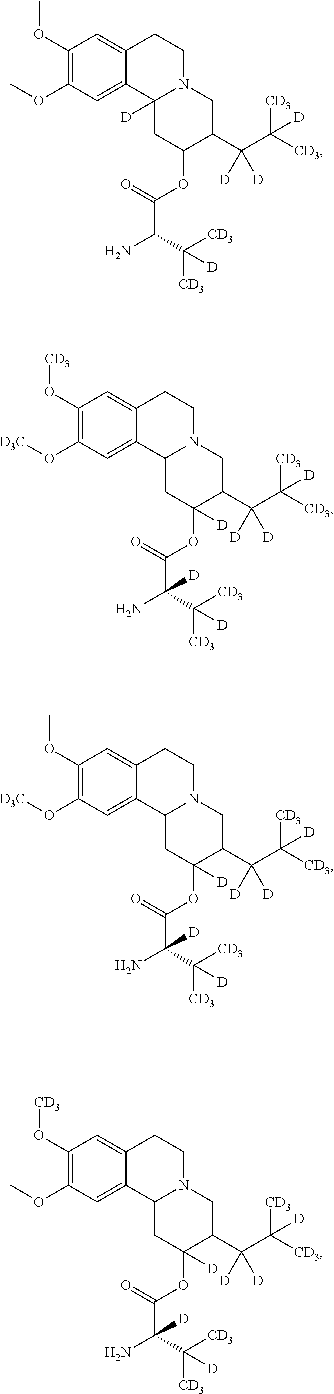

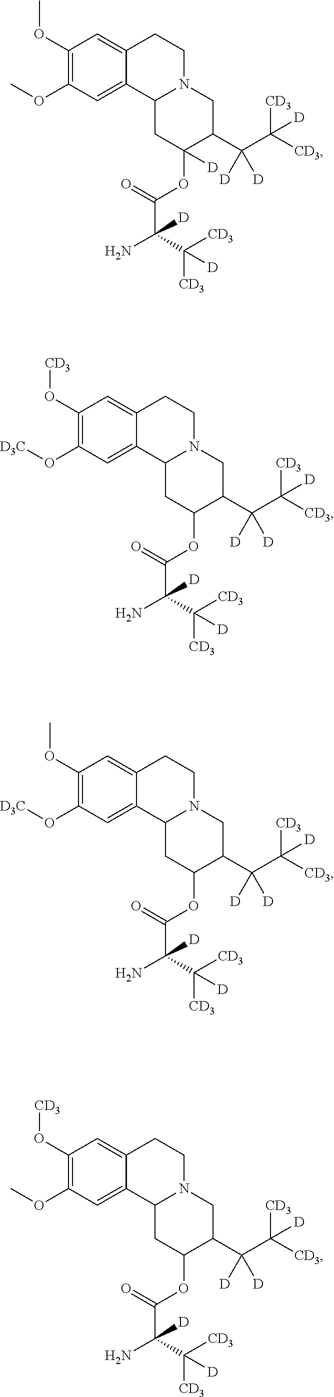

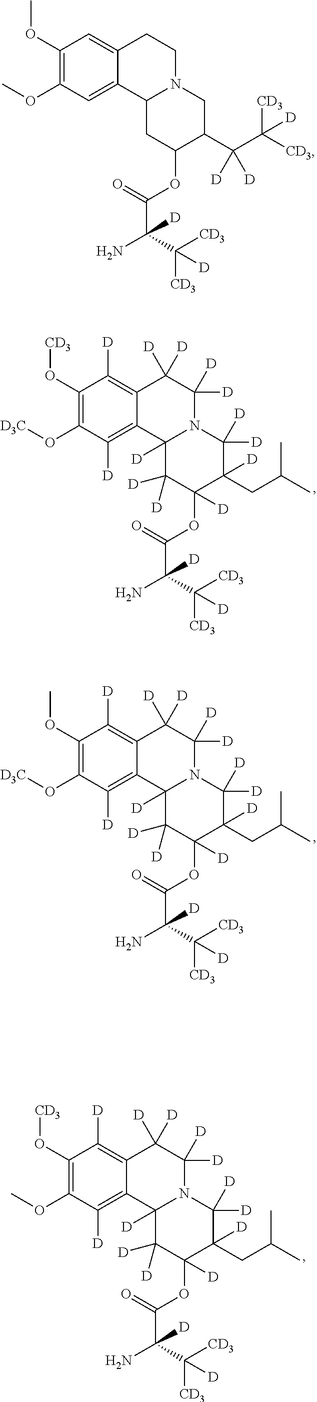

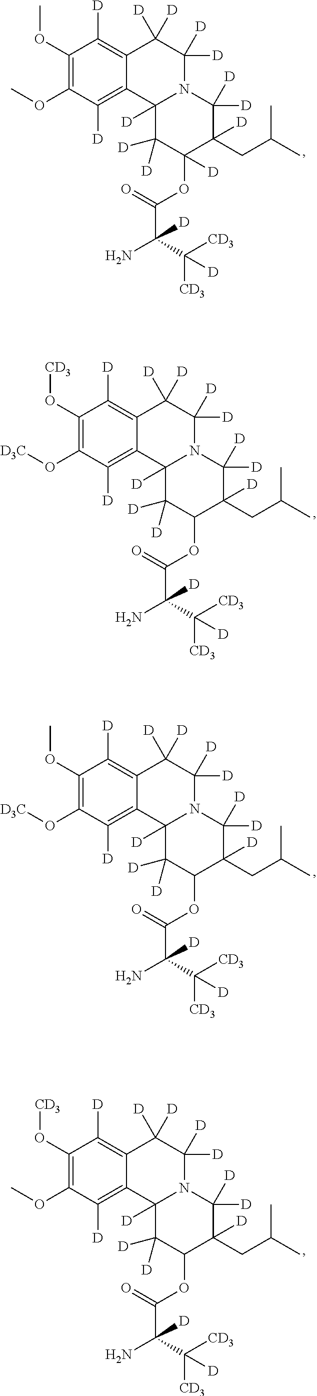

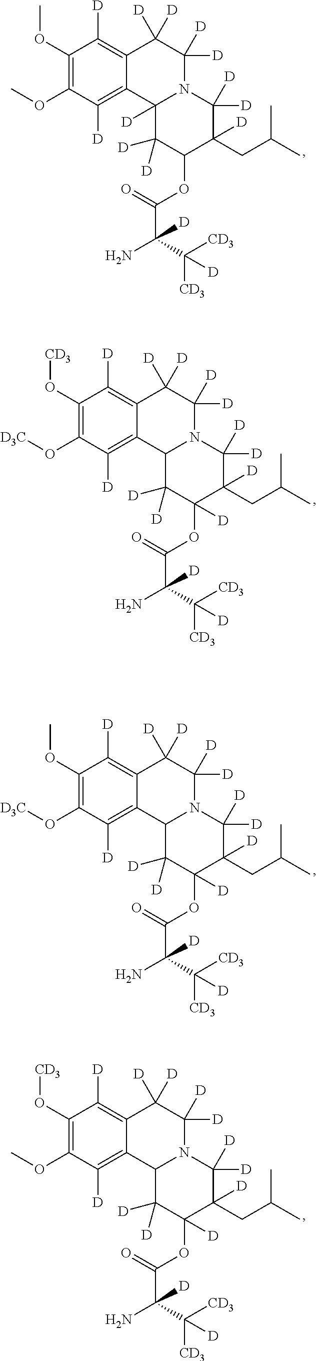

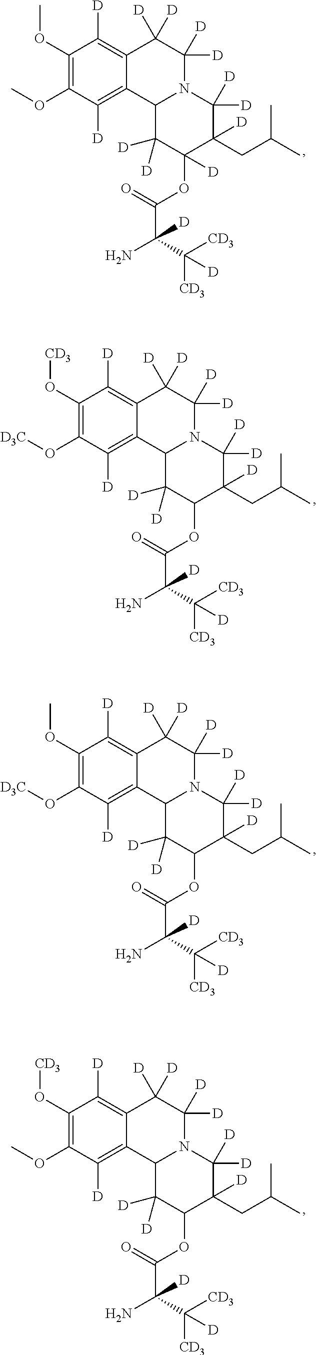

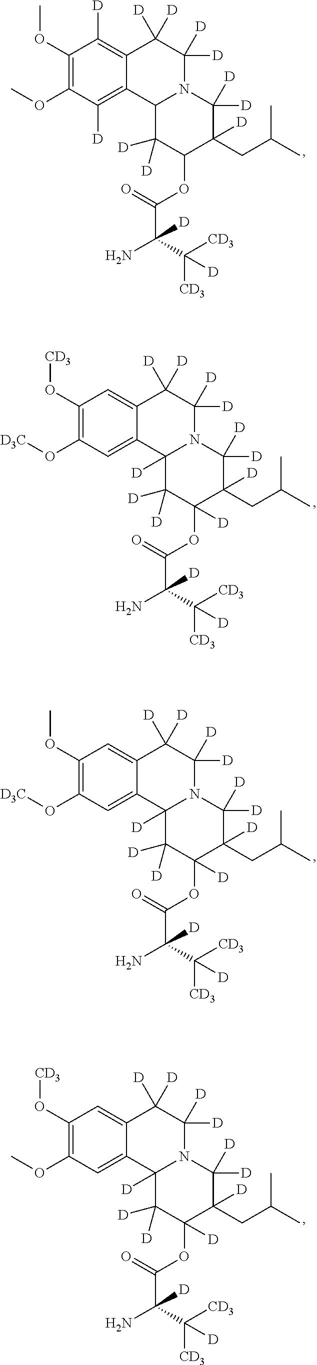

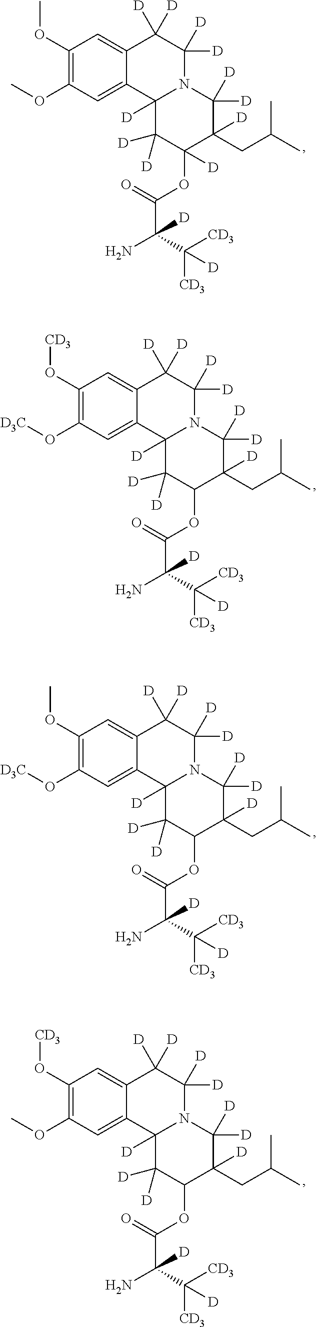

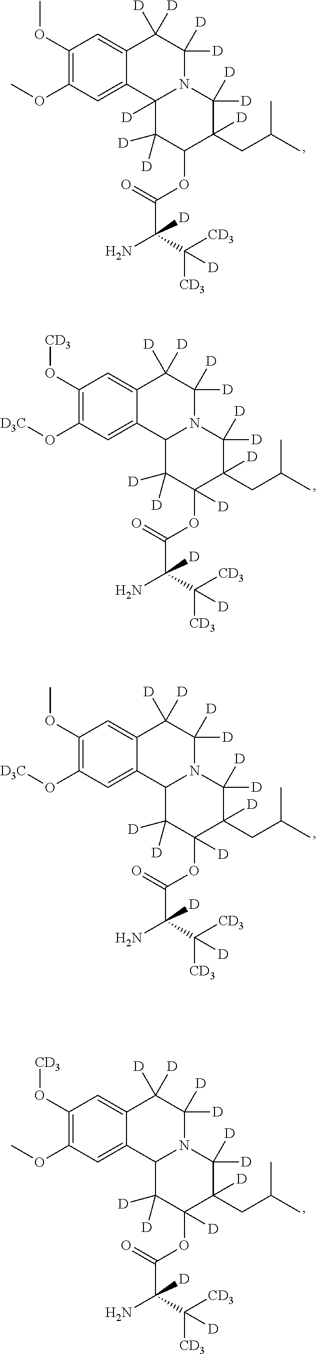

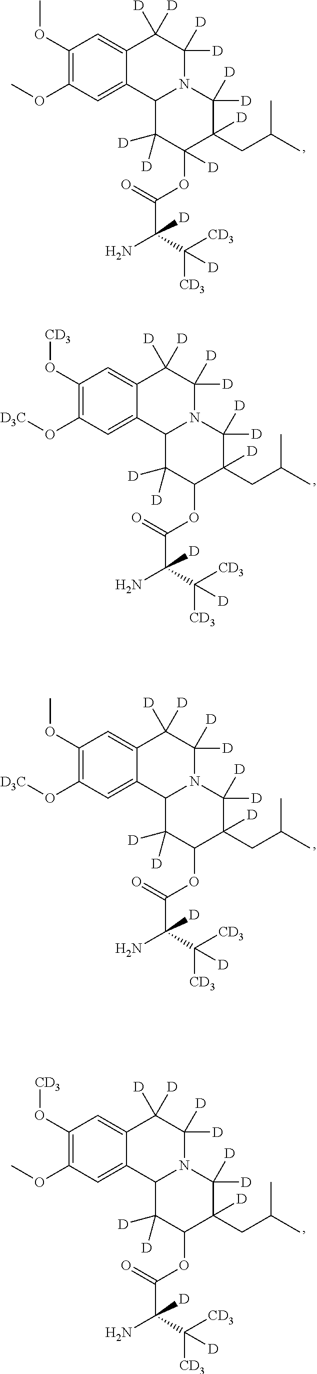

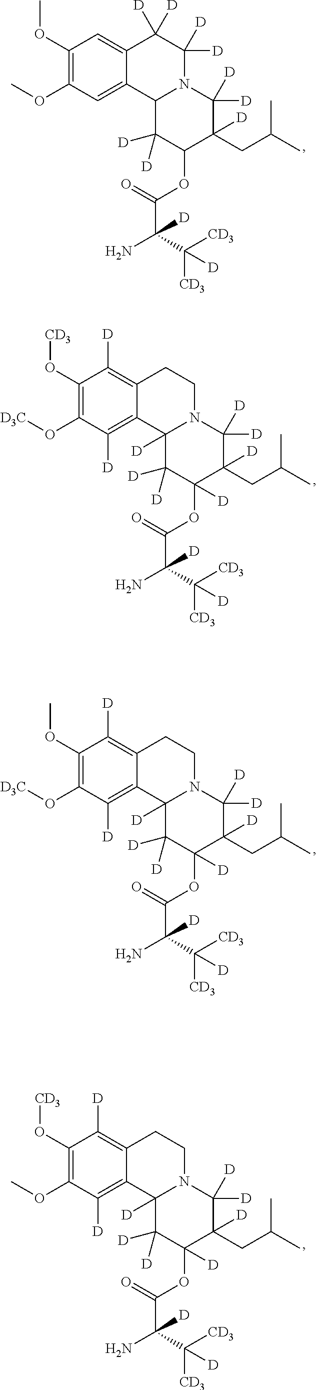

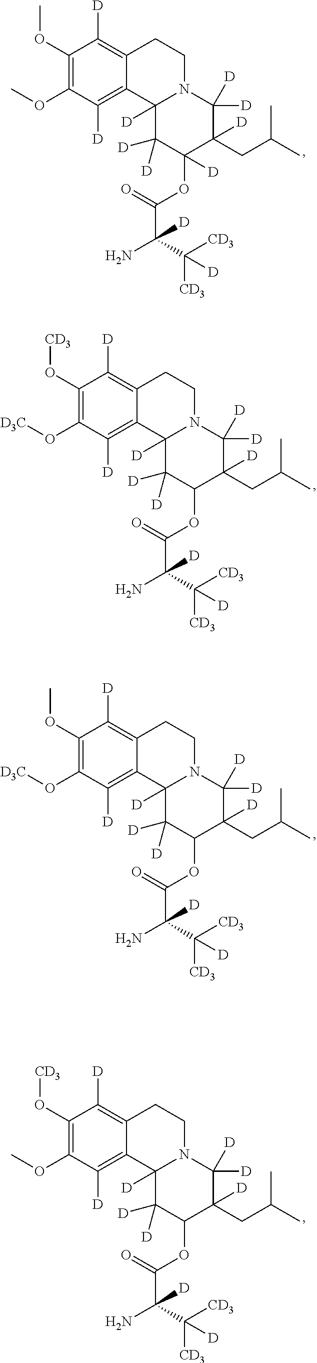

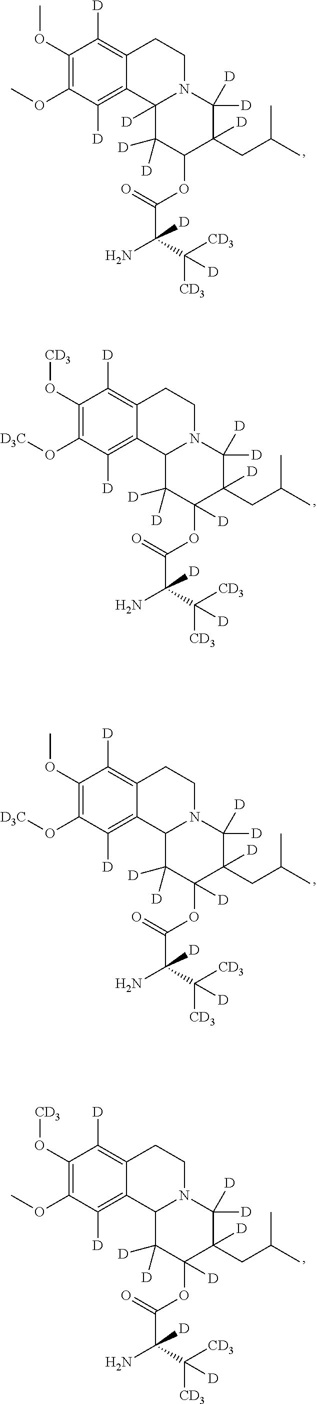

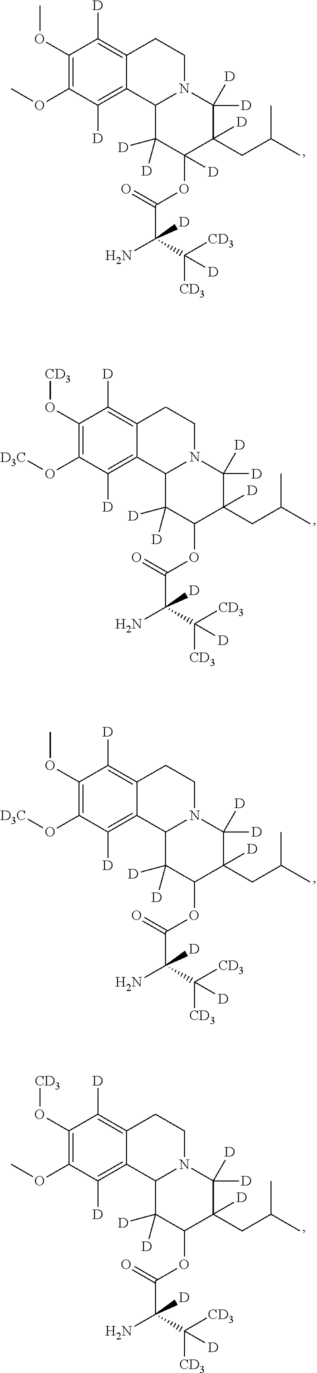

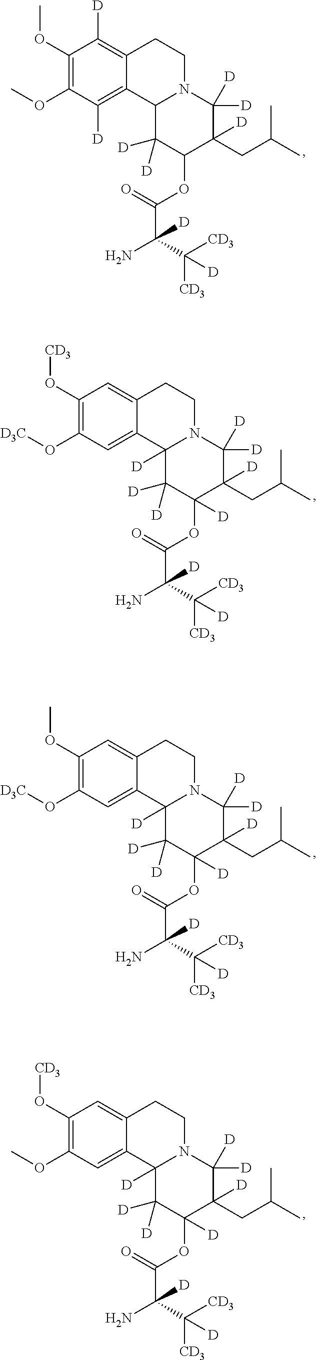

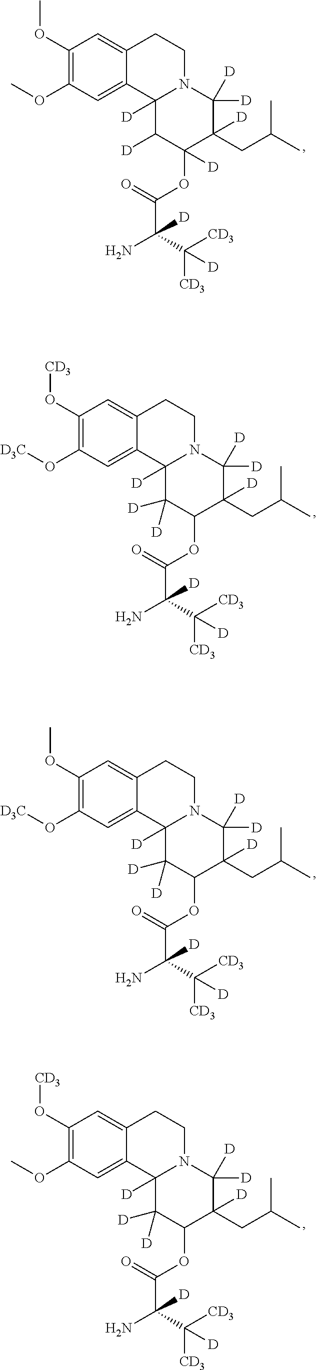

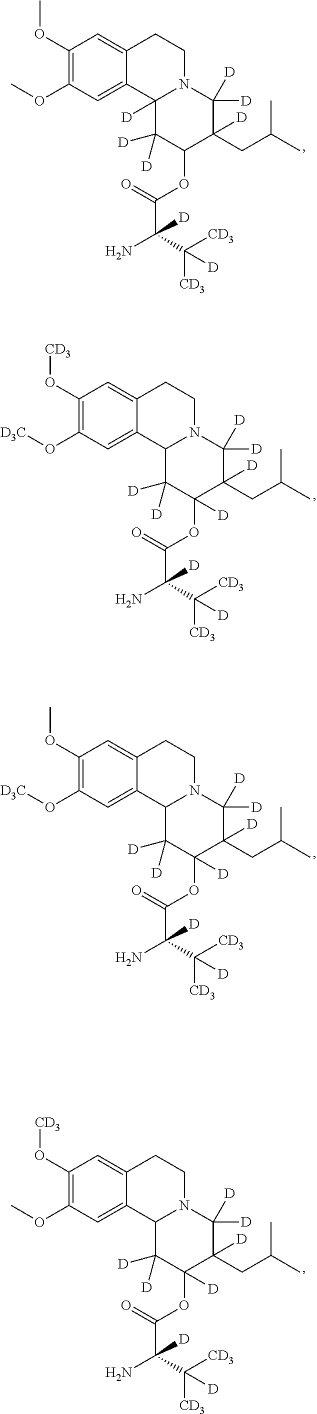

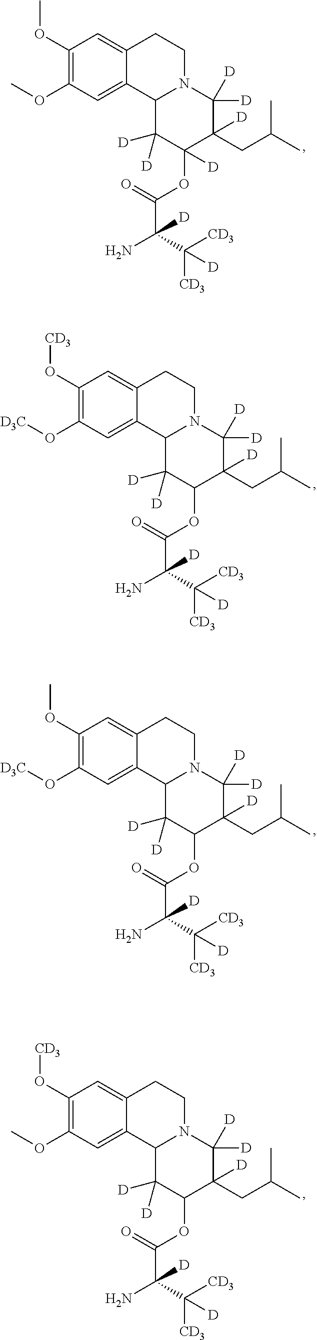

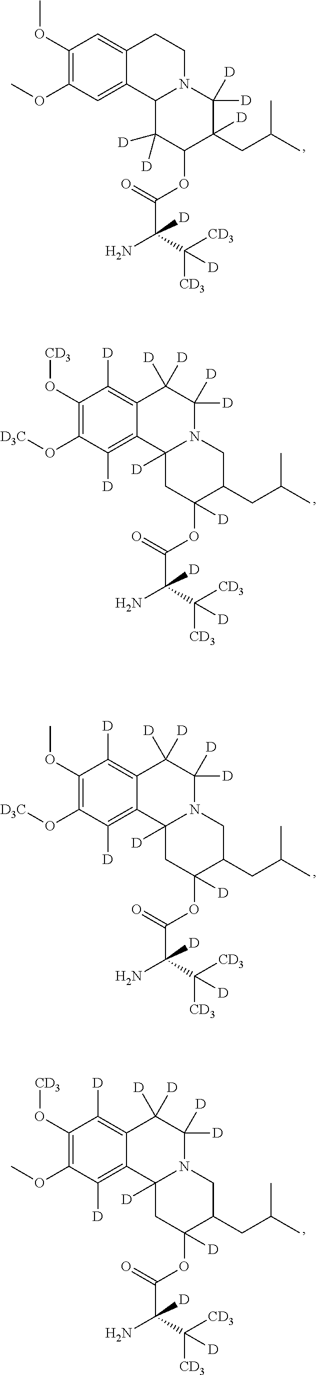

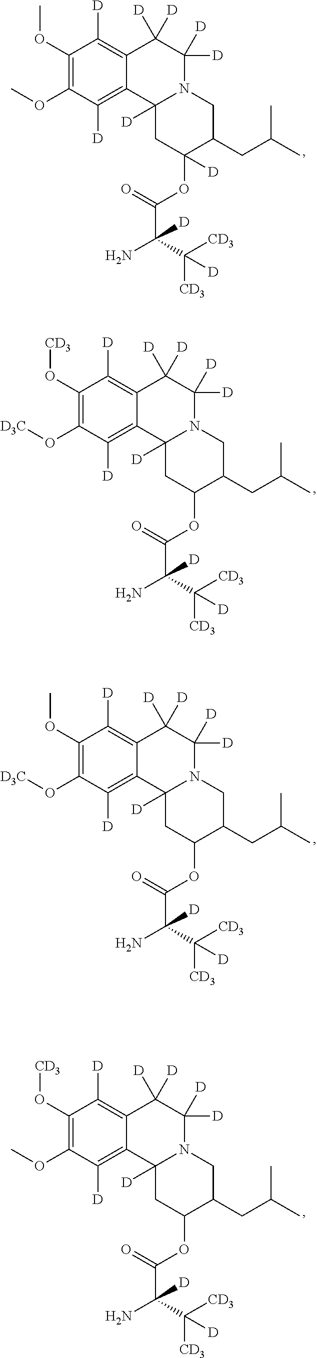

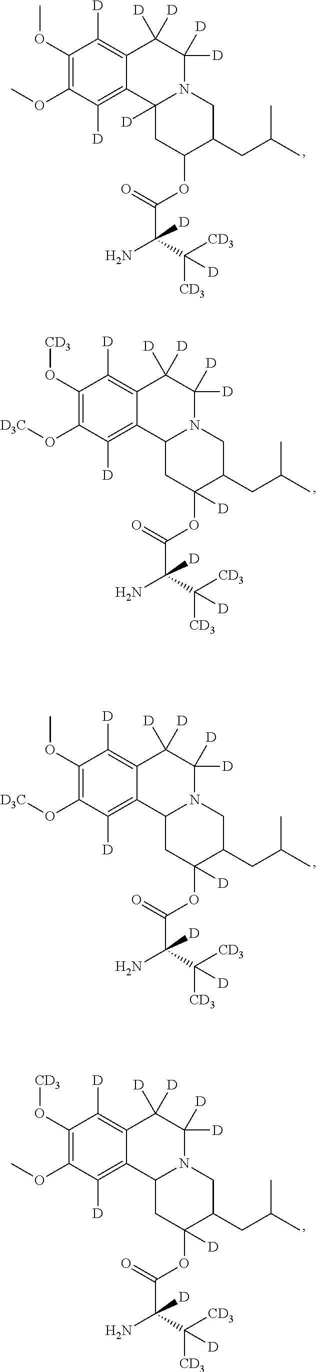

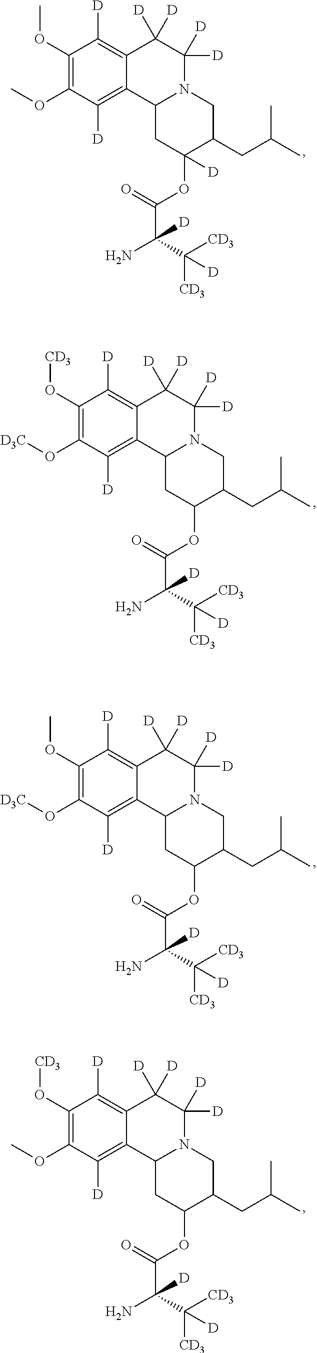

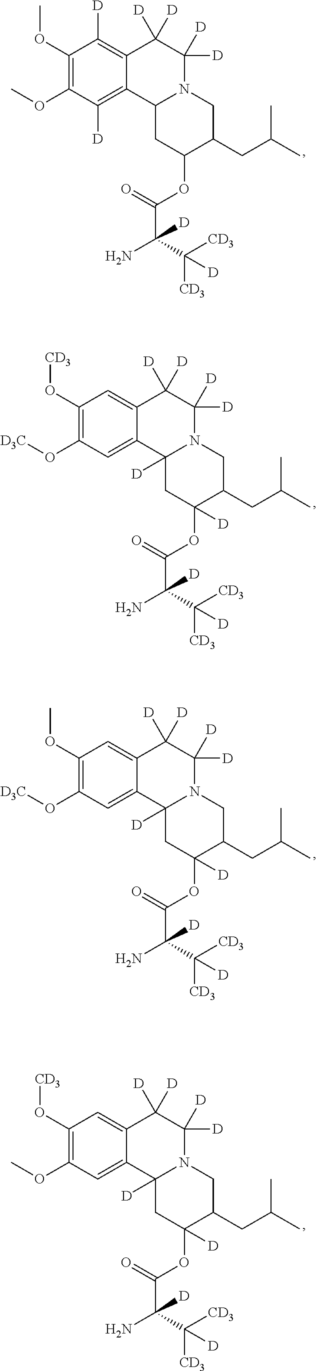

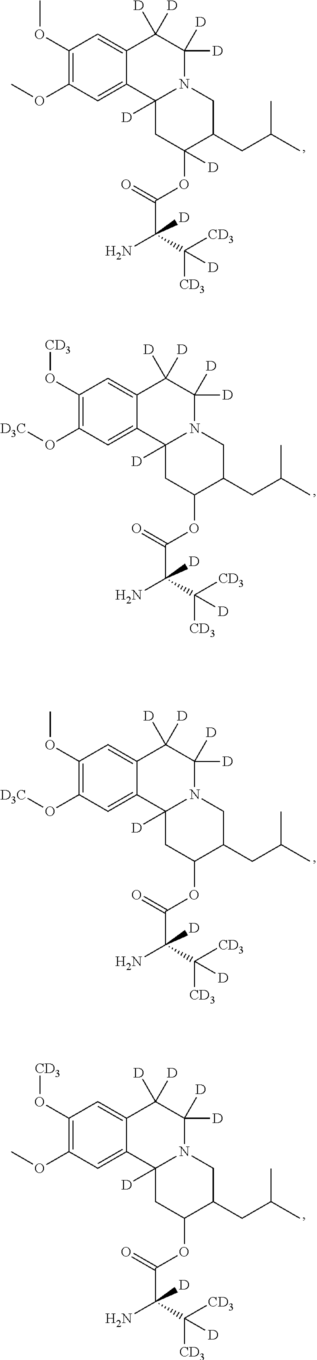

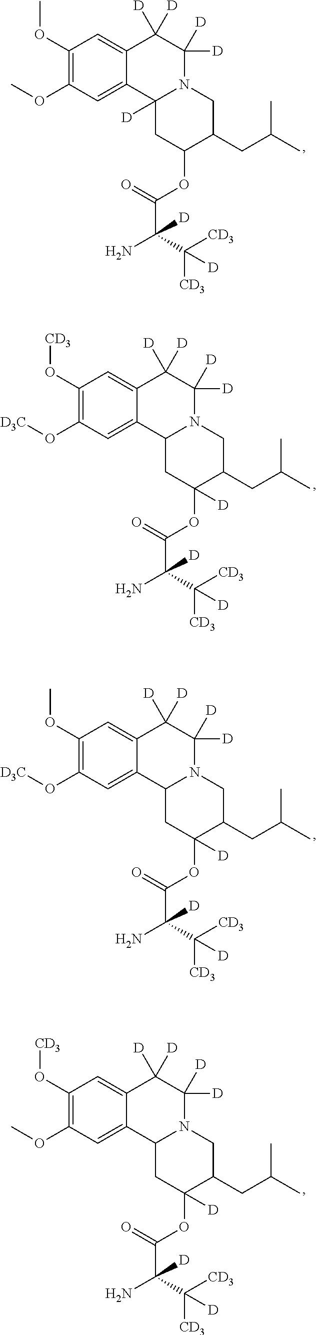

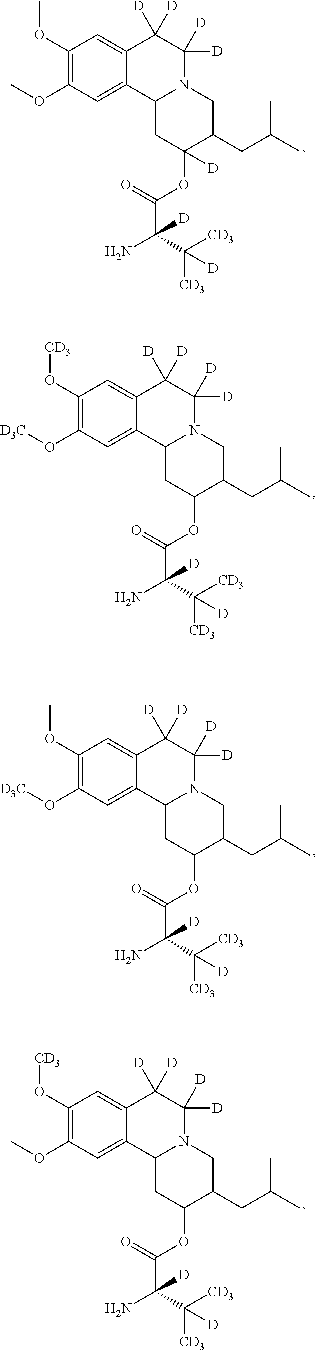

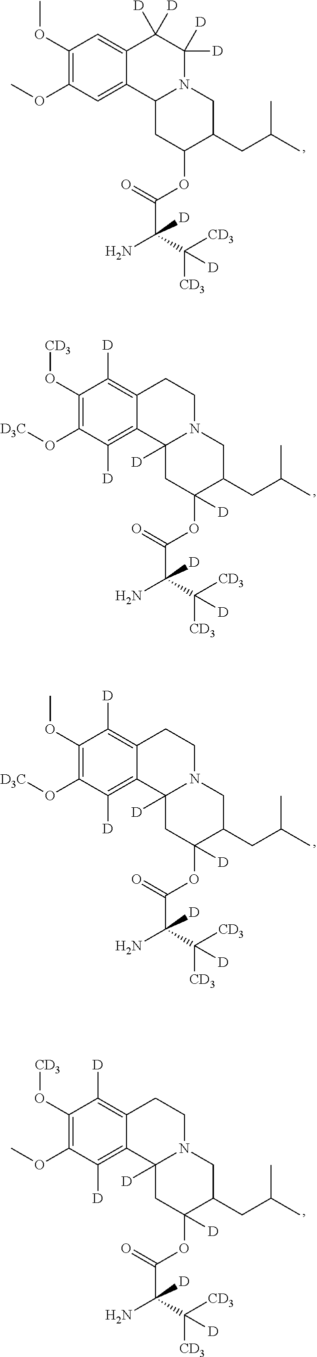

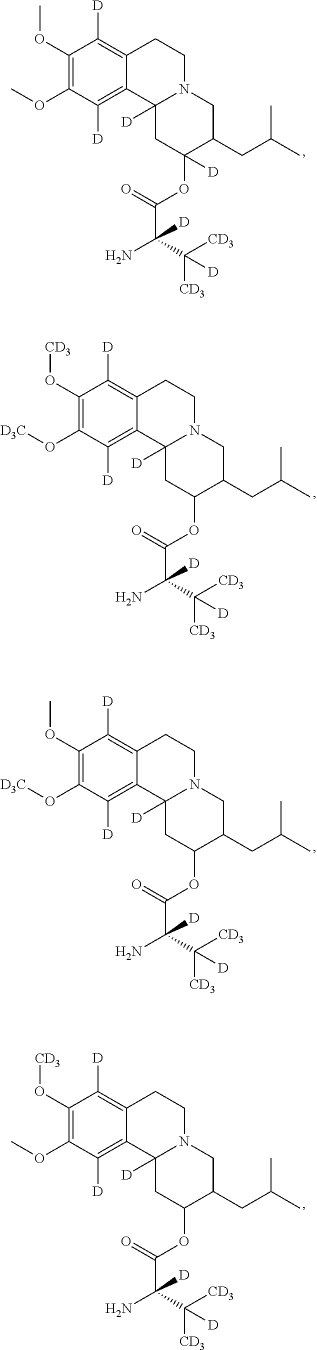

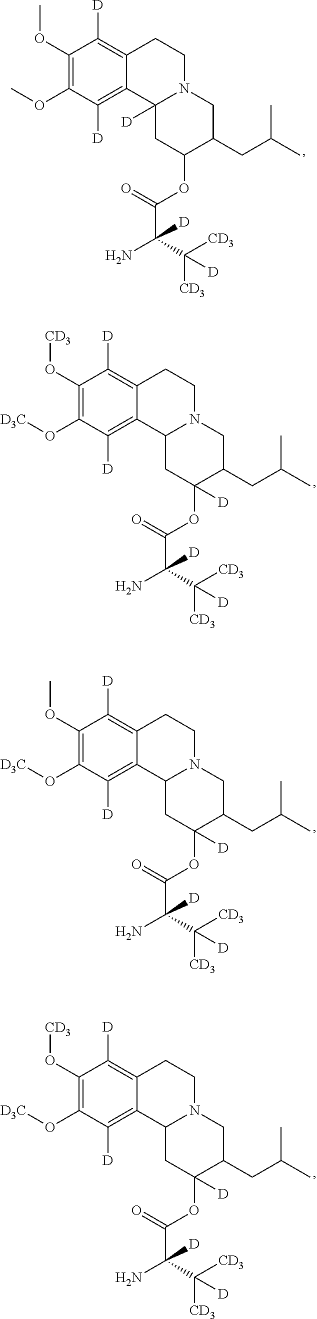

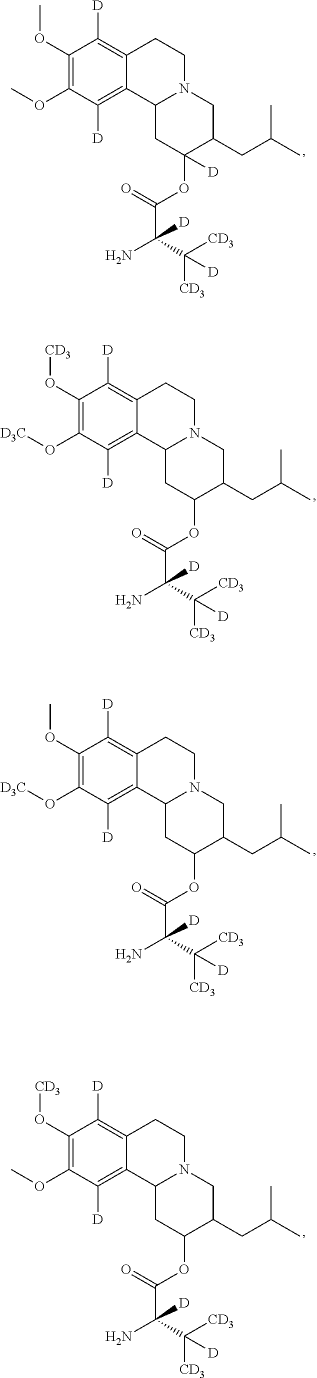

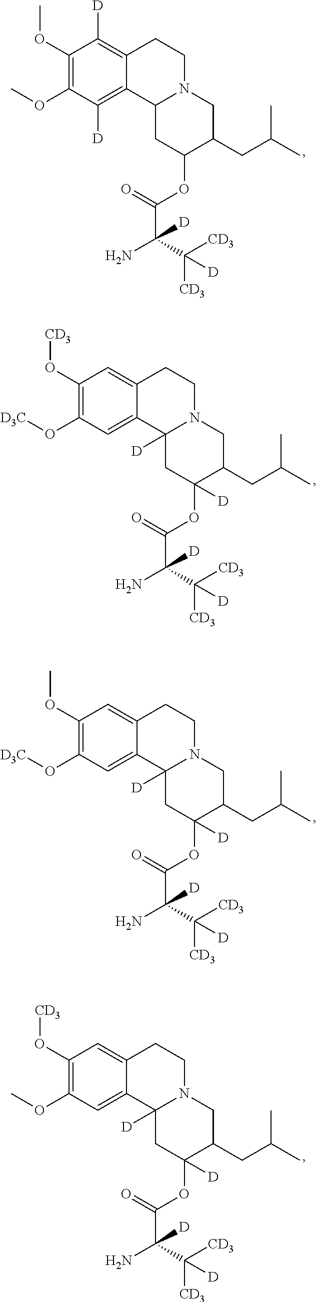

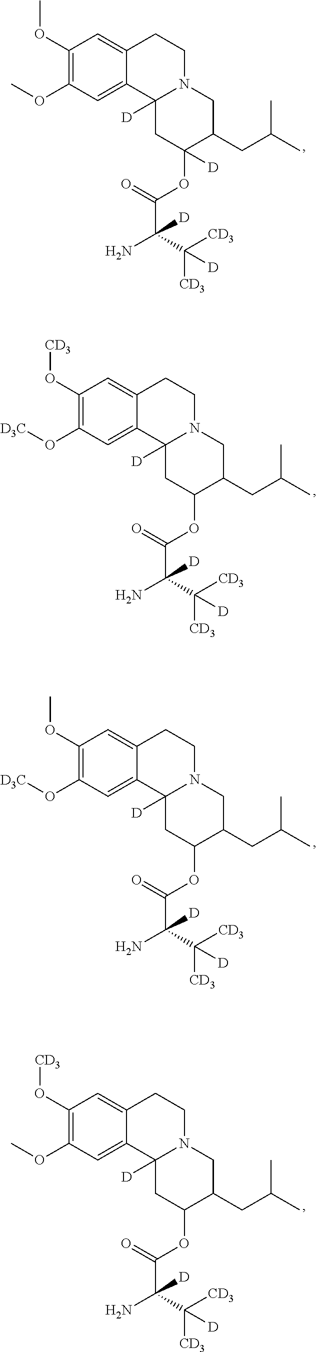

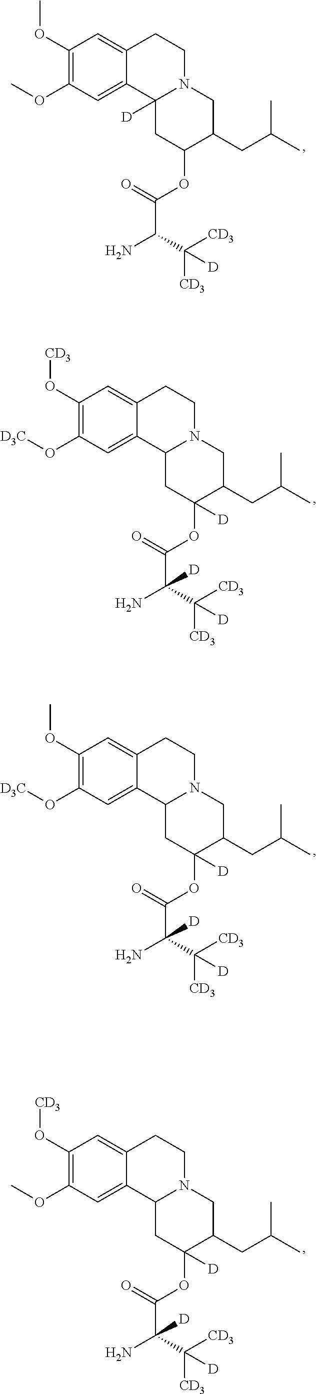

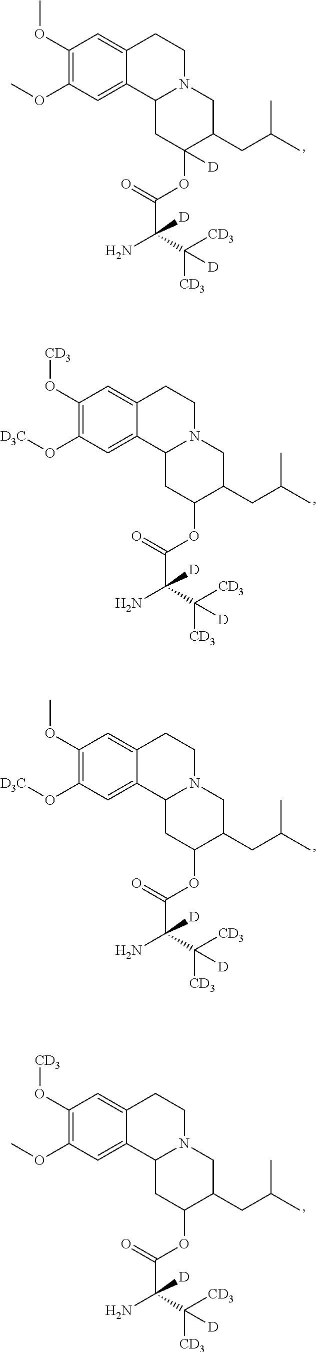

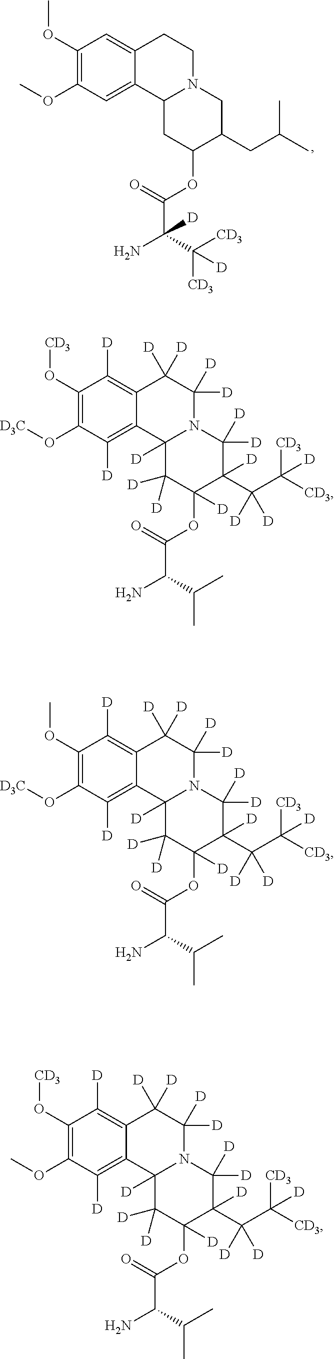

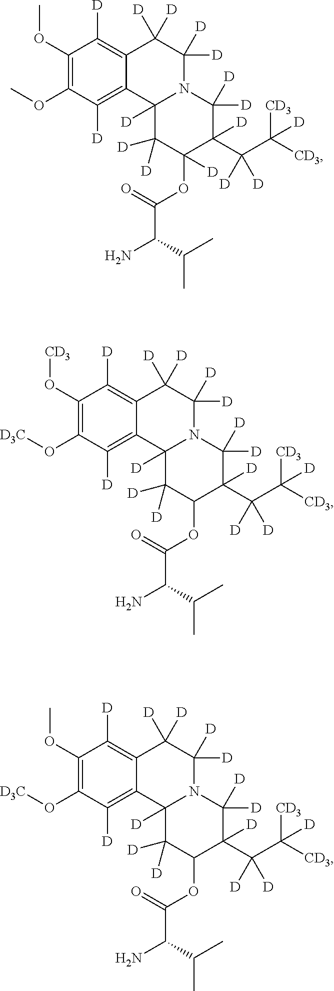

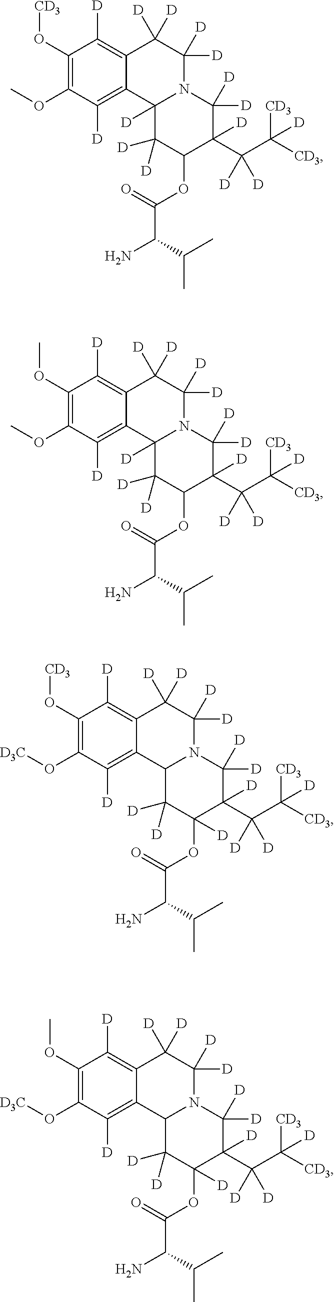

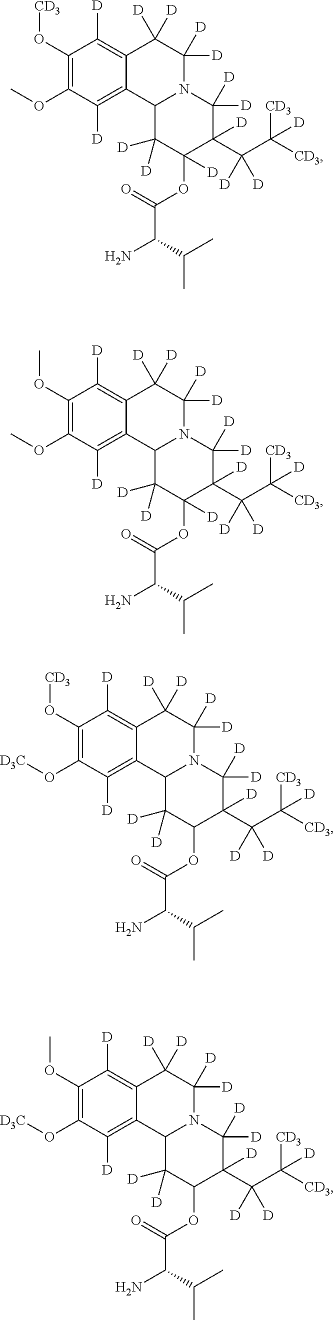

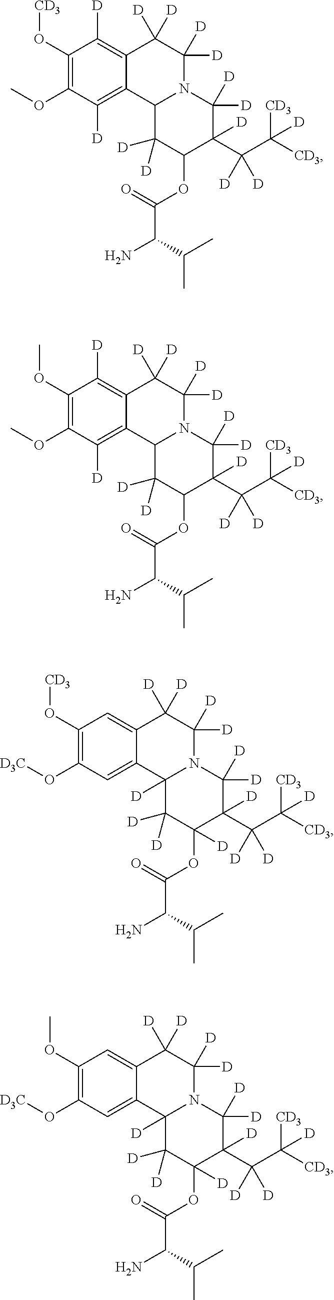

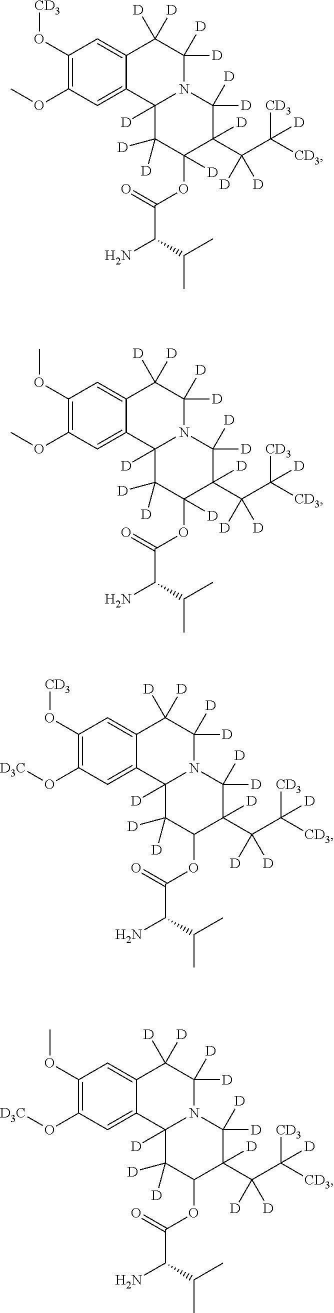

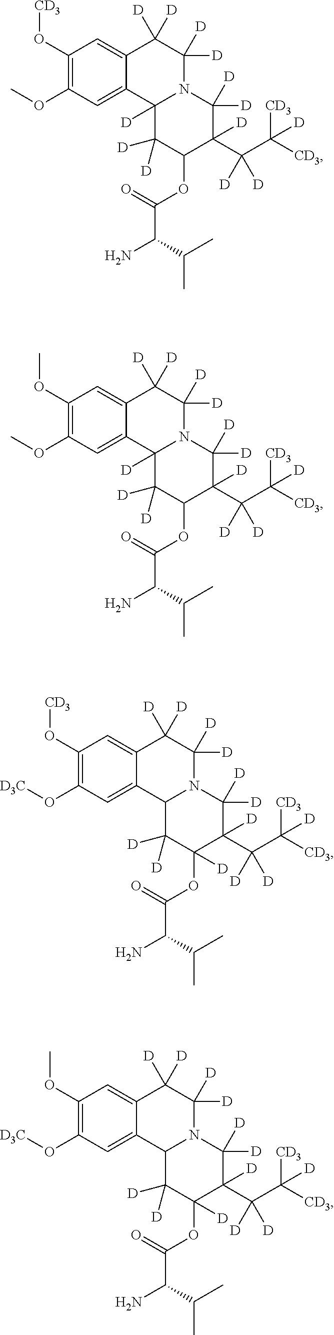

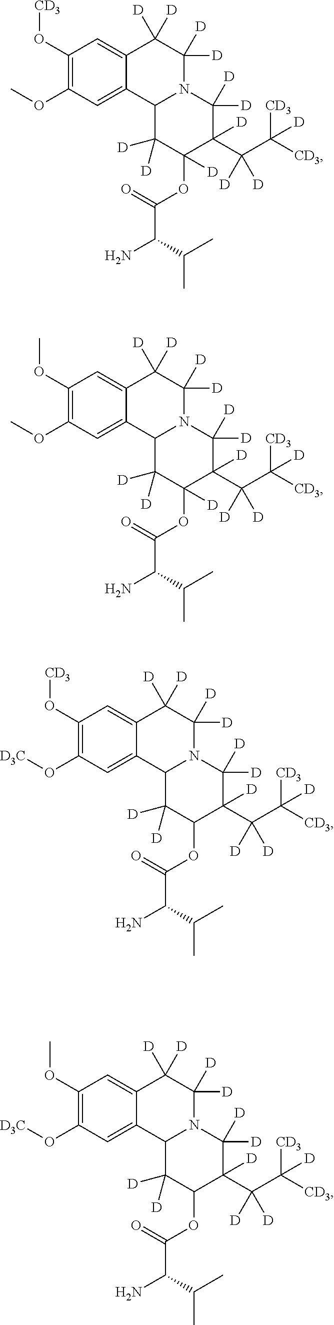

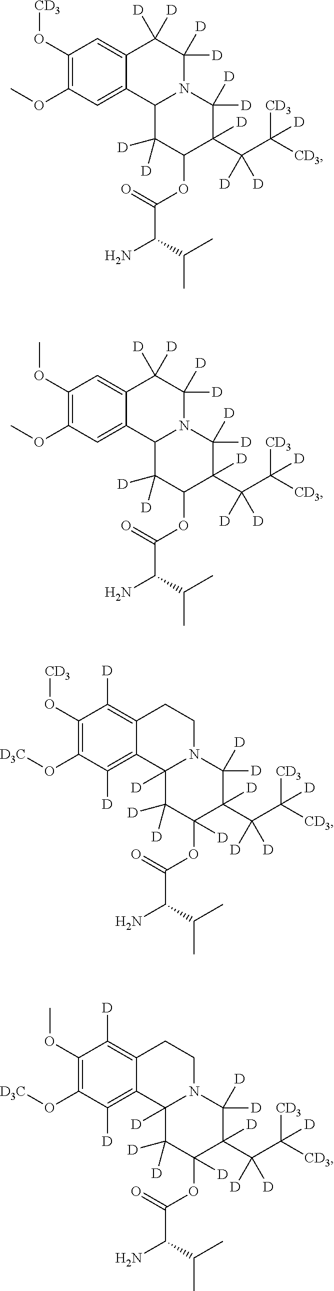

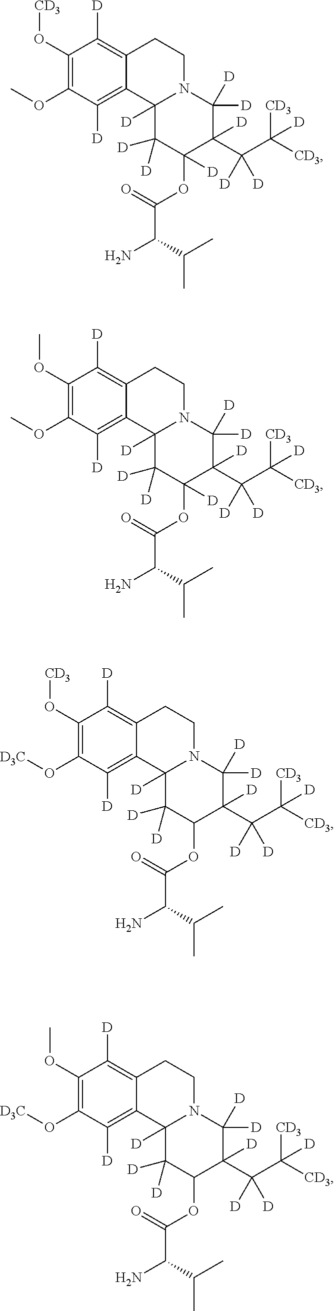

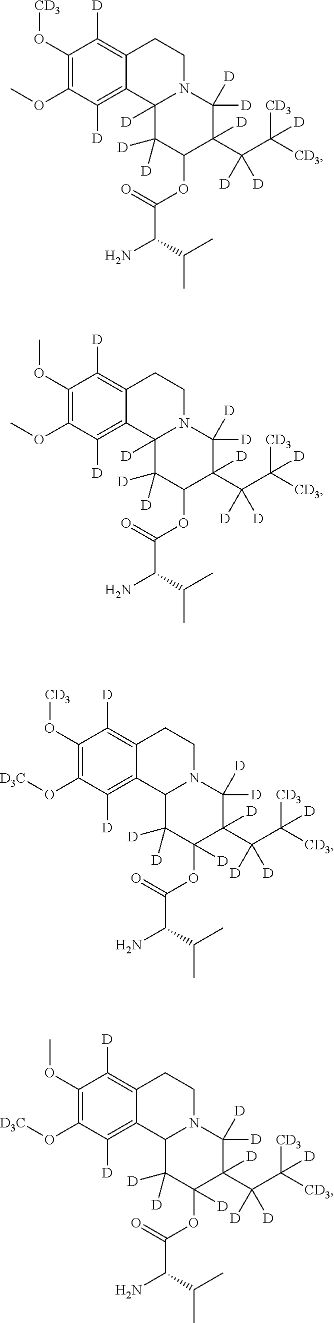

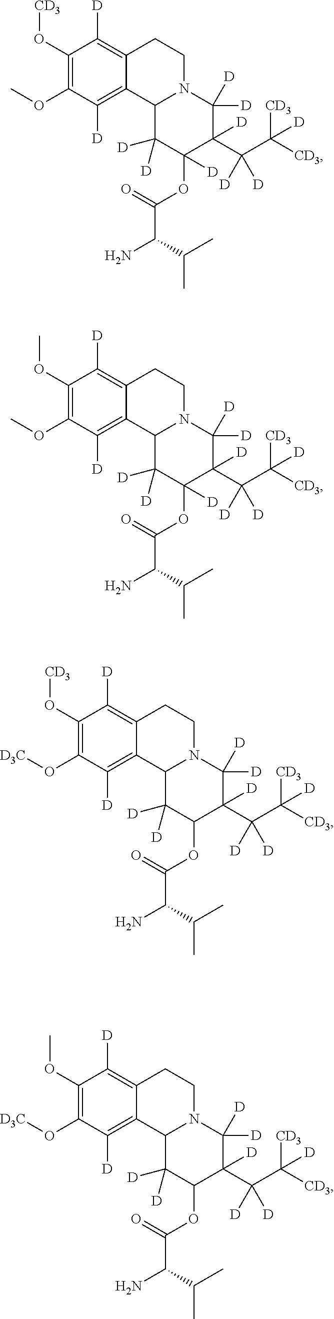

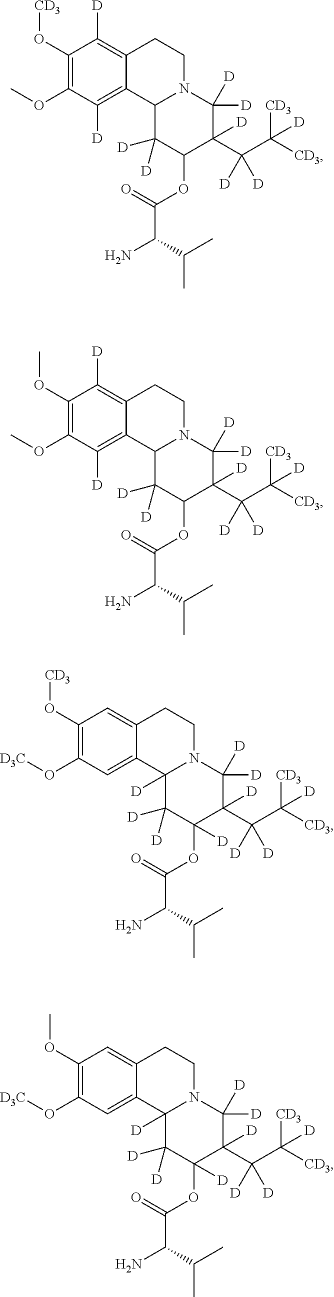

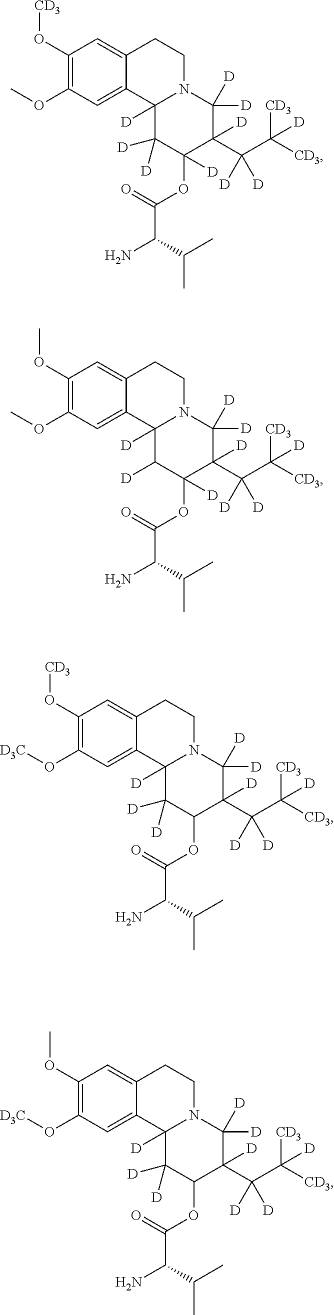

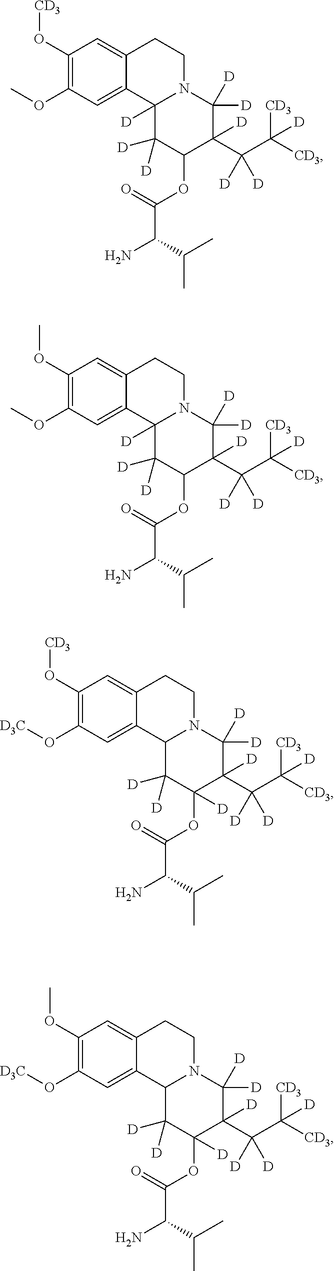

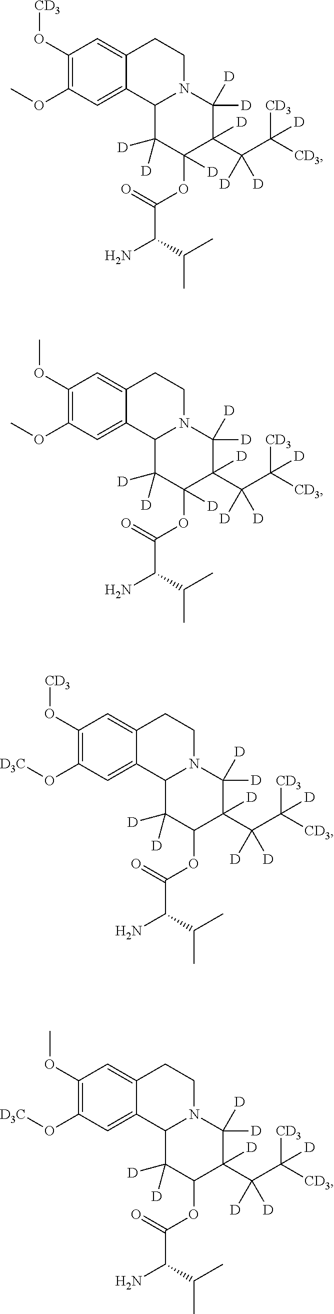

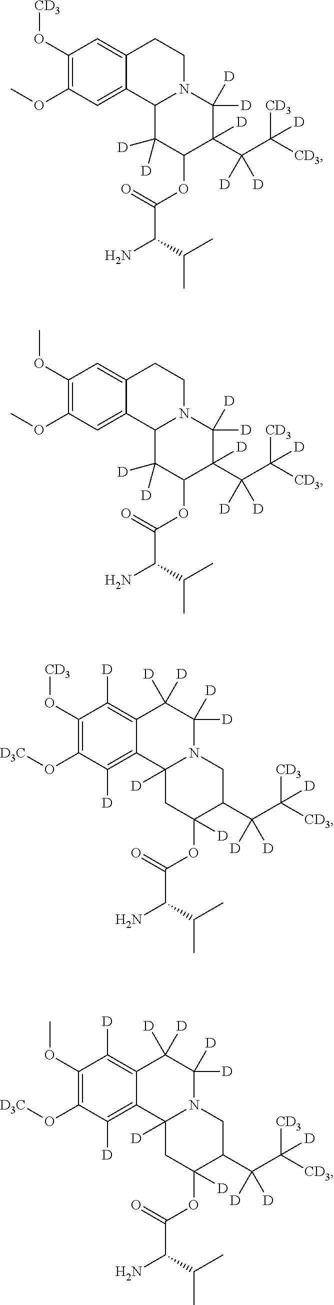

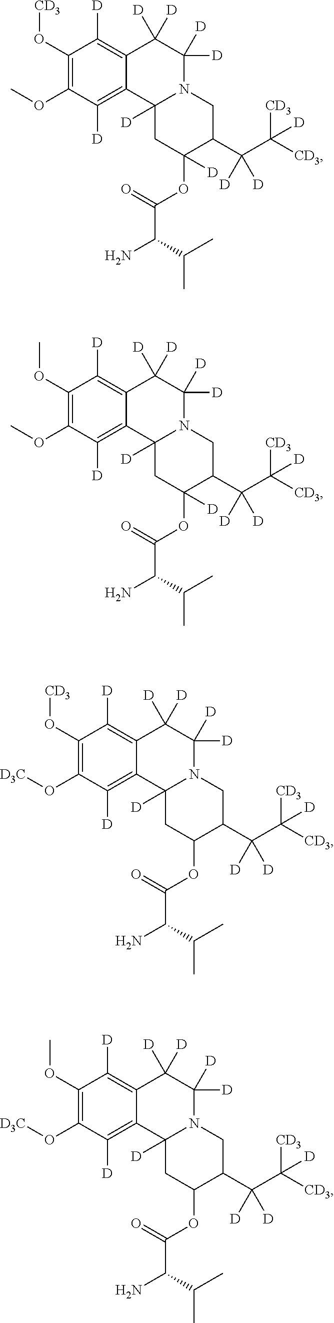

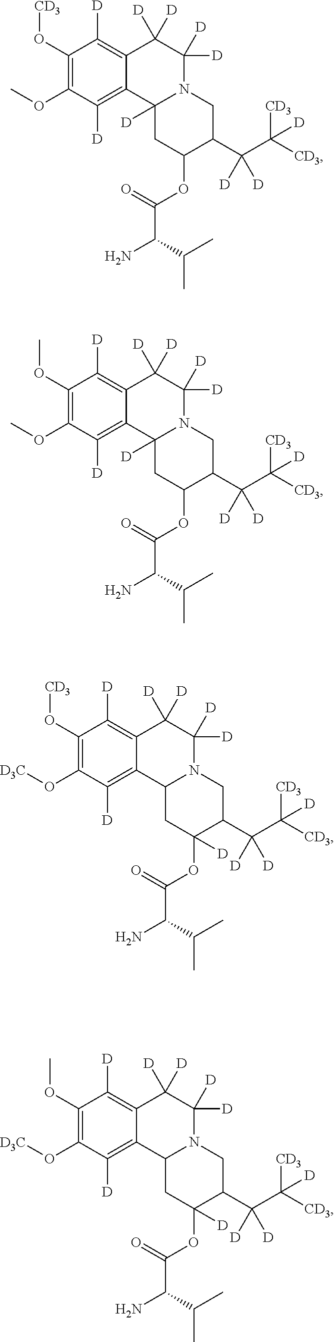

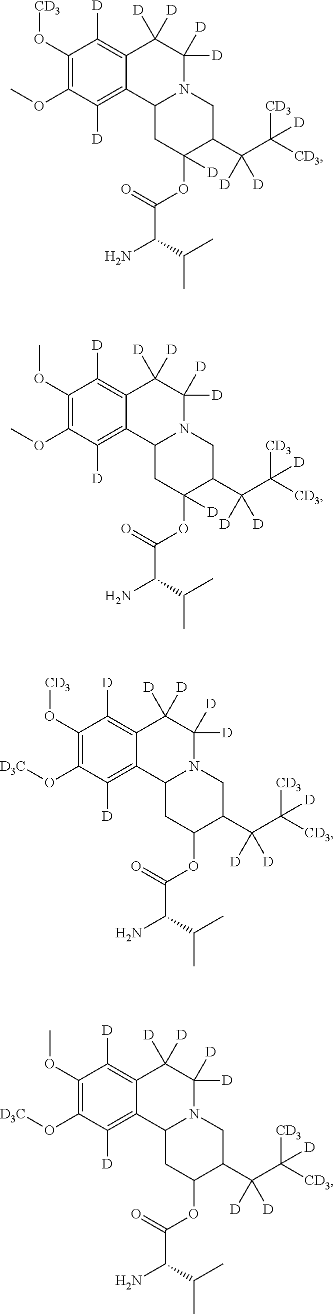

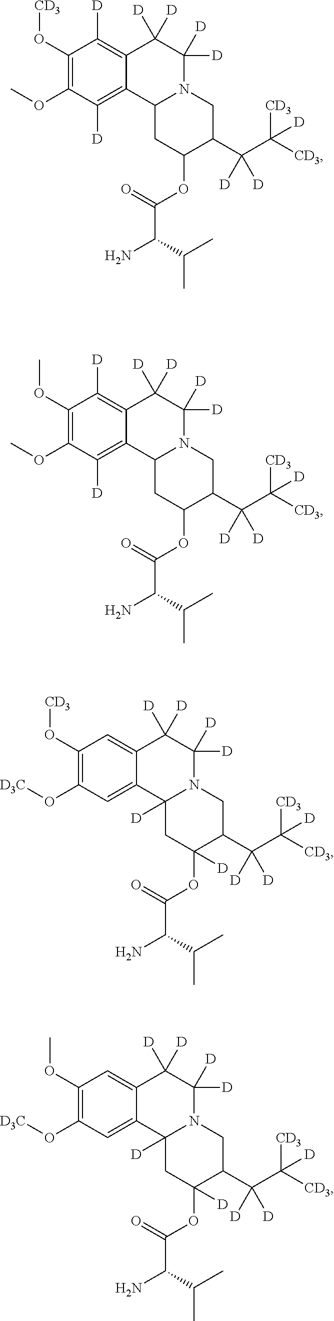

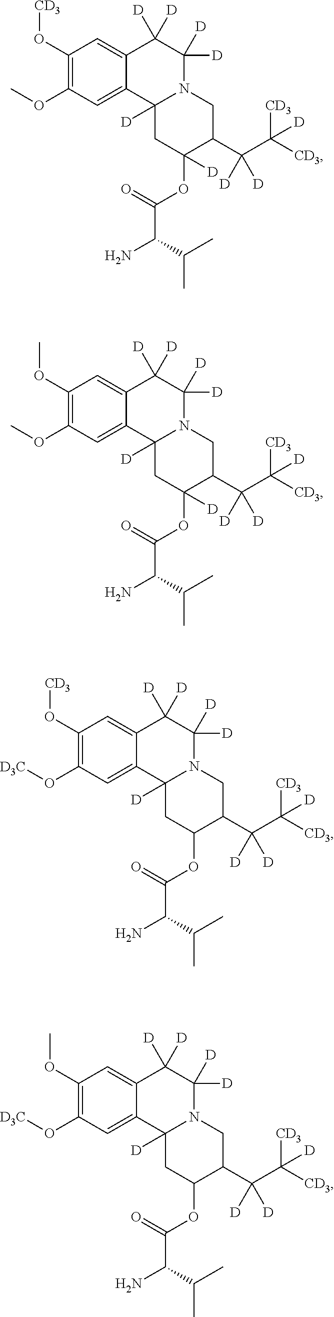

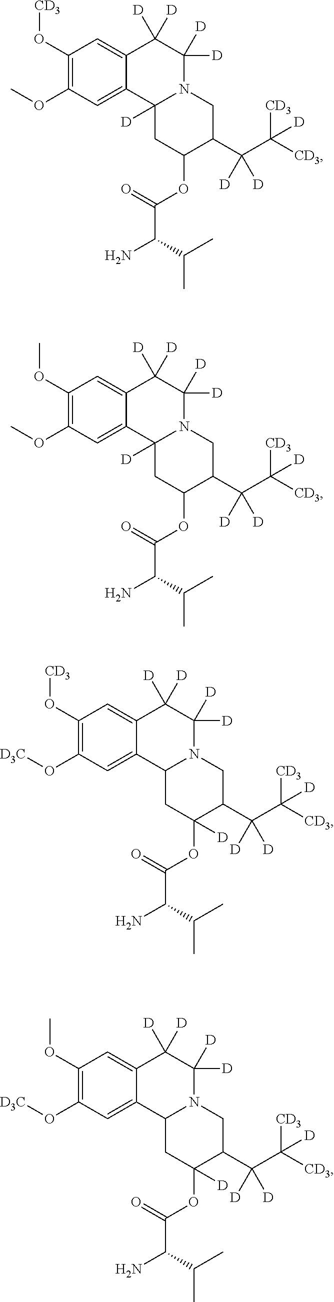

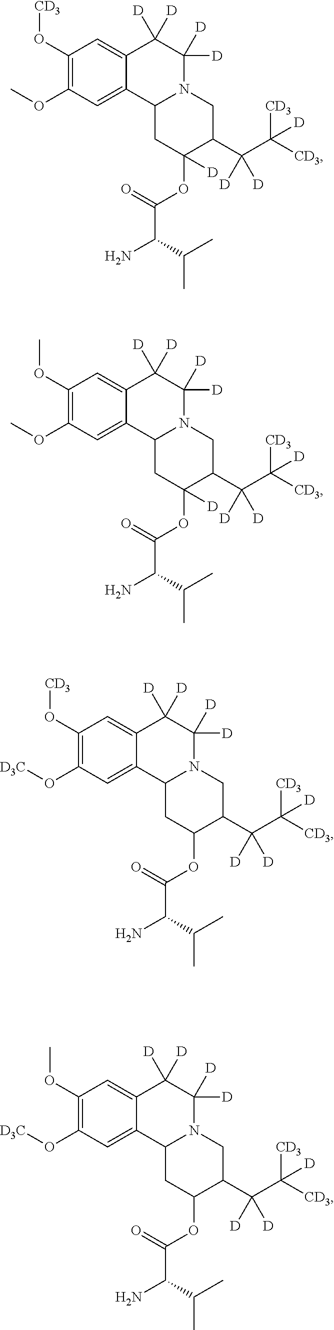

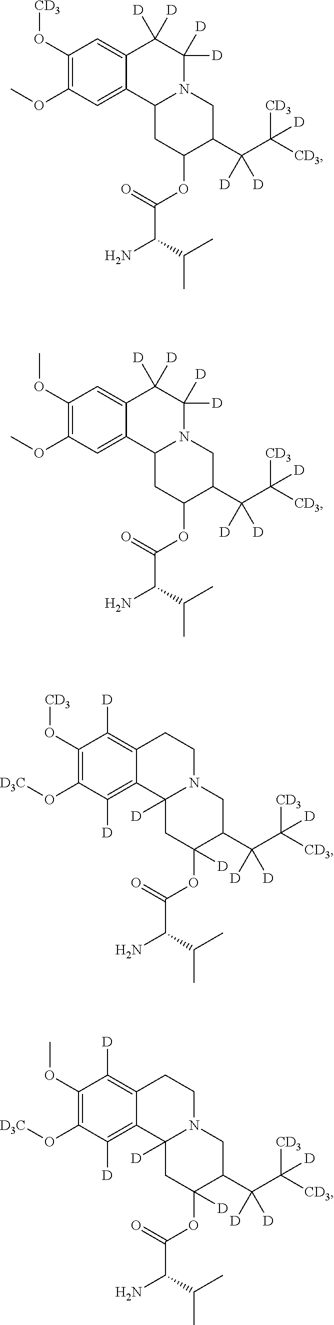

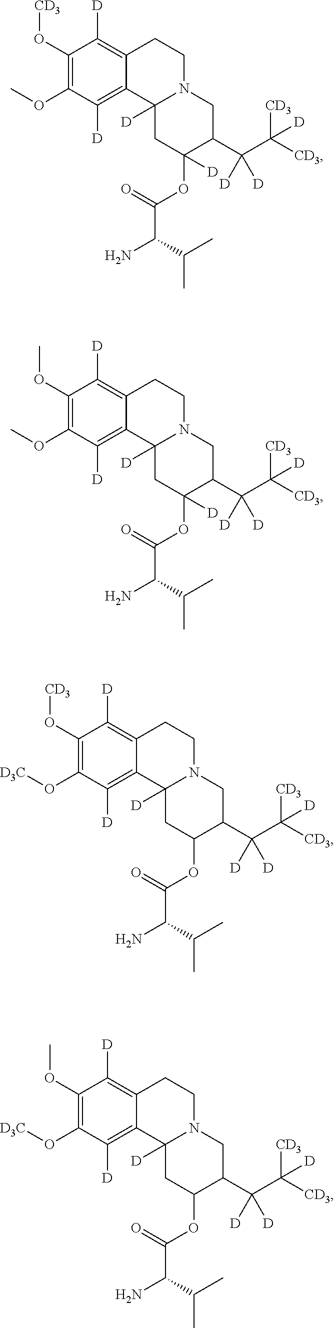

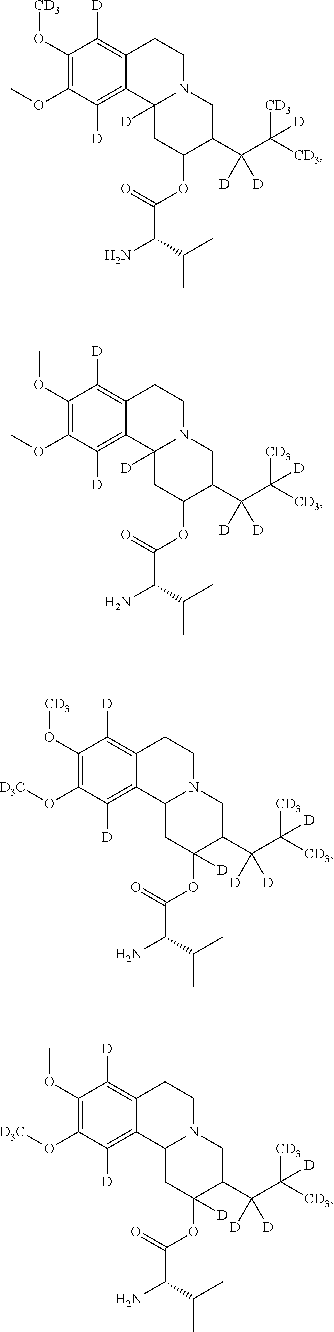

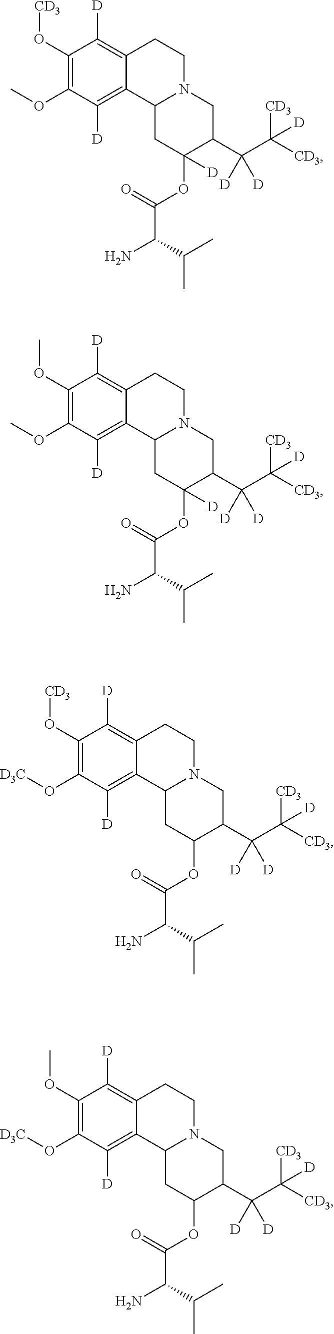

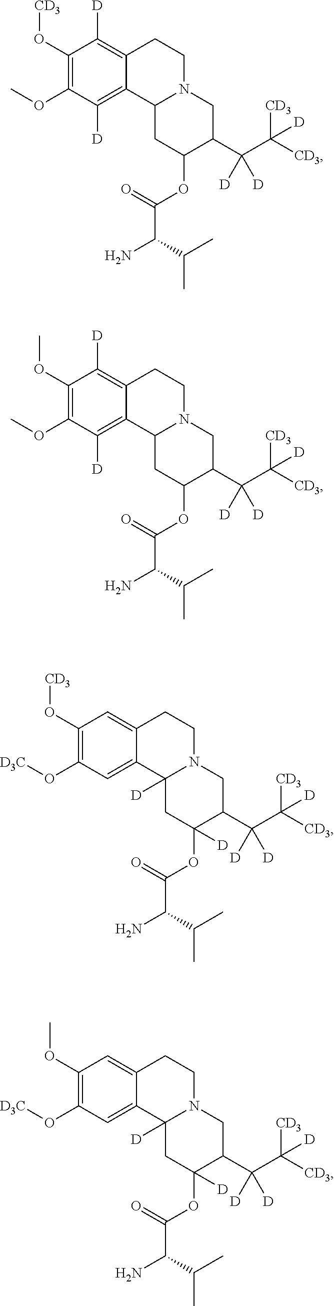

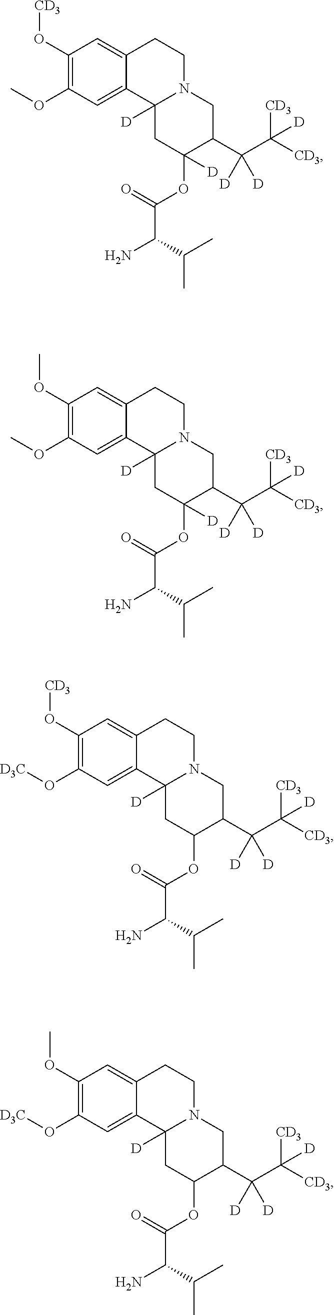

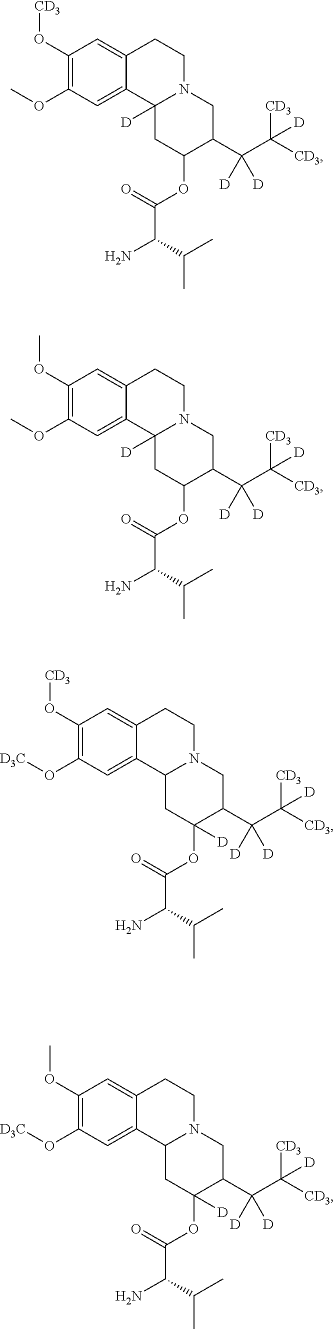

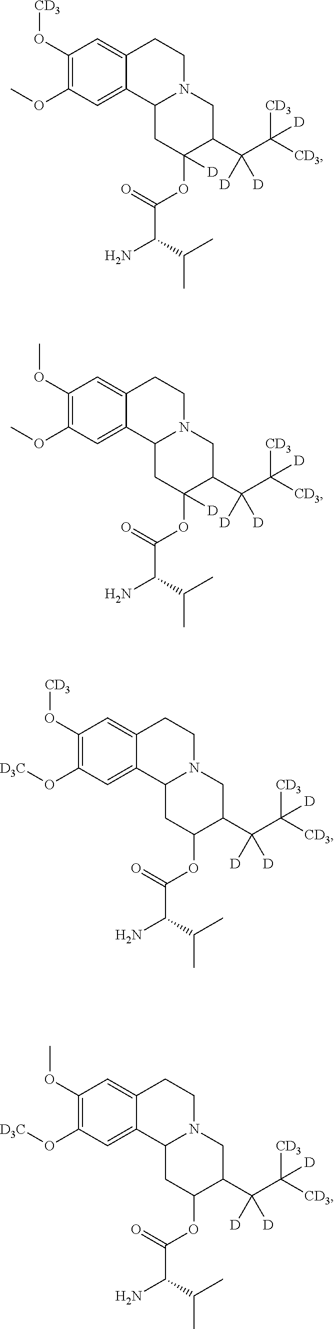

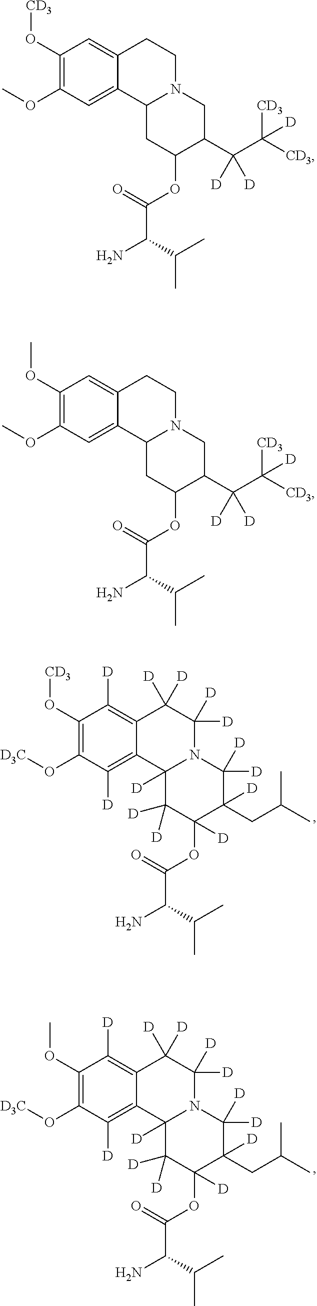

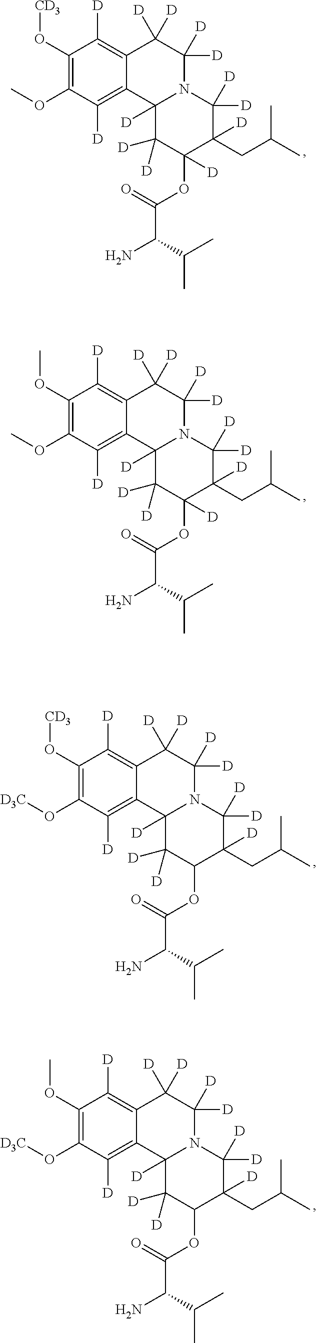

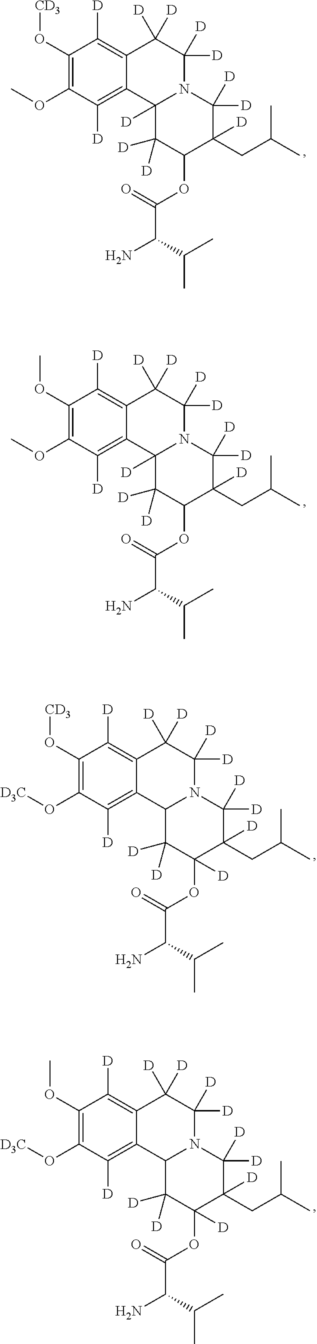

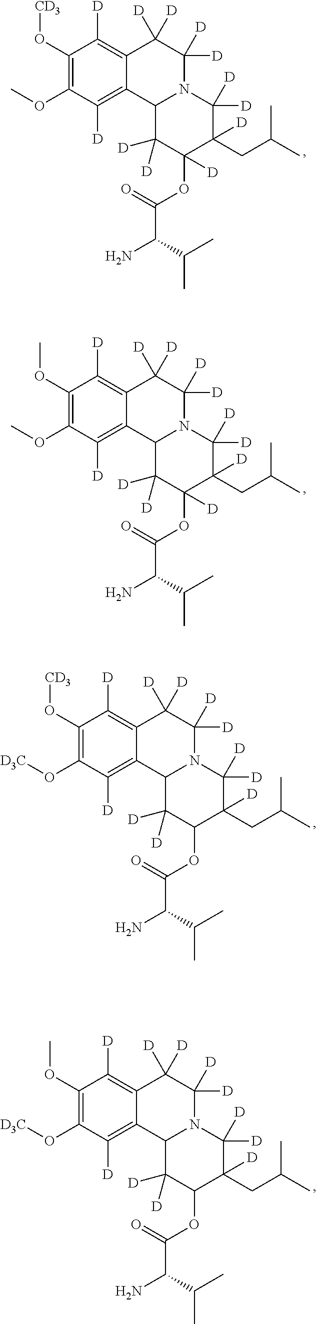

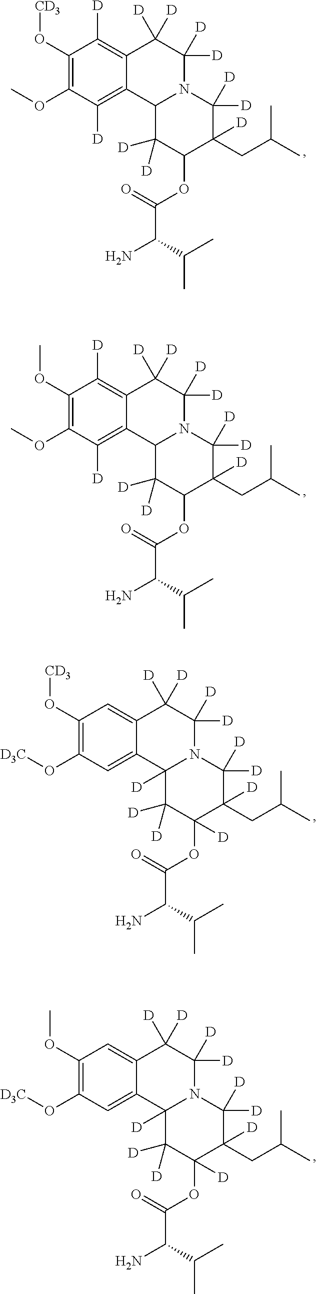

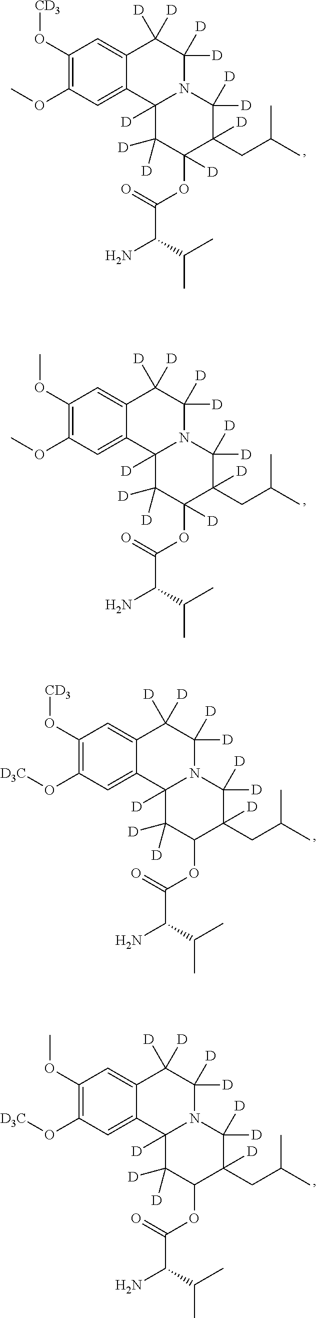

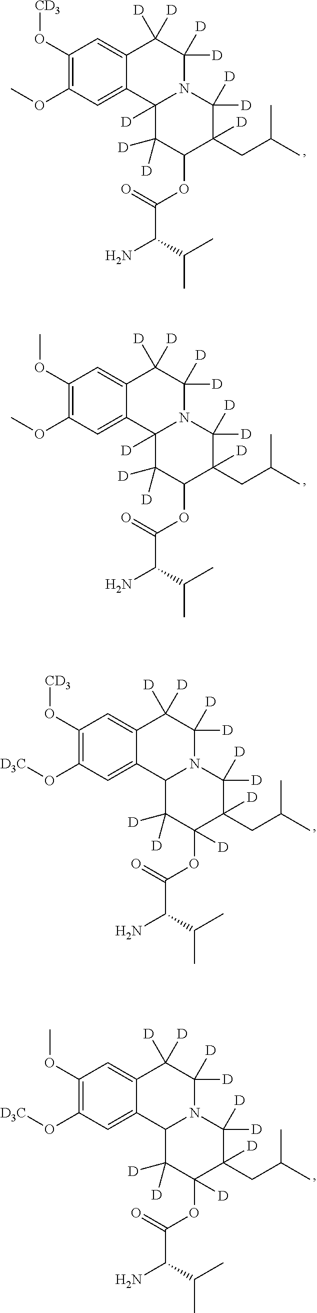

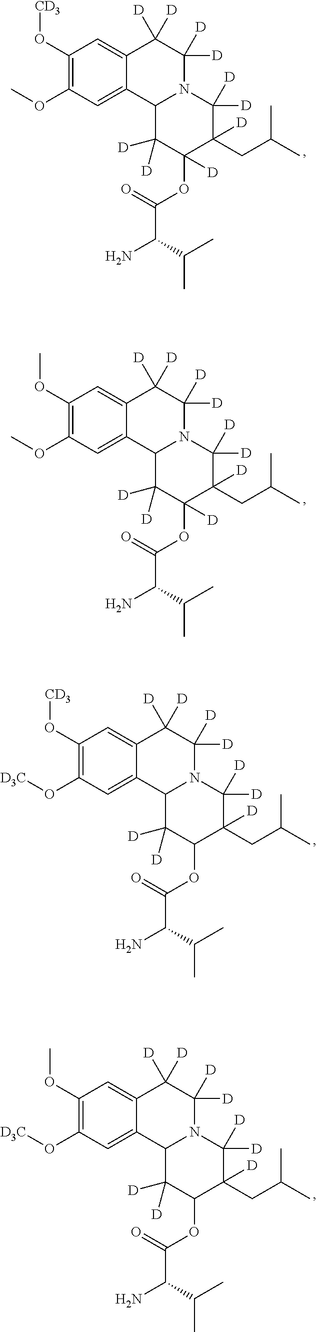

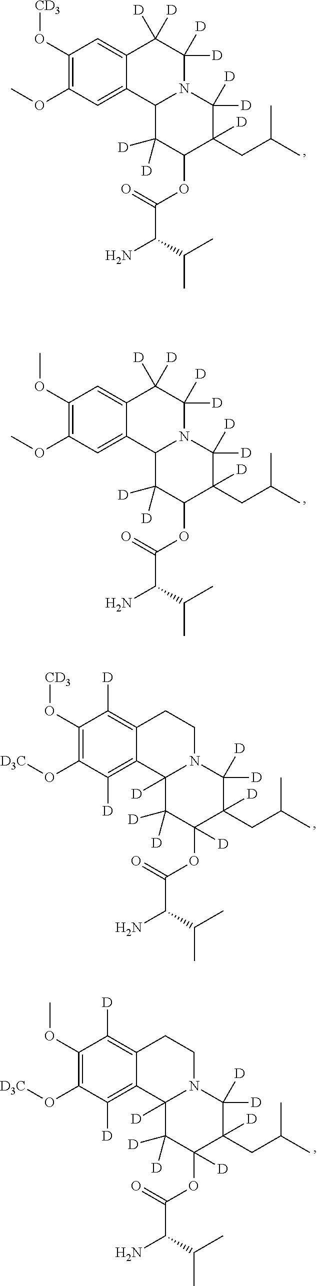

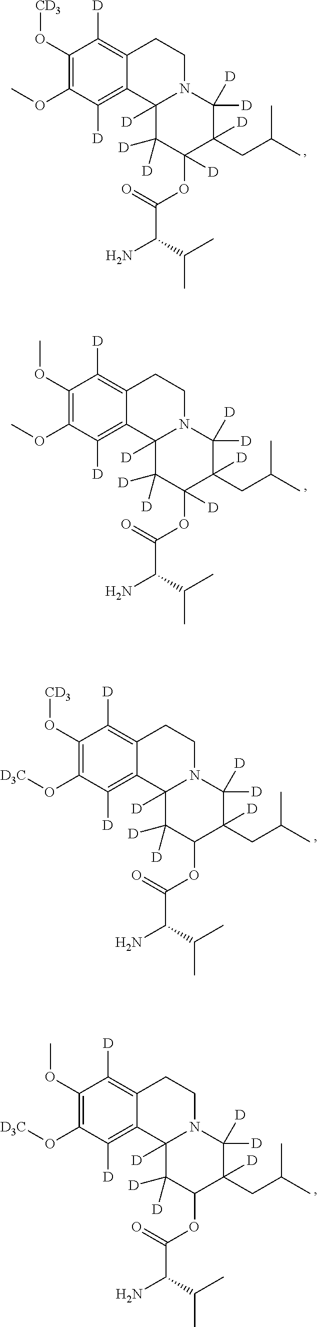

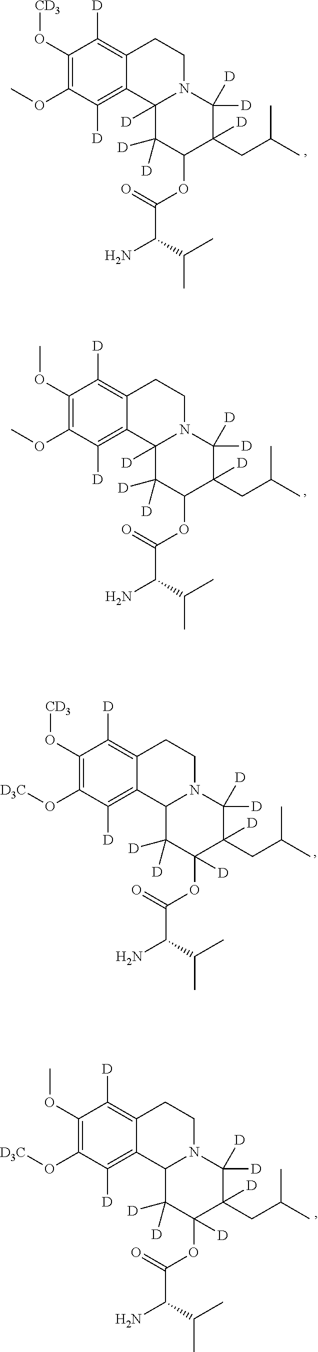

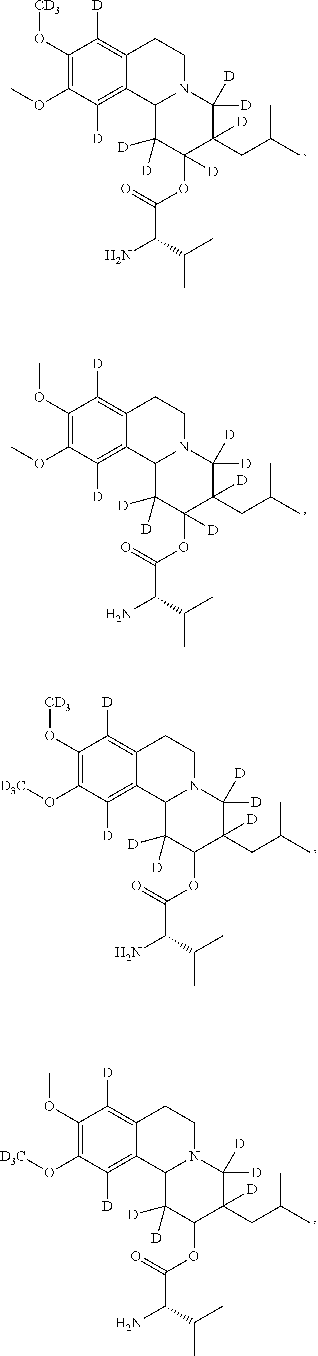

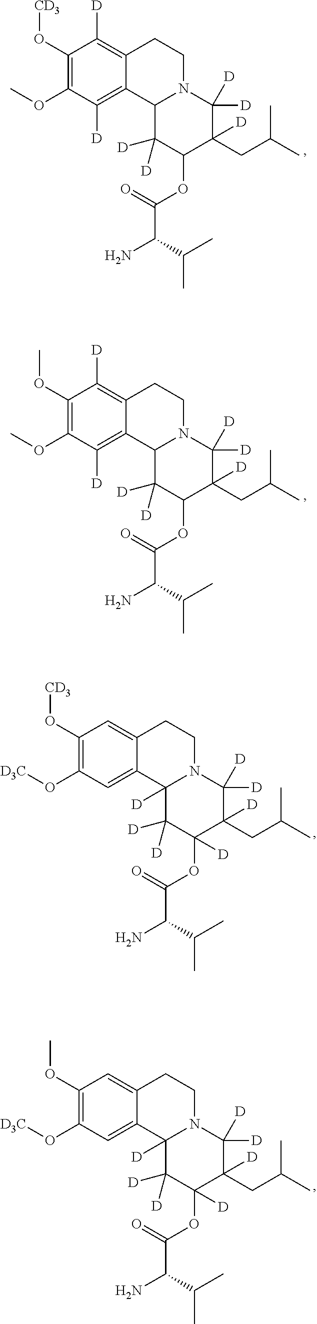

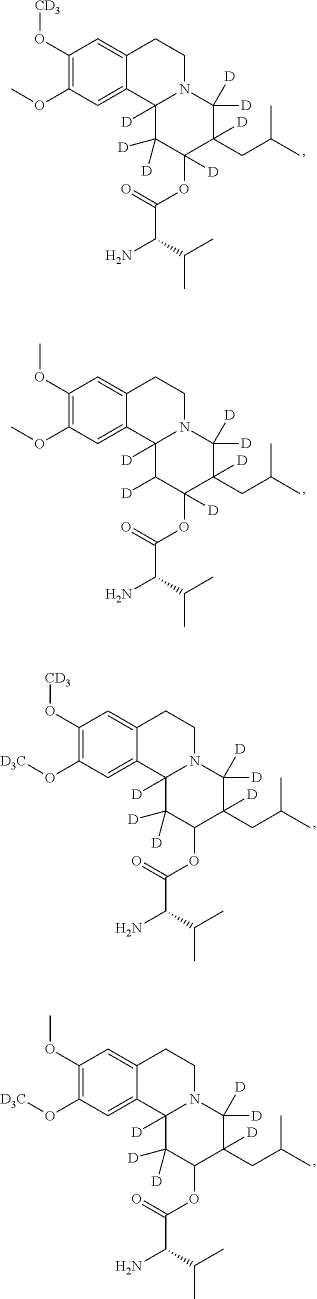

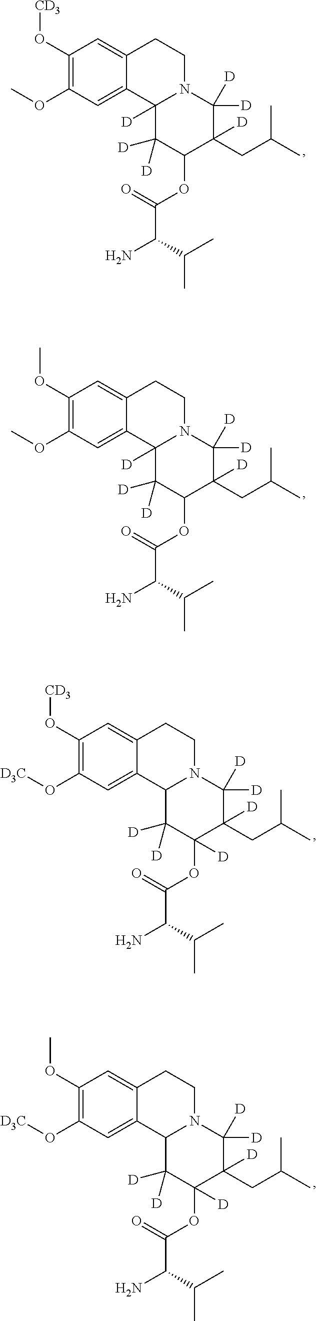

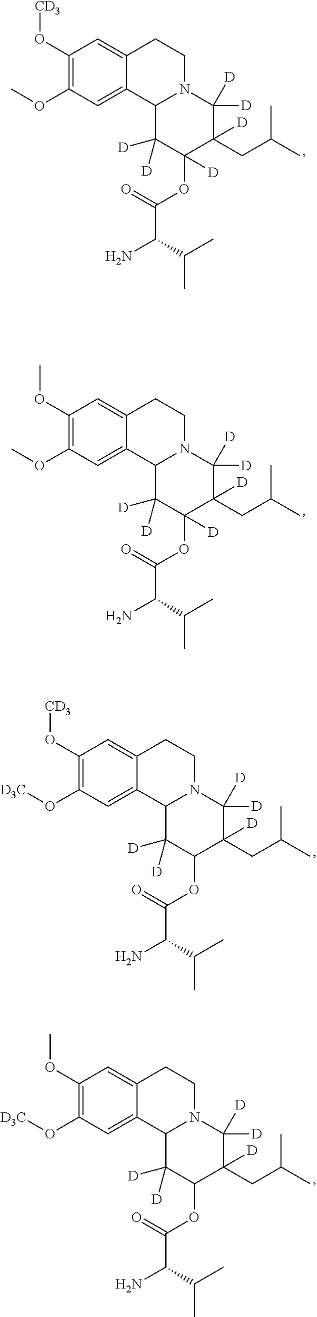

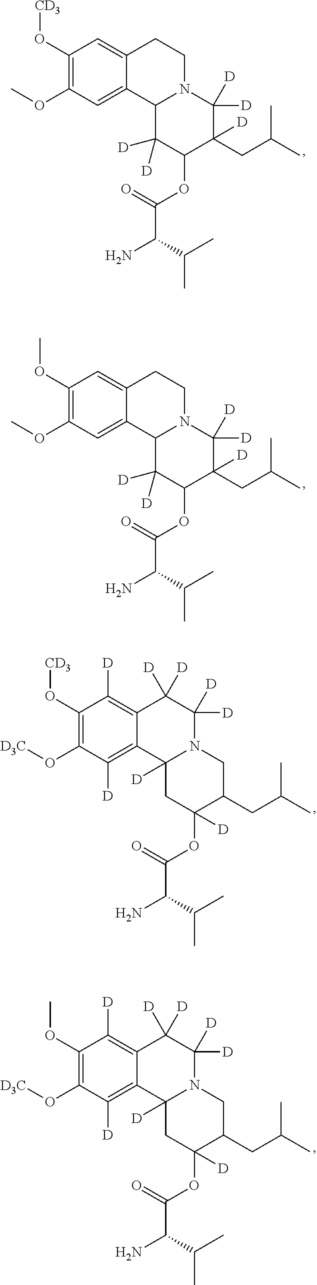

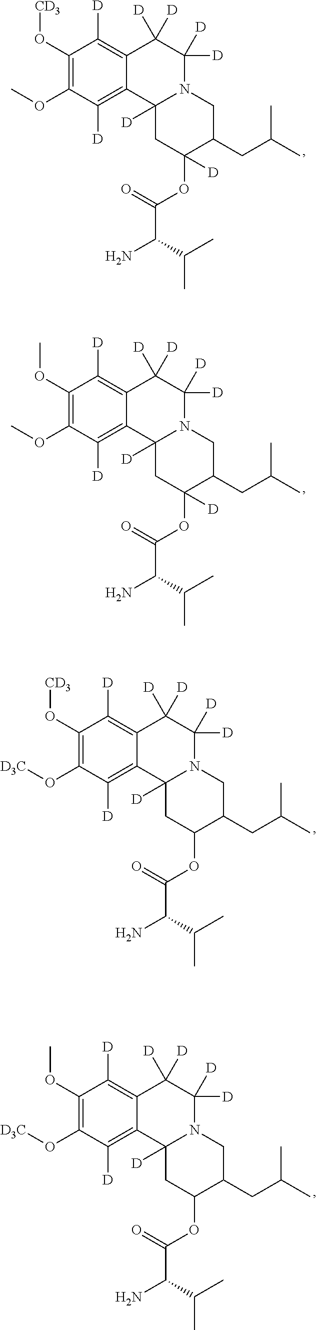

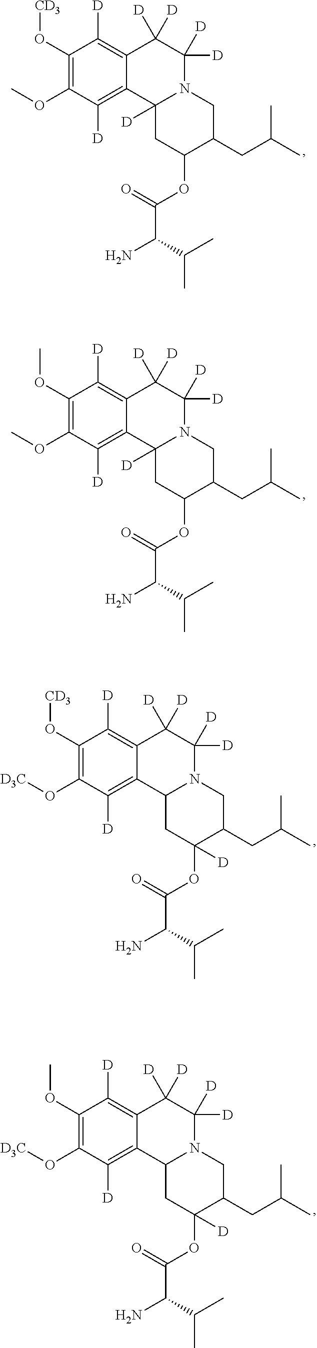

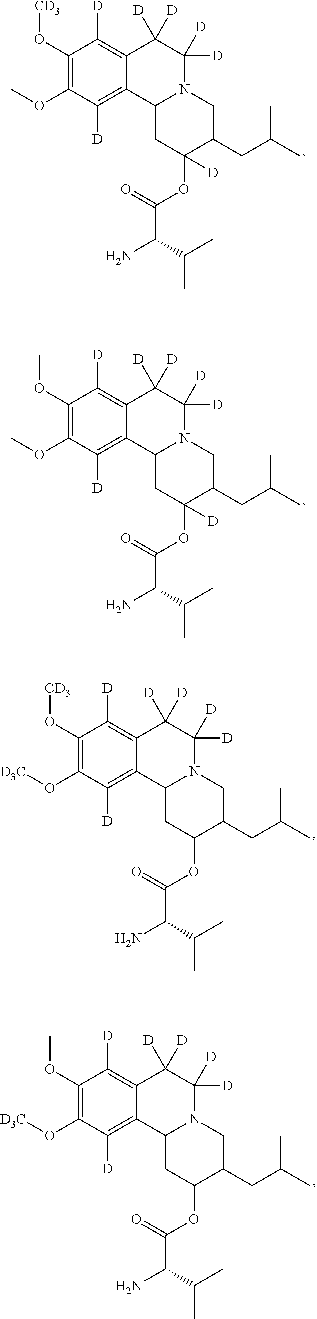

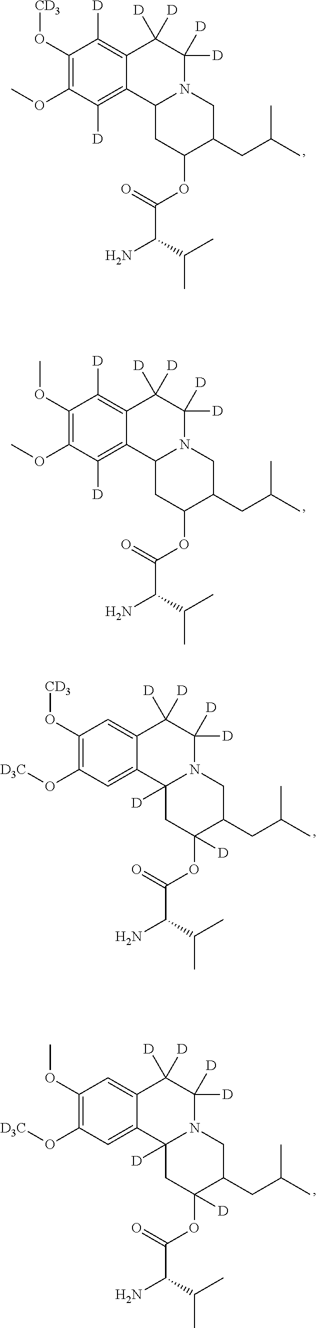

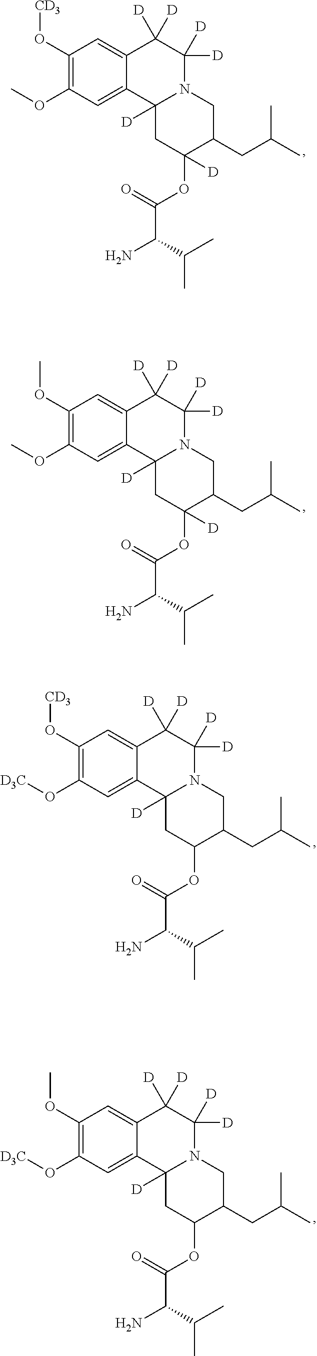

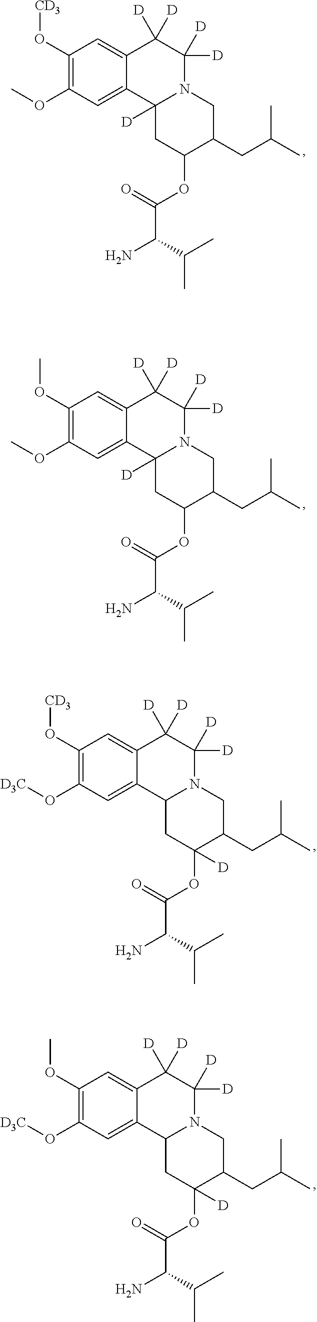

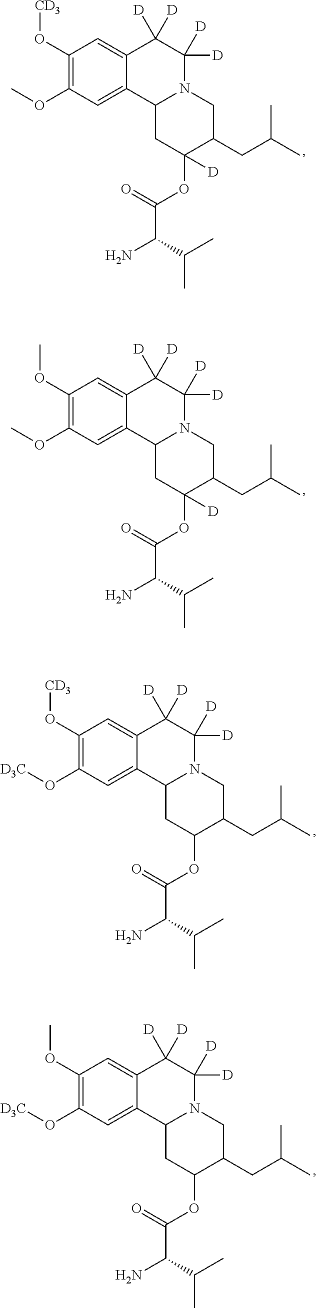

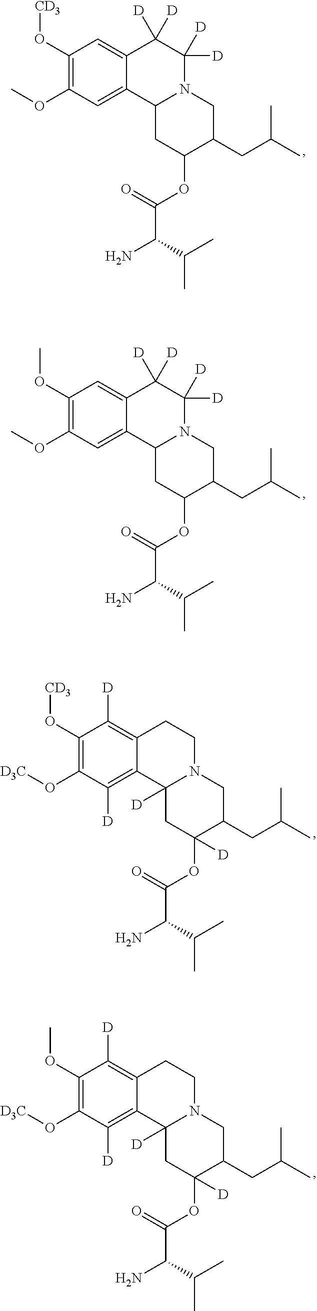

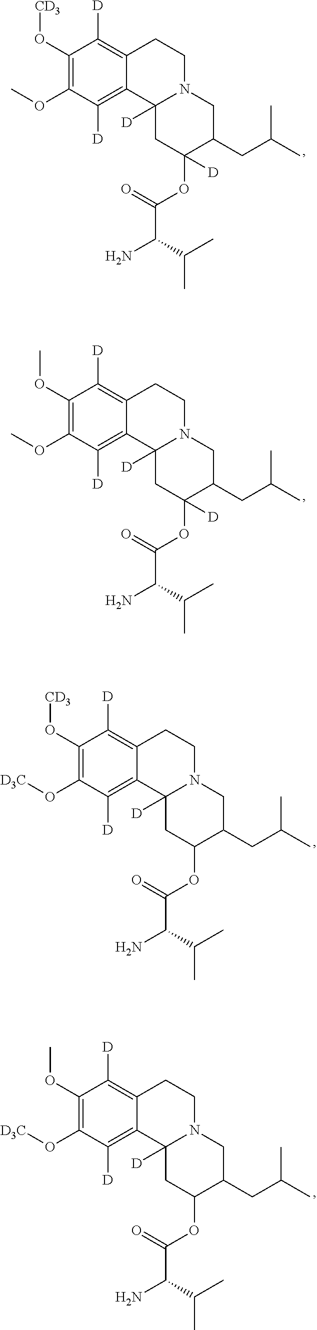

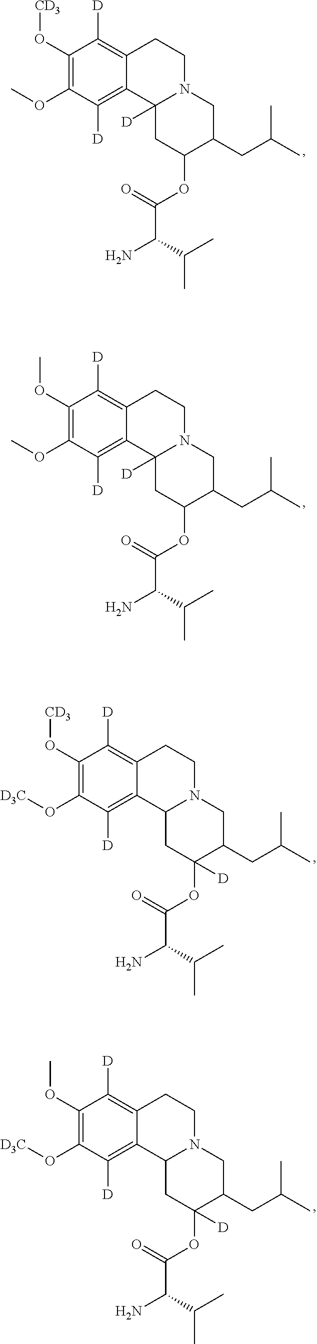

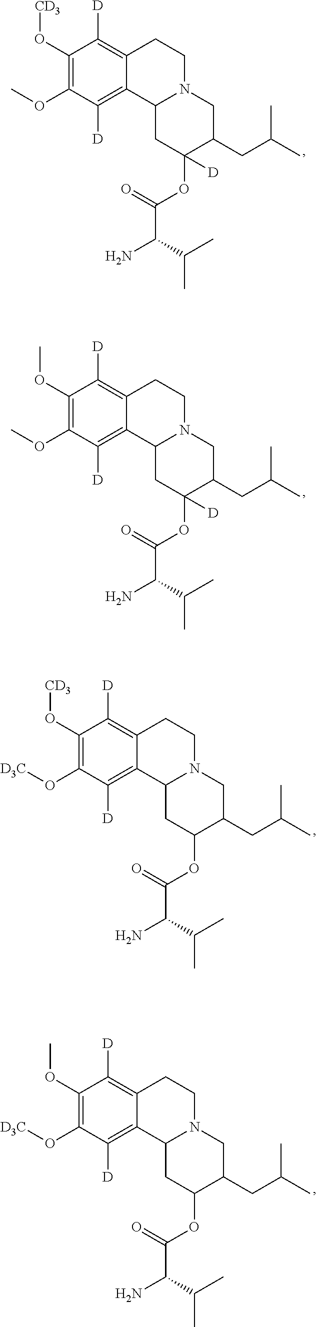

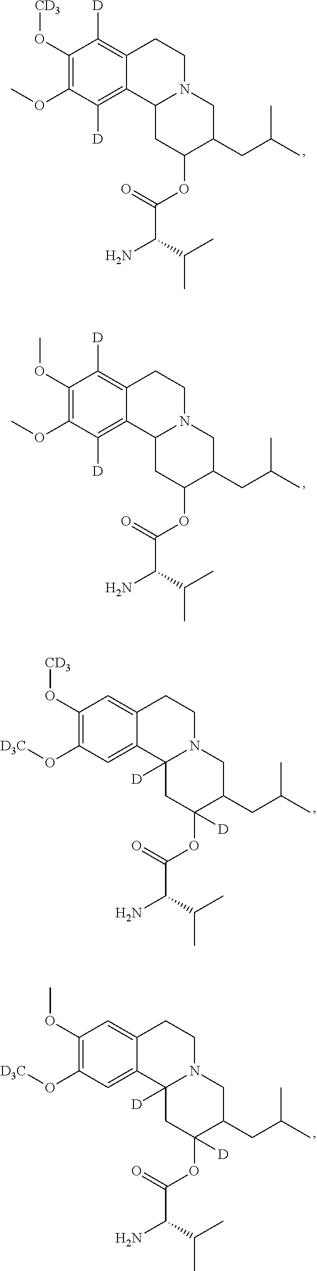

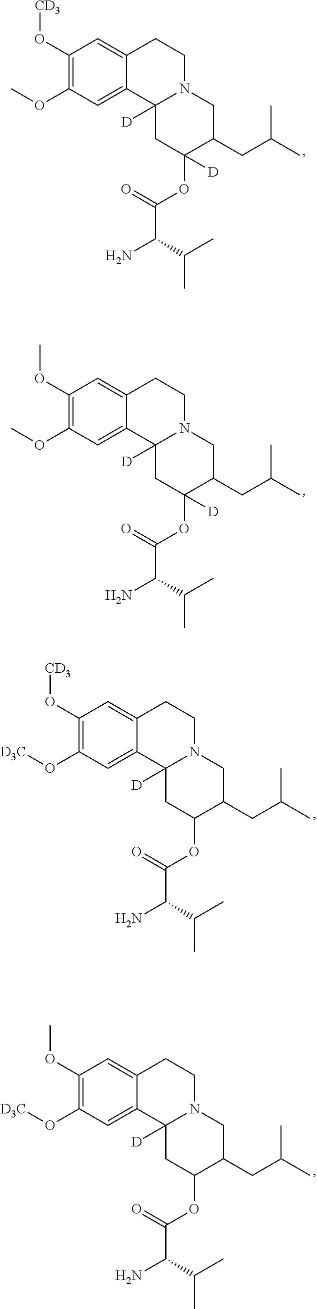

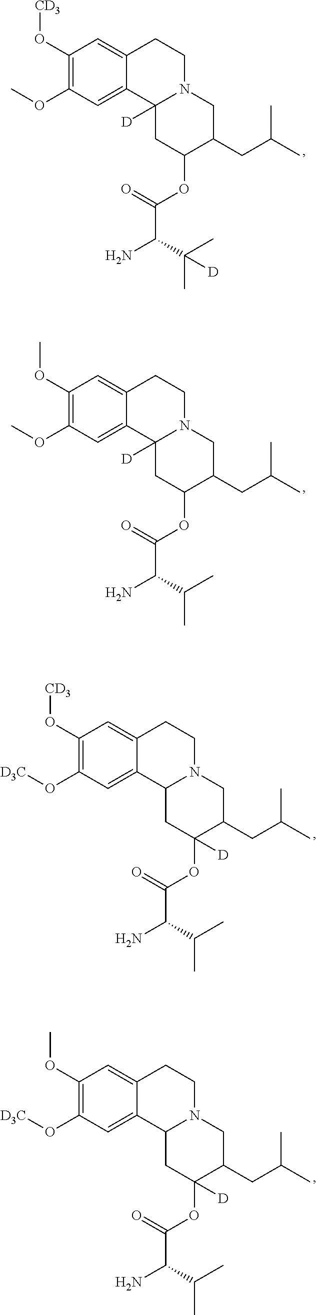

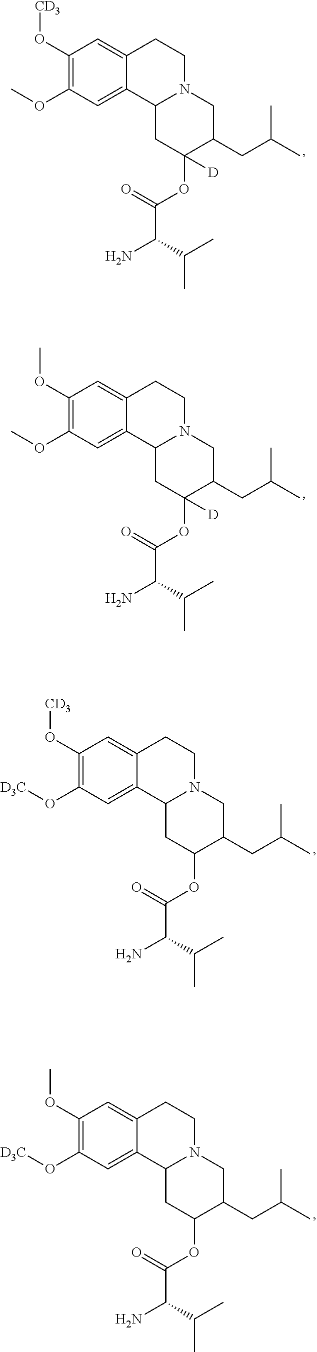

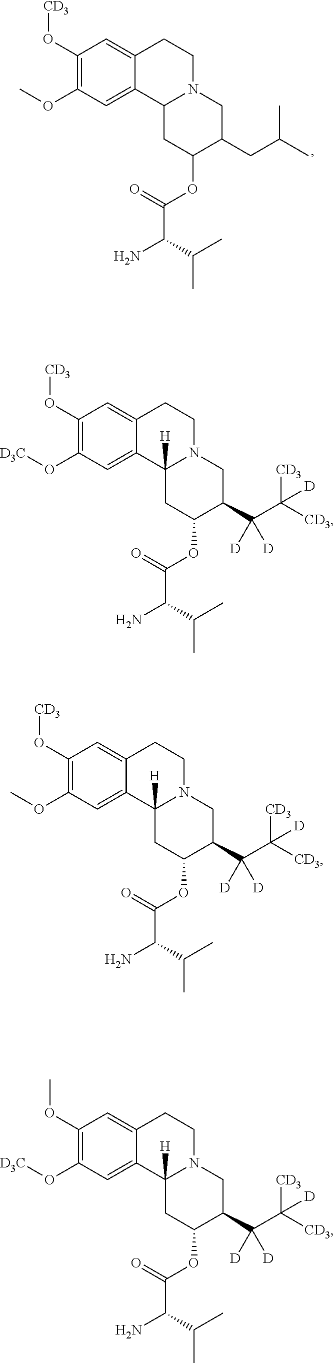

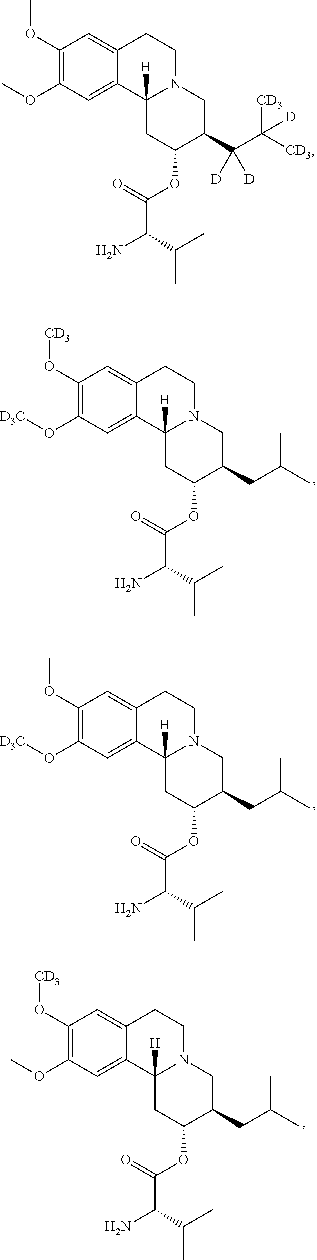

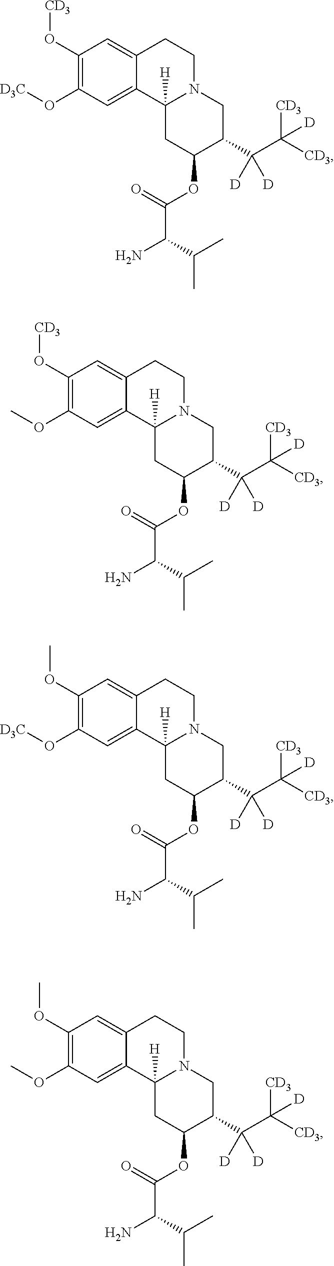

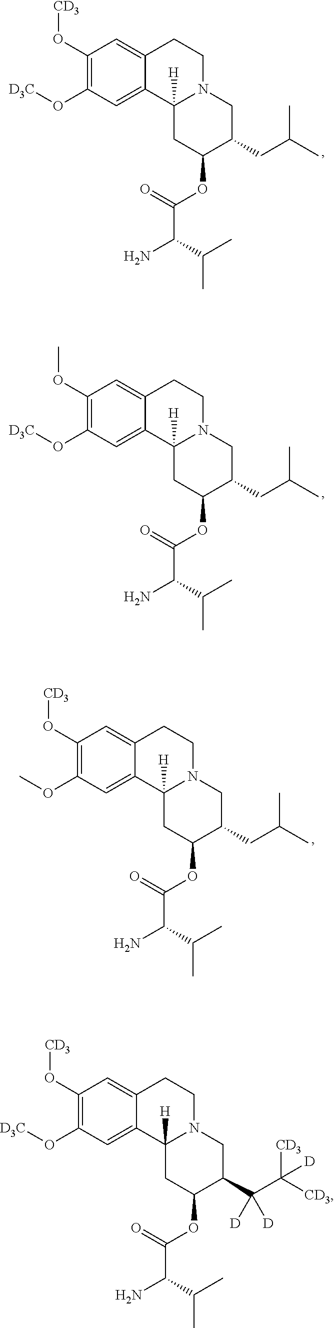

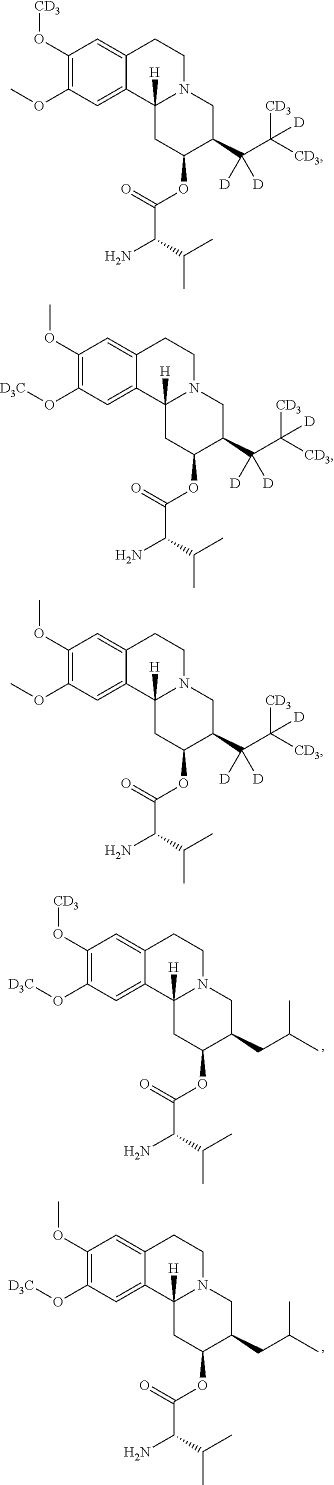

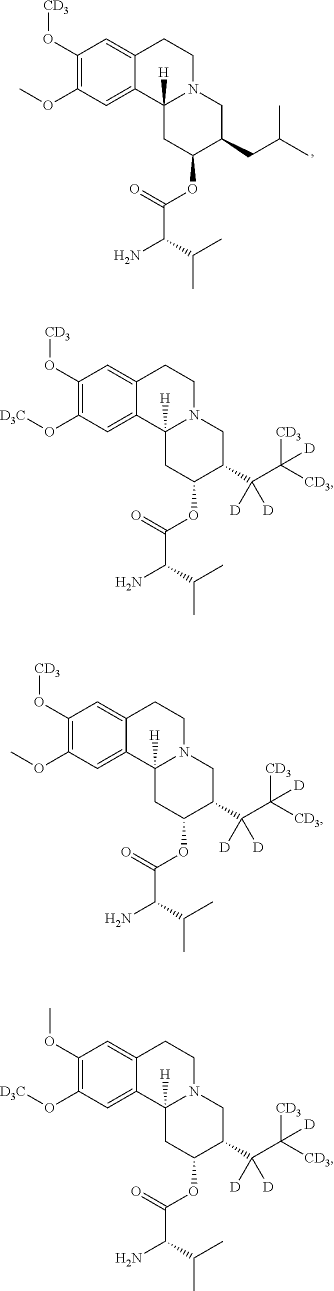

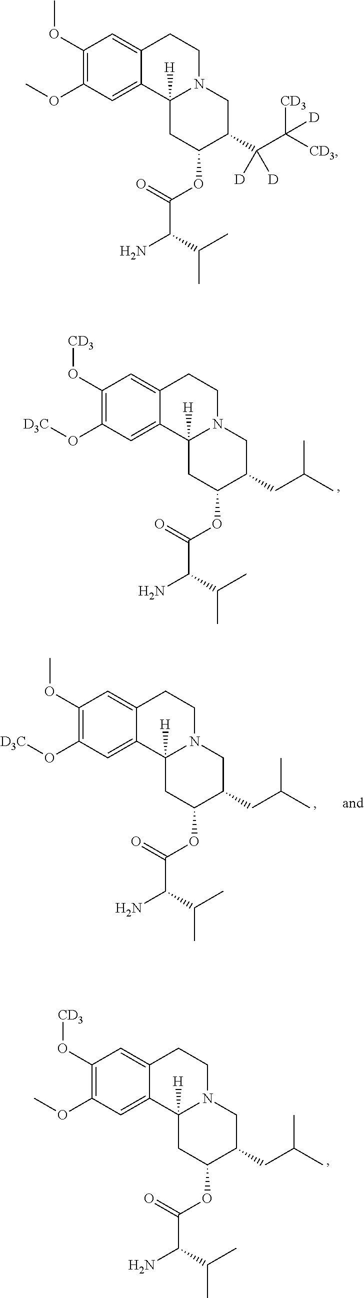

The following compounds can generally be made using the methods described above. It is expected that these compounds when made will have activity similar to those described in the examples above.

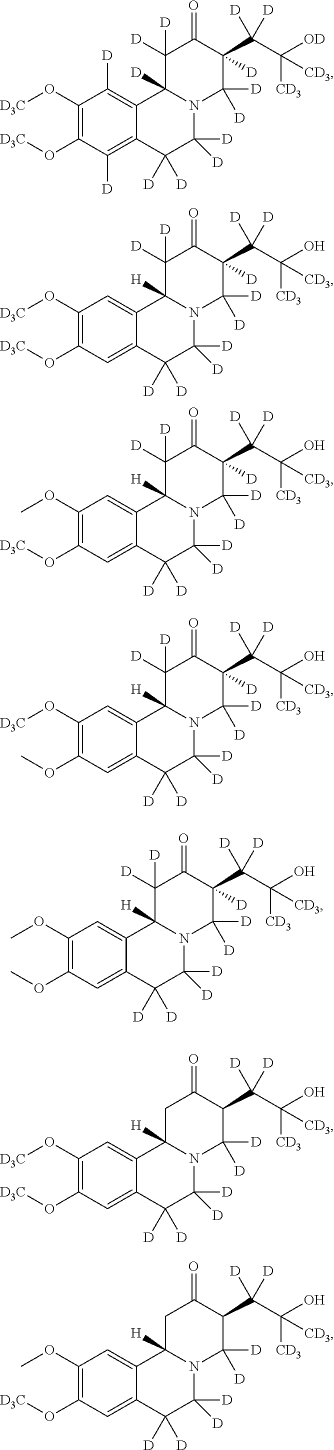

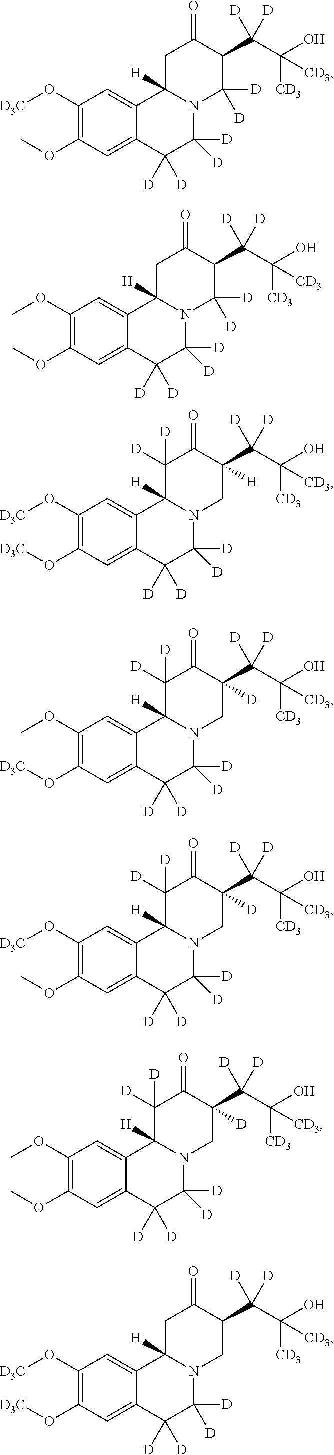

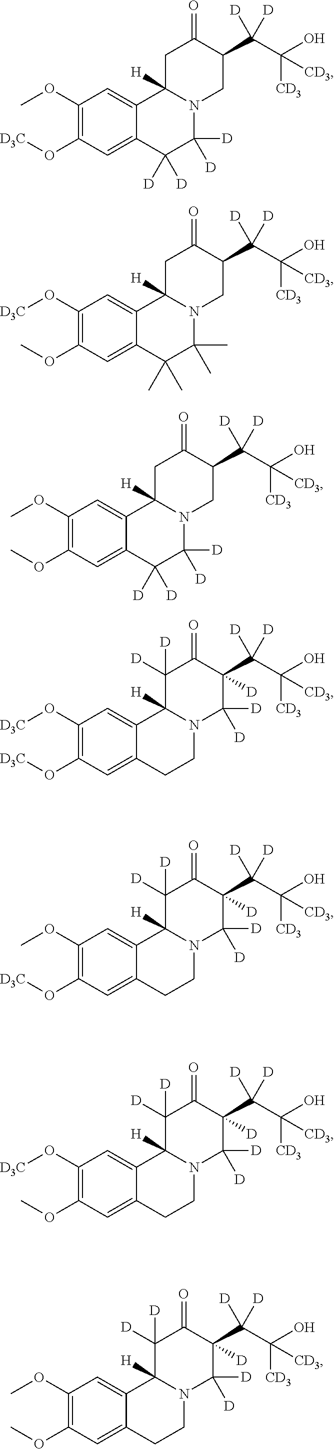

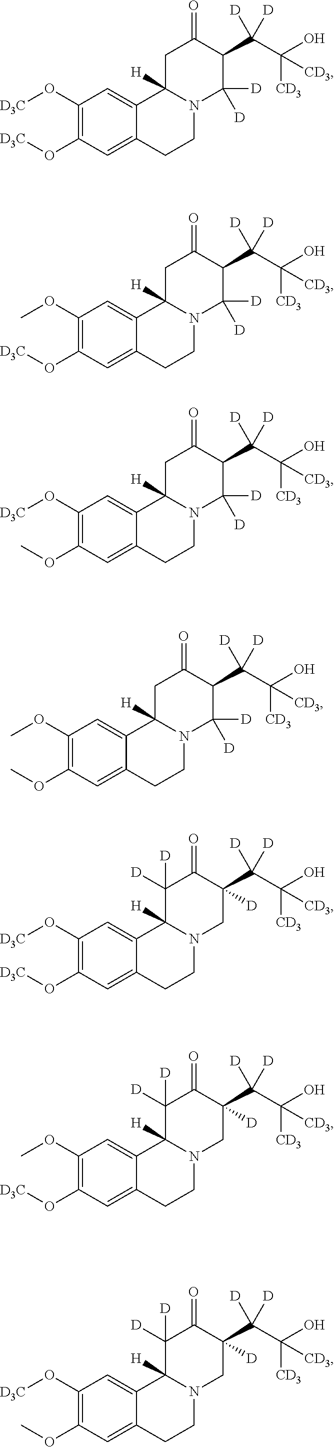

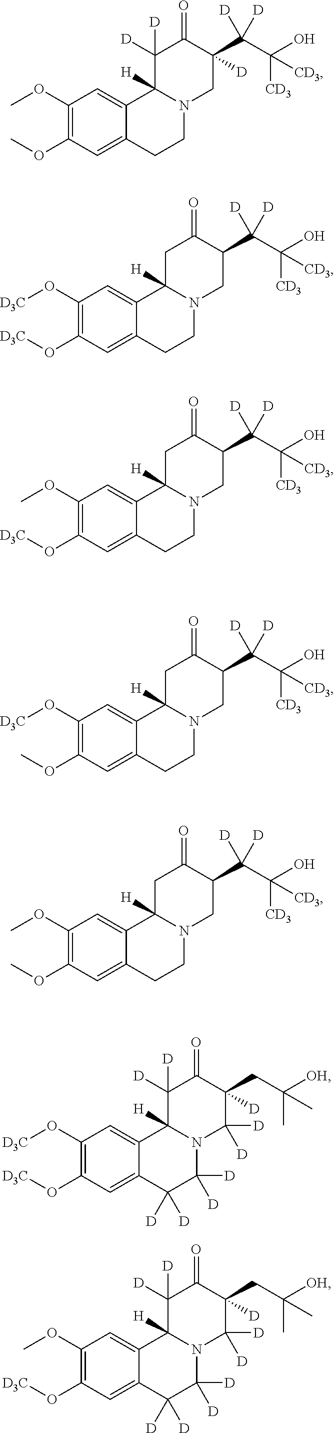

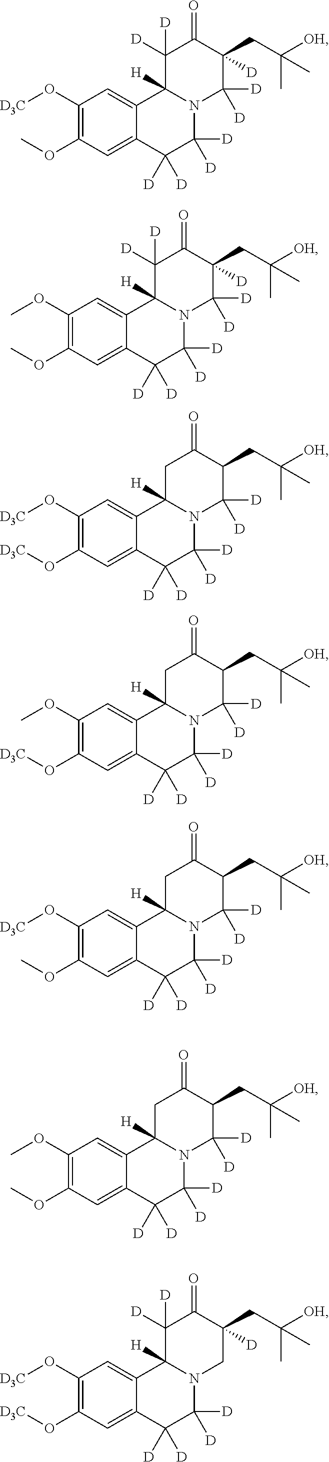

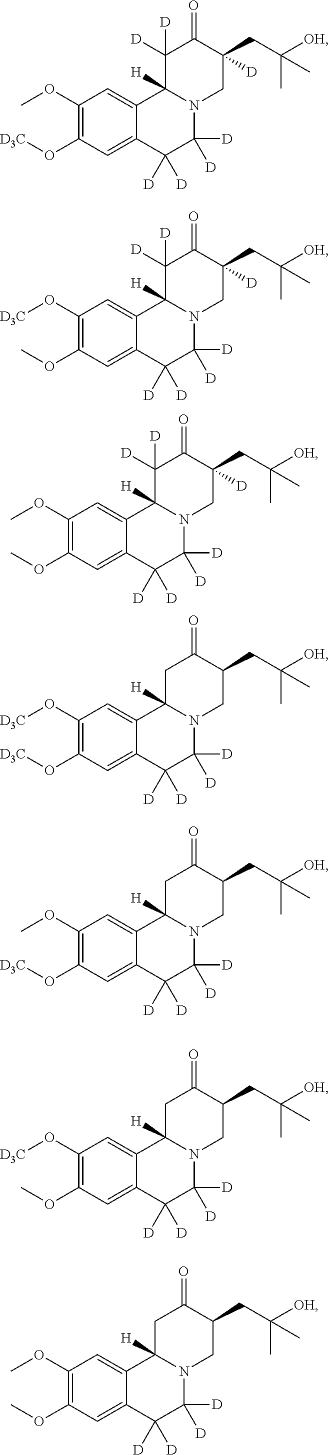

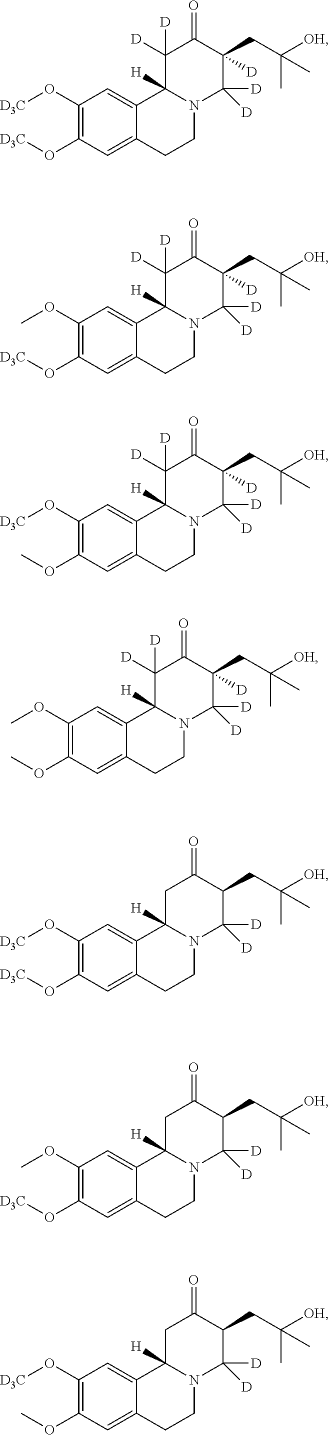

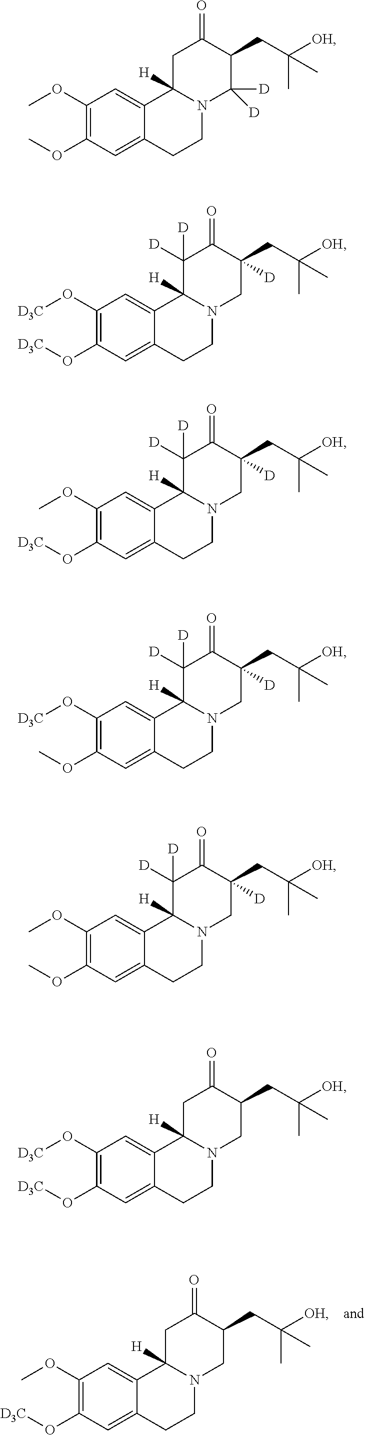

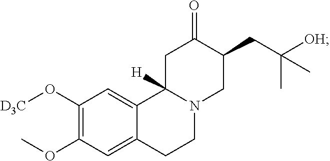 [SMILES: [2H]C([2H])([2H])Oc1cc2c(cc1OC)CCN1C[C@H](CC(C)(C)O)C(=O)C[C@@H]21]

or the 3S,11bS enantiomer, or a racemic mixture of the 3S,11bS and 3R,11bR enantiomers.

The following compounds can generally be made using the methods described above. It is expected that these compounds when made will have activity similar to those described in the examples above.

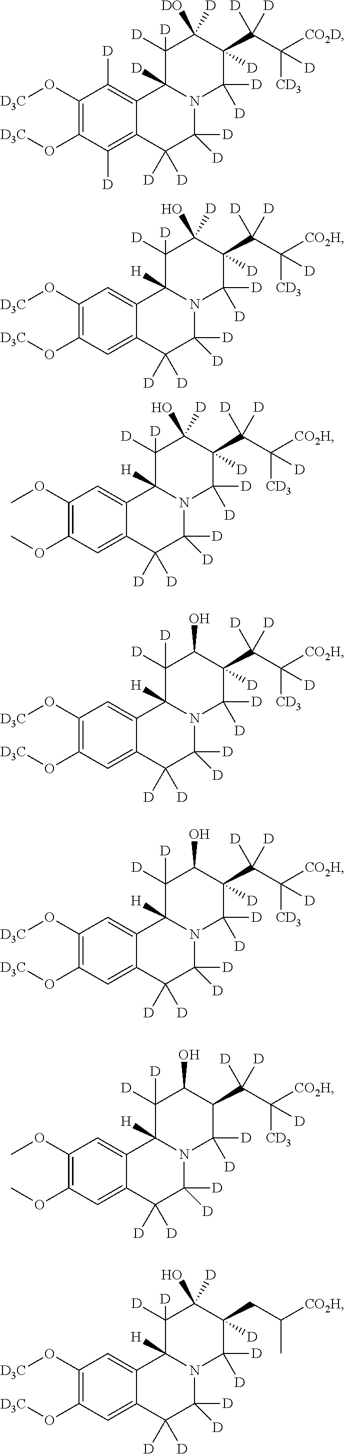

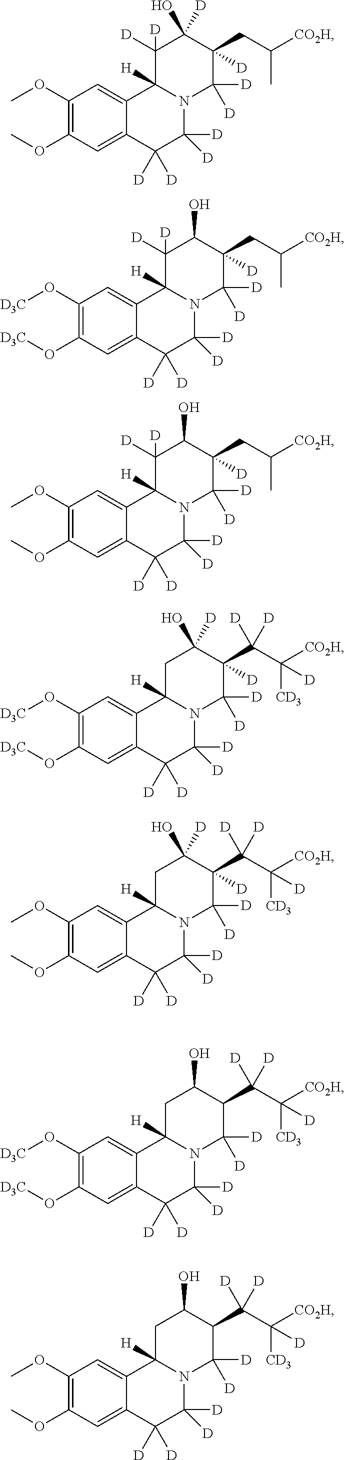

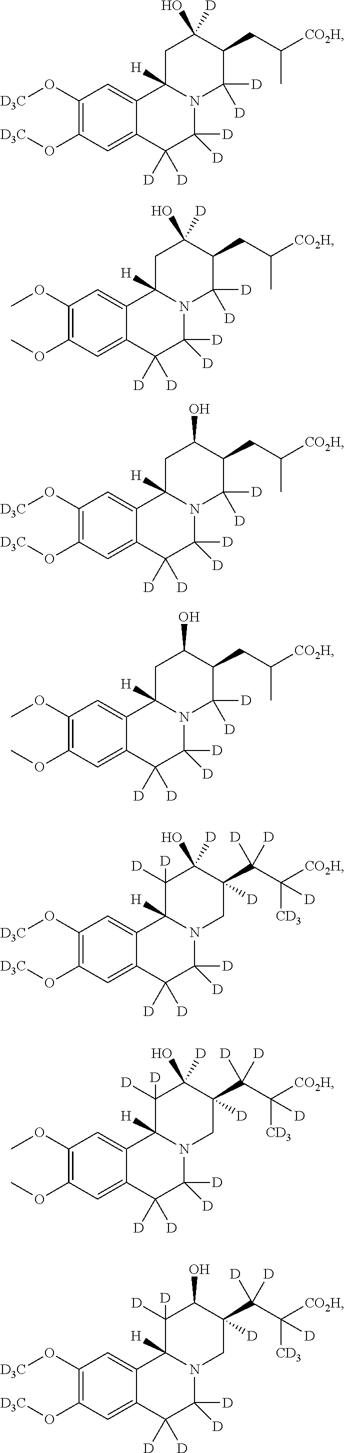

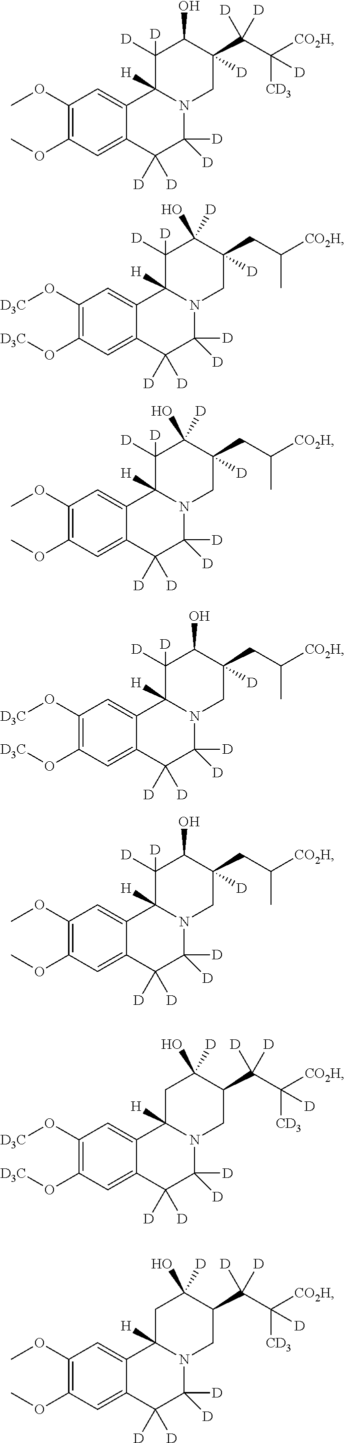

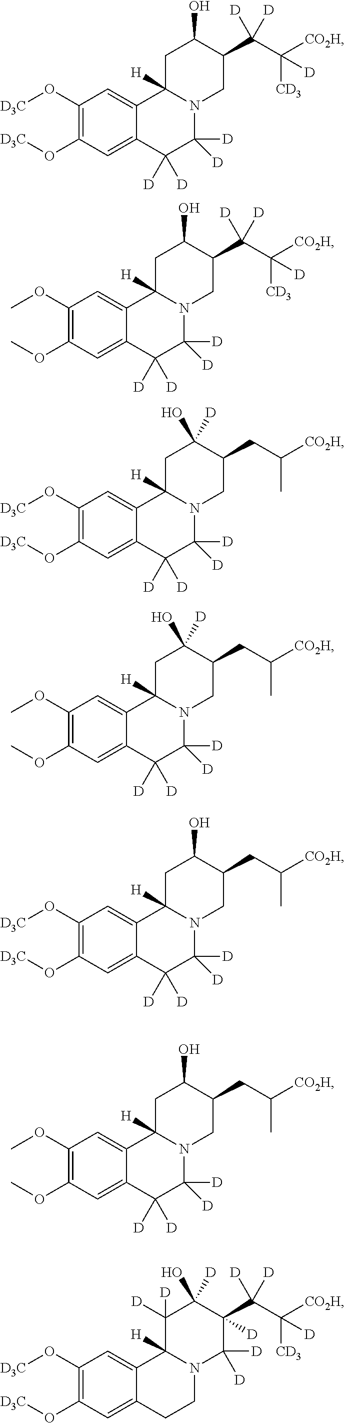

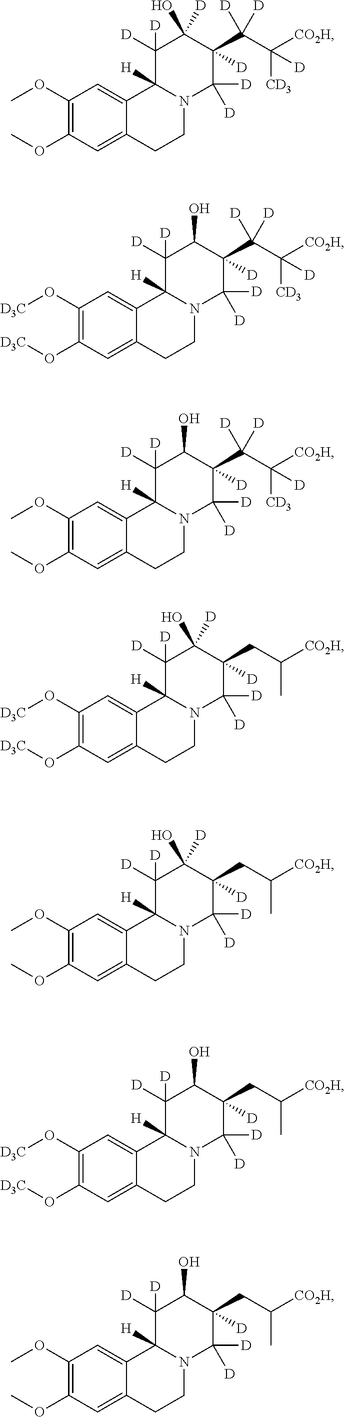

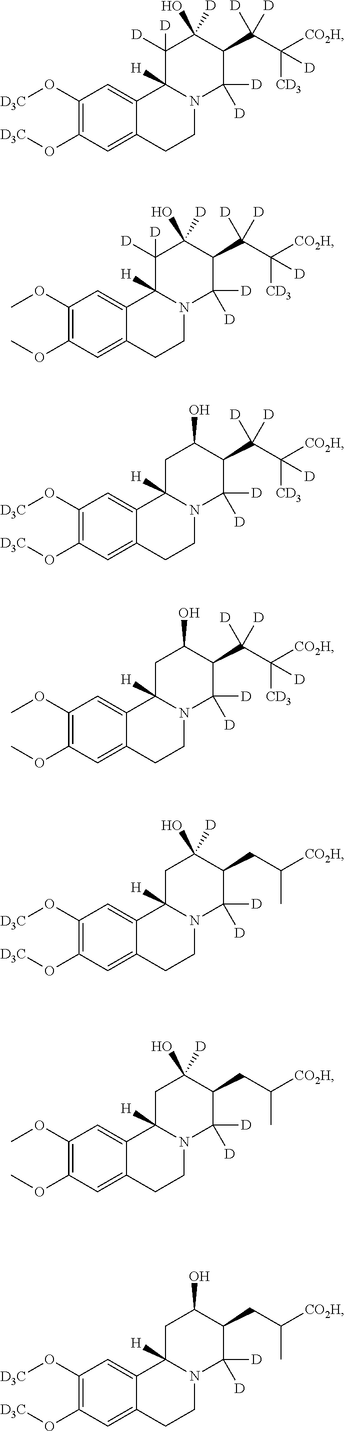

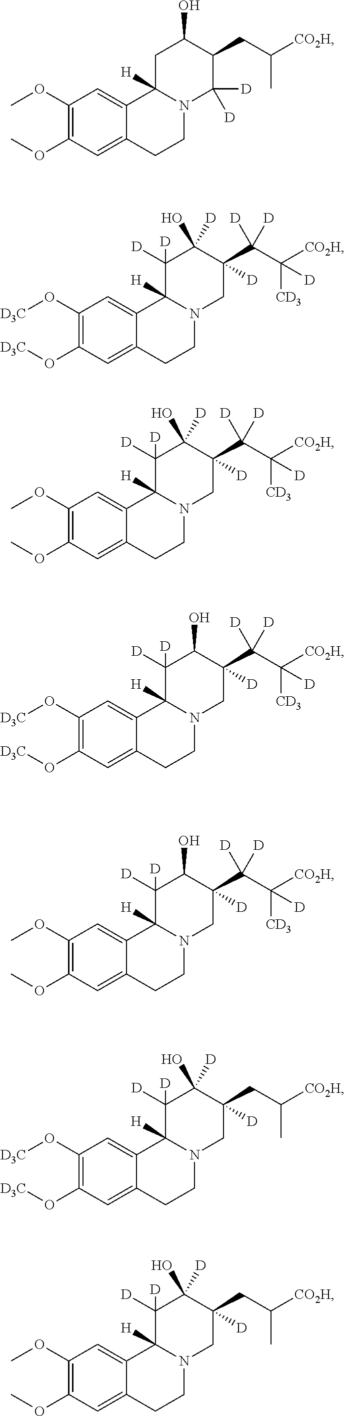

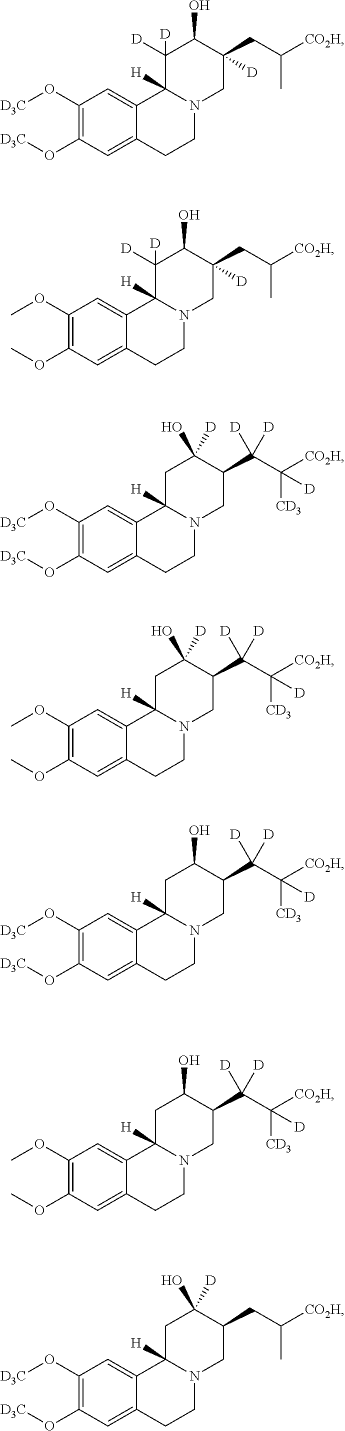

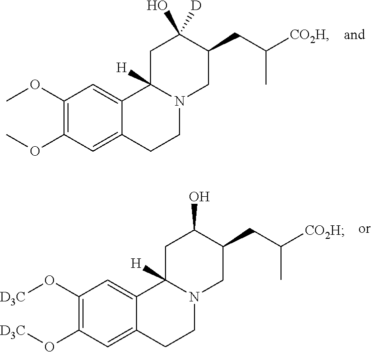 [SMILES: [2H][C@@]1(O)C[C@H]2c3cc(OC)c(OC)cc3CCN2C[C@@H]1CC(C)C(=O)O ; "and" ; [2H]C([2H])([2H])Oc1cc2c(cc1OC([2H])([2H])[2H])[C@@H]1C[C@@H](O)[C@@H](CC(C)C(=O)O)CN1CC2 ; "or"]

a diastereomer, or mixture of diastereomers thereof.

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

### Biological Activity Assays

### Clinical Study Protocol for the Measurement and Quantification of Huntington's Disease Symptoms

Materials:

PAMSys™ is a precise platform for long-term objective evaluation of physical activity during everyday life (1). PAMSys™ allows for the collection of posture (sitting, standing, walking, or lying down), postural transitions (duration, time of occurrence), gait (duration, number of steps, cadence and step time variability), and fall (number of falls, time of occurrence) information. The PAMSys™ technology is based on over 10 years of research supported in part by the National Institutes of Health (NIH) and uses advanced signal processing algorithms and novel biomechanical models of human motion to identify a complete physical activity map for the user from data measured by a single, lightweight, wearable motion sensor. PAMSys-X™ allows for synchronized monitoring of multiple body segment movements.

An approximately three-month study of 20 participants, 15 participants clinically diagnosed with choreic HD and 5 participants without Huntington's Disease. The participants are adult volunteers who will complete in-clinic and remote assessments over a one-week period.

To begin the study, all participants will visit the clinic to provide informed consent prior to completing a baseline assessment. The baseline assessment will utilize surveys to obtain demographic information, medical and HD history, current medications, and familiarity with technology. These surveys will be completed by participants while on-site and will be stored using the secure, web-based REDCap (Research Electronic Data Capture) survey application (2). Research staff will place 1 PAMSys™ (near the chest) and 4 PAMSys-X™ (on the wrists and ankles) sensors on the subjects. Participants will then complete the Q-motor finger-tap and force transducer assessments (3), the motor portion of the UHDRS (4), and the Montreal Cognitive Assessment (MoCA) (5). Participants will also be video-recorded while performing a standardized motor assessment wearing the five BioSensics mobile sensors. (Table 1)

The standardized motor assessment for the BioSensics sensors will consist of six tasks that participants will perform while wearing the equipment. When wearing the five BioSensics sensors participants will complete the following tasks: 20 seconds each of static sitting and standing, a Timed Up and Go (TUG) test (6), 30 seconds of tandem walking, 15 seconds of finger tapping, 5 instances of a drinking motion using a cup or glass, and 15 seconds of pronation and supination of the hands. When wearing only the trunk sensor, participants will only complete the first three standard assessments (static sitting/standing, Timed Up and Go test, and tandem walking) The standard assessments will be completed twice per day during the week of study

Following the baseline assessment, participants will continue to wear the BioSensics sensors for a total period of 24 hours as they go about their daily activities following the in-clinic assessment. Subjects will then be instructed to remove these 5 sensors and put on a second PAMSys shirt with trunk sensor only, which will be worn for 6 days.

Participants will finish the study by returning to the clinic on Day 7 to assess adverse events, concomitant medications and discuss any issues with wearing the sensors, complete the same clinical assessments performed at baseline (except physical and neurological exam), and complete a REDCap survey on adherence, perceived utility, benefits and limitations of the study. After the Day 7 assessments are completed, sensors will be returned to BioSensics in a prepaid, pre-addressed package. The schedule of activities describes the required sequence of events for participants. (Table 2)

TABLE 1
Required assessments for BioSensics mobile sensors

| | Five mobile sensors | Trunk sensor | Duration of |
|---|---|---|---|
| | (Baseline) | (Day 7) | assessment |
| Standard | 1) At rest 5 min | 1) Sit/stand [static] | 1) 20 seconds |
| assessments | 2) Sit/stand [static] - 2 | 2) Timed Up and | each Variable |
| | or 3 reps? 30 sec to | Go | 2) 30 seconds |
| | 1 min | 3) Tandem walking | 15 seconds |
| | 3) Timed Up and Go | | 5 motions |
| | 4) Tandem walking - | | 15 seconds |
| | replace if necessary | | |
| | with regular walking | | |
| | 5) Finger tapping | | |
| | 6) Drinking motion - | | |
| | immediately after | | |
| | rest | | |
| | 7) Pronation/supination | | |

TABLE 2
Schedule of Activities

| | Screen/ | | | |
|---|---|---|---|---|
| | Baseline | Week 1 | | End Study |
| Assessment location | Clinic | Remote | | Clinic |
| Days after baseline assessment | 0 | 1 | 2-7 | 7 + ~3 |
| Informed consent | X | | | |
| Demographics, medical history | X | | | |
| Baseline technology survey | X | | | |
| Physical/neuro exam | X | | | |
| Motor UHDRS | X | | | X |
| MoCA | X | | | |
| Video-recorded standardized | X | | | X |
| BioSensics assessment | | | | |
| Conduct Q-motor exam (finger tap | X | | | X |
| and force transducer) assessments | | | | |
| Wear BioSensics sensors (5) | X | X | | |
| Participant to return 5 sensors | | | X | |
| Wear BioSensics sensor (1 - Trunk) | | X | X | X |
| Conduct survey on adherence, | | | | X |
| perceived utility, benefits, and | | | | |
| limitations of study | | | | |
| Assess AEs | | | | X |
| Assess Concomitant Medications | X | | | X |

Outcome Measures:

The principal outcome measures include remote data collected from the BioSensics devices from two in-person assessments. Clinical outcome measures include clinical characteristics (i.e. UHDRS and Q-motor exams) and comparison of data from individuals with HD to controls.

1. A method of treating abnormal muscular activity in a subject in need thereof comprising the steps of:
a. measuring muscular activity data in the subject with at least one accelerometer;
b. processing the measured muscular activity data to distinguish between normal muscular activity and abnormal muscular activity in the subject;
c. transmitting the processed muscular activity data to a remote access unit;
d. retrieving the processed muscular activity data from the remote access unit;
e. determining a level of abnormal muscular activity in the subject; and
f. treating the subject based upon the level of the subject's abnormal muscular activity as determined in step e.

2. The method of claim 1 wherein the abnormal muscular activity is associated with at least one of bradykinesia, dyskinesia, and hyperkinesia.

3. The method as recited in claim 1 wherein the abnormal muscular activity is associated with Huntington's disease.

4. The method of claim 1 wherein treating the subject comprises administering a therapeutically effective amount of a therapeutic agent to the subject.

5. The method of claim 4 wherein the therapeutic agent is tetrabenazine.

6. The method of claim 4 wherein the therapeutic agent is a compound of structural Formula I 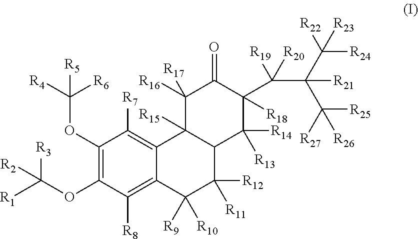
or a salt, stereoisomer, or racemic mixture thereof, wherein:
R₁-R₂₇ are independently selected from the group consisting of hydrogen and deuterium; and
at least one of R₁-R₂₇ is deuterium.

7. The method of claim 6 wherein at least one of R₁-R₂₇ independently has deuterium enrichment of no less than about 10%.

8. The method of claim 6 wherein at least one of R₁-R₂₇ independently has deuterium enrichment of no less than about 50%.

9. The method of claim 6 wherein at least one of R₁-R₂₇ independently has deuterium enrichment of no less than about 90%.

10. The method of claim 6 wherein at least one of R₁-R₂₇ independently has deuterium enrichment of no less than about 98%.

11. The method of claim 6 wherein the compound has the structural formula:

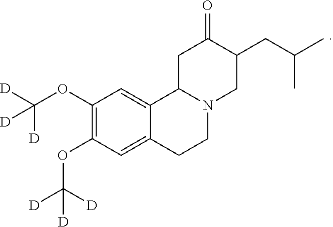 [SMILES: [2H]C([2H])([2H])Oc1cc2c(cc1OC([2H])([2H])[2H])C1CC(=O)C(CC(C)C)CN1CC2]

12. The method of claim 4 wherein the therapeutic agent is a compound of structural Formula II 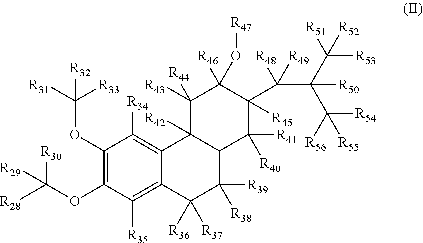
or a salt, stereoisomer, or racemic mixture thereof, wherein:
R₂₈-R₅₆ are independently selected from the group consisting of hydrogen and deuterium; and
at least one of R₂₈-R₅₆ is deuterium.

13. The method of claim 12 wherein at least one of R₂₈-R₅₆ independently has deuterium enrichment of no less than about 10%.

14. The method of claim 12 wherein at least one of R₂₈-R₅₆ independently has deuterium enrichment of no less than about 50%.

15. The method of claim 12 wherein at least one of R₂₈-R₅₆ independently has deuterium enrichment of no less than about 90%.

16. The method of claim 12 wherein at least one of R₂₈-R₅₆ independently has deuterium enrichment of no less than about 98%.

17. The method of claim 12 wherein the compound has the structural formula:

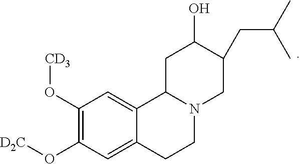

18. The method of claim 12, wherein the compound is the alpha stereoisomer.

19. The method of claim 12, wherein the compound is the beta stereoisomer.

20. The method of claim 12 wherein the compound has the structural formula:

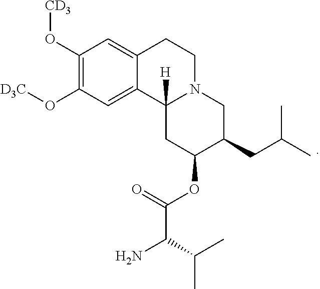

21. The method of claim 4 wherein the therapeutic agent is a compound of structural Formula III 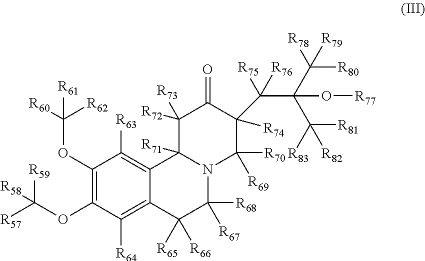
or a salt, stereoisomer, or racemic mixture thereof, wherein:
R₅₇-R₈₃ are independently selected from the group consisting of hydrogen and deuterium; and
at least one of R₅₇-R₈₃ is deuterium.

22. The method of claim 21 wherein at least one of R₅₇-R₈₃ independently has deuterium enrichment of no less than about 10%.

23. The method of claim 21 wherein at least one of R₅₇-R₈₃ independently has deuterium enrichment of no less than about 50%.

24. The method of claim 21 wherein at least one of R₅₇-R₈₃ independently has deuterium enrichment of no less than about 90%.

25. The method of claim 21 wherein at least one of R₅₇-R₈₃ independently has deuterium enrichment of no less than about 98%.

26. The method of claim 21 wherein the compound has the structural formula: 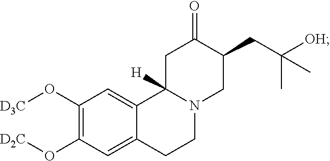
or the 3S,11bS enantiomer, 3R,11bR enantiomer, or a racemic mixture of the 3S,11bS and 3R,11bR enantiomers.

27. The method of claim 4 wherein the therapeutic agent is a compound of structural Formula IV 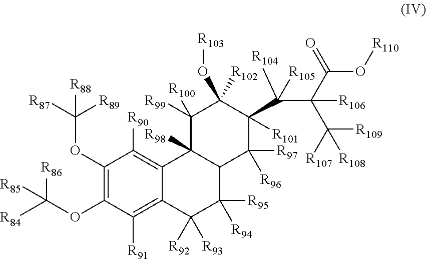
or a salt, stereoisomer, or racemic mixture thereof, wherein:
R₈₄-R₁₁₀ are independently selected from the group consisting of hydrogen and deuterium; and
at least one of R₈₄-R₁₁₀ is deuterium.

28. The method of claim 27 wherein at least one of R₈₄-R₁₁₀ independently has deuterium enrichment of no less than about 10%.

29. The method of claim 27 wherein at least one of R₈₄-R₁₁₀ independently has deuterium enrichment of no less than about 50%.

30. The method of claim 27 wherein at least one of R₈₄-R₁₁₀ independently has deuterium enrichment of no less than about 90%.

31. The method of claim 27 wherein at least one of R₈₄-R₁₁₀ independently has deuterium enrichment of no less than about 98%.

32. The method of claim 27 wherein the compound has the structural formula: 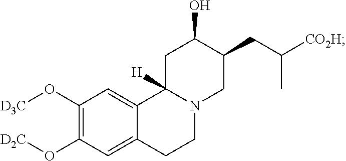
or a diastereomer, or mixture of diastereomers thereof.

33. The method of claim any one of claims 6, 12, 21, and 27 wherein each position represented as D has deuterium enrichment of no less than about 10%.

34. The method of claim 33 wherein each position represented as D has deuterium enrichment of no less than about 50%.

35. The method of claim any one of claims 11, 17, 20, 26, and 32 wherein each position represented as D has deuterium enrichment of no less than about 90%.

36. The method of claim 35 wherein each position represented as D has deuterium enrichment of no less than about 98%.

37. The method of claim 4 wherein treating the subject comprises administration of an additional therapeutic agent.

38. The method as recited in claim 37 wherein said additional therapeutic agent is selected from the group consisting of dopamine precursors, DOPA decarboxylase inhibitors, catechol-O-methyl transferase (COMT) inhibitors, dopamine receptor agonists, neuroprotective agents, NMDA antagonists, and anti-psychotics.

39. The method as recited in claim 38 wherein said dopamine precursor is levodopa.

40. The method as recited in claim 38 wherein said dopamine precursor is deuterated L-DOPA.

41. The method as recited in claim 40 wherein said deuterated L-DOPA has the structural formula:

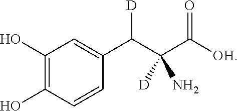 [SMILES: [2H]C(c1ccc(O)c(O)c1)[C@]([2H])(N)C(=O)O]

42. The method as recited in claim 40 wherein said deuterated L-DOPA has the structural formula:

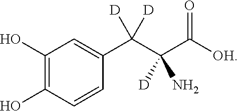 [SMILES: [2H]C([2H])(c1ccc(O)c(O)c1)[C@]([2H])(N)C(=O)O]

43. The method as recited in claim 40 wherein said deuterated L-DOPA comprises a composition of compounds of structural formula V 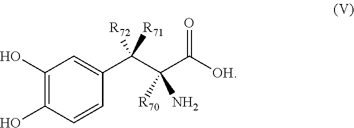
or a salt thereof, wherein:
in each compound of Formula V, R₇₀-R₇₂ are independently selected from the group consisting of hydrogen and deuterium;
the composition has deuterium enrichment of at least 10% at each of the positions R₇₀-R₇₂ in the compounds of Formula I;
the deuterium enrichment at the positions R₇₁ and R₇₂ is different from each other by at least 5%.

44. The composition as recited in claim 43 wherein R₇₀ has deuterium enrichment of no less than 90%.

45. The composition as recited in claim 44 wherein R₇₀ has deuterium enrichment of no less than 98%.

46. The composition as recited in claim 43 wherein R₇₂ has deuterium enrichment of no less than 90%.

47. The composition as recited in claim 45 wherein R₇₂ has deuterium enrichment of no less than 98%.

48. The composition as recited in claim 47 wherein R₇₁ has deuterium enrichment of between about 78% and about 95%.

49. The composition as recited in claim 47 wherein R₇₁ has deuterium enrichment of between about 78% and about 82%.

50. The composition as recited in claim 47 wherein R₇₁ has deuterium enrichment of between about 88% and about 92%.

51. The method as recited in claim 38 wherein said DOPA decarboxylase inhibitor is carbidopa.

52. The method as recited in claim 38 wherein said catechol-O-methyl transferase (COMT) inhibitor is selected from the group consisting of entacapone and tolcapone.

53. The method as recited in claim 38 wherein said dopamine receptor agonist is selected from the group consisting of apomorphine, bromocriptine, ropinirole, and pramipexole.

54. The method as recited in claim 38 wherein said neuroprotective agent is selected from the group consisting of selegeline and riluzole.

55. The method as recited in claim 38 wherein said NMDA antagonist is amantidine.

56. The method as recited in claim 38 wherein said anti-psychotic is clozapine.
